SEQUENCE LISTING (744 sequences)

SEQ ID NO: 1 - RNA, 2753 nt, Homo sapiens
  source 1..2753: mol_type = mRNA
agagagcgcttgcgggcgccgggcggagctgctgcggatcaggacccgagccgattcccgatcccgacccagatcctaacccgcgcccccgccccgccgccgccgccatgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctggagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatccccttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtgagtcacttgaggaggcgagtctagcagattctttcagaggtgctaaagtttcccatctttgtgcagctacctccgcattgctgtgtagtgacccctgcctgtgacgtggaggatcccagcctctgagctgagttggttttatgaaaagctaggaagcaacctttcgcctgtgcagcggtccagcacttaactctaatacatcagcatgcgttaattcagctggttgggaaatgacaccaggaagcccagtgcagagggtcccttactgactgtttcgtggccctattaatggtcagactgttccagcatgaggttcttagaatgacaggtgtttggatgggtgggggccttgtgatggggggtaggctggcccatgtgtgatcttgtggggtggagggaagagaatagcatgatcccacttccccatgctgtgggaaggggtgcagttcgtccccaagaacgacactgcctgtcaggtggtctgcaaagatgataaccttgactactaaaaacgtctccatggcgggggtaacaagatgataatctacttaattttagaacacctttttcacctaactaaaataatgtttaaagagttttgtataaaaatgtaaggaagcgttgttacctgttgaattttgtattatgtgaatcagtgagatgttagtagaataagccttaaaaaaaaaaaaatcggttgggtgcagtggcacacggctgtaatcccagcactttgggaggccaaggttggcagatcacctgaggtcaggagttcaagaccagtctggccaacatagcaaaaccctgtctctactaaaaatacaaaaattatctgggcatggtggtgcatgcctgtaatcccagctattcggaaggctgaggcaggagaatcacttgaacccaggaggcggaggttgcggtgagctgagattgcaccatttcattccagcctgggcaacatgagtgaaagtctgactcaaaaaaaaaaaatttaaaaaacaaaataatctagtgtgcagggcattcacctcagccccccaggcaggagccaagcacagcaggagcttccgcctcctctccactggagcacacaacttgaacctggcttattttctgcagggaccagccccacatggtcagtgagtttctccccatgtgtggcgatgagagagtgtagaaataaagacacaagacaaagaga

SEQ ID NO: 2 - RNA, 2753 nt, Homo sapiens
  source 1..2753: mol_type = mRNA
tctctttgtcttgtgtctttatttctacactctctcatcgccacacatggggagaaactcactgaccatgtggggctggtccctgcagaaaataagccaggttcaagttgtgtgctccagtggagaggaggcggaagctcctgctgtgcttggctcctgcctggggggctgaggtgaatgccctgcacactagattattttgttttttaaattttttttttttgagtcagactttcactcatgttgcccaggctggaatgaaatggtgcaatctcagctcaccgcaacctccgcctcctgggttcaagtgattctcctgcctcagccttccgaatagctgggattacaggcatgcaccaccatgcccagataatttttgtatttttagtagagacagggttttgctatgttggccagactggtcttgaactcctgacctcaggtgatctgccaaccttggcctcccaaagtgctgggattacagccgtgtgccactgcacccaaccgatttttttttttttaaggcttattctactaacatctcactgattcacataatacaaaattcaacaggtaacaacgcttccttacatttttatacaaaactctttaaacattattttagttaggtgaaaaaggtgttctaaaattaagtagattatcatcttgttacccccgccatggagacgtttttagtagtcaaggttatcatctttgcagaccacctgacaggcagtgtcgttcttggggacgaactgcaccccttcccacagcatggggaagtgggatcatgctattctcttccctccaccccacaagatcacacatgggccagcctaccccccatcacaaggcccccacccatccaaacacctgtcattctaagaacctcatgctggaacagtctgaccattaatagggccacgaaacagtcagtaagggaccctctgcactgggcttcctggtgtcatttcccaaccagctgaattaacgcatgctgatgtattagagttaagtgctggaccgctgcacaggcgaaaggttgcttcctagcttttcataaaaccaactcagctcagaggctgggatcctccacgtcacaggcaggggtcactacacagcaatgcggaggtagctgcacaaagatgggaaactttagcacctctgaaagaatctgctagactcgcctcctcaagtgactcacagactcttctctagtgaaaaactgggaaaggtggagagcccctcagcaccagcaggtactttattgcccaagaagaagttcaggctggacctgaggatggaccgcggggtggcctctgctttggtctctgctggacagcccttgggggagcagggagtccagcagggccagtcctgctcaggtgtgcatggggaggcctgttggctgctcactggcatttgggacctggaggcggggagcagacacatcagcactcgagtgaacacctgtgaggtcacccactgcaaccacaggacatcgtcgggcatatctggaagccatgtcaccagtctctggacaatcgcaatggcagattccacaggcagggtacagggcagcattacataagacattatcctaatgggtagcaagttgcaaatcttgctcatgtatccacctttgtctttcatttcttcactcagtgctgtagcgagcctgggcgagagggtgtccaggatgctctcatcccagggcaggatgctgagacgcaggtggtctagcagctcatctccctccagcctcacagccaaggcagccgactccggggaagaatccagactcatgtttgcccagctgggcatggcgacctcaggatccatcccttctgaggatgacttcaggcctggctggggcctgttgcagatgcccttctcttccaagaacctgaatgcatccaaatatcctcgaaggcatatctctcccagcaccttgagatccggggggacaaaagctctcgagagaaggtagaggttccctgtgcagaggcgtagactgagcttggtgatgtccacatgaagaaagttcgtggacttgactttagggcagatgtcgtactccccatagaagggggacacggtgatggttgttttggcatcaatgaagggtacgttgtcactcactcctccatccacatatcgcacgcctctgaaggaaggagggataaggccactgtagaaggggatgaagcaggaacataccaaggcatccacgacttcgtctttggaccgaaagtcagacaccagaacgttttccccatcagacactctggtaagagagatgcctattttgccggagatgagctggtggacattggccgggaggcatttgcagagaccctgtcggaggaacttgcttaagttgaaggatggatggaagatgccaatgttccgactcctggccttccgcacaagatctgagaggacctgcagagtctgctccagcgggataccggagaggacgccgacgcagtgcaacgccccggccgaagcgccgaacaacatgcgcgcgtcgcggaggaggtgcggggcgtgctcgctcaggcagcgggtcgccccgacgtggtagaagcccaggaagccgcagcccgcgaaggacaagctccagccgcgctctgcgtcgtacatggcggcggcggcggggcgggggcgcgggttaggatctgggtcgggatcgggaatcggctcgggtcctgatccgcagcagctccgcccggcgcccgcaagcgctctct

SEQ ID NO: 3 - RNA, 2222 nt, Homo sapiens
  source 1..2222: mol_type = mRNA
gtagagagcgcttgcgggcgccgggcggagctgctgcggatcaggacccgagccgattcccgatcccgacccagatcctaacccgcgcccccgccccgccgccgccgccatgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccgagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatccccttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtgagtcacttgaggaggcgagtctagcagattctttcagaggtgctaaagtttcccatctttgtgcagctacctccgcattgctgtgtagtgacccctgcctgtgacgtggaggatcccagcctctgagctgagttggttttatgaaaagctaggaagcaacctttcgcctgtgcagcggtccagcacttaactctaatacatcagcatgcgttaattcagctggttgggaaatgacaccaggaagcccagtgcagagggtcccttactgactgtttcgtggccctattaatggtcagactgttccagcatgaggttcttagaatgacaggtgtttggatgggtgggggccttgtgatggggggtaggctggcccatgtgtgatcttgtggggtggagggaagagaatagcatgatcccacttccccatgctgtgggaaggggtgcagttcgtccccaagaacgacactgcctgtcaggtggtctgcaaagatgataaccttgactactaaaaacgtctccatggcgggggtaacaagatgataatctacttaattttagaacacctttttcacctaactaaaataatgtttaaagagttttgtataaaaatgtaaggaagcgttgttacctgttgaattttgtattatgtgaatcagtgagatgttagtagaataagcctt

SEQ ID NO: 4 - RNA, 2222 nt, Homo sapiens
  source 1..2222: mol_type = mRNA
aaggcttattctactaacatctcactgattcacataatacaaaattcaacaggtaacaacgcttccttacatttttatacaaaactctttaaacattattttagttaggtgaaaaaggtgttctaaaattaagtagattatcatcttgttacccccgccatggagacgtttttagtagtcaaggttatcatctttgcagaccacctgacaggcagtgtcgttcttggggacgaactgcaccccttcccacagcatggggaagtgggatcatgctattctcttccctccaccccacaagatcacacatgggccagcctaccccccatcacaaggcccccacccatccaaacacctgtcattctaagaacctcatgctggaacagtctgaccattaatagggccacgaaacagtcagtaagggaccctctgcactgggcttcctggtgtcatttcccaaccagctgaattaacgcatgctgatgtattagagttaagtgctggaccgctgcacaggcgaaaggttgcttcctagcttttcataaaaccaactcagctcagaggctgggatcctccacgtcacaggcaggggtcactacacagcaatgcggaggtagctgcacaaagatgggaaactttagcacctctgaaagaatctgctagactcgcctcctcaagtgactcacagactcttctctagtgaaaaactgggaaaggtggagagcccctcagcaccagcaggtactttattgcccaagaagaagttcaggctggacctgaggatggaccgcggggtggcctctgctttggtctctgctggacagcccttgggggagcagggagtccagcagggccagtcctgctcaggtgtgcatggggaggcctgttggctgctcactggcatttgggacctggaggcggggagcagacacatcagcactcgagtgaacacctgtgaggtcacccactgcaaccacaggacatcgtcgggcatatctggaagccatgtcaccagtctctggacaatcgcaatggcagattccacaggcagggtacagggcagcattacataagacattatcctaatgggtagcaagttgcaaatcttgctcatgtatccacctttgtctttcatttcttcactcagtgctgtagcgagcctgggcgagagggtgtccaggatgctctcatcccagggcaggatgctgagacgcaggtggtctagcagctcatctccctccagcctcacagccaaggcagccgactccggggaagaatccagactcatgtttgcccagctgggcatggcgacctcaggatccatcccttctgaggatgacttcaggcctggctggggcctgttgcagatgcccttctcttccaagaacctgaatgcatccaaatatcctcgaaggcatatctctcccagcaccttgagatccggggggacaaaagctctcgagagaaggtagaggttccctgtgcagaggcgtagactgagcttggtgatgtccacatgaagaaagttcgtggacttgactttagggcagatgtcgtactccccatagaagggggacacggtgatggttgttttggcatcaatgaagggtacgttgtcactcactcctccatccacatatcgcacgcctctgaaggaaggagggataaggccactgtagaaggggatgaagcaggaacataccaaggcatccacgacttcgtctttggaccgaaagtcagacaccagaacgttttccccatcagacactctggtaagagagatgcctattttgccggagatgagctggtggacattggccgggaggcatttgcagagaccctgtcggaggaacttgcttaagttgaaggatggatggaagatgccaatgttccgactcctggccttccgcacaagatctgagaggacctgcagagtctgctcggagaggacgccgacgcagtgcaacgccccggccgaagcgccgaacaacatgcgcgcgtcgcggaggaggtgcggggcgtgctcgctcaggcagcgggtcgccccgacgtggtagaagcccaggaagccgcagcccgcgaaggacaagctccagccgcgctctgcgtcgtacatggcggcggcggcggggcgggggcgcgggttaggatctgggtcgggatcgggaatcggctcgggtcctgatccgcagcagctccgcccggcgcccgcaagcgctctctac

SEQ ID NO: 5 - RNA, 2294 nt, Macaca fascicularis
  source 1..2294: mol_type = mRNA
cgagggaggcggggcggacgtcgcgcgtggaaagcccgggcggagacgcggcggctgggtcacgagcgcttgcgggcgcccggcggagctgctgcggatcaggacccgagccgatccccgatcccgactccgatccggatccgcgcccccgcccccgccccgccatgtacgacgccgagcgcggctggagcttgtccttcgcgggctgcggcttcctgggcttctaccacgtcggggcgacccggtgcctgagcgagcacgccccgcacctcctccgcgacgcgcgcatgttgttcggcgcctcggccggggcgttgcactgcgtcggcgtcctctccgggatcccgctggagcagactctgcaggtcctctcagatcttgtccggaaggccaggagtcggaacattggtatcttccatccatccttcaacataggcaagttcctccgacaggatctctacaaatacctcccggccaatgtccaccagctcatctctggcaaaatatgcgtctcactcaccagagtgtctgatggggaaaacgttctggtgtctgactttcagtccaaagacgaagtcgtggatgccttgatttgttcctgcttcatccctttctacagtggccttatccctccttccttcagaggcgtgcgatatgtggatggaggagcgagtgacaacgtacccttcattgatgccaagacaaccatcaccgtgtcgcccttctatggggagtacgacatctgccctaaagtcaagtccaccaactttcttcatgtggacatcaccaagctcagcctacgcctctgcacagggaacctctaccttctctcaagagcgtttgtccccccggatctcaaggtgctgggagagatatgccttcgaggatatttggacgcgttcaggttcttggaagagaagggcatctgcaacaagccccagcggggtctgaagtcatcctcagaagggatggattctgaggtcactgcgcccggctgggaaaacacaagtctggattcttccccggagccggctgccttggctatgaggctggatggagatgagctgctagaccacctgcgtctcagcatcctgccctgggatgagagcatcctggacaccctgtcgcccgagctcgctacagcagtgagtgaagcaatgaaagacaaaggtggatacatgagcaagatttgcaacttgctacccattaggataatatcttatgtgatgctgccctgtaccctgcctgtggagtctgccattgcgattgtccagagactggtgacatggcttccagatatgcccgacgatgtgcagtggctgcagtgggtgacctcacaggtcttcactcgagcgctgatgtgtctgcttcccgcctccaggtcccaaatgccagtgagcagcgaacaggcctccccatgcaaaccggagcaggactggcactgctggactccctgctcccccgaggactgtcctgcagaggccaaagcagaggctaccccacggtccatcctcaggtccagcctgaacttcttctggggcaataaagtacctgctggtgctgaggggctctccacctttcccagtttttcactggagaagaatttgtgagtcatttgaggaggcgagtctaggagattctttcagaggtgctaaagcttcccatctttgtgcagctacctccgcattgccgtgtagtgacccctgcctgtgacgtggaggatcccagcctctgagctgagttggttttatgaaaagctaggaagcaatgtttggtctgtgcagcagtccagcacttaagtctaatacgtcagcatgcgttagttcagctggttgggaaatgacaccgggaagcctagcgcagagggtcccttactgactatttcatggtcctattaatggtcagactgttccagtgtgaggttcttagaatgactagtgtttggatgggtgggggccttgtggtggggggtgggctggcctatgtgtgatcttgtggggtggaaggaagagagtagcacaatcccacctccccatgccgtgggaaggggtgcacttggttcccaagaaggacactgcctgtcaggtggcctgcaaatataataaccttgacaactaaaaacctctccatgggggtgggaggtaccaagataataaccgatttacattttagagcacctttttcacctaactaaaataatgtttaaagagttttatataaaaatgtaaggaagagttgttatctgttgaattttgtattatatgaatcagtgagatgttaatagaataagcctt

SEQ ID NO: 6 - RNA, 2294 nt, Macaca fascicularis
  source 1..2294: mol_type = mRNA
aaggcttattctattaacatctcactgattcatataatacaaaattcaacagataacaactcttccttacatttttatataaaactctttaaacattattttagttaggtgaaaaaggtgctctaaaatgtaaatcggttattatcttggtacctcccacccccatggagaggtttttagttgtcaaggttattatatttgcaggccacctgacaggcagtgtccttcttgggaaccaagtgcaccccttcccacggcatggggaggtgggattgtgctactctcttccttccaccccacaagatcacacataggccagcccaccccccaccacaaggcccccacccatccaaacactagtcattctaagaacctcacactggaacagtctgaccattaataggaccatgaaatagtcagtaagggaccctctgcgctaggcttcccggtgtcatttcccaaccagctgaactaacgcatgctgacgtattagacttaagtgctggactgctgcacagaccaaacattgcttcctagcttttcataaaaccaactcagctcagaggctgggatcctccacgtcacaggcaggggtcactacacggcaatgcggaggtagctgcacaaagatgggaagctttagcacctctgaaagaatctcctagactcgcctcctcaaatgactcacaaattcttctccagtgaaaaactgggaaaggtggagagcccctcagcaccagcaggtactttattgccccagaagaagttcaggctggacctgaggatggaccgtggggtagcctctgctttggcctctgcaggacagtcctcgggggagcagggagtccagcagtgccagtcctgctccggtttgcatggggaggcctgttcgctgctcactggcatttgggacctggaggcgggaagcagacacatcagcgctcgagtgaagacctgtgaggtcacccactgcagccactgcacatcgtcgggcatatctggaagccatgtcaccagtctctggacaatcgcaatggcagactccacaggcagggtacagggcagcatcacataagatattatcctaatgggtagcaagttgcaaatcttgctcatgtatccacctttgtctttcattgcttcactcactgctgtagcgagctcgggcgacagggtgtccaggatgctctcatcccagggcaggatgctgagacgcaggtggtctagcagctcatctccatccagcctcatagccaaggcagccggctccggggaagaatccagacttgtgttttcccagccgggcgcagtgacctcagaatccatcccttctgaggatgacttcagaccccgctggggcttgttgcagatgcccttctcttccaagaacctgaacgcgtccaaatatcctcgaaggcatatctctcccagcaccttgagatccggggggacaaacgctcttgagagaaggtagaggttccctgtgcagaggcgtaggctgagcttggtgatgtccacatgaagaaagttggtggacttgactttagggcagatgtcgtactccccatagaagggcgacacggtgatggttgtcttggcatcaatgaagggtacgttgtcactcgctcctccatccacatatcgcacgcctctgaaggaaggagggataaggccactgtagaaagggatgaagcaggaacaaatcaaggcatccacgacttcgtctttggactgaaagtcagacaccagaacgttttccccatcagacactctggtgagtgagacgcatattttgccagagatgagctggtggacattggccgggaggtatttgtagagatcctgtcggaggaacttgcctatgttgaaggatggatggaagataccaatgttccgactcctggccttccggacaagatctgagaggacctgcagagtctgctccagcgggatcccggagaggacgccgacgcagtgcaacgccccggccgaggcgccgaacaacatgcgcgcgtcgcggaggaggtgcggggcgtgctcgctcaggcaccgggtcgccccgacgtggtagaagcccaggaagccgcagcccgcgaaggacaagctccagccgcgctcggcgtcgtacatggcggggcgggggcgggggcgcggatccggatcggagtcgggatcggggatcggctcgggtcctgatccgcagcagctccgccgggcgcccgcaagcgctcgtgacccagccgccgcgtctccgcccgggctttccacgcgcgacgtccgccccgcctccctcg

SEQ ID NO: 7 - RNA, 2291 nt, Macaca fascicularis
  source 1..2291: mol_type = mRNA
cgagggaggcggggcggacgtcgcgcgtggaaagcccgggcggagacgcggcggctgggtcacgagcgcttgcgggcgcccggcggagctgctgcggatcaggacccgagccgatccccgatcccgactccgatccggatccgcgcccccgcccccgccccgccatgtacgacgccgagcgcggctggagcttgtccttcgcgggctgcggcttcctgggcttctaccacgtcggggcgacccggtgcctgagcgagcacgccccgcacctcctccgcgacgcgcgcatgttgttcggcgcctcggccggggcgttgcactgcgtcggcgtcctctccgggatcccgctggagcagactctgcaggtcctctcagatcttgtccggaaggccaggagtcggaacattggtatcttccatccatccttcaacataggcaagttcctccgacaggatctctacaaatacctcccggccaatgtccaccagctcatctctggcaaaatatgcgtctcactcaccagagtgtctgatggggaaaacgttctggtgtctgactttcagtccaaagacgaagtcgtggatgccttgatttgttcctgcttcatccctttctacagtggccttatccctccttccttcagaggcgtgcgatatgtggatggaggagcgagtgacaacgtacccttcattgatgccaagacaaccatcaccgtgtcgcccttctatggggagtacgacatctgccctaaagtcaagtccaccaactttcttcatgtggacatcaccaagctcagcctacgcctctgcacagggaacctctaccttctctcaagagcgtttgtccccccggatctcaaggtgctgggagagatatgccttcgaggatatttggacgcgttcaggttcttggaagagaagggcatctgcaacaagccccagcggggtctgaagtcatcctcagaagggatggattctgaggtcactgcgcccggctgggaaaacacaagtctggattcttccccggagccggctgccttggctatgaggctggatggagatgagctgctagaccacctgcgtctcagcatcctgccctgggatgagagcatcctggacaccctgtcgcccgagctcgctacagtgagtgaagcaatgaaagacaaaggtggatacatgagcaagatttgcaacttgctacccattaggataatatcttatgtgatgctgccctgtaccctgcctgtggagtctgccattgcgattgtccagagactggtgacatggcttccagatatgcccgacgatgtgcagtggctgcagtgggtgacctcacaggtcttcactcgagcgctgatgtgtctgcttcccgcctccaggtcccaaatgccagtgagcagcgaacaggcctccccatgcaaaccggagcaggactggcactgctggactccctgctcccccgaggactgtcctgcagaggccaaagcagaggctaccccacggtccatcctcaggtccagcctgaacttcttctggggcaataaagtacctgctggtgctgaggggctctccacctttcccagtttttcactggagaagaatttgtgagtcatttgaggaggcgagtctaggagattctttcagaggtgctaaagcttcccatctttgtgcagctacctccgcattgccgtgtagtgacccctgcctgtgacgtggaggatcccagcctctgagctgagttggttttatgaaaagctaggaagcaatgtttggtctgtgcagcagtccagcacttaagtctaatacgtcagcatgcgttagttcagctggttgggaaatgacaccgggaagcctagcgcagagggtcccttactgactatttcatggtcctattaatggtcagactgttccagtgtgaggttcttagaatgactagtgtttggatgggtgggggccttgtggtggggggtgggctggcctatgtgtgatcttgtggggtggaaggaagagagtagcacaatcccacctccccatgccgtgggaaggggtgcacttggttcccaagaaggacactgcctgtcaggtggcctgcaaatataataaccttgacaactaaaaacctctccatgggggtgggaggtaccaagataataaccgatttacattttagagcacctttttcacctaactaaaataatgtttaaagagttttatataaaaatgtaaggaagagttgttatctgttgaattttgtattatatgaatcagtgagatgttaatagaataagcctt

SEQ ID NO: 8 - RNA, 2291 nt, Macaca fascicularis
  source 1..2291: mol_type = mRNA
aaggcttattctattaacatctcactgattcatataatacaaaattcaacagataacaactcttccttacatttttatataaaactctttaaacattattttagttaggtgaaaaaggtgctctaaaatgtaaatcggttattatcttggtacctcccacccccatggagaggtttttagttgtcaaggttattatatttgcaggccacctgacaggcagtgtccttcttgggaaccaagtgcaccccttcccacggcatggggaggtgggattgtgctactctcttccttccaccccacaagatcacacataggccagcccaccccccaccacaaggcccccacccatccaaacactagtcattctaagaacctcacactggaacagtctgaccattaataggaccatgaaatagtcagtaagggaccctctgcgctaggcttcccggtgtcatttcccaaccagctgaactaacgcatgctgacgtattagacttaagtgctggactgctgcacagaccaaacattgcttcctagcttttcataaaaccaactcagctcagaggctgggatcctccacgtcacaggcaggggtcactacacggcaatgcggaggtagctgcacaaagatgggaagctttagcacctctgaaagaatctcctagactcgcctcctcaaatgactcacaaattcttctccagtgaaaaactgggaaaggtggagagcccctcagcaccagcaggtactttattgccccagaagaagttcaggctggacctgaggatggaccgtggggtagcctctgctttggcctctgcaggacagtcctcgggggagcagggagtccagcagtgccagtcctgctccggtttgcatggggaggcctgttcgctgctcactggcatttgggacctggaggcgggaagcagacacatcagcgctcgagtgaagacctgtgaggtcacccactgcagccactgcacatcgtcgggcatatctggaagccatgtcaccagtctctggacaatcgcaatggcagactccacaggcagggtacagggcagcatcacataagatattatcctaatgggtagcaagttgcaaatcttgctcatgtatccacctttgtctttcattgcttcactcactgtagcgagctcgggcgacagggtgtccaggatgctctcatcccagggcaggatgctgagacgcaggtggtctagcagctcatctccatccagcctcatagccaaggcagccggctccggggaagaatccagacttgtgttttcccagccgggcgcagtgacctcagaatccatcccttctgaggatgacttcagaccccgctggggcttgttgcagatgcccttctcttccaagaacctgaacgcgtccaaatatcctcgaaggcatatctctcccagcaccttgagatccggggggacaaacgctcttgagagaaggtagaggttccctgtgcagaggcgtaggctgagcttggtgatgtccacatgaagaaagttggtggacttgactttagggcagatgtcgtactccccatagaagggcgacacggtgatggttgtcttggcatcaatgaagggtacgttgtcactcgctcctccatccacatatcgcacgcctctgaaggaaggagggataaggccactgtagaaagggatgaagcaggaacaaatcaaggcatccacgacttcgtctttggactgaaagtcagacaccagaacgttttccccatcagacactctggtgagtgagacgcatattttgccagagatgagctggtggacattggccgggaggtatttgtagagatcctgtcggaggaacttgcctatgttgaaggatggatggaagataccaatgttccgactcctggccttccggacaagatctgagaggacctgcagagtctgctccagcgggatcccggagaggacgccgacgcagtgcaacgccccggccgaggcgccgaacaacatgcgcgcgtcgcggaggaggtgcggggcgtgctcgctcaggcaccgggtcgccccgacgtggtagaagcccaggaagccgcagcccgcgaaggacaagctccagccgcgctcggcgtcgtacatggcggggcgggggcgggggcgcggatccggatcggagtcgggatcggggatcggctcgggtcctgatccgcagcagctccgccgggcgcccgcaagcgctcgtgacccagccgccgcgtctccgcccgggctttccacgcgcgacgtccgccccgcctccctcg

SEQ ID NO: 9 - RNA, 1722 nt, Macaca fascicularis
  source 1..1722: mol_type = mRNA
ccccgtccccgccccatccccctcccagaccccgtccccgccccatccccctccaagcctcgcccgggagggcgtcctgcccgctcattgggcctcgctccggcttatttgcatggtccgagggaggcggggcggacgtcgcgcgtggaaagcccgggcggagacgcggcggctgggtcacgagcgcttgcgggcgcccggcggagctgctgcggatcaggacccgagccgatccccgatccccgatcccgactccgatccggatccgcgcccccgcccccgccccgccatgtacgacgccgagcgcggctggagcttgtccttcgcgggctgcggcttcctgggcttctaccacgtcggggcgacccgctgcctgagcgagcacgcccgcacctcctccgcgacgcgcgcatgttgttcggcgcctcggccggggcgttgcactgcgtcggcgtcctctccgggatcccgctggagcagactctgcaggtcctctcagatcttgtccagaaggccaggagtcggaacattggtatcttccatccatccttcaacataggcaagttcctccgacgggatctctacaaatacctcccggccaatgtccaccagctcatctctggcaaaatatgcgtctcactcaccagagtgtctgatggggaaaacgttctggtgtctgactttcagtccaaagacgaagtcgtggatgccttgatatgttcctgcttcatccctttctacagtggccttatccctccttccttcagaggcgtgcgatatgtggatggaggagcgagtgacaacgtacccttcattgatgccaagacaaccatcaccgtgtcgcccttctatggggagtacgacatctgccctaaagtcaagtccaccaactttcttcatgtggacatcaccaagctcagcctacgcctctgcacagggaacctctaccttctctcaagagcgtttgtccccccggatctcaaggtgctgggagagatatgccttcgaggatatttggacgcgttcaggttcttggaagagaagggcatctgcaacaagccccagcggggtctgaagtcatcctcagaagggatggattctgaggtcactgcgcccggctgggaaaacacaagtctggattcttccccggaaccggctgccttggctatgaggctggatggagatgagctgctagaccacctgcgtctcagcatcctgccctgggatgagagcatcctggacaccctgtcgcccgagctcgctacagcagtgagtgaagcaatgaaagacaaaggtggatacatgagcaagatttgcaacttgctacccattaggataatgtcttatgtgatgctgccctgtaccctgcctgtggagtctgccattgcgattgtccagagactggtgacatggcttccggatatgcccgacgatgtgcagtggctgcagtgggtgacctcacaggtcttcactcgagcgctgatgtgtctgcttcccgcctccaggtcccaaatgccagtgagcagcgaacaggcctccccatgcaaaccggagcaggactggcactgctggactccctgctcccccgaggactgtcctgcagaggctaccccacggtccatcctcaggtccagcctgaacttcttctggggcaataaagtacctgctggtgctgaggggctctccacctttcccagtttttcactggagaagaatttgtga

SEQ ID NO: 10 - RNA, 1722 nt, Macaca fascicularis
  source 1..1722: mol_type = mRNA
tcacaaattcttctccagtgaaaaactgggaaaggtggagagcccctcagcaccagcaggtactttattgccccagaagaagttcaggctggacctgaggatggaccgtggggtagcctctgcaggacagtcctcgggggagcagggagtccagcagtgccagtcctgctccggtttgcatggggaggcctgttcgctgctcactggcatttgggacctggaggcgggaagcagacacatcagcgctcgagtgaagacctgtgaggtcacccactgcagccactgcacatcgtcgggcatatccggaagccatgtcaccagtctctggacaatcgcaatggcagactccacaggcagggtacagggcagcatcacataagacattatcctaatgggtagcaagttgcaaatcttgctcatgtatccacctttgtctttcattgcttcactcactgctgtagcgagctcgggcgacagggtgtccaggatgctctcatcccagggcaggatgctgagacgcaggtggtctagcagctcatctccatccagcctcatagccaaggcagccggttccggggaagaatccagacttgtgttttcccagccgggcgcagtgacctcagaatccatcccttctgaggatgacttcagaccccgctggggcttgttgcagatgcccttctcttccaagaacctgaacgcgtccaaatatcctcgaaggcatatctctcccagcaccttgagatccggggggacaaacgctcttgagagaaggtagaggttccctgtgcagaggcgtaggctgagcttggtgatgtccacatgaagaaagttggtggacttgactttagggcagatgtcgtactccccatagaagggcgacacggtgatggttgtcttggcatcaatgaagggtacgttgtcactcgctcctccatccacatatcgcacgcctctgaaggaaggagggataaggccactgtagaaagggatgaagcaggaacatatcaaggcatccacgacttcgtctttggactgaaagtcagacaccagaacgttttccccatcagacactctggtgagtgagacgcatattttgccagagatgagctggtggacattggccgggaggtatttgtagagatcccgtcggaggaacttgcctatgttgaaggatggatggaagataccaatgttccgactcctggccttctggacaagatctgagaggacctgcagagtctgctccagcgggatcccggagaggacgccgacgcagtgcaacgccccggccgaggcgccgaacaacatgcgcgcgtcgcggaggaggtgcgggcgtgctcgctcaggcagcgggtcgccccgacgtggtagaagcccaggaagccgcagcccgcgaaggacaagctccagccgcgctcggcgtcgtacatggcggggcgggggcgggggcgcggatccggatcggagtcgggatcggggatcggggatcggctcgggtcctgatccgcagcagctccgccgggcgcccgcaagcgctcgtgacccagccgccgcgtctccgcccgggctttccacgcgcgacgtccgccccgcctccctcggaccatgcaaataagccggagcgaggcccaatgagcgggcaggacgccctcccgggcgaggcttggagggggatggggcggggacggggtctgggagggggatggggcggggacgggg

SEQ ID NO: 11 - RNA, 2291 nt, Macaca mulatta
  source 1..2291: mol_type = mRNA
cgagggaggcggggcggacgtcgcgcgtggaaagcccgggcggagacgcggcggctgggtcacgagcgcttgcgggcgcccggcggagctgctgcggatcaggacccgagccgatccccgatcccgactccgatccggatccgcgcccccgcccccgccccgccatgtacgacgccgagcgcggctggagcttgtccttcgcgggctgcggcttcctgggcttctaccacgtcggggcgacccgctgcctgagcgagcacgccccgcacctcctccgcgacgcgcgcatgttgttcggcgcctcggccggggcgttgcactgcgtcggcgtcctctccgggatcccgctggagcagactctgcaggtcctctcagatcttgtccggaaggccaggagtcggaacattggtatcttccatccatccttcaacataggcaagttcctccgacaggatctctacaaatacctcccggccaatgtccaccagctcatctctggcaaaatatgcgtctcactcaccagagtgtctgatggggaaaacgttctggtgtctgactttcagtccaaagacgaagtcgtggatgccttgatttgttcctgcttcatccctttctacagtggccttatccctccttccttcagaggcgtgcgatatgtggatggaggagcgagtgacaacgtacccttcattgatgccaagacaaccatcaccgtgtcgcccttctatggggagtacgacatctgccctaaagtcaagtccaccaactttcttcatgtggacatcaccaagctcagcctacgcctctgcacagggaacctctaccttctctcaagagcgtttgtccccccggatctcaaggtgctgggagagatatgccttcgaggatatttggacgcgttcaggttcttggaagagaagggcatctgcaacaagccccagcggggtctgaagtcatcctcagaagggatggattctgaggtcactgcgcccggctgggaaaacacaagtctggattcttccccggagccggctgccttggctatgaggctggatggagatgagctgctagaccacctgcgtctcagcatcctgccctgggatgagagcatcctggacaccctgtcgcccgagctcgctacagcagtgagtgaagcaatgaaagacaaaggtggatacatgagcaagatttgcaacttgctacccattaggataatgtcttatgtgatgctgccctgtaccctgcctgtggagtctgccattgcgattgtccagagactggtgacatggcttccggatatgcccgacgatgtgcagtggctgcagtgggtgacctcacaggtcttcactcgagcgctgatgtgtctgcttcccgcctccaggtcccaaatgccagtgagcagcgaacaggcctccccatgcaaaccggagcaggactggcactgctggactccctgctcccccgaggactgtcctgcagaggccaaagcagaggctaccccacggtccatcctcaggtccagcctgaacttcttctggggcaataaagtacctgctggtgctgaggggctctccacctttcccagtttttcactggagaagaatttgtgagtcatttgaggaggcgagtctaggagattctttcagaggtgctaaagcttcccatctttgtgcagctacctccgcgttgccgtgtagtgacctctgcctgtgacgtggaggatcccagcctctgagctgagttggttttatgaaaagctaggaagcaatgtttggtctgtgcagcagtccagcacttaagtctaatacgtcagcatgcgttagttcagctggttgggaaatgacaccgggaagcctagcgcagagggtcccttactgactatttcatggtcctattaatggtcagactgttccagtgtgaggttcttagaatgactagtgtttggatgggtgggggccttgtggtggggggtgggctggcctatgtgtgatcttgtggggtggaaggaagagagtagcacaatcccacctccccatgccgtgggaaggggtgcacttggttcccaagaaggacactgcctgtcaggtggcctgcaaatataataaccttaacaactaaaaacctctccatgggggtggggggtaccaagataataaccgatttacattttagagcacctttttcacctaactaaaataatgtttaaagagttttatataaaaatgtaaggaattgttatctgttgaattttgtattatatgaatcagtgagatgttaatagaataagcctt

SEQ ID NO: 12 - RNA, 2291 nt, Macaca mulatta
  source 1..2291: mol_type = mRNA
aaggcttattctattaacatctcactgattcatataatacaaaattcaacagataacaattccttacatttttatataaaactctttaaacattattttagttaggtgaaaaaggtgctctaaaatgtaaatcggttattatcttggtaccccccacccccatggagaggtttttagttgttaaggttattatatttgcaggccacctgacaggcagtgtccttcttgggaaccaagtgcaccccttcccacggcatggggaggtgggattgtgctactctcttccttccaccccacaagatcacacataggccagcccaccccccaccacaaggcccccacccatccaaacactagtcattctaagaacctcacactggaacagtctgaccattaataggaccatgaaatagtcagtaagggaccctctgcgctaggcttcccggtgtcatttcccaaccagctgaactaacgcatgctgacgtattagacttaagtgctggactgctgcacagaccaaacattgcttcctagcttttcataaaaccaactcagctcagaggctgggatcctccacgtcacaggcagaggtcactacacggcaacgcggaggtagctgcacaaagatgggaagctttagcacctctgaaagaatctcctagactcgcctcctcaaatgactcacaaattcttctccagtgaaaaactgggaaaggtggagagcccctcagcaccagcaggtactttattgccccagaagaagttcaggctggacctgaggatggaccgtggggtagcctctgctttggcctctgcaggacagtcctcgggggagcagggagtccagcagtgccagtcctgctccggtttgcatggggaggcctgttcgctgctcactggcatttgggacctggaggcgggaagcagacacatcagcgctcgagtgaagacctgtgaggtcacccactgcagccactgcacatcgtcgggcatatccggaagccatgtcaccagtctctggacaatcgcaatggcagactccacaggcagggtacagggcagcatcacataagacattatcctaatgggtagcaagttgcaaatcttgctcatgtatccacctttgtctttcattgcttcactcactgctgtagcgagctcgggcgacagggtgtccaggatgctctcatcccagggcaggatgctgagacgcaggtggtctagcagctcatctccatccagcctcatagccaaggcagccggctccggggaagaatccagacttgtgttttcccagccgggcgcagtgacctcagaatccatcccttctgaggatgacttcagaccccgctggggcttgttgcagatgcccttctcttccaagaacctgaacgcgtccaaatatcctcgaaggcatatctctcccagcaccttgagatccggggggacaaacgctcttgagagaaggtagaggttccctgtgcagaggcgtaggctgagcttggtgatgtccacatgaagaaagttggtggacttgactttagggcagatgtcgtactccccatagaagggcgacacggtgatggttgtcttggcatcaatgaagggtacgttgtcactcgctcctccatccacatatcgcacgcctctgaaggaaggagggataaggccactgtagaaagggatgaagcaggaacaaatcaaggcatccacgacttcgtctttggactgaaagtcagacaccagaacgttttccccatcagacactctggtgagtgagacgcatattttgccagagatgagctggtggacattggccgggaggtatttgtagagatcctgtcggaggaacttgcctatgttgaaggatggatggaagataccaatgttccgactcctggccttccggacaagatctgagaggacctgcagagtctgctccagcgggatcccggagaggacgccgacgcagtgcaacgccccggccgaggcgccgaacaacatgcgcgcgtcgcggaggaggtgcggggcgtgctcgctcaggcagcgggtcgccccgacgtggtagaagcccaggaagccgcagcccgcgaaggacaagctccagccgcgctcggcgtcgtacatggcggggcgggggcgggggcgcggatccggatcggagtcgggatcggggatcggctcgggtcctgatccgcagcagctccgccgggcgcccgcaagcgctcgtgacccagccgccgcgtctccgcccgggctttccacgcgcgacgtccgccccgcctccctcg

SEQ ID NO: 13 - RNA, 2288 nt, Macaca mulatta
  source 1..2288: mol_type = mRNA
cgagggaggcggggcggacgtcgcgcgtggaaagcccgggcggagacgcggcggctgggtcacgagcgcttgcgggcgcccggcggagctgctgcggatcaggacccgagccgatccccgatcccgactccgatccggatccgcgcccccgcccccgccccgccatgtacgacgccgagcgcggctggagcttgtccttcgcgggctgcggcttcctgggcttctaccacgtcggggcgacccgctgcctgagcgagcacgccccgcacctcctccgcgacgcgcgcatgttgttcggcgcctcggccggggcgttgcactgcgtcggcgtcctctccgggatcccgctggagcagactctgcaggtcctctcagatcttgtccggaaggccaggagtcggaacattggtatcttccatccatccttcaacataggcaagttcctccgacaggatctctacaaatacctcccggccaatgtccaccagctcatctctggcaaaatatgcgtctcactcaccagagtgtctgatggggaaaacgttctggtgtctgactttcagtccaaagacgaagtcgtggatgccttgatttgttcctgcttcatccctttctacagtggccttatccctccttccttcagaggcgtgcgatatgtggatggaggagcgagtgacaacgtacccttcattgatgccaagacaaccatcaccgtgtcgcccttctatggggagtacgacatctgccctaaagtcaagtccaccaactttcttcatgtggacatcaccaagctcagcctacgcctctgcacagggaacctctaccttctctcaagagcgtttgtccccccggatctcaaggtgctgggagagatatgccttcgaggatatttggacgcgttcaggttcttggaagagaagggcatctgcaacaagccccagcggggtctgaagtcatcctcagaagggatggattctgaggtcactgcgcccggctgggaaaacacaagtctggattcttccccggagccggctgccttggctatgaggctggatggagatgagctgctagaccacctgcgtctcagcatcctgccctgggatgagagcatcctggacaccctgtcgcccgagctcgctacagtgagtgaagcaatgaaagacaaaggtggatacatgagcaagatttgcaacttgctacccattaggataatgtcttatgtgatgctgccctgtaccctgcctgtggagtctgccattgcgattgtccagagactggtgacatggcttccggatatgcccgacgatgtgcagtggctgcagtgggtgacctcacaggtcttcactcgagcgctgatgtgtctgcttcccgcctccaggtcccaaatgccagtgagcagcgaacaggcctccccatgcaaaccggagcaggactggcactgctggactccctgctcccccgaggactgtcctgcagaggccaaagcagaggctaccccacggtccatcctcaggtccagcctgaacttcttctggggcaataaagtacctgctggtgctgaggggctctccacctttcccagtttttcactggagaagaatttgtgagtcatttgaggaggcgagtctaggagattctttcagaggtgctaaagcttcccatctttgtgcagctacctccgcgttgccgtgtagtgacctctgcctgtgacgtggaggatcccagcctctgagctgagttggttttatgaaaagctaggaagcaatgtttggtctgtgcagcagtccagcacttaagtctaatacgtcagcatgcgttagttcagctggttgggaaatgacaccgggaagcctagcgcagagggtcccttactgactatttcatggtcctattaatggtcagactgttccagtgtgaggttcttagaatgactagtgtttggatgggtgggggccttgtggtggggggtgggctggcctatgtgtgatcttgtggggtggaaggaagagagtagcacaatcccacctccccatgccgtgggaaggggtgcacttggttcccaagaaggacactgcctgtcaggtggcctgcaaatataataaccttaacaactaaaaacctctccatgggggtggggggtaccaagataataaccgatttacattttagagcacctttttcacctaactaaaataatgtttaaagagttttatataaaaatgtaaggaattgttatctgttgaattttgtattatatgaatcagtgagatgttaatagaataagcctt

SEQ ID NO: 14 - RNA, 2288 nt, Macaca mulatta
  source 1..2288: mol_type = mRNA
aaggcttattctattaacatctcactgattcatataatacaaaattcaacagataacaattccttacatttttatataaaactctttaaacattattttagttaggtgaaaaaggtgctctaaaatgtaaatcggttattatcttggtaccccccacccccatggagaggtttttagttgttaaggttattatatttgcaggccacctgacaggcagtgtccttcttgggaaccaagtgcaccccttcccacggcatggggaggtgggattgtgctactctcttccttccaccccacaagatcacacataggccagcccaccccccaccacaaggcccccacccatccaaacactagtcattctaagaacctcacactggaacagtctgaccattaataggaccatgaaatagtcagtaagggaccctctgcgctaggcttcccggtgtcatttcccaaccagctgaactaacgcatgctgacgtattagacttaagtgctggactgctgcacagaccaaacattgcttcctagcttttcataaaaccaactcagctcagaggctgggatcctccacgtcacaggcagaggtcactacacggcaacgcggaggtagctgcacaaagatgggaagctttagcacctctgaaagaatctcctagactcgcctcctcaaatgactcacaaattcttctccagtgaaaaactgggaaaggtggagagcccctcagcaccagcaggtactttattgccccagaagaagttcaggctggacctgaggatggaccgtggggtagcctctgctttggcctctgcaggacagtcctcgggggagcagggagtccagcagtgccagtcctgctccggtttgcatggggaggcctgttcgctgctcactggcatttgggacctggaggcgggaagcagacacatcagcgctcgagtgaagacctgtgaggtcacccactgcagccactgcacatcgtcgggcatatccggaagccatgtcaccagtctctggacaatcgcaatggcagactccacaggcagggtacagggcagcatcacataagacattatcctaatgggtagcaagttgcaaatcttgctcatgtatccacctttgtctttcattgcttcactcactgtagcgagctcgggcgacagggtgtccaggatgctctcatcccagggcaggatgctgagacgcaggtggtctagcagctcatctccatccagcctcatagccaaggcagccggctccggggaagaatccagacttgtgttttcccagccgggcgcagtgacctcagaatccatcccttctgaggatgacttcagaccccgctggggcttgttgcagatgcccttctcttccaagaacctgaacgcgtccaaatatcctcgaaggcatatctctcccagcaccttgagatccggggggacaaacgctcttgagagaaggtagaggttccctgtgcagaggcgtaggctgagcttggtgatgtccacatgaagaaagttggtggacttgactttagggcagatgtcgtactccccatagaagggcgacacggtgatggttgtcttggcatcaatgaagggtacgttgtcactcgctcctccatccacatatcgcacgcctctgaaggaaggagggataaggccactgtagaaagggatgaagcaggaacaaatcaaggcatccacgacttcgtctttggactgaaagtcagacaccagaacgttttccccatcagacactctggtgagtgagacgcatattttgccagagatgagctggtggacattggccgggaggtatttgtagagatcctgtcggaggaacttgcctatgttgaaggatggatggaagataccaatgttccgactcctggccttccggacaagatctgagaggacctgcagagtctgctccagcgggatcccggagaggacgccgacgcagtgcaacgccccggccgaggcgccgaacaacatgcgcgcgtcgcggaggaggtgcggggcgtgctcgctcaggcagcgggtcgccccgacgtggtagaagcccaggaagccgcagcccgcgaaggacaagctccagccgcgctcggcgtcgtacatggcggggcgggggcgggggcgcggatccggatcggagtcgggatcggggatcggctcgggtcctgatccgcagcagctccgccgggcgcccgcaagcgctcgtgacccagccgccgcgtctccgcccgggctttccacgcgcgacgtccgccccgcctccctcg

SEQ ID NO: 15 - RNA, 1680 nt, Macaca mulatta
  source 1..1680: mol_type = mRNA
catccccctccaagcctcgcccgggagggcgtcctgcccgctcattgggcctcgctccggcttatttgcatggtccgagggaggcggggcggacgtcgcgcgtggaaagcccgggcggagacgcggcggctgggtcacgagcgcttgcgggcgcccggcggagctgctgcggatcaggacccgagccgatccccgatcccgactccgatccggatccgcgcccccgcccccgccccgccatgtacgacgccgagcgcggctggagcttgtccttcgcgggctgcggcttcctgggcttctaccacgtcggggcgacccgctgcctgagcgagcacgccccgcacctcctccgcgacgcgcgcatgttgttcggcgcctcggccggggcgttgcactgcgtcggcgtcctctccgggatcccgctggagcagactctgcaggtcctctcagatcttgtccggaaggccaggagtcggaacattggtatcttccatccatccttcaacataggcaagttcctccgacaggatctctacaaatacctcccggccaatgtccaccagctcatctctggcaaaatatgcgtctcactcaccagagtgtctgatggggaaaacgttctggtgtctgactttcagtccaaagacgaagtcgtggatgccttgatttgttcctgcttcatccctttctacagtggccttatccctccttccttcagaggcgtgcgatatgtggatggaggagcgagtgacaacgtacccttcattgatgccaagacaaccatcaccgtgtcgcccttctatggggagtacgacatctgccctaaagtcaagtccaccaactttcttcatgtggacatcaccaagctcagcctacgcctctgcacagggaacctctaccttctctcaagagcgtttgtccccccggatctcaaggtgctgggagagatatgccttcgaggatatttggacgcgttcaggttcttggaagagaagggcatctgcaacaagccccagcggggtctgaagtcatcctcagaagggatggattctgaggtcactgcgcccggctgggaaaacacaagtctggattcttccccggagccggctgccttggctatgaggctggatggagatgagctgctagaccacctgcgtctcagcatcctgccctgggatgagagcatcctggacaccctgtcgcccgagctcgctacagcagtgagtgaagcaatgaaagacaaaggtggatacatgagcaagatttgcaacttgctacccattaggataatgtcttatgtgatgctgccctgtaccctgcctgtggagtctgccattgcgattgtccagagactggtgacatggcttccggatatgcccgacgatgtgcagtggctgcagtgggtgacctcacaggtcttcactcgagcgctgatgtgtctgcttcccgcctccaggtcccaaatgccagtgagcagcgaacaggcctccccatgcaaaccggagcaggactggcactgctggactccctgctcccccgaggactgtcctgcagaggccaaagcagaggctaccccacggtccatcctcaggtccagcctgaacttcttctggggcaataaagtacctgctggtgctgaggggctctccacctttcccagtttttcactggagaagaatt

SEQ ID NO: 16 - RNA, 1680 nt, Macaca mulatta
  source 1..1680: mol_type = mRNA
aattcttctccagtgaaaaactgggaaaggtggagagcccctcagcaccagcaggtactttattgccccagaagaagttcaggctggacctgaggatggaccgtggggtagcctctgctttggcctctgcaggacagtcctcgggggagcagggagtccagcagtgccagtcctgctccggtttgcatggggaggcctgttcgctgctcactggcatttgggacctggaggcgggaagcagacacatcagcgctcgagtgaagacctgtgaggtcacccactgcagccactgcacatcgtcgggcatatccggaagccatgtcaccagtctctggacaatcgcaatggcagactccacaggcagggtacagggcagcatcacataagacattatcctaatgggtagcaagttgcaaatcttgctcatgtatccacctttgtctttcattgcttcactcactgctgtagcgagctcgggcgacagggtgtccaggatgctctcatcccagggcaggatgctgagacgcaggtggtctagcagctcatctccatccagcctcatagccaaggcagccggctccggggaagaatccagacttgtgttttcccagccgggcgcagtgacctcagaatccatcccttctgaggatgacttcagaccccgctggggcttgttgcagatgcccttctcttccaagaacctgaacgcgtccaaatatcctcgaaggcatatctctcccagcaccttgagatccggggggacaaacgctcttgagagaaggtagaggttccctgtgcagaggcgtaggctgagcttggtgatgtccacatgaagaaagttggtggacttgactttagggcagatgtcgtactccccatagaagggcgacacggtgatggttgtcttggcatcaatgaagggtacgttgtcactcgctcctccatccacatatcgcacgcctctgaaggaaggagggataaggccactgtagaaagggatgaagcaggaacaaatcaaggcatccacgacttcgtctttggactgaaagtcagacaccagaacgttttccccatcagacactctggtgagtgagacgcatattttgccagagatgagctggtggacattggccgggaggtatttgtagagatcctgtcggaggaacttgcctatgttgaaggatggatggaagataccaatgttccgactcctggccttccggacaagatctgagaggacctgcagagtctgctccagcgggatcccggagaggacgccgacgcagtgcaacgccccggccgaggcgccgaacaacatgcgcgcgtcgcggaggaggtgcggggcgtgctcgctcaggcagcgggtcgccccgacgtggtagaagcccaggaagccgcagcccgcgaaggacaagctccagccgcgctcggcgtcgtacatggcggggcgggggcgggggcgcggatccggatcggagtcgggatcggggatcggctcgggtcctgatccgcagcagctccgccgggcgcccgcaagcgctcgtgacccagccgccgcgtctccgcccgggctttccacgcgcgacgtccgccccgcctccctcggaccatgcaaataagccggagcgaggcccaatgagcgggcaggacgccctcccgggcgaggcttggagggggatg

SEQ ID NO: 17 - RNA, 4649 nt, Mus musculus
  source 1..4649: mol_type = mRNA
agagcagcaacaccgggagcagagctgaactgcagcgccgcccggagcttcaagcaccatgtatgacccagagcgccgctggagcctgtcgtttgcaggctgcggcttcctgggcttctaccacgtcggggctacgctatgtctgagcgagcgcgccccgcacctcctccgcgatgcgcgcactttctttggctgctcggccggtgcactgcacgcggtcaccttcgtgtgcagtctccctctcggccgtataatggagatcctcatggacctcgtgcggaaagccaggagccgcaacatcggcaccctccacccgttcttcaacattaacaagtgcatcagagacgggctccaggagagcctcccagacaatgtccaccaggtcatttctggcaaggttcacatctcactcaccagggtgtcggatggggagaacgtgctggtgtctgagttccattccaaagacgaagtcgtggatgccctggtgtgttcctgcttcattcccctcttctctggcctaatccctccttccttccgaggcgagcggtacgtggacggaggagtgagcgacaacgtccctgtgctggatgccaaaaccaccatcacggtgtcacctttctacggtgagcatgacatctgccccaaagtcaagtccaccaacttcttccacgtgaatatcaccaacctcagcctccgcctctgcactgggaacctccaacttctgaccagagcgctcttcccgtctgatgtgaaggtgatgggagagctgtgctatcaagggtacctggacgccttccggttcctggaggagaatggcatctgtaacgggccacagcgcagcctgagtctgtccttggtggcgccagaagcctgcttggaaaatggcaaacttgtgggagacaaggtgccagtcagcctatgctttacagatgagaacatctgggagacactgtcccccgagctcagcacagctctgagtgaagcgattaaggacagggagggctacctgagcaaagtctgcaacctcctgcccgtcaggatcctgtcctacatcatgctgccctgcagtctgcccgtggagtcggctatcgctgcagtccacaggctggtgacatggctccctgatatccaggatgatatccagtggctacaatgggcgacatcccaggtttgtgcccgaatgacgatgtgcctgctcccctctaccaggtaaatacttgggcccagggtgtgtgggccagataggcatccctcccggttgttcccagagctcttagggtcagagcttgggtggtgacagccttaacaagccaggctcagccgcctgtccccagcatgccattaaagaaaccggtagcagagaaagcaggtttattcgaatataaaaaggttcaagcccccacccggttaatctttaagataccaacaggaggcttaagtttaaacagagttacacataaacagtctgaatcagggcgtggtcctgcccaccattgtctgggcttcaaggttccttctttctctccctagcatgagattcctgggacaatcccaattccttggcctccattgtatcaaagggctgaaaaccaaagggaaggcacagctgtctcttcagcatgcctcttctgccagaaccactgcaaggtttggtgctcaggctgtgcaaacattctagcaatgtttgactcagtgtcaagcaggtgacaaggaacatggtgctgtgtggggggaacccatggcccaggtgagggcttattggtgggtgaagctgtgggtgttcaggtggtggagaaggccttaagggatgggactgacacctcagcactgaaggcaggaggaagctgtggctctgggttgcacccctgcctggctccaccctctctggcatctgtagaagttacagctggttcttcctctcagccccatgctcccagaaataagactcagacccaaattatagttacaaataccttggccatatagctaggctcttctcagactagctcataacttaactcattaattttaacctccatcctgccacatggctggtggcctgtgctcaggtaccatgagtccagctcttcacatctttccggatgaatcttccataattctttctgcctcctggatgttccaccttctattccaccttttcctataggccatggttttgtttttgttttttttttccaaatttaatttaattaattaatttatttatttttggtttttcgagacagggtttctctgtatcgccctggctgtcctggaactcactatgtaagccaggctggcctcaaactcagaaatccgcctgcctctgcctcctgagtgctgggattaaaggcgtgcgcaaccatgcccggtgtggtttttttttttttttaattgacaggtggatgcatctatataatccataacatattctctctacaggtatctattaggttttgggtgaggtgtggagttctagggaactctgagagaaattcctggggagtaagtggtttatcaagttgattggaggagtttttaatgctatggacagacagacagaaggacaacagcatagtcggggctaccagggagttcaggccccggcatcggagatagaagcaggatggggtctttgaagagattctgagcccacacagcagaggagggactctctctttagagcttttgaggatgagggaggttgactgcaagagcctacagccaggctcgaggcaggcagggggtggggagcaggatgtaaaccccttcgatgctgacagactcacttctggggtaaaatattatgagatgcctgtcagtgtctgtgaagagacctgagcagagtctggattctgacatcaatcatgttcttacaatactgaagacctgagagcctgcaatcttggtttgtaaattgctggtctccgtgcttccagtgaacttggacattcttctcatggttggtccaggagaggccaaagctgagggcaccctgccttccacccccagtccagcttgaccttttatctggagcaacagtgtctagatgatgggtgggtgaggggtgctatactgtctgtccctctgggaagggttctgttacttttggaggcagctaggaagtttctctgtgcagctgccccctggtgctgtgtggtgacctcattgcctgtgaccccaggatcacaggatctgggctaaagtggtagtccatagaaaccaaagacaatgatttggtgtttagaaagctactcttggtctgggtgaagtctggtgcttaagggctatcacaaagagcgtgtcaaaccatctctcagcctgtgagtcagtggggagcccaagggcatcagtgtttggaaactggaatccaaaccgggcaatctcggaaggaaactgtttaggaattgtgatgggacgggccgtggctgtctctgaaaagggcctgccagataacttattacttttaaggacacctttggctcttactaatttataaagcattttatataaacacaccagggagtgcatggtgaactacacgtatgatcagttaagtggggctagaattaggtagggagagcatcggacctctgcctcctcaacctcaacttgcttgctttctccactggctccaaatctttgtatagtcatcagccatgaccacctctctccctccccatctactaccagcagcgttaatgggaataagtacccacttctctcaggtgtactatacagctgtgggtgtggtgtgtgtttcctgtaattcacactttagaaaggaaacaagcaaacaaaagaaaccaggtgctgcccatactcctaagtgtagacagtgaaggtgtgtgtctcccatgcctgagtctcctggaggcctagtgagctccaggttcatgcaagcacatcaggaggaatcatataatctcagcacggttgatccagatgggataagaaaggactctgggagagagaatgtggttctagagacaaagtgtctaggctacacagaagataagactgtcccaaggaaagaaaagaaaccaggaactagggtgcagctcagttgtcagaggacttctctaggcttgaagcccagagtccaatctcagcaccttataaactgtggagtgacaggcagtgacatcggcctgtaatcccaacactcaagcagtagaggcaagaggatcataagttcaaggtcttccttggctatttagggagttggaggttagctctggctacatgagaccctgtctcaaaaaaaaaaaaaaaaaaaagtagaaacttctgccttgctttgagctgcccctttctggacgtttctcatcagtagagaatattcctgccaccctatcagacaaaactcccactggtttggagtctctccattctcaggaacacctcaggagtcagacagtgagcagcagggagcaatgtcttgacttgtaagccccttagcaaggctggttcatttgtttattaaaagcaggtgtgggtgaatttatgcaaatgagtatgcaaactagtggaacagcagaaggattgaatggatacaccaaaaataaccacaactgtttaagggaaaagggtccataataaatgtggggaacaaaaaacaaataaatgtgattttttttagaaaaatg

SEQ ID NO: 18 - RNA, 4649 nt, Mus musculus
  source 1..4649: mol_type = mRNA
catttttctaaaaaaaatcacatttatttgttttttgttccccacatttattatggacccttttcccttaaacagttgtggttatttttggtgtatccattcaatccttctgctgttccactagtttgcatactcatttgcataaattcacccacacctgcttttaataaacaaatgaaccagccttgctaaggggcttacaagtcaagacattgctccctgctgctcactgtctgactcctgaggtgttcctgagaatggagagactccaaaccagtgggagttttgtctgatagggtggcaggaatattctctactgatgagaaacgtccagaaaggggcagctcaaagcaaggcagaagtttctacttttttttttttttttttttgagacagggtctcatgtagccagagctaacctccaactccctaaatagccaaggaagaccttgaacttatgatcctcttgcctctactgcttgagtgttgggattacaggccgatgtcactgcctgtcactccacagtttataaggtgctgagattggactctgggcttcaagcctagagaagtcctctgacaactgagctgcaccctagttcctggtttcttttctttccttgggacagtcttatcttctgtgtagcctagacactttgtctctagaaccacattctctctcccagagtcctttcttatcccatctggatcaaccgtgctgagattatatgattcctcctgatgtgcttgcatgaacctggagctcactaggcctccaggagactcaggcatgggagacacacaccttcactgtctacacttaggagtatgggcagcacctggtttcttttgtttgcttgtttcctttctaaagtgtgaattacaggaaacacacaccacacccacagctgtatagtacacctgagagaagtgggtacttattcccattaacgctgctggtagtagatggggagggagagaggtggtcatggctgatgactatacaaagatttggagccagtggagaaagcaagcaagttgaggttgaggaggcagaggtccgatgctctccctacctaattctagccccacttaactgatcatacgtgtagttcaccatgcactccctggtgtgtttatataaaatgctttataaattagtaagagccaaaggtgtccttaaaagtaataagttatctggcaggcccttttcagagacagccacggcccgtcccatcacaattcctaaacagtttccttccgagattgcccggtttggattccagtttccaaacactgatgcccttgggctccccactgactcacaggctgagagatggtttgacacgctctttgtgatagcccttaagcaccagacttcacccagaccaagagtagctttctaaacaccaaatcattgtctttggtttctatggactaccactttagcccagatcctgtgatcctggggtcacaggcaatgaggtcaccacacagcaccagggggcagctgcacagagaaacttcctagctgcctccaaaagtaacagaacccttcccagagggacagacagtatagcacccctcacccacccatcatctagacactgttgctccagataaaaggtcaagctggactgggggtggaaggcagggtgccctcagctttggcctctcctggaccaaccatgagaagaatgtccaagttcactggaagcacggagaccagcaatttacaaaccaagattgcaggctctcaggtcttcagtattgtaagaacatgattgatgtcagaatccagactctgctcaggtctcttcacagacactgacaggcatctcataatattttaccccagaagtgagtctgtcagcatcgaaggggtttacatcctgctccccaccccctgcctgcctcgagcctggctgtaggctcttgcagtcaacctccctcatcctcaaaagctctaaagagagagtccctcctctgctgtgtgggctcagaatctcttcaaagaccccatcctgcttctatctccgatgccggggcctgaactccctggtagccccgactatgctgttgtccttctgtctgtctgtccatagcattaaaaactcctccaatcaacttgataaaccacttactccccaggaatttctctcagagttccctagaactccacacctcacccaaaacctaatagatacctgtagagagaatatgttatggattatatagatgcatccacctgtcaattaaaaaaaaaaaaaaaccacaccgggcatggttgcgcacgcctttaatcccagcactcaggaggcagaggcaggcggatttctgagtttgaggccagcctggcttacatagtgagttccaggacagccagggcgatacagagaaaccctgtctcgaaaaaccaaaaataaataaattaattaattaaattaaatttggaaaaaaaaaacaaaaacaaaaccatggcctataggaaaaggtggaatagaaggtggaacatccaggaggcagaaagaattatggaagattcatccggaaagatgtgaagagctggactcatggtacctgagcacaggccaccagccatgtggcaggatggaggttaaaattaatgagttaagttatgagctagtctgagaagagcctagctatatggccaaggtatttgtaactataatttgggtctgagtcttatttctgggagcatggggctgagaggaagaaccagctgtaacttctacagatgccagagagggtggagccaggcaggggtgcaacccagagccacagcttcctcctgccttcagtgctgaggtgtcagtcccatcccttaaggccttctccaccacctgaacacccacagcttcacccaccaataagccctcacctgggccatgggttccccccacacagcaccatgttccttgtcacctgcttgacactgagtcaaacattgctagaatgtttgcacagcctgagcaccaaaccttgcagtggttctggcagaagaggcatgctgaagagacagctgtgccttccctttggttttcagccctttgatacaatggaggccaaggaattgggattgtcccaggaatctcatgctagggagagaaagaaggaaccttgaagcccagacaatggtgggcaggaccacgccctgattcagactgtttatgtgtaactctgtttaaacttaagcctcctgttggtatcttaaagattaaccgggtgggggcttgaacctttttatattcgaataaacctgctttctctgctaccggtttctttaatggcatgctggggacaggcggctgagcctggcttgttaaggctgtcaccacccaagctctgaccctaagagctctgggaacaaccgggagggatgcctatctggcccacacaccctgggcccaagtatttacctggtagaggggagcaggcacatcgtcattcgggcacaaacctgggatgtcgcccattgtagccactggatatcatcctggatatcagggagccatgtcaccagcctgtggactgcagcgatagccgactccacgggcagactgcagggcagcatgatgtaggacaggatcctgacgggcaggaggttgcagactttgctcaggtagccctccctgtccttaatcgcttcactcagagctgtgctgagctcgggggacagtgtctcccagatgttctcatctgtaaagcataggctgactggcaccttgtctcccacaagtttgccattttccaagcaggcttctggcgccaccaaggacagactcaggctgcgctgtggcccgttacagatgccattctcctccaggaaccggaaggcgtccaggtacccttgatagcacagctctcccatcaccttcacatcagacgggaagagcgctctggtcagaagttggaggttcccagtgcagaggcggaggctgaggttggtgatattcacgtggaagaagttggtggacttgactttggggcagatgtcatgctcaccgtagaaaggtgacaccgtgatggtggttttggcatccagcacagggacgttgtcgctcactcctccgtccacgtaccgctcgcctcggaaggaaggagggattaggccagagaagaggggaatgaagcaggaacacaccagggcatccacgacttcgtctttggaatggaactcagacaccagcacgttctccccatccgacaccctggtgagtgagatgtgaaccttgccagaaatgacctggtggacattgtctgggaggctctcctggagcccgtctctgatgcacttgttaatgttgaagaacgggtggagggtgccgatgttgcggctcctggctttccgcacgaggtccatgaggatctccattatacggccgagagggagactgcacacgaaggtgaccgcgtgcagtgcaccggccgagcagccaaagaaagtgcgcgcatcgcggaggaggtgcggggcgcgctcgctcagacatagcgtagccccgacgtggtagaagcccaggaagccgcagcctgcaaacgacaggctccagcggcgctctgggtcatacatggtgcttgaagctccgggcggcgctgcagttcagctctgctcccggtgttgctgctct

SEQ ID NO: 19 - RNA, 1313 nt, Mus musculus
  source 1..1313: mol_type = mRNA
gagctgaactgcagcgccgcccggagcttcaagcaccatgtatgacccagagcgccgctggagcctgtcgtttgcaggctgcggcttcctgggcttctaccacgtcggggctacgctatgtctgagcgagcgcgccccgcacctcctccgcgatgcgcgcactttctttggctgctcggccggtgcactgcacgcggtcaccttcgtgtgcagtctccctctcggccgtataatggagatcctcatggacctcgtgcggaaagccaggagccgcaacatcggcaccctccacccgttcttcaacattaacaagtgcatcagagacgggctccaggagagcctcccagacaatgtccaccaggtcatttctggcaaggttcacatctcactcaccagggtgtcggatggggagaacgtgctggtgtctgagttccattccaaagacgaagtcgtggatgccctggtgtgttcctgcttcattcccctcttctctggcctaatccctccttccttccgaggcgagcggtacgtggacggaggagtgagcgacaacgtccctgtgctggatgccaaaaccaccatcacggtgtcacctttctacggtgagcatgacatctgccccaaagtcaagtccaccaacttcttccacgtgaatatcaccaacctcagcctccgcctctgcactgggaacctccaacttctgaccagagcgctcttcccgtctgatgtgaaggtgatgggagagctgtgctatcaagggtacctggacgccttccggttcctggaggagaatggcatctgtaacgggccacagcgcagcctgagtctgtccttggtggcgccagaagcctgcttggaaaatggcaaacttgtgggagacaaggtgccagtcagcctatgctttacagatgagaacatctgggagacactgtcccccgagctcagcacagctctgagtgaagcgattaaggacagggagggctacctgagcaaagtctgcaacctcctgcccgtcaggatcctgtcctacatcatgctgccctgcagtctgcccgtggagtcggctatcgctgcagtccacaggctggtgacatggctccctgatatccaggatgatatccagtggctacaatgggcgacatcccaggtttgtgcccgaatgacgatgtgcctgctcccctctaccaggtaaatacttgggcccagggtgtgtgggccagataggcatccctcccggttgttcccagagctcttagggtcagagcttgggtggtgacagccttaacaagccaggctcagccgcctgtccccag

SEQ ID NO: 20 - RNA, 1313 nt, Mus musculus
  source 1..1313: mol_type = mRNA
ctggggacaggcggctgagcctggcttgttaaggctgtcaccacccaagctctgaccctaagagctctgggaacaaccgggagggatgcctatctggcccacacaccctgggcccaagtatttacctggtagaggggagcaggcacatcgtcattcgggcacaaacctgggatgtcgcccattgtagccactggatatcatcctggatatcagggagccatgtcaccagcctgtggactgcagcgatagccgactccacgggcagactgcagggcagcatgatgtaggacaggatcctgacgggcaggaggttgcagactttgctcaggtagccctccctgtccttaatcgcttcactcagagctgtgctgagctcgggggacagtgtctcccagatgttctcatctgtaaagcataggctgactggcaccttgtctcccacaagtttgccattttccaagcaggcttctggcgccaccaaggacagactcaggctgcgctgtggcccgttacagatgccattctcctccaggaaccggaaggcgtccaggtacccttgatagcacagctctcccatcaccttcacatcagacgggaagagcgctctggtcagaagttggaggttcccagtgcagaggcggaggctgaggttggtgatattcacgtggaagaagttggtggacttgactttggggcagatgtcatgctcaccgtagaaaggtgacaccgtgatggtggttttggcatccagcacagggacgttgtcgctcactcctccgtccacgtaccgctcgcctcggaaggaaggagggattaggccagagaagaggggaatgaagcaggaacacaccagggcatccacgacttcgtctttggaatggaactcagacaccagcacgttctccccatccgacaccctggtgagtgagatgtgaaccttgccagaaatgacctggtggacattgtctgggaggctctcctggagcccgtctctgatgcacttgttaatgttgaagaacgggtggagggtgccgatgttgcggctcctggctttccgcacgaggtccatgaggatctccattatacggccgagagggagactgcacacgaaggtgaccgcgtgcagtgcaccggccgagcagccaaagaaagtgcgcgcatcgcggaggaggtgcggggcgcgctcgctcagacatagcgtagccccgacgtggtagaagcccaggaagccgcagcctgcaaacgacaggctccagcggcgctctgggtcatacatggtgcttgaagctccgggcggcgctgcagttcagctc

SEQ ID NO: 21 - RNA, 2759 nt, Rattus norvegicus
  source 1..2759: mol_type = mRNA
cccggagcagaattgagctgcatcgccttccggagcctccagcgccatgtacgacccagagcgccgctggagcctgtcgttcgcaggctgcggcttcctaggcttctaccacatcggggctacgctatgtctgagcgagcgcgctccgcacatcctccgcgaagcgcgcactttcttcggctgctcggccggtgcactgcacgcggtcaccttcgtgtgcagtctccctctcgatcacatcatggagatcctcatggacctcgtgcggaaagccaggagccgcaacatcggcaccctccacccgttcttcaacattaacaagtgcgtcagagacggccttcaggagaccctcccagacaacgtccaccagatcatttctggcaaggtttacatctcactcaccagagtgtccgatggggagaacgtgctggtgtctgagttccattccaaagacgaagtggtggatgccctggtgtgctcctgcttcattcctctcttctctggcctaatccctccttccttccgaggtgagcggtacgtggatggaggagtgagtgacaacgtccctgtgctggacgccaaaaccaccatcacggtgtcccctttctatggtgagcatgacatctgtcccaaagtgaagtccaccaacttcctccaggtgaatatcaccaacctcagtcttcgtctctgcactgggaaccttcatcttctgaccagagcactcttcccatctgatgtgaaggtgatgggagagctgtgctttcaagggtacctggacgccttccggttcctggaagagaacggcatctgtaatgggccacagcgcagcctgagtctgtccttggagaaggaaatggcgccagaaaccatgataccctgcttggaaaatggccaccttgtagcagggaacaaggtgccagtaagctgtgtatgccttacagctgtgccgtcggatgagagcatctgggagatgctgtcccccaagctcagcacagctctgactgaagcgattaaagacagggggggctacctgaacaaagtctgcaacctcctgcccattaggatcctgtcctacatcttgctgccctgcactctgcccgtggagtcggccatcgctgcagtccacaggctggtgatgtggctccctgatatccatgaagatatccagtggctacagtgggcaacatcccaggtgtgtgcccgaatgaccatgtgcctgctcccctctaccagatccagagcatccaaggataaccatcaaacactcaagcatggatatcacccatctctccacaaaccccaaggcagctctgccggtttgtaaattgctggtctccgtgcttccgatgaacttgggcattctccctgtggatggttccaggagaggccatagctgaaggcactctgccttccaccccaagtccagtttgacctttatctagagcaacagtgtctagatgataggtgggtggggggtgctgtctctctgtttccctctgggaagggttctgttaacttttggaggcagctaggaaatttctctccaggagctgagcctgtgcagctgcccccttggtgctgtgtggtaacctcattgcctgtgaccctaggatcataggatctgggctaaataggtagttcatagaaaccaaagacaataatttggtgtttagaaaactacttttggtctgggtgaagtctggtgcttgagagttagtgcagagagaacggtcaaaccgtctctcagcctgtggatctatggggattccaagggcttcagtgtttggaaacggcaatccaaacgggcaatcttgtgcaatcttggaaggagaactgttcaggaagtgtgatgggatgagctgtggctgtctctgaaaagggcctaccatataacttattactttcaaggatacctttggctcttactaaaatagtttataaagcattttatagaaacacaccagggaatgcgtggtgaactacatgtatgatcagtgaactgtgactagaattaaccttaaaatctcttgtatgtggggccagagcaacacaggtgggaaacgcagcggacctctgcctcctcggcctcaacatgaacttggcttgctttctccaccgtctccaaatctttgtatagtcatcgaccattaccacctctcctttcccatctactacagcagccttaatggggataagtacccccttttctcaggtgtccgaataagctgtgggtgtggcctgtgtttcctgtaattctgaggttagattggaacataagcaagcagacaaacaagcagacaaacaaacaaggttctactcatattcctaagcagtgacagtgaaggcatgtgtctcccatgcctgagtctcctagggtcctagtgagctctgggttcatgcaagcacttccggaggaattgcaccctccatggaacacataatctccactgggttgatcctgattggataagaaaggatctcggggagagaatgtggttccagaggcaaagtgtctaggctacacagaaaaggtaagactgtccccaagggaagaaaacaaactgggagctggggtccagctcaattgttaagagtgcttctctagtatgcgtgaagcccagagtccaatctcagtaccagatacacggtacaggcagtgacatatgcctgtaatcccaaccctcaagcagtagaggcaagaggatcagaagttcatggtcatccttgactacttatacttagggagttggaggtcagcc

SEQ ID NO: 22 - RNA, 2759 nt, Rattus norvegicus
  source 1..2759: mol_type = mRNA
ggctgacctccaactccctaagtataagtagtcaaggatgaccatgaacttctgatcctcttgcctctactgcttgagggttgggattacaggcatatgtcactgcctgtaccgtgtatctggtactgagattggactctgggcttcacgcatactagagaagcactcttaacaattgagctggaccccagctcccagtttgttttcttcccttggggacagtcttaccttttctgtgtagcctagacactttgcctctggaaccacattctctccccgagatcctttcttatccaatcaggatcaacccagtggagattatgtgttccatggagggtgcaattcctccggaagtgcttgcatgaacccagagctcactaggaccctaggagactcaggcatgggagacacatgccttcactgtcactgcttaggaatatgagtagaaccttgtttgtttgtctgcttgtttgtctgcttgcttatgttccaatctaacctcagaattacaggaaacacaggccacacccacagcttattcggacacctgagaaaagggggtacttatccccattaaggctgctgtagtagatgggaaaggagaggtggtaatggtcgatgactatacaaagatttggagacggtggagaaagcaagccaagttcatgttgaggccgaggaggcagaggtccgctgcgtttcccacctgtgttgctctggccccacatacaagagattttaaggttaattctagtcacagttcactgatcatacatgtagttcaccacgcattccctggtgtgtttctataaaatgctttataaactattttagtaagagccaaaggtatccttgaaagtaataagttatatggtaggcccttttcagagacagccacagctcatcccatcacacttcctgaacagttctccttccaagattgcacaagattgcccgtttggattgccgtttccaaacactgaagcccttggaatccccatagatccacaggctgagagacggtttgaccgttctctctgcactaactctcaagcaccagacttcacccagaccaaaagtagttttctaaacaccaaattattgtctttggtttctatgaactacctatttagcccagatcctatgatcctagggtcacaggcaatgaggttaccacacagcaccaagggggcagctgcacaggctcagctcctggagagaaatttcctagctgcctccaaaagttaacagaacccttcccagagggaaacagagagacagcaccccccacccacctatcatctagacactgttgctctagataaaggtcaaactggacttggggtggaaggcagagtgccttcagctatggcctctcctggaaccatccacagggagaatgcccaagttcatcggaagcacggagaccagcaatttacaaaccggcagagctgccttggggtttgtggagagatgggtgatatccatgcttgagtgtttgatggttatccttggatgctctggatctggtagaggggagcaggcacatggtcattcgggcacacacctgggatgttgcccactgtagccactggatatcttcatggatatcagggagccacatcaccagcctgtggactgcagcgatggccgactccacgggcagagtgcagggcagcaagatgtaggacaggatcctaatgggcaggaggttgcagactttgttcaggtagccccccctgtctttaatcgcttcagtcagagctgtgctgagcttgggggacagcatctcccagatgctctcatccgacggcacagctgtaaggcatacacagcttactggcaccttgttccctgctacaaggtggccattttccaagcagggtatcatggtttctggcgccatttccttctccaaggacagactcaggctgcgctgtggcccattacagatgccgttctcttccaggaaccggaaggcgtccaggtacccttgaaagcacagctctcccatcaccttcacatcagatgggaagagtgctctggtcagaagatgaaggttcccagtgcagagacgaagactgaggttggtgatattcacctggaggaagttggtggacttcactttgggacagatgtcatgctcaccatagaaaggggacaccgtgatggtggttttggcgtccagcacagggacgttgtcactcactcctccatccacgtaccgctcacctcggaaggaaggagggattaggccagagaagagaggaatgaagcaggagcacaccagggcatccaccacttcgtctttggaatggaactcagacaccagcacgttctccccatcggacactctggtgagtgagatgtaaaccttgccagaaatgatctggtggacgttgtctgggagggtctcctgaaggccgtctctgacgcacttgttaatgttgaagaacgggtggagggtgccgatgttgcggctcctggctttccgcacgaggtccatgaggatctccatgatgtgatcgagagggagactgcacacgaaggtgaccgcgtgcagtgcaccggccgagcagccgaagaaagtgcgcgcttcgcggaggatgtgcggagcgcgctcgctcagacatagcgtagccccgatgtggtagaagcctaggaagccgcagcctgcgaacgacaggctccagcggcgctctgggtcgtacatggcgctggaggctccggaaggcgatgcagctcaattctgctccggg

SEQ ID NO: 23 - RNA, 2759 nt, Rattus norvegicus; identical to SEQ ID NO: 21
  source 1..2759: mol_type = mRNA

SEQ ID NO: 24 - RNA, 2759 nt, Rattus norvegicus; identical to SEQ ID NO: 22
  source 1..2759: mol_type = mRNA

SEQ ID NO: 25 - RNA, 21 nt, synthetic construct
  source 1..21: mol_type = other RNA
gaggtcctctcagatcttgta

SEQ ID NO: 26 - RNA, 21 nt, synthetic construct
  source 1..21: mol_type = other RNA
gtcttccatccatccttcaaa

SEQ ID NO: 27 - RNA, 21 nt, synthetic construct
  source 1..21: mol_type = other RNA
gaatgtccaccagctcatcta

SEQ ID NO: 28 - RNA, 21 nt, synthetic construct
  source 1..21: mol_type = other RNA
gcttaccagagtgtctgatga

SEQ ID NO: 29 - RNA, 21 nt, synthetic construct
  source 1..21: mol_type = other RNA
cagtgtctgatggggaaaaca

SEQ ID NO: 30 - RNA, 21 nt, synthetic construct
  source 1..21: mol_type = other RNA
ctgtctgatggggaaaacgta

SEQ ID NO: 31 - RNA, 21 nt, synthetic construct
  source 1..21: mol_type = other RNA
ggtctgatggggaaaacgtta

SEQ ID NO: 32 - RNA, 21 nt, synthetic construct
  source 1..21: mol_type = other RNA
gctgatggggaaaacgttcta

SEQ ID NO: 33 - RNA, 21 nt, synthetic construct
  source 1..21: mol_type = other RNA
catggggaaaacgttctggta

SEQ ID NO: 34 - RNA, 21 nt, synthetic construct
  source 1..21: mol_type = other RNA
caaaacgttctggtgtctgaa

SEQ ID NO: 35 - RNA, 21 nt, synthetic construct
  source 1..21: mol_type = other RNA
gaacgttctggtgtctgacta

SEQ ID NO: 36 - RNA, 21 nt, synthetic construct
  source 1..21: mol_type = other RNA
gggtccaaagacgaagtcgta

SEQ ID NO: 37 - RNA, 21 nt, synthetic construct
  source 1..21: mol_type = other RNA
gttcagaggcgtgcgatatga

SEQ ID NO: 38 - RNA, 21 nt, synthetic construct
  source 1..21: mol_type = other RNA
ggtgacaacgtacccttcata

SEQ ID NO: 39 - RNA, 21 nt, synthetic construct
  source 1..21: mol_type = other RNA
ctgacaacgtacccttcatta

SEQ ID NO: 40 - RNA, 21 nt, synthetic construct
  source 1..21: mol_type = other RNA
cacaacgtacccttcattgaa

SEQ ID NO: 41 - RNA, 21 nt, synthetic construct
  source 1..21: mol_type = other RNA
gtctatggggagtacgacata

SEQ ID NO: 42 - RNA, 21 nt, synthetic construct
  source 1..21: mol_type = other RNA
ggtacgacatctgccctaaaa

SEQ ID NO: 43 - RNA, 21 nt, synthetic construct
  source 1..21: mol_type = other RNA
ctacgacatctgccctaaaga

SEQ ID NO: 44 - RNA, 21 nt, synthetic construct
  source 1..21: mol_type = other RNA
ggacatctgccctaaagtcaa

SEQ ID NO: 45 - RNA, 21 nt, synthetic construct
  source 1..21: mol_type = other RNA
cacatctgccctaaagtcaaa

SEQ ID NO: 46 - RNA, 21 nt, synthetic construct
  source 1..21: mol_type = other RNA
gcatctgccctaaagtcaaga

SEQ ID NO: 47 - RNA, 21 nt, synthetic construct
  source 1..21: mol_type = other RNA
gtctgccctaaagtcaagtca

SEQ ID NO: 48 - RNA, 21 nt, synthetic construct
  source 1..21: mol_type = other RNA
gcgaactttcttcatgtggaa

SEQ ID NO: 49 - RNA, 21 nt, synthetic construct
  source 1..21: mol_type = other RNA
gtttcttcatgtggacatcaa

SEQ ID NO: 50 - RNA, 21 nt, synthetic construct
  source 1..21: mol_type = other RNA
gttcttcatgtggacatcaca

SEQ ID NO: 51 - RNA, 21 nt, synthetic construct
  source 1..21: mol_type = other RNA
gttcatgtggacatcaccaaa

SEQ ID NO: 52 - RNA, 21 nt, synthetic construct
  source 1..21: mol_type = other RNA
ggtggacatcaccaagctcaa

SEQ ID NO: 53 - RNA, 21 nt, synthetic construct
  source 1..21: mol_type = other RNA
ggatatgccttcgaggatata

SEQ ID NO: 54 - RNA, 21 nt, synthetic construct
  source 1..21: mol_type = other RNA
catatgccttcgaggatatta

SEQ ID NO: 55 - RNA, 21 nt, synthetic construct
  source 1..21: mol_type = other RNA
gtatgccttcgaggatattta

SEQ ID NO: 56 - RNA, 21 nt, synthetic construct
  source 1..21: mol_type = other RNA
gatgccttcgaggatatttga

SEQ ID NO: 57 - RNA, 21 nt, synthetic construct
  source 1..21: mol_type = other RNA
ccattcaggttcttggaagaa

SEQ ID NO: 58 - RNA, 21 nt, synthetic construct
  source 1..21: mol_type = other RNA
gtgaaagacaaaggtggataa

SEQ ID NO: 59 - RNA, 21 nt, synthetic construct
  source 1..21: mol_type = other RNA
caaagacaaaggtggatacaa

SEQ ID NO: 60 - RNA, 21 nt, synthetic construct
  source 1..21: mol_type = other RNA
cacaaaggtggatacatgaga

SEQ ID NO: 61 - RNA, 21 nt, synthetic construct
  source 1..21: mol_type = other RNA
gaaggtggatacatgagcaaa

SEQ ID NO: 62 - RNA, 21 nt, synthetic construct
  source 1..21: mol_type = other RNA
gggatacatgagcaagattta

SEQ ID NO: 63 - RNA, 21 nt, synthetic construct
  source 1..21: mol_type = other RNA
gacatgagcaagatttgcaaa

SEQ ID NO: 64 - RNA, 21 nt, synthetic construct
  source 1..21: mol_type = other RNA
gatgagcaagatttgcaacta

SEQ ID NO: 65 - RNA, 21 nt, synthetic construct
  source 1..21: mol_type = other RNA
gtttgcaacttgctacccata

SEQ ID NO: 66 - RNA, 21 nt, synthetic construct
  source 1..21: mol_type = other RNA
gtgcaacttgctacccattaa

SEQ ID NO: 67 - RNA, 21 nt, synthetic construct
  source 1..21: mol_type = other RNA
ggcaacttgctacccattaga

SEQ ID NO: 68 - RNA, 21 nt, synthetic construct
  source 1..21: mol_type = other RNA
ccaacttgctacccattagga

SEQ ID NO: 69 - RNA, 21 nt, synthetic construct
  source 1..21: mol_type = other RNA
gaacttgctacccattaggaa

SEQ ID NO: 70 - RNA, 21 nt, synthetic construct
  source 1..21: mol_type = other RNA
gacttgctacccattaggata

SEQ ID NO: 71 - RNA, 21 nt, synthetic construct
  source 1..21: mol_type = other RNA
gtgctacccattaggataata

SEQ ID NO: 72 - RNA, 21 nt, synthetic construct
  source 1..21: mol_type = other RNA
caatctgccattgcgattgta

SEQ ID NO: 73 - RNA, 21 nt, synthetic construct
  source 1..21: mol_type = other RNA
gttgcgattgtccagagacta

SEQ ID NO: 74 - RNA, 21 nt, synthetic construct
  source 1..21: mol_type = other RNA
cgtgacatggcttccagataa

SEQ ID NO: 75 - RNA, 21 nt, synthetic construct
  source 1..21: mol_type = other RNA
ctgacatggcttccagatata

SEQ ID NO: 76 - RNA, 21 nt, synthetic construct
  source 1..21: mol_type = other RNA
ggacatggcttccagatatga

SEQ ID NO: 77 - RNA, 21 nt, synthetic construct
  source 1..21: mol_type = other RNA
gcttgggcaataaagtaccta

SEQ ID NO: 78 - RNA, 21 nt, synthetic construct
  source 1..21: mol_type = other RNA
gccacctttcccagtttttca

SEQ ID NO: 79 - RNA, 21 nt, synthetic construct
  source 1..21: mol_type = other RNA
gacctttcccagtttttcaca

SEQ ID NO: 80 - RNA, 21 nt, synthetic construct
  source 1..21: mol_type = other RNA
gttctttcagaggtgctaaaa

SEQ ID NO: 81 - RNA, 21 nt, synthetic construct
  source 1..21: mol_type = other RNA
ccctctgagctgagttggtta

SEQ ID NO: 82 - RNA, 21 nt, synthetic construct
  source 1..21: mol_type = other RNA
ggagctgagttggttttatga

SEQ ID NO: 83 - RNA, 21 nt, synthetic construct
  source 1..21: mol_type = other RNA
cctgagttggttttatgaaaa

SEQ ID NO: 84 - RNA, 21 nt, synthetic construct
  source 1..21: mol_type = other RNA
gtttatgaaaagctaggaaga

SEQ ID NO: 85 - RNA, 21 nt, synthetic construct
  source 1..21: mol_type = other RNA
gattcagctggttgggaaata

SEQ ID NO: 86 - RNA, 21 nt, synthetic construct
  source 1..21: mol_type = other RNA
gttcagctggttgggaaatga

SEQ ID NO: 87 - RNA, 21 nt, synthetic construct
  source 1..21: mol_type = other RNA
gagagggtcccttactgacta

SEQ ID NO: 88 - RNA, 21 nt, synthetic construct
  source 1..21: mol_type = other RNA
cccctattaatggtcagacta

SEQ ID NO: 89 - RNA, 21 nt, synthetic construct
  source 1..21: mol_type = other RNA
gctattaatggtcagactgta

SEQ ID NO: 90 - RNA, 21 nt, synthetic construct
  source 1..21: mol_type = other RNA
gattaatggtcagactgttca

SEQ ID NO: 91 - RNA, 21 nt, synthetic construct
  source 1..21: mol_type = other RNA
gtaatggtcagactgttccaa

SEQ ID NO: 92 - RNA, 21 nt, synthetic construct
  source 1..21: mol_type = other RNA
gacacctttttcacctaacta

SEQ ID NO: 93 - RNA, 21 nt, synthetic construct
  source 1..21: mol_type = other RNA
gacctttttcacctaactaaa

SEQ ID NO: 94 - RNA, 21 nt, synthetic construct
  source 1..21: mol_type = other RNA
gctttttcacctaactaaaaa

SEQ ID NO: 95 - RNA, 21 nt, synthetic construct
  source 1..21: mol_type = other RNA
gaagacaaaggtggatacata

SEQ ID NO: 96 - RNA, 21 nt, synthetic construct
  source 1..21: mol_type = other RNA
cgtggatacatgagcaagata

SEQ ID NO: 97 - RNA, 21 nt, synthetic construct
  source 1..21: mol_type = other RNA
ctggatacatgagcaagatta

SEQ ID NO: 98 - RNA, 21 nt, synthetic construct
  source 1..21: mol_type = other RNA
gttgctacccattaggataaa

SEQ ID NO: 99 - RNA, 21 nt, synthetic construct
  source 1..21: mol_type = other RNA
gctgagctgagttggttttaa

SEQ ID NO: 100 - RNA, 21 nt, synthetic construct
  source 1..21: mol_type = other RNA
gtggttttatgaaaagctaga

SEQ ID NO: 101 - RNA, 19 nt, synthetic construct
  source 1..19: mol_type = other RNA
ggtcctctcagatcttgta

SEQ ID NO: 102 - RNA, 19 nt, synthetic construct
  source 1..19: mol_type = other RNA
cttccatccatccttcaaa

SEQ ID NO: 103 - RNA, 19 nt, synthetic construct
  source 1..19: mol_type = other RNA
atgtccaccagctcatcta

SEQ ID NO: 104 - RNA, 19 nt, synthetic construct
  source 1..19: mol_type = other RNA
ttaccagagtgtctgatga

SEQ ID NO: 105 - RNA, 19 nt, synthetic construct
  source 1..19: mol_type = other RNA
gtgtctgatggggaaaaca

SEQ ID NO: 106 - RNA, 19 nt, synthetic construct
  source 1..19: mol_type = other RNA
gtctgatggggaaaacgta

SEQ ID NO: 107 - RNA, 19 nt, synthetic construct
  source 1..19: mol_type = other RNA
tctgatggggaaaacgtta

SEQ ID NO: 108 - RNA, 19 nt, synthetic construct
  source 1..19: mol_type = other RNA
tgatggggaaaacgttcta

SEQ ID NO: 109 - RNA, 19 nt, synthetic construct
  source 1..19: mol_type = other RNA
tggggaaaacgttctggta

SEQ ID NO: 110 - RNA, 19 nt, synthetic construct
  source 1..19: mol_type = other RNA
aaacgttctggtgtctgaa

SEQ ID NO: 111 - RNA, 19 nt, synthetic construct
  source 1..19: mol_type = other RNA
acgttctggtgtctgacta

SEQ ID NO: 112 - RNA, 19 nt, synthetic construct
  source 1..19: mol_type = other RNA
gtccaaagacgaagtcgta

SEQ ID NO: 113 - RNA, 19 nt, synthetic construct
  source 1..19: mol_type = other RNA
tcagaggcgtgcgatatga

SEQ ID NO: 114 - RNA, 19 nt, synthetic construct
  source 1..19: mol_type = other RNA
tgacaacgtacccttcata

SEQ ID NO: 115 - RNA, 19 nt, synthetic construct
  source 1..19: mol_type = other RNA
gacaacgtacccttcatta

SEQ ID NO: 116 - RNA, 19 nt, synthetic construct
  source 1..19: mol_type = other RNA
caacgtacccttcattgaa

SEQ ID NO: 117 - RNA, 19 nt, synthetic construct
  source 1..19: mol_type = other RNA
ctatggggagtacgacata

SEQ ID NO: 118 - RNA, 19 nt, synthetic construct
  source 1..19: mol_type = other RNA
tacgacatctgccctaaaa

SEQ ID NO: 119 - RNA, 19 nt, synthetic construct
  source 1..19: mol_type = other RNA
acgacatctgccctaaaga

SEQ ID NO: 120 - RNA, 19 nt, synthetic construct
  source 1..19: mol_type = other RNA
acatctgccctaaagtcaa

SEQ ID NO: 121 - RNA, 19 nt, synthetic construct
  source 1..19: mol_type = other RNA
catctgccctaaagtcaaa

SEQ ID NO: 122 - RNA, 19 nt, synthetic construct
  source 1..19: mol_type = other RNA
atctgccctaaagtcaaga

SEQ ID NO: 123 - RNA, 19 nt, synthetic construct
  source 1..19: mol_type = other RNA
ctgccctaaagtcaagtca

SEQ ID NO: 124 - RNA, 19 nt, synthetic construct
  source 1..19: mol_type = other RNA
gaactttcttcatgtggaa

SEQ ID NO: 125 - RNA, 19 nt, synthetic construct
  source 1..19: mol_type = other RNA
ttcttcatgtggacatcaa

SEQ ID NO: 126 - RNA, 19 nt, synthetic construct
  source 1..19: mol_type = other RNA
tcttcatgtggacatcaca

SEQ ID NO: 127 - RNA, 19 nt, synthetic construct
  source 1..19: mol_type = other RNA
tcatgtggacatcaccaaa

SEQ ID NO: 128 - RNA, 19 nt, synthetic construct
  source 1..19: mol_type = other RNA
tggacatcaccaagctcaa

SEQ ID NO: 129 - RNA, 19 nt, synthetic construct
  source 1..19: mol_type = other RNA
atatgccttcgaggatata

SEQ ID NO: 130 - RNA, 19 nt, synthetic construct
  source 1..19: mol_type = other RNA
tatgccttcgaggatatta

SEQ ID NO: 131 - RNA, 19 nt, synthetic construct
  source 1..19: mol_type = other RNA
atgccttcgaggatattta

SEQ ID NO: 132 - RNA, 19 nt, synthetic construct
  source 1..19: mol_type = other RNA
tgccttcgaggatatttga

SEQ ID NO: 133 - RNA, 19 nt, synthetic construct
  source 1..19: mol_type = other RNA
attcaggttcttggaagaa

SEQ ID NO: 134 - RNA, 19 nt, synthetic construct
  source 1..19: mol_type = other RNA
gaaagacaaaggtggataa

SEQ ID NO: 135 - RNA, 19 nt, synthetic construct
  source 1..19: mol_type = other RNA
aagacaaaggtggatacaa

SEQ ID NO: 136 - RNA, 19 nt, synthetic construct
  source 1..19: mol_type = other RNA
caaaggtggatacatgaga

SEQ ID NO: 137 - RNA, 19 nt, synthetic construct
  source 1..19: mol_type = other RNA
aggtggatacatgagcaaa

SEQ ID NO: 138 - RNA, 19 nt, synthetic construct
  source 1..19: mol_type = other RNA
gatacatgagcaagattta

SEQ ID NO: 139 - RNA, 19 nt, synthetic construct
  source 1..19: mol_type = other RNA
catgagcaagatttgcaaa

SEQ ID NO: 140 - RNA, 19 nt, synthetic construct
  source 1..19: mol_type = other RNA
tgagcaagatttgcaacta

SEQ ID NO: 141 - RNA, 19 nt, synthetic construct
  source 1..19: mol_type = other RNA
ttgcaacttgctacccata

SEQ ID NO: 142 - RNA, 19 nt, synthetic construct
  source 1..19: mol_type = other RNA
gcaacttgctacccattaa

SEQ ID NO: 143 - RNA, 19 nt, synthetic construct
  source 1..19: mol_type = other RNA
caacttgctacccattaga

SEQ ID NO: 144 - RNA, 19 nt, synthetic construct
  source 1..19: mol_type = other RNA
aacttgctacccattagga

SEQ ID NO: 145 - RNA, 19 nt, synthetic construct
  source 1..19: mol_type = other RNA
acttgctacccattaggaa

SEQ ID NO: 146 - RNA, 19 nt, synthetic construct
  source 1..19: mol_type = other RNA
cttgctacccattaggata

SEQ ID NO: 147 - RNA, 19 nt, synthetic construct
  source 1..19: mol_type = other RNA
gctacccattaggataata

SEQ ID NO: 148 - RNA, 19 nt, synthetic construct
  source 1..19: mol_type = other RNA
atctgccattgcgattgta

SEQ ID NO: 149 - RNA, 19 nt, synthetic construct
  source 1..19: mol_type = other RNA
tgcgattgtccagagacta

SEQ ID NO: 150 - RNA, 19 nt, synthetic construct
  source 1..19: mol_type = other RNA
tgacatggcttccagataa

SEQ ID NO: 151 - RNA, 19 nt, synthetic construct
  source 1..19: mol_type = other RNA
gacatggcttccagatata

SEQ ID NO: 152 - RNA, 19 nt, synthetic construct
  source 1..19: mol_type = other RNA
acatggcttccagatatga

SEQ ID NO: 153 - RNA, 19 nt, synthetic construct
  source 1..19: mol_type = other RNA
ttgggcaataaagtaccta

SEQ ID NO: 154 - RNA, 19 nt, synthetic construct
  source 1..19: mol_type = other RNA
cacctttcccagtttttca

SEQ ID NO: 155 - RNA, 19 nt, synthetic construct
  source 1..19: mol_type = other RNA
cctttcccagtttttcaca

SEQ ID NO: 156 - RNA, 19 nt, synthetic construct
  source 1..19: mol_type = other RNA
tctttcagaggtgctaaaa

SEQ ID NO: 157 - RNA, 19 nt, synthetic construct
  source 1..19: mol_type = other RNA
ctctgagctgagttggtta

SEQ ID NO: 158 - RNA, 19 nt, synthetic construct
  source 1..19: mol_type = other RNA
agctgagttggttttatga

SEQ ID NO: 159 - RNA, 19 nt, synthetic construct
  source 1..19: mol_type = other RNA
tgagttggttttatgaaaa

SEQ ID NO: 160 - RNA, 19 nt, synthetic construct
  source 1..19: mol_type = other RNA
ttatgaaaagctaggaaga

SEQ ID NO: 161 - RNA, 19 nt, synthetic construct
  source 1..19: mol_type = other RNA
ttcagctggttgggaaata

SEQ ID NO: 162 - RNA, 19 nt, synthetic construct
  source 1..19: mol_type = other RNA
tcagctggttgggaaatga

SEQ ID NO: 163 - RNA, 19 nt, synthetic construct
  source 1..19: mol_type = other RNA
gagggtcccttactgacta

SEQ ID NO: 164 - RNA, 19 nt, synthetic construct
  source 1..19: mol_type = other RNA
cctattaatggtcagacta

SEQ ID NO: 165 - RNA, 19 nt, synthetic construct
  source 1..19: mol_type = other RNA
tattaatggtcagactgta

SEQ ID NO: 166 - RNA, 19 nt, synthetic construct
  source 1..19: mol_type = other RNA
ttaatggtcagactgttca

SEQ ID NO: 167 - RNA, 19 nt, synthetic construct
  source 1..19: mol_type = other RNA
aatggtcagactgttccaa

SEQ ID NO: 168 - RNA, 19 nt, synthetic construct
  source 1..19: mol_type = other RNA
cacctttttcacctaacta

SEQ ID NO: 169 - RNA, 19 nt, synthetic construct
  source 1..19: mol_type = other RNA
cctttttcacctaactaaa

SEQ ID NO: 170 - RNA, 19 nt, synthetic construct
  source 1..19: mol_type = other RNA
tttttcacctaactaaaaa

SEQ ID NO: 171 - RNA, 19 nt, synthetic construct
  source 1..19: mol_type = other RNA
agacaaaggtggatacata

SEQ ID NO: 172 - RNA, 19 nt, synthetic construct
  source 1..19: mol_type = other RNA
tggatacatgagcaagata

SEQ ID NO: 173 - RNA, 19 nt, synthetic construct
  source 1..19: mol_type = other RNA
ggatacatgagcaagatta

SEQ ID NO: 174 - RNA, 19 nt, synthetic construct
  source 1..19: mol_type = other RNA
tgctacccattaggataaa

SEQ ID NO: 175 - RNA, 19 nt, synthetic construct
  source 1..19: mol_type = other RNA
tgagctgagttggttttaa

SEQ ID NO: 176 - RNA, 19 nt, synthetic construct
  source 1..19: mol_type = other RNA
ggttttatgaaaagctaga

SEQ ID NO: 177 - RNA, 21 nt, synthetic construct
  source 1..21: mol_type = other RNA
nnngtcctctcagatcttgtn

SEQ ID NO: 178 - RNA, 21 nt, synthetic construct
  source 1..21: mol_type = other RNA
nnnttccatccatccttcaan

SEQ ID NO: 179 - RNA, 21 nt, synthetic construct
  source 1..21: mol_type = other RNA
nnntgtccaccagctcatctn

SEQ ID NO: 180 - RNA, 21 nt, synthetic construct
  source 1..21: mol_type = other RNA
nnntaccagagtgtctgatgn

SEQ ID NO: 181 - RNA, 21 nt, synthetic construct
  source 1..21: mol_type = other RNA
nnntgtctgatggggaaaacn

SEQ ID NO: 182 - RNA, 21 nt, synthetic construct
  source 1..21: mol_type = other RNA
nnntctgatggggaaaacgtn

SEQ ID NO: 183 - RNA, 21 nt, synthetic construct
  source 1..21: mol_type = other RNA
nnnctgatggggaaaacgttn

SEQ ID NO: 184 - RNA, 21 nt, synthetic construct
  source 1..21: mol_type = other RNA
nnngatggggaaaacgttctn

SEQ ID NO: 185 - RNA, 21 nt, synthetic construct
  source 1..21: mol_type = other RNA
nnnggggaaaacgttctggtn

SEQ ID NO: 186 - RNA, 21 nt, synthetic construct
  source 1..21: mol_type = other RNA
nnnaacgttctggtgtctgan

SEQ ID NO: 187 - RNA, 21 nt, synthetic construct
  source 1..21: mol_type = other RNA
nnncgttctggtgtctgactn

SEQ ID NO: 188 - RNA, 21 nt, synthetic construct
  source 1..21: mol_type = other RNA
nnntccaaagacgaagtcgtn

SEQ ID NO: 189 - RNA, 21 nt, synthetic construct
  source 1..21: mol_type = other RNA
nnncagaggcgtgcgatatgn

SEQ ID NO: 190 - RNA, 21 nt, synthetic construct
  source 1..21: mol_type = other RNA
nnngacaacgtacccttcatn

SEQ ID NO: 191 - RNA, 21 nt, synthetic construct
  source 1..21: mol_type = other RNA
nnnacaacgtacccttcattn

SEQ ID NO: 192 - RNA, 21 nt, synthetic construct
  source 1..21: mol_type = other RNA
nnnaacgtacccttcattgan

SEQ ID NO: 193 - RNA, 21 nt, synthetic construct
  source 1..21: mol_type = other RNA
nnntatggggagtacgacatn

SEQ ID NO: 194 - RNA, 21 nt, synthetic construct
  source 1..21: mol_type = other RNA
nnnacgacatctgccctaaan

SEQ ID NO: 195 - RNA, 21 nt, synthetic construct
  source 1..21: mol_type = other RNA
nnncgacatctgccctaaagn

SEQ ID NO: 196 - RNA, 21 nt, synthetic construct
  source 1..21: mol_type = other RNA
nnncatctgccctaaagtcan

SEQ ID NO: 197 - RNA, 21 nt, synthetic construct
  source 1..21: mol_type = other RNA
nnnatctgccctaaagtcaan

SEQ ID NO: 198 - RNA, 21 nt, synthetic construct
  source 1..21: mol_type = other RNA
nnntctgccctaaagtcaagn

SEQ ID NO: 199 - RNA, 21 nt, synthetic construct
  source 1..21: mol_type = other RNA
nnntgccctaaagtcaagtcn

SEQ ID NO: 200 - RNA, 21 nt, synthetic construct
  source 1..21: mol_type = other RNA
nnnaactttcttcatgtggan

SEQ ID NO: 201 - RNA, 21 nt, synthetic construct
  source 1..21: mol_type = other RNA
nnntcttcatgtggacatcan

SEQ ID NO: 202 - RNA, 21 nt, synthetic construct
  source 1..21: mol_type = other RNA
nnncttcatgtggacatcacn

SEQ ID NO: 203 - RNA, 21 nt, synthetic construct
  source 1..21: mol_type = other RNA
nnncatgtggacatcaccaan

SEQ ID NO: 204 - RNA, 21 nt, synthetic construct
  source 1..21: mol_type = other RNA
nnnggacatcaccaagctcan

SEQ ID NO: 205 - RNA, 21 nt, synthetic construct
  source 1..21: mol_type = other RNA
nnntatgccttcgaggatatn

SEQ ID NO: 206 - RNA, 21 nt, synthetic construct
  source 1..21: mol_type = other RNA
nnnatgccttcgaggatattn

SEQ ID NO: 207 - RNA, 21 nt, synthetic construct
  source 1..21: mol_type = other RNA
nnntgccttcgaggatatttn

SEQ ID NO: 208 - RNA, 21 nt, synthetic construct
  source 1..21: mol_type = other RNA
nnngccttcgaggatatttgn

SEQ ID NO: 209 - RNA, 21 nt, synthetic construct
  source 1..21: mol_type = other RNA
nnnttcaggttcttggaagan

SEQ ID NO: 210 - RNA, 21 nt, synthetic construct
  source 1..21: mol_type = other RNA
nnnaaagacaaaggtggatan

SEQ ID NO: 211 - RNA, 21 nt, synthetic construct
  source 1..21: mol_type = other RNA
nnnagacaaaggtggatacan

SEQ ID NO: 212 - RNA, 21 nt, synthetic construct
  source 1..21: mol_type = other RNA
nnnaaaggtggatacatgagn

SEQ ID NO: 213 - RNA, 21 nt, synthetic construct
  source 1..21: mol_type = other RNA
nnnggtggatacatgagcaan

SEQ ID NO: 214 - RNA, 21 nt, synthetic construct
  source 1..21: mol_type = other RNA
nnnatacatgagcaagatttn

SEQ ID NO: 215 - RNA, 21 nt, synthetic construct
  source 1..21: mol_type = other RNA
nnnatgagcaagatttgcaan

SEQ ID NO: 216 - RNA, 21 nt, synthetic construct
  source 1..21: mol_type = other RNA
nnngagcaagatttgcaactn

SEQ ID NO: 217 - RNA, 21 nt, synthetic construct
  source 1..21: mol_type = other RNA
nnntgcaacttgctacccatn

SEQ ID NO: 218 - RNA, 21 nt, synthetic construct
  source 1..21: mol_type = other RNA
nnncaacttgctacccattan

SEQ ID NO: 219 - RNA, 21 nt, synthetic construct
  source 1..21: mol_type = other RNA
nnnaacttgctacccattagn

SEQ ID NO: 220 - RNA, 21 nt, synthetic construct
  source 1..21: mol_type = other RNA
nnnacttgctacccattaggn

SEQ ID NO: 221 - RNA, 21 nt, synthetic construct
  source 1..21: mol_type = other RNA
nnncttgctacccattaggan

SEQ ID NO: 222 - RNA, 21 nt, synthetic construct
  source 1..21: mol_type = other RNA
nnnttgctacccattaggatn

SEQ ID NO: 223 - RNA, 21 nt, synthetic construct
  source 1..21: mol_type = other RNA
nnnctacccattaggataatn

SEQ ID NO: 224 - RNA, 21 nt, synthetic construct
  source 1..21: mol_type = other RNA
nnntctgccattgcgattgtn

SEQ ID NO: 225 - RNA, 21 nt, synthetic construct
  source 1..21: mol_type = other RNA
nnngcgattgtccagagactn

SEQ ID NO: 226 - RNA, 21 nt, synthetic construct
  source 1..21: mol_type = other RNA
nnngacatggcttccagatan

SEQ ID NO: 227 - RNA, 21 nt, synthetic construct
  source 1..21: mol_type = other RNA
nnnacatggcttccagatatn

SEQ ID NO: 228 - RNA, 21 nt, synthetic construct
  source 1..21: mol_type = other RNA
nnncatggcttccagatatgn

SEQ ID NO: 229 - RNA, 21 nt, synthetic construct
  source 1..21: mol_type = other RNA
nnntgggcaataaagtacctn

SEQ ID NO: 230 - RNA, 21 nt, synthetic construct
  source 1..21: mol_type = other RNA
nnnacctttcccagtttttcn

SEQ ID NO: 231 - RNA, 21 nt, synthetic construct
  source 1..21: mol_type = other RNA
nnnctttcccagtttttcacn

SEQ ID NO: 232 - RNA, 21 nt, synthetic construct
  source 1..21: mol_type = other RNA
nnnctttcagaggtgctaaan

SEQ ID NO: 233 - RNA, 21 nt, synthetic construct
  source 1..21: mol_type = other RNA
nnntctgagctgagttggttn

SEQ ID NO: 234 - RNA, 21 nt, synthetic construct
  source 1..21: mol_type = other RNA
nnngctgagttggttttatgn

SEQ ID NO: 235 - RNA, 21 nt, synthetic construct
  source 1..21: mol_type = other RNA
nnngagttggttttatgaaan

SEQ ID NO: 236 - RNA, 21 nt, synthetic construct
  source 1..21: mol_type = other RNA
nnntatgaaaagctaggaagn

SEQ ID NO: 237 - RNA, 21 nt, synthetic construct
  source 1..21: mol_type = other RNA
nnntcagctggttgggaaatn

SEQ ID NO: 238 - RNA, 21 nt, synthetic construct
  source 1..21: mol_type = other RNA
nnncagctggttgggaaatgn

SEQ ID NO: 239 - RNA, 21 nt, synthetic construct
  source 1..21: mol_type = other RNA
nnnagggtcccttactgactn

SEQ ID NO: 240 - RNA, 21 nt, synthetic construct
  source 1..21: mol_type = other RNA
nnnctattaatggtcagactn

SEQ ID NO: 241 - RNA, 21 nt, synthetic construct
  source 1..21: mol_type = other RNA
nnnattaatggtcagactgtn

SEQ ID NO: 242 - RNA, 21 nt, synthetic construct
  source 1..21: mol_type = other RNA
nnntaatggtcagactgttcn

SEQ ID NO: 243 - RNA, 21 nt, synthetic construct
  source 1..21: mol_type = other RNA
nnnatggtcagactgttccan

SEQ ID NO: 244 - RNA, 21 nt, synthetic construct
  source 1..21: mol_type = other RNA
nnnacctttttcacctaactn

SEQ ID NO: 245 - RNA, 21 nt, synthetic construct
  source 1..21: mol_type = other RNA
nnnctttttcacctaactaan

SEQ ID NO: 246 - RNA, 21 nt, synthetic construct
  source 1..21: mol_type = other RNA
nnnttttcacctaactaaaan

SEQ ID NO: 247 - RNA, 21 nt, synthetic construct
  source 1..21: mol_type = other RNA
nnngacaaaggtggatacatn

SEQ ID NO: 248 - RNA, 21 nt, synthetic construct
  source 1..21: mol_type = other RNA
nnnggatacatgagcaagatn

SEQ ID NO: 249 - RNA, 21 nt, synthetic construct
  source 1..21: mol_type = other RNA
nnngatacatgagcaagattn

SEQ ID NO: 250 - RNA, 21 nt, synthetic construct
  source 1..21: mol_type = other RNA
nnngctacccattaggataan

SEQ ID NO: 251 - RNA, 21 nt, synthetic construct
  source 1..21: mol_type = other RNA
nnngagctgagttggttttan

SEQ ID NO: 252 - RNA, 21 nt, synthetic construct
  source 1..21: mol_type = other RNA
nnngttttatgaaaagctagn

SEQ ID NO: 253 - RNA, 21 nt, synthetic construct
  source 1..21: mol_type = other RNA
tacaagatctgagaggacctc

SEQ ID NO: 254 - RNA, 21 nt, synthetic construct
  source 1..21: mol_type = other RNA
tttgaaggatggatggaagac

SEQ ID NO: 255 - RNA, 21 nt, synthetic construct
  source 1..21: mol_type = other RNA
tagatgagctggtggacattc

SEQ ID NO: 256 - RNA, 21 nt, synthetic construct
  source 1..21: mol_type = other RNA
tcatcagacactctggtaagc

SEQ ID NO: 257 - RNA, 21 nt, synthetic construct
  source 1..21: mol_type = other RNA
tgttttccccatcagacactg

SEQ ID NO: 258 - RNA, 21 nt, synthetic construct
  source 1..21: mol_type = other RNA
tacgttttccccatcagacag

SEQ ID NO: 259 - RNA, 21 nt, synthetic construct
  source 1..21: mol_type = other RNA
taacgttttccccatcagacc

SEQ ID NO: 260 - RNA, 21 nt, synthetic construct
  source 1..21: mol_type = other RNA
tagaacgttttccccatcagc

SEQ ID NO: 261 - RNA, 21 nt, synthetic construct
  source 1..21: mol_type = other RNA
taccagaacgttttccccatg

SEQ ID NO: 262 - RNA, 21 nt, synthetic construct
  source 1..21: mol_type = other RNA
ttcagacaccagaacgttttg

SEQ ID NO: 263 - RNA, 21 nt, synthetic construct
  source 1..21: mol_type = other RNA
tagtcagacaccagaacgttc

SEQ ID NO: 264 - RNA, 21 nt, synthetic construct
  source 1..21: mol_type = other RNA
tacgacttcgtctttggaccc

SEQ ID NO: 265 - RNA, 21 nt, synthetic construct
  source 1..21: mol_type = other RNA
tcatatcgcacgcctctgaac

SEQ ID NO: 266 - RNA, 21 nt, synthetic construct
  source 1..21: mol_type = other RNA
tatgaagggtacgttgtcacc

SEQ ID NO: 267 - RNA, 21 nt, synthetic construct
  source 1..21: mol_type = other RNA
taatgaagggtacgttgtcag

SEQ ID NO: 268 - RNA, 21 nt, synthetic construct
  source 1..21: mol_type = other RNA
ttcaatgaagggtacgttgtg

SEQ ID NO: 269 - RNA, 21 nt, synthetic construct
  source 1..21: mol_type = other RNA
tatgtcgtactccccatagac

SEQ ID NO: 270 - RNA, 21 nt, synthetic construct
  source 1..21: mol_type = other RNA
ttttagggcagatgtcgtacc

SEQ ID NO: 271 - RNA, 21 nt, synthetic construct
  source 1..21: mol_type = other RNA
tctttagggcagatgtcgtag

SEQ ID NO: 272 - RNA, 21 nt, synthetic construct
  source 1..21: mol_type = other RNA
ttgactttagggcagatgtcc

SEQ ID NO: 273 - RNA, 21 nt, synthetic construct
  source 1..21: mol_type = other RNA
tttgactttagggcagatgtg

SEQ ID NO: 274 - RNA, 21 nt, synthetic construct
  source 1..21: mol_type = other RNA
tcttgactttagggcagatgc

SEQ ID NO: 275 - RNA, 21 nt, synthetic construct
  source 1..21: mol_type = other RNA
tgacttgactttagggcagac

SEQ ID NO: 276 - RNA, 21 nt, synthetic construct
  source 1..21: mol_type = other RNA
ttccacatgaagaaagttcgc

SEQ ID NO: 277 - RNA, 21 nt, synthetic construct
  source 1..21: mol_type = other RNA
ttgatgtccacatgaagaaac

SEQ ID NO: 278 - RNA, 21 nt, synthetic construct
  source 1..21: mol_type = other RNA
tgtgatgtccacatgaagaac

SEQ ID NO: 279 - RNA, 21 nt, synthetic construct
  source 1..21: mol_type = other RNA
tttggtgatgtccacatgaac

SEQ ID NO: 280 - RNA, 21 nt, synthetic construct
  source 1..21: mol_type = other RNA
ttgagcttggtgatgtccacc

SEQ ID NO: 281 - RNA, 21 nt, synthetic construct
  source 1..21: mol_type = other RNA
tatatcctcgaaggcatatcc

SEQ ID NO: 282 - RNA, 21 nt, synthetic construct
  source 1..21: mol_type = other RNA
taatatcctcgaaggcatatg

SEQ ID NO: 283 - RNA, 21 nt, synthetic construct
  source 1..21: mol_type = other RNA
taaatatcctcgaaggcatac

SEQ ID NO: 284 - RNA, 21 nt, synthetic construct
  source 1..21: mol_type = other RNA
tcaaatatcctcgaaggcatc

SEQ ID NO: 285 - RNA, 21 nt, synthetic construct
  source 1..21: mol_type = other RNA
ttcttccaagaacctgaatgg

SEQ ID NO: 286 - RNA, 21 nt, synthetic construct
  source 1..21: mol_type = other RNA
ttatccacctttgtctttcac

SEQ ID NO: 287 - RNA, 21 nt, synthetic construct
  source 1..21: mol_type = other RNA
ttgtatccacctttgtctttg

SEQ ID NO: 288 - RNA, 21 nt, synthetic construct
  source 1..21: mol_type = other RNA
tctcatgtatccacctttgtg

SEQ ID NO: 289 - RNA, 21 nt, synthetic construct
  source 1..21: mol_type = other RNA
tttgctcatgtatccaccttc

SEQ ID NO: 290 - RNA, 21 nt, synthetic construct
  source 1..21: mol_type = other RNA
taaatcttgctcatgtatccc

SEQ ID NO: 291 - RNA, 21 nt, synthetic construct
  source 1..21: mol_type = other RNA
tttgcaaatcttgctcatgtc

SEQ ID NO: 292 - RNA, 21 nt, synthetic construct
  source 1..21: mol_type = other RNA
tagttgcaaatcttgctcatc

SEQ ID NO: 293 - RNA, 21 nt, synthetic construct
  source 1..21: mol_type = other RNA
tatgggtagcaagttgcaaac

SEQ ID NO: 294 - RNA, 21 nt, synthetic construct
  source 1..21: mol_type = other RNA
ttaatgggtagcaagttgcac

SEQ ID NO: 295 - RNA, 21 nt, synthetic construct
  source 1..21: mol_type = other RNA
tctaatgggtagcaagttgcc

SEQ ID NO: 296 - RNA, 21 nt, synthetic construct
  source 1..21: mol_type = other RNA
tcctaatgggtagcaagttgg

SEQ ID NO: 297 - RNA, 21 nt, synthetic construct
  source 1..21: mol_type = other RNA
ttcctaatgggtagcaagttc

SEQ ID NO: 298 - RNA, 21 nt, synthetic construct
  source 1..21: mol_type = other RNA
tatcctaatgggtagcaagtc

SEQ ID NO: 299 - RNA, 21 nt, synthetic construct
  source 1..21: mol_type = other RNA
tattatcctaatgggtagcac

SEQ ID NO: 300 - RNA, 21 nt, synthetic construct
  source 1..21: mol_type = other RNA
tacaatcgcaatggcagattg

SEQ ID NO: 301 - RNA, 21 nt, synthetic construct
  source 1..21: mol_type = other RNA
tagtctctggacaatcgcaac

SEQ ID NO: 302 - RNA, 21 nt, synthetic construct
  source 1..21: mol_type = other RNA
ttatctggaagccatgtcacg

SEQ ID NO: 303 - RNA, 21 nt, synthetic construct
  source 1..21: mol_type = other RNA
tatatctggaagccatgtcag

SEQ ID NO: 304 - RNA, 21 nt, synthetic construct
  source 1..21: mol_type = other RNA
tcatatctggaagccatgtcc

SEQ ID NO: 305 - RNA, 21 nt, synthetic construct
  source 1..21: mol_type = other RNA
taggtactttattgcccaagc

SEQ ID NO: 306 - RNA, 21 nt, synthetic construct
  source 1..21: mol_type = other RNA
tgaaaaactgggaaaggtggc

SEQ ID NO: 307 - RNA, 21 nt, synthetic construct
  source 1..21: mol_type = other RNA
tgtgaaaaactgggaaaggtc

SEQ ID NO: 308 - RNA, 21 nt, synthetic construct
  source 1..21: mol_type = other RNA
ttttagcacctctgaaagaac

SEQ ID NO: 309 - RNA, 21 nt, synthetic construct
  source 1..21: mol_type = other RNA
taaccaactcagctcagaggg

SEQ ID NO: 310 - RNA, 21 nt, synthetic construct
  source 1..21: mol_type = other RNA
tcataaaaccaactcagctcc

SEQ ID NO: 311 - RNA, 21 nt, synthetic construct
  source 1..21: mol_type = other RNA
ttttcataaaaccaactcagg

SEQ ID NO: 312 - RNA, 21 nt, synthetic construct
  source 1..21: mol_type = other RNA
tcttcctagcttttcataaac

SEQ ID NO: 313 - RNA, 21 nt, synthetic construct
  source 1..21: mol_type = other RNA
tatttcccaaccagctgaatc

SEQ ID NO: 314 - RNA, 21 nt, synthetic construct
  source 1..21: mol_type = other RNA
tcatttcccaaccagctgaac

SEQ ID NO: 315 - RNA, 21 nt, synthetic construct
  source 1..21: mol_type = other RNA
tagtcagtaagggaccctctc

SEQ ID NO: 316 - RNA, 21 nt, synthetic construct
  source 1..21: mol_type = other RNA
tagtctgaccattaatagggg

SEQ ID NO: 317 - RNA, 21 nt, synthetic construct
  source 1..21: mol_type = other RNA
tacagtctgaccattaatagc

SEQ ID NO: 318 - RNA, 21 nt, synthetic construct
  source 1..21: mol_type = other RNA
tgaacagtctgaccattaatc

SEQ ID NO: 319 - RNA, 21 nt, synthetic construct
  source 1..21: mol_type = other RNA
ttggaacagtctgaccattac

SEQ ID NO: 320 - RNA, 21 nt, synthetic construct
  source 1..21: mol_type = other RNA
tagttaggtgaaaaaggtgtc

SEQ ID NO: 321 - RNA, 21 nt, synthetic construct
  source 1..21: mol_type = other RNA
tttagttaggtgaaaaaggtc

SEQ ID NO: 322 - RNA, 21 nt, synthetic construct
  source 1..21: mol_type = other RNA
tttttagttaggtgaaaaagc

SEQ ID NO: 323 - RNA, 21 nt, synthetic construct
  source 1..21: mol_type = other RNA
tatgtatccacctttgtcttc

SEQ ID NO: 324 - RNA, 21 nt, synthetic construct
  source 1..21: mol_type = other RNA
tatcttgctcatgtatccacg

SEQ ID NO: 325 - RNA, 21 nt, synthetic construct
  source 1..21: mol_type = other RNA
taatcttgctcatgtatccag

SEQ ID NO: 326 - RNA, 21 nt, synthetic construct
  source 1..21: mol_type = other RNA
tttatcctaatgggtagcaac

SEQ ID NO: 327 - RNA, 21 nt, synthetic construct
  source 1..21: mol_type = other RNA
ttaaaaccaactcagctcagc

SEQ ID NO: 328 - RNA, 21 nt, synthetic construct
  source 1..21: mol_type = other RNA
tctagcttttcataaaaccac

SEQ ID NO: 329 - RNA, 19 nt, synthetic construct
  source 1..19: mol_type = other RNA
tacaagatctgagaggacc

SEQ ID NO: 330 - RNA, 19 nt, synthetic construct
  source 1..19: mol_type = other RNA
tttgaaggatggatggaag

SEQ ID NO: 331 - RNA, 19 nt, synthetic construct
  source 1..19: mol_type = other RNA
tagatgagctggtggacat

SEQ ID NO: 332 - RNA, 19 nt, synthetic construct
  source 1..19: mol_type = other RNA
tcatcagacactctggtaa

SEQ ID NO: 333 - RNA, 19 nt, synthetic construct
  source 1..19: mol_type = other RNA
tgttttccccatcagacac

SEQ ID NO: 334 - RNA, 19 nt, synthetic construct
  source 1..19: mol_type = other RNA
tacgttttccccatcagac

SEQ ID NO: 335 - RNA, 19 nt, synthetic construct
  source 1..19: mol_type = other RNA
taacgttttccccatcaga

SEQ ID NO: 336 - RNA, 19 nt, synthetic construct
  source 1..19: mol_type = other RNA
tagaacgttttccccatca

SEQ ID NO: 337 - RNA, 19 nt, synthetic construct
  source 1..19: mol_type = other RNA
taccagaacgttttcccca

SEQ ID NO: 338 - RNA, 19 nt, synthetic construct
  source 1..19: mol_type = other RNA
ttcagacaccagaacgttt

SEQ ID NO: 339 - RNA, 19 nt, synthetic construct
  source 1..19: mol_type = other RNA
tagtcagacaccagaacgt

SEQ ID NO: 340 - RNA, 19 nt, synthetic construct
  source 1..19: mol_type = other RNA
tacgacttcgtctttggac

SEQ ID NO: 341 - RNA, 19 nt, synthetic construct
  source 1..19: mol_type = other RNA
tcatatcgcacgcctctga

SEQ ID NO: 342 - RNA, 19 nt, synthetic construct
  source 1..19: mol_type = other RNA
tatgaagggtacgttgtca

SEQ ID NO: 343 - RNA, 19 nt, synthetic construct
  source 1..19: mol_type = other RNA
taatgaagggtacgttgtc

SEQ ID NO: 344 - RNA, 19 nt, synthetic construct
  source 1..19: mol_type = other RNA
ttcaatgaagggtacgttg

SEQ ID NO: 345 - RNA, 19 nt, synthetic construct
  source 1..19: mol_type = other RNA
tatgtcgtactccccatag

SEQ ID NO: 346 - RNA, 19 nt, synthetic construct
  source 1..19: mol_type = other RNA
ttttagggcagatgtcgta

SEQ ID NO: 347 - RNA, 19 nt, synthetic construct
  source 1..19: mol_type = other RNA
tctttagggcagatgtcgt

SEQ ID NO: 348 - RNA, 19 nt, synthetic construct
  source 1..19: mol_type = other RNA
ttgactttagggcagatgt

SEQ ID NO: 349 - RNA, 19 nt, synthetic construct
  source 1..19: mol_type = other RNA
tttgactttagggcagatg

SEQ ID NO: 350 - RNA, 19 nt, synthetic construct
  source 1..19: mol_type = other RNA
tcttgactttagggcagat

SEQ ID NO: 351 - RNA, 19 nt, synthetic construct
  source 1..19: mol_type = other RNA
tgacttgactttagggcag

SEQ ID NO: 352 - RNA, 19 nt, synthetic construct
  source 1..19: mol_type = other RNA
ttccacatgaagaaagttc

SEQ ID NO: 353 - RNA, 19 nt, synthetic construct
  source 1..19: mol_type = other RNA
ttgatgtccacatgaagaa

SEQ ID NO: 354 - RNA, 19 nt, synthetic construct
  source 1..19: mol_type = other RNA
tgtgatgtccacatgaaga

SEQ ID NO: 355 - RNA, 19 nt, synthetic construct
  source 1..19: mol_type = other RNA
tttggtgatgtccacatga

SEQ ID NO: 356 - RNA, 19 nt, synthetic construct
  source 1..19: mol_type = other RNA
ttgagcttggtgatgtcca

SEQ ID NO: 357 - RNA, 19 nt, synthetic construct
  source 1..19: mol_type = other RNA
tatatcctcgaaggcatat

SEQ ID NO: 358 - RNA, 19 nt, synthetic construct
  source 1..19: mol_type = other RNA
taatatcctcgaaggcata

SEQ ID NO: 359 - RNA, 19 nt, synthetic construct
  source 1..19: mol_type = other RNA
taaatatcctcgaaggcat

SEQ ID NO: 360 - RNA, 19 nt, synthetic construct
  source 1..19: mol_type = other RNA
tcaaatatcctcgaaggca

SEQ ID NO: 361 - RNA, 19 nt, synthetic construct
  source 1..19: mol_type = other RNA
ttcttccaagaacctgaat

SEQ ID NO: 362 - RNA, 19 nt, synthetic construct
  source 1..19: mol_type = other RNA
ttatccacctttgtctttc

SEQ ID NO: 363 - RNA, 19 nt, synthetic construct
  source 1..19: mol_type = other RNA
ttgtatccacctttgtctt

SEQ ID NO: 364 - RNA, 19 nt, synthetic construct
  source 1..19: mol_type = other RNA
tctcatgtatccacctttg

SEQ ID NO: 365 - RNA, 19 nt, synthetic construct
  source 1..19: mol_type = other RNA
tttgctcatgtatccacct

SEQ ID NO: 366 - RNA, 19 nt, synthetic construct
  source 1..19: mol_type = other RNA
taaatcttgctcatgtatc

SEQ ID NO: 367 - RNA, 19 nt, synthetic construct
  source 1..19: mol_type = other RNA
tttgcaaatcttgctcatg

SEQ ID NO: 368 - RNA, 19 nt, synthetic construct
  source 1..19: mol_type = other RNA
tagttgcaaatcttgctca

SEQ ID NO: 369 - RNA, 19 nt, synthetic construct
  source 1..19: mol_type = other RNA
tatgggtagcaagttgcaa

SEQ ID NO: 370 - RNA, 19 nt, synthetic construct
  source 1..19: mol_type = other RNA
ttaatgggtagcaagttgc

SEQ ID NO: 371 - RNA, 19 nt, synthetic construct
  source 1..19: mol_type = other RNA
tctaatgggtagcaagttg

SEQ ID NO: 372 - RNA, 19 nt, synthetic construct
  source 1..19: mol_type = other RNA
tcctaatgggtagcaagtt

SEQ ID NO: 373 - RNA, 19 nt, synthetic construct
  source 1..19: mol_type = other RNA
ttcctaatgggtagcaagt

SEQ ID NO: 374 - RNA, 19 nt, synthetic construct
  source 1..19: mol_type = other RNA
tatcctaatgggtagcaag

SEQ ID NO: 375 - RNA, 19 nt, synthetic construct
  source 1..19: mol_type = other RNA
tattatcctaatgggtagc

SEQ ID NO: 376 - RNA, 19 nt, synthetic construct
  source 1..19: mol_type = other RNA
tacaatcgcaatggcagat

SEQ ID NO: 377 - RNA, 19 nt, synthetic construct
  source 1..19: mol_type = other RNA
tagtctctggacaatcgca

SEQ ID NO: 378 - RNA, 19 nt, synthetic construct
  source 1..19: mol_type = other RNA
ttatctggaagccatgtca

SEQ ID NO: 379 - RNA, 19 nt, synthetic construct
  source 1..19: mol_type = other RNA
tatatctggaagccatgtc

SEQ ID NO: 380 - RNA, 19 nt, synthetic construct
  source 1..19: mol_type = other RNA
tcatatctggaagccatgt

SEQ ID NO: 381 - RNA, 19 nt, synthetic construct
  source 1..19: mol_type = other RNA
taggtactttattgcccaa

SEQ ID NO: 382 - RNA, 19 nt, synthetic construct
  source 1..19: mol_type = other RNA
tgaaaaactgggaaaggtg

SEQ ID NO: 383 - RNA, 19 nt, synthetic construct
  source 1..19: mol_type = other RNA
tgtgaaaaactgggaaagg

SEQ ID NO: 384 - RNA, 19 nt, synthetic construct
  source 1..19: mol_type = other RNA
ttttagcacctctgaaaga

SEQ ID NO: 385 - RNA, 19 nt, synthetic construct
  source 1..19: mol_type = other RNA
taaccaactcagctcagag

SEQ ID NO: 386 - RNA, 19 nt, synthetic construct
  source 1..19: mol_type = other RNA
tcataaaaccaactcagct

SEQ ID NO: 387 - RNA, 19 nt, synthetic construct
  source 1..19: mol_type = other RNA
ttttcataaaaccaactca

SEQ ID NO: 388 - RNA, 19 nt, synthetic construct
  source 1..19: mol_type = other RNA
tcttcctagcttttcataa

SEQ ID NO: 389 - RNA, 19 nt, synthetic construct
  source 1..19: mol_type = other RNA
tatttcccaaccagctgaa

SEQ ID NO: 390 - RNA, 19 nt, synthetic construct
  source 1..19: mol_type = other RNA
tcatttcccaaccagctga

SEQ ID NO: 391 - RNA, 19 nt, synthetic construct
  source 1..19: mol_type = other RNA
tagtcagtaagggaccctc

SEQ ID NO: 392 - RNA, 19 nt, synthetic construct
  source 1..19: mol_type = other RNA
tagtctgaccattaatagg

SEQ ID NO: 393 - RNA, 19 nt, synthetic construct
  source 1..19: mol_type = other RNA
tacagtctgaccattaata

SEQ ID NO: 394 - RNA, 19 nt, synthetic construct
  source 1..19: mol_type = other RNA
tgaacagtctgaccattaa

SEQ ID NO: 395 - RNA, 19 nt, synthetic construct
  source 1..19: mol_type = other RNA
ttggaacagtctgaccatt

SEQ ID NO: 396 - RNA, 19 nt, synthetic construct
  source 1..19: mol_type = other RNA
tagttaggtgaaaaaggtg

SEQ ID NO: 397 - RNA, 19 nt, synthetic construct
  source 1..19: mol_type = other RNA
tttagttaggtgaaaaagg

SEQ ID NO: 398 - RNA, 19 nt, synthetic construct
  source 1..19: mol_type = other RNA
tttttagttaggtgaaaaa

SEQ ID NO: 399 - RNA, 19 nt, synthetic construct
  source 1..19: mol_type = other RNA
tatgtatccacctttgtct

SEQ ID NO: 400 - RNA, 19 nt, synthetic construct
  source 1..19: mol_type = other RNA
tatcttgctcatgtatcca

SEQ ID NO: 401 - RNA, 19 nt, synthetic construct
  source 1..19: mol_type = other RNA
taatcttgctcatgtatcc

SEQ ID NO: 402 - RNA, 19 nt, synthetic construct
  source 1..19: mol_type = other RNA
tttatcctaatgggtagca

SEQ ID NO: 403 - RNA, 19 nt, synthetic construct
  source 1..19: mol_type = other RNA
ttaaaaccaactcagctca

SEQ ID NO: 404 - RNA, 19 nt, synthetic construct
  source 1..19: mol_type = other RNA
tctagcttttcataaaacc

SEQ ID NO: 405 - RNA, 21 nt, synthetic construct
  source 1..21: mol_type = other RNA
nacaagatctgagaggacnnn

SEQ ID NO: 406 - RNA, 21 nt, synthetic construct
  source 1..21: mol_type = other RNA
nttgaaggatggatggaannn

SEQ ID NO: 407 - RNA, 21 nt, synthetic construct
  source 1..21: mol_type = other RNA
nagatgagctggtggacannn

SEQ ID NO: 408 - RNA, 21 nt, synthetic construct
  source 1..21: mol_type = other RNA
ncatcagacactctggtannn

SEQ ID NO: 409 - RNA, 21 nt, synthetic construct
  source 1..21: mol_type = other RNA
ngttttccccatcagacannn

SEQ ID NO: 410 - RNA, 21 nt, synthetic construct
  source 1..21: mol_type = other RNA
nacgttttccccatcagannn

SEQ ID NO: 411 - RNA, 21 nt, synthetic construct
  source 1..21: mol_type = other RNA
naacgttttccccatcagnnn

SEQ ID NO: 412 - RNA, 21 nt, synthetic construct
  source 1..21: mol_type = other RNA
nagaacgttttccccatcnnn

SEQ ID NO: 413 - RNA, 21 nt, synthetic construct
  source 1..21: mol_type = other RNA
naccagaacgttttccccnnn

SEQ ID NO: 414 - RNA, 21 nt, synthetic construct
  source 1..21: mol_type = other RNA
ntcagacaccagaacgttnnn

SEQ ID NO: 415 - RNA, 21 nt, synthetic construct
  source 1..21: mol_type = other RNA
nagtcagacaccagaacgnnn

SEQ ID NO: 416 - RNA, 21 nt, synthetic construct
  source 1..21: mol_type = other RNA
nacgacttcgtctttggannn

SEQ ID NO: 417 - RNA, 21 nt, synthetic construct
  source 1..21: mol_type = other RNA
ncatatcgcacgcctctgnnn

SEQ ID NO: 418 - RNA, 21 nt, synthetic construct
  source 1..21: mol_type = other RNA
natgaagggtacgttgtcnnn

SEQ ID NO: 419 - RNA, 21 nt, synthetic construct
  source 1..21: mol_type = other RNA
naatgaagggtacgttgtnnn

SEQ ID NO: 420 - RNA, 21 nt, synthetic construct
  source 1..21: mol_type = other RNA
ntcaatgaagggtacgttnnn

SEQ ID NO: 421 - RNA, 21 nt, synthetic construct
  source 1..21: mol_type = other RNA
natgtcgtactccccatannn

SEQ ID NO: 422 - RNA, 21 nt, synthetic construct
  source 1..21: mol_type = other RNA
ntttagggcagatgtcgtnnn

SEQ ID NO: 423 - RNA, 21 nt, synthetic construct
  source 1..21: mol_type = other RNA
nctttagggcagatgtcgnnn

SEQ ID NO: 424 - RNA, 21 nt, synthetic construct
  source 1..21: mol_type = other RNA
ntgactttagggcagatgnnn

SEQ ID NO: 425 - RNA, 21 nt, synthetic construct
  source 1..21: mol_type = other RNA
nttgactttagggcagatnnn

SEQ ID NO: 426 - RNA, 21 nt, synthetic construct
  source 1..21: mol_type = other RNA
ncttgactttagggcagannn

SEQ ID NO: 427 - RNA, 21 nt, synthetic construct
  source 1..21: mol_type = other RNA
ngacttgactttagggcannn

SEQ ID NO: 428 - RNA, 21 nt, synthetic construct
  source 1..21: mol_type = other RNA
ntccacatgaagaaagttnnn

SEQ ID NO: 429 - RNA, 21 nt, synthetic construct
  source 1..21: mol_type = other RNA
ntgatgtccacatgaagannn

SEQ ID NO: 430 - RNA, 21 nt, synthetic construct
  source 1..21: mol_type = other RNA
ngtgatgtccacatgaagnnn

SEQ ID NO: 431 - RNA, 21 nt, synthetic construct
  source 1..21: mol_type = other RNA
nttggtgatgtccacatgnnn

SEQ ID NO: 432 - RNA, 21 nt, synthetic construct
  source 1..21: mol_type = other RNA
ntgagcttggtgatgtccnnn

SEQ ID NO: 433 - RNA, 21 nt, synthetic construct
  source 1..21: mol_type = other RNA
natatcctcgaaggcatannn

SEQ ID NO: 434 - RNA, 21 nt, synthetic construct
  source 1..21: mol_type = other RNA
naatatcctcgaaggcatnnn

SEQ ID NO: 435 - RNA, 21 nt, synthetic construct
  source 1..21: mol_type = other RNA
naaatatcctcgaaggcannn

SEQ ID NO: 436 - RNA, 21 nt, synthetic construct
  source 1..21: mol_type = other RNA
ncaaatatcctcgaaggcnnn

SEQ ID NO: 437 - RNA, 21 nt, synthetic construct
  source 1..21: mol_type = other RNA
ntcttccaagaacctgaannn

SEQ ID NO: 438 - RNA, 21 nt, synthetic construct
  source 1..21: mol_type = other RNA
ntatccacctttgtctttnnn

SEQ ID NO: 439 - RNA, 21 nt, synthetic construct
  source 1..21: mol_type = other RNA
ntgtatccacctttgtctnnn

SEQ ID NO: 440 - RNA, 21 nt, synthetic construct
  source 1..21: mol_type = other RNA
nctcatgtatccacctttnnn

SEQ ID NO: 441 - RNA, 21 nt, synthetic construct
  source 1..21: mol_type = other RNA
nttgctcatgtatccaccnnn

SEQ ID NO: 442 - RNA, 21 nt, synthetic construct
  source 1..21: mol_type = other RNA
naaatcttgctcatgtatnnn

SEQ ID NO: 443 - RNA, 21 nt, synthetic construct
  source 1..21: mol_type = other RNA
nttgcaaatcttgctcatnnn

SEQ ID NO: 444 - RNA, 21 nt, synthetic construct
  source 1..21: mol_type = other RNA
nagttgcaaatcttgctcnnn

SEQ ID NO: 445 - RNA, 21 nt, synthetic construct
  source 1..21: mol_type = other RNA
natgggtagcaagttgcannn

SEQ ID NO: 446 - RNA, 21 nt, synthetic construct
  source 1..21: mol_type = other RNA
ntaatgggtagcaagttgnnn

SEQ ID NO: 447 - RNA, 21 nt, synthetic construct
  source 1..21: mol_type = other RNA
nctaatgggtagcaagttnnn

SEQ ID NO: 448 - RNA, 21 nt, synthetic construct
  source 1..21: mol_type = other RNA
ncctaatgggtagcaagtnnn

SEQ ID NO: 449 - RNA, 21 nt, synthetic construct
  source 1..21: mol_type = other RNA
ntcctaatgggtagcaagnnn

SEQ ID NO: 450 - RNA, 21 nt, synthetic construct
  source 1..21: mol_type = other RNA
natcctaatgggtagcaannn

SEQ ID NO: 451 - RNA, 21 nt, synthetic construct
  source 1..21: mol_type = other RNA
nattatcctaatgggtagnnn

SEQ ID NO: 452 - RNA, 21 nt, synthetic construct
  source 1..21: mol_type = other RNA
nacaatcgcaatggcagannn

SEQ ID NO: 453 - RNA, 21 nt, synthetic construct
  source 1..21: mol_type = other RNA
nagtctctggacaatcgcnnn

SEQ ID NO: 454 - RNA, 21 nt, synthetic construct
  source 1..21: mol_type = other RNA
ntatctggaagccatgtcnnn

SEQ ID NO: 455 - RNA, 21 nt, synthetic construct
  source 1..21: mol_type = other RNA
natatctggaagccatgtnnn

SEQ ID NO: 456 - RNA, 21 nt, synthetic construct
  source 1..21: mol_type = other RNA
ncatatctggaagccatgnnn

SEQ ID NO: 457 - RNA, 21 nt, synthetic construct
  source 1..21: mol_type = other RNA
naggtactttattgcccannn

SEQ ID NO: 458 - RNA, 21 nt, synthetic construct
  source 1..21: mol_type = other RNA
ngaaaaactgggaaaggtnnn

SEQ ID NO: 459 - RNA, 21 nt, synthetic construct
  source 1..21: mol_type = other RNA
ngtgaaaaactgggaaagnnn

SEQ ID NO: 460 - RNA, 21 nt, synthetic construct
  source 1..21: mol_type = other RNA
ntttagcacctctgaaagnnn

SEQ ID NO: 461 - RNA, 21 nt, synthetic construct
  source 1..21: mol_type = other RNA
naaccaactcagctcagannn

SEQ ID NO: 462 - RNA, 21 nt, synthetic construct
  source 1..21: mol_type = other RNA
ncataaaaccaactcagcnnn

SEQ ID NO: 463 - RNA, 21 nt, synthetic construct
  source 1..21: mol_type = other RNA
ntttcataaaaccaactcnnn

SEQ ID NO: 464 - RNA, 21 nt, synthetic construct
  source 1..21: mol_type = other RNA
ncttcctagcttttcatannn

SEQ ID NO: 465 - RNA, 21 nt, synthetic construct
  source 1..21: mol_type = other RNA
natttcccaaccagctgannn

SEQ ID NO: 466 - RNA, 21 nt, synthetic construct
  source 1..21: mol_type = other RNA
ncatttcccaaccagctgnnn

SEQ ID NO: 467 - RNA, 21 nt, synthetic construct
  source 1..21: mol_type = other RNA
nagtcagtaagggaccctnnn

SEQ ID NO: 468 - RNA, 21 nt, synthetic construct
  source 1..21: mol_type = other RNA
nagtctgaccattaatagnnn

SEQ ID NO: 469 - RNA, 21 nt, synthetic construct
  source 1..21: mol_type = other RNA
nacagtctgaccattaatnnn

SEQ ID NO: 470 - RNA, 21 nt, synthetic construct
  source 1..21: mol_type = other RNA
ngaacagtctgaccattannn

SEQ ID NO: 471 - RNA, 21 nt, synthetic construct
  source 1..21: mol_type = other RNA
ntggaacagtctgaccatnnn

SEQ ID NO: 472 - RNA, 21 nt, synthetic construct
  source 1..21: mol_type = other RNA
nagttaggtgaaaaaggtnnn

SEQ ID NO: 473 - RNA, 21 nt, synthetic construct
  source 1..21: mol_type = other RNA
nttagttaggtgaaaaagnnn

SEQ ID NO: 474 - RNA, 21 nt, synthetic construct
  source 1..21: mol_type = other RNA
nttttagttaggtgaaaannn

SEQ ID NO: 475 - RNA, 21 nt, synthetic construct
  source 1..21: mol_type = other RNA
natgtatccacctttgtcnnn

SEQ ID NO: 476 - RNA, 21 nt, synthetic construct
  source 1..21: mol_type = other RNA
natcttgctcatgtatccnnn

SEQ ID NO: 477 - RNA, 21 nt, synthetic construct
  source 1..21: mol_type = other RNA
naatcttgctcatgtatcnnn

SEQ ID NO: 478 - RNA, 21 nt, synthetic construct
  source 1..21: mol_type = other RNA
nttatcctaatgggtagcnnn

SEQ ID NO: 479 - RNA, 21 nt, synthetic construct
  source 1..21: mol_type = other RNA
ntaaaaccaactcagctcnnn

SEQ ID NO: 480 - RNA, 21 nt, synthetic construct
  source 1..21: mol_type = other RNA
nctagcttttcataaaacnnn

SEQ ID NO: 481 - RNA, 21 nt, synthetic construct
  source 1..21: mol_type = other RNA
gaggtcctctcagatcttgta

SEQ ID NO: 482 - RNA, 21 nt, synthetic construct
  source 1..21: mol_type = other RNA
gtcttccatccatccttcaaa

SEQ ID NO: 483 - RNA, 21 nt, synthetic construct
  source 1..21: mol_type = other RNA
gaatgtccaccagctcatcta

SEQ ID NO: 484 - RNA, 21 nt, synthetic construct
  source 1..21: mol_type = other RNA
gcttaccagagtgtctgatga

SEQ ID NO: 485 - RNA, 21 nt, synthetic construct
  source 1..21: mol_type = other RNA
cagtgtctgatggggaaaaca

SEQ ID NO: 486 - RNA, 21 nt, synthetic construct
  source 1..21: mol_type = other RNA
ctgtctgatggggaaaacgta

SEQ ID NO: 487 - RNA, 21 nt, synthetic construct
  source 1..21: mol_type = other RNA
ggtctgatggggaaaacgtta

SEQ ID NO: 488 - RNA, 21 nt, synthetic construct
  source 1..21: mol_type = other RNA
gctgatggggaaaacgttcta

SEQ ID NO: 489 - RNA, 21 nt, synthetic construct
  source 1..21: mol_type = other RNA
catggggaaaacgttctggta

SEQ ID NO: 490 - RNA, 21 nt, synthetic construct
  source 1..21: mol_type = other RNA
caaaacgttctggtgtctgaa

SEQ ID NO: 491 - RNA, 21 nt, synthetic construct
  source 1..21: mol_type = other RNA
gaacgttctggtgtctgacta

SEQ ID NO: 492 - RNA, 21 nt, synthetic construct
  source 1..21: mol_type = other RNA
gggtccaaagacgaagtcgta

SEQ ID NO: 493 - RNA, 21 nt, synthetic construct
  source 1..21: mol_type = other RNA
gttcagaggcgtgcgatatga

SEQ ID NO: 494 - RNA, 21 nt, synthetic construct
  source 1..21: mol_type = other RNA
ggtgacaacgtacccttcata

SEQ ID NO: 495 - RNA, 21 nt, synthetic construct
  source 1..21: mol_type = other RNA
ctgacaacgtacccttcatta

SEQ ID NO: 496 - RNA, 21 nt, synthetic construct
  source 1..21: mol_type = other RNA
cacaacgtacccttcattgaa

SEQ ID NO: 497 - RNA, 21 nt, synthetic construct
  source 1..21: mol_type = other RNA
gtctatggggagtacgacata

SEQ ID NO: 498 - RNA, 21 nt, synthetic construct
  source 1..21: mol_type = other RNA
ggtacgacatctgccctaaaa

SEQ ID NO: 499 - RNA, 21 nt, synthetic construct
  source 1..21: mol_type = other RNA
ctacgacatctgccctaaaga

SEQ ID NO: 500 - RNA, 21 nt, synthetic construct
  source 1..21: mol_type = other RNA
ggacatctgccctaaagtcaa

SEQ ID NO: 501 - RNA, 21 nt, synthetic construct
  source 1..21: mol_type = other RNA
cacatctgccctaaagtcaaa

SEQ ID NO: 502 - RNA, 21 nt, synthetic construct
  source 1..21: mol_type = other RNA
gcatctgccctaaagtcaaga

SEQ ID NO: 503 - RNA, 21 nt, synthetic construct
  source 1..21: mol_type = other RNA
gtctgccctaaagtcaagtca

SEQ ID NO: 504 - RNA, 21 nt, synthetic construct
  source 1..21: mol_type = other RNA
gcgaactttcttcatgtggaa

SEQ ID NO: 505 - RNA, 21 nt, synthetic construct
  source 1..21: mol_type = other RNA
gtttcttcatgtggacatcaa

SEQ ID NO: 506 - RNA, 21 nt, synthetic construct
  source 1..21: mol_type = other RNA
gttcttcatgtggacatcaca

SEQ ID NO: 507 - RNA, 21 nt, synthetic construct
  source 1..21: mol_type = other RNA
gttcatgtggacatcaccaaa

SEQ ID NO: 508 - RNA, 21 nt, synthetic construct
  source 1..21: mol_type = other RNA
ggtggacatcaccaagctcaa

SEQ ID NO: 509 - RNA, 21 nt, synthetic construct
  source 1..21: mol_type = other RNA
ggatatgccttcgaggatata

SEQ ID NO: 510 - RNA, 21 nt, synthetic construct
  source 1..21: mol_type = other RNA
catatgccttcgaggatatta

SEQ ID NO: 511 - RNA, 21 nt, synthetic construct
  source 1..21: mol_type = other RNA
gtatgccttcgaggatattta

SEQ ID NO: 512 - RNA, 21 nt, synthetic construct
  source 1..21: mol_type = other RNA
gatgccttcgaggatatttga

SEQ ID NO: 513 - RNA, 21 nt, synthetic construct
  source 1..21: mol_type = other RNA
ccattcaggttcttggaagaa

SEQ ID NO: 514 - RNA, 21 nt, synthetic construct
  source 1..21: mol_type = other RNA
gtgaaagacaaaggtggataa

SEQ ID NO: 515 - RNA, 21 nt, synthetic construct
  source 1..21: mol_type = other RNA
caaagacaaaggtggatacaa

SEQ ID NO: 516 - RNA, 21 nt, synthetic construct
  source 1..21: mol_type = other RNA
cacaaaggtggatacatgaga

SEQ ID NO: 517 - RNA, 21 nt, synthetic construct
  source 1..21: mol_type = other RNA
gaaggtggatacatgagcaaa

SEQ ID NO: 518 - RNA, 21 nt, synthetic construct
  source 1..21: mol_type = other RNA
gggatacatgagcaagattta

SEQ ID NO: 519 - RNA, 21 nt, synthetic construct
  source 1..21: mol_type = other RNA
gacatgagcaagatttgcaaa

SEQ ID NO: 520 - RNA, 21 nt, synthetic construct
  source 1..21: mol_type = other RNA
gatgagcaagatttgcaacta

SEQ ID NO: 521 - RNA, 21 nt, synthetic construct
  source 1..21: mol_type = other RNA
gtttgcaacttgctacccata

SEQ ID NO: 522 - RNA, 21 nt, synthetic construct
  source 1..21: mol_type = other RNA
gtgcaacttgctacccattaa

SEQ ID NO: 523 - RNA, 21 nt, synthetic construct
  source 1..21: mol_type = other RNA
ggcaacttgctacccattaga

SEQ ID NO: 524 - RNA, 21 nt, synthetic construct
  source 1..21: mol_type = other RNA
ccaacttgctacccattagga

SEQ ID NO: 525 - RNA, 21 nt, synthetic construct
  source 1..21: mol_type = other RNA
gaacttgctacccattaggaa

SEQ ID NO: 526 - RNA, 21 nt, synthetic construct
  source 1..21: mol_type = other RNA
gacttgctacccattaggata

SEQ ID NO: 527 - RNA, 21 nt, synthetic construct
  source 1..21: mol_type = other RNA
gtgctacccattaggataata

SEQ ID NO: 528 - RNA, 21 nt, synthetic construct
  source 1..21: mol_type = other RNA
caatctgccattgcgattgta

SEQ ID NO: 529 - RNA, 21 nt, synthetic construct
  source 1..21: mol_type = other RNA
gttgcgattgtccagagacta

SEQ ID NO: 530 - RNA, 21 nt, synthetic construct
  source 1..21: mol_type = other RNA
cgtgacatggcttccagataa

SEQ ID NO: 531 - RNA, 21 nt, synthetic construct
  source 1..21: mol_type = other RNA
ctgacatggcttccagatata

SEQ ID NO: 532 - RNA, 21 nt, synthetic construct
  source 1..21: mol_type = other RNA
ggacatggcttccagatatga

SEQ ID NO: 533 - RNA, 21 nt, synthetic construct
  source 1..21: mol_type = other RNA
gcttgggcaataaagtaccta

SEQ ID NO: 534 - RNA, 21 nt, synthetic construct
  source 1..21: mol_type = other RNA
gccacctttcccagtttttca

SEQ ID NO: 535 - RNA, 21 nt, synthetic construct
  source 1..21: mol_type = other RNA
gacctttcccagtttttcaca

SEQ ID NO: 536 - RNA, 21 nt, synthetic construct
  source 1..21: mol_type = other RNA
gttctttcagaggtgctaaaa

SEQ ID NO: 537 - RNA, 21 nt, synthetic construct
  source 1..21: mol_type = other RNA
ccctctgagctgagttggtta

SEQ ID NO: 538 - RNA, 21 nt, synthetic construct
  source 1..21: mol_type = other RNA
ggagctgagttggttttatga

SEQ ID NO: 539 - RNA, 21 nt, synthetic construct
  source 1..21: mol_type = other RNA
cctgagttggttttatgaaaa

SEQ ID NO: 540 - RNA, 21 nt, synthetic construct
  source 1..21: mol_type = other RNA
gtttatgaaaagctaggaaga

SEQ ID NO: 541 - RNA, 21 nt, synthetic construct
  source 1..21: mol_type = other RNA
gattcagctggttgggaaata

SEQ ID NO: 542 - RNA, 21 nt, synthetic construct
  source 1..21: mol_type = other RNA
gttcagctggttgggaaatga

SEQ ID NO: 543 - RNA, 21 nt, synthetic construct
  source 1..21: mol_type = other RNA
gagagggtcccttactgacta

SEQ ID NO: 544 - RNA, 21 nt, synthetic construct
  source 1..21: mol_type = other RNA
cccctattaatggtcagacta

SEQ ID NO: 545 - RNA, 21 nt, synthetic construct
  source 1..21: mol_type = other RNA
gctattaatggtcagactgta

SEQ ID NO: 546 - RNA, 21 nt, synthetic construct
  source 1..21: mol_type = other RNA
gattaatggtcagactgttca

SEQ ID NO: 547 - RNA, 21 nt, synthetic construct
  source 1..21: mol_type = other RNA
gtaatggtcagactgttccaa

SEQ ID NO: 548 - RNA, 21 nt, synthetic construct
  source 1..21: mol_type = other RNA
gacacctttttcacctaacta

SEQ ID NO: 549 - RNA, 21 nt, synthetic construct
  source 1..21: mol_type = other RNA
gacctttttcacctaactaaa

SEQ ID NO: 550 - RNA, 21 nt, synthetic construct
  source 1..21: mol_type = other RNA
gctttttcacctaactaaaaa

SEQ ID NO: 551 - RNA, 21 nt, synthetic construct
  source 1..21: mol_type = other RNA
gaagacaaaggtggatacata

SEQ ID NO: 552 - RNA, 21 nt, synthetic construct
  source 1..21: mol_type = other RNA
cacaaaggtggatacatgaga

SEQ ID NO: 553 - RNA, 21 nt, synthetic construct
  source 1..21: mol_type = other RNA
gaaggtggatacatgagcaaa

SEQ ID NO: 554 - RNA, 21 nt, synthetic construct
  source 1..21: mol_type = other RNA
cgtggatacatgagcaagata

SEQ ID NO: 555 - RNA, 21 nt, synthetic construct
  source 1..21: mol_type = other RNA
ctggatacatgagcaagatta

SEQ ID NO: 556 - RNA, 21 nt, synthetic construct
  source 1..21: mol_type = other RNA
gtttgcaacttgctacccata

SEQ ID NO: 557 - RNA, 21 nt, synthetic construct
  source 1..21: mol_type = other RNA
gtgcaacttgctacccattaa

SEQ ID NO: 558 - RNA, 21 nt, synthetic construct
  source 1..21: mol_type = other RNA
ggcaacttgctacccattaga

SEQ ID NO: 559 - RNA, 21 nt, synthetic construct
  source 1..21: mol_type = other RNA
ccaacttgctacccattagga

SEQ ID NO: 560 - RNA, 21 nt, synthetic construct
  source 1..21: mol_type = other RNA
gttgctacccattaggataaa

SEQ ID NO: 561 - RNA, 21 nt, synthetic construct
  source 1..21: mol_type = other RNA
gtgctacccattaggataata

SEQ ID NO: 562 - RNA, 21 nt, synthetic construct
  source 1..21: mol_type = other RNA
gctgagctgagttggttttaa

SEQ ID NO: 563 - RNA, 21 nt, synthetic construct
  source 1..21: mol_type = other RNA
ggagctgagttggttttatga

SEQ ID NO: 564 - RNA, 21 nt, synthetic construct
  source 1..21: mol_type = other RNA
gtggttttatgaaaagctaga

SEQ ID NO: 565 - RNA, 21 nt, synthetic construct
  source 1..21: mol_type = other RNA
tacaagatctgagaggacctc

SEQ ID NO: 566 - RNA, 21 nt, synthetic construct
  source 1..21: mol_type = other RNA
tttgaaggatggatggaagac

SEQ ID NO: 567 - RNA, 21 nt, synthetic construct
  source 1..21: mol_type = other RNA
tagatgagctggtggacattc

SEQ ID NO: 568 - RNA, 21 nt, synthetic construct
  source 1..21: mol_type = other RNA
tcatcagacactctggtaagc

SEQ ID NO: 569 - RNA, 21 nt, synthetic construct
  source 1..21: mol_type = other RNA
tgttttccccatcagacactg

SEQ ID NO: 570 - RNA, 21 nt, synthetic construct
  source 1..21: mol_type = other RNA
tacgttttccccatcagacag

SEQ ID NO: 571 - RNA, 21 nt, synthetic construct
  source 1..21: mol_type = other RNA
taacgttttccccatcagacc

SEQ ID NO: 572 - RNA, 21 nt, synthetic construct
  source 1..21: mol_type = other RNA
tagaacgttttccccatcagc

SEQ ID NO: 573 - RNA, 21 nt, synthetic construct
  source 1..21: mol_type = other RNA
taccagaacgttttccccatg

SEQ ID NO: 574 - RNA, 21 nt, synthetic construct
  source 1..21: mol_type = other RNA
ttcagacaccagaacgttttg

SEQ ID NO: 575 - RNA, 21 nt, synthetic construct
  source 1..21: mol_type = other RNA
tagtcagacaccagaacgttc

SEQ ID NO: 576 - RNA, 21 nt, synthetic construct
  source 1..21: mol_type = other RNA
tacgacttcgtctttggaccc

SEQ ID NO: 577 - RNA, 21 nt, synthetic construct
  source 1..21: mol_type = other RNA
tcatatcgcacgcctctgaac

SEQ ID NO: 578 - RNA, 21 nt, synthetic construct
  source 1..21: mol_type = other RNA
tatgaagggtacgttgtcacc

SEQ ID NO: 579 - RNA, 21 nt, synthetic construct
  source 1..21: mol_type = other RNA
taatgaagggtacgttgtcag

SEQ ID NO: 580 - RNA, 21 nt, synthetic construct
  source 1..21: mol_type = other RNA
ttcaatgaagggtacgttgtg

SEQ ID NO: 581 - RNA, 21 nt, synthetic construct
  source 1..21: mol_type = other RNA
tatgtcgtactccccatagac

SEQ ID NO: 582 - RNA, 21 nt, synthetic construct
  source 1..21: mol_type = other RNA
ttttagggcagatgtcgtacc

SEQ ID NO: 583 - RNA, 21 nt, synthetic construct
  source 1..21: mol_type = other RNA
tctttagggcagatgtcgtag

SEQ ID NO: 584 - RNA, 21 nt, synthetic construct
  source 1..21: mol_type = other RNA
ttgactttagggcagatgtcc

SEQ ID NO: 585 - RNA, 21 nt, synthetic construct
  source 1..21: mol_type = other RNA
tttgactttagggcagatgtg

SEQ ID NO: 586 - RNA, 21 nt, synthetic construct
  source 1..21: mol_type = other RNA
tcttgactttagggcagatgc

SEQ ID NO: 587 - RNA, 21 nt, synthetic construct
  source 1..21: mol_type = other RNA
tgacttgactttagggcagac

SEQ ID NO: 588 - RNA, 21 nt, synthetic construct
  source 1..21: mol_type = other RNA
ttccacatgaagaaagttcgc

SEQ ID NO: 589 - RNA, 21 nt, synthetic construct
  source 1..21: mol_type = other RNA
ttgatgtccacatgaagaaac

SEQ ID NO: 590 - RNA, 21 nt, synthetic construct
  source 1..21: mol_type = other RNA
tgtgatgtccacatgaagaac

SEQ ID NO: 591 - RNA, 21 nt, synthetic construct
  source 1..21: mol_type = other RNA
tttggtgatgtccacatgaac

SEQ ID NO: 592 - RNA, 21 nt, synthetic construct
  source 1..21: mol_type = other RNA
ttgagcttggtgatgtccacc

SEQ ID NO: 593 - RNA, 21 nt, synthetic construct
  source 1..21: mol_type = other RNA
tatatcctcgaaggcatatcc

SEQ ID NO: 594 - RNA, 21 nt, synthetic construct
  source 1..21: mol_type = other RNA
taatatcctcgaaggcatatg

SEQ ID NO: 595 - RNA, 21 nt, synthetic construct
  source 1..21: mol_type = other RNA
taaatatcctcgaaggcatac

SEQ ID NO: 596 - RNA, 21 nt, synthetic construct
  source 1..21: mol_type = other RNA
tcaaatatcctcgaaggcatc

SEQ ID NO: 597 - RNA, 21 nt, synthetic construct
  source 1..21: mol_type = other RNA
ttcttccaagaacctgaatgg

SEQ ID NO: 598 - RNA, 21 nt, synthetic construct
  source 1..21: mol_type = other RNA
ttatccacctttgtctttcac

SEQ ID NO: 599 - RNA, 21 nt, synthetic construct
  source 1..21: mol_type = other RNA
ttgtatccacctttgtctttg

SEQ ID NO: 600 - RNA, 21 nt, synthetic construct
  source 1..21: mol_type = other RNA
tctcatgtatccacctttgtg

SEQ ID NO: 601 - RNA, 21 nt, synthetic construct
  source 1..21: mol_type = other RNA
tttgctcatgtatccaccttc

SEQ ID NO: 602 - RNA, 21 nt, synthetic construct
  source 1..21: mol_type = other RNA
taaatcttgctcatgtatccc

SEQ ID NO: 603 - RNA, 21 nt, synthetic construct
  source 1..21: mol_type = other RNA
tttgcaaatcttgctcatgtc

SEQ ID NO: 604 - RNA, 21 nt, synthetic construct
  source 1..21: mol_type = other RNA
tagttgcaaatcttgctcatc

SEQ ID NO: 605 - RNA, 21 nt, synthetic construct
  source 1..21: mol_type = other RNA
tatgggtagcaagttgcaaac

SEQ ID NO: 606 - RNA, 21 nt, synthetic construct
  source 1..21: mol_type = other RNA
ttaatgggtagcaagttgcac

SEQ ID NO: 607 - RNA, 21 nt, synthetic construct
  source 1..21: mol_type = other RNA
tctaatgggtagcaagttgcc

SEQ ID NO: 608 - RNA, 21 nt, synthetic construct
  source 1..21: mol_type = other RNA
tcctaatgggtagcaagttgg

SEQ ID NO: 609 - RNA, 21 nt, synthetic construct
  source 1..21: mol_type = other RNA
ttcctaatgggtagcaagttc

SEQ ID NO: 610 - RNA, 21 nt, synthetic construct
  source 1..21: mol_type = other RNA
tatcctaatgggtagcaagtc

SEQ ID NO: 611 - RNA, 21 nt, synthetic construct
  source 1..21: mol_type = other RNA
tattatcctaatgggtagcac

SEQ ID NO: 612 - RNA, 21 nt, synthetic construct
  source 1..21: mol_type = other RNA
tacaatcgcaatggcagattg

SEQ ID NO: 613 - RNA, 21 nt, synthetic construct
  source 1..21: mol_type = other RNA
tagtctctggacaatcgcaac

SEQ ID NO: 614 - RNA, 21 nt, synthetic construct
  source 1..21: mol_type = other RNA
ttatctggaagccatgtcacg

SEQ ID NO: 615 - RNA, 21 nt, synthetic construct
  source 1..21: mol_type = other RNA
tatatctggaagccatgtcag

SEQ ID NO: 616 - RNA, 21 nt, synthetic construct
  source 1..21: mol_type = other RNA
tcatatctggaagccatgtcc

SEQ ID NO: 617 - RNA, 21 nt, synthetic construct
  source 1..21: mol_type = other RNA
taggtactttattgcccaagc

SEQ ID NO: 618 - RNA, 21 nt, synthetic construct
  source 1..21: mol_type = other RNA
tgaaaaactgggaaaggtggc

SEQ ID NO: 619 - RNA, 21 nt, synthetic construct
  source 1..21: mol_type = other RNA
tgtgaaaaactgggaaaggtc

SEQ ID NO: 620 - RNA, 21 nt, synthetic construct
  source 1..21: mol_type = other RNA
ttttagcacctctgaaagaac

SEQ ID NO: 621 - RNA, 21 nt, synthetic construct
  source 1..21: mol_type = other RNA
taaccaactcagctcagaggg

SEQ ID NO: 622 - RNA, 21 nt, synthetic construct
  source 1..21: mol_type = other RNA
tcataaaaccaactcagctcc

SEQ ID NO: 623 - RNA, 21 nt, synthetic construct
  source 1..21: mol_type = other RNA
ttttcataaaaccaactcagg

SEQ ID NO: 624 - RNA, 21 nt, synthetic construct
  source 1..21: mol_type = other RNA
tcttcctagcttttcataaac

SEQ ID NO: 625 - RNA, 21 nt, synthetic construct
  source 1..21: mol_type = other RNA
tatttcccaaccagctgaatc

SEQ ID NO: 626 - RNA, 21 nt, synthetic construct
  source 1..21: mol_type = other RNA
tcatttcccaaccagctgaac

SEQ ID NO: 627 - RNA, 21 nt, synthetic construct
  source 1..21: mol_type = other RNA
tagtcagtaagggaccctctc

SEQ ID NO: 628 - RNA, 21 nt, synthetic construct
  source 1..21: mol_type = other RNA
tagtctgaccattaatagggg

SEQ ID NO: 629 - RNA, 21 nt, synthetic construct
  source 1..21: mol_type = other RNA
tacagtctgaccattaatagc

SEQ ID NO: 630 - RNA, 21 nt, synthetic construct
  source 1..21: mol_type = other RNA
tgaacagtctgaccattaatc

SEQ ID NO: 631 - RNA, 21 nt, synthetic construct
  source 1..21: mol_type = other RNA
ttggaacagtctgaccattac

SEQ ID NO: 632 - RNA, 21 nt, synthetic construct
  source 1..21: mol_type = other RNA
tagttaggtgaaaaaggtgtc

SEQ ID NO: 633 - RNA, 21 nt, synthetic construct
  source 1..21: mol_type = other RNA
tttagttaggtgaaaaaggtc

SEQ ID NO: 634 - RNA, 21 nt, synthetic construct
  source 1..21: mol_type = other RNA
tttttagttaggtgaaaaagc

SEQ ID NO: 635 - RNA, 21 nt, synthetic construct
  source 1..21: mol_type = other RNA
tatgtatccacctttgtcttc

SEQ ID NO: 636 - RNA, 21 nt, synthetic construct
  source 1..21: mol_type = other RNA
tctcatgtatccacctttgtg

SEQ ID NO: 637 - RNA, 21 nt, synthetic construct
  source 1..21: mol_type = other RNA
tttgctcatgtatccaccttc

SEQ ID NO: 638 - RNA, 21 nt, synthetic construct
  source 1..21: mol_type = other RNA
tatcttgctcatgtatccacg

SEQ ID NO: 639 - RNA, 21 nt, synthetic construct
  source 1..21: mol_type = other RNA
taatcttgctcatgtatccag

SEQ ID NO: 640 - RNA, 21 nt, synthetic construct
  source 1..21: mol_type = other RNA
tatgggtagcaagttgcaaac

SEQ ID NO: 641 - RNA, 21 nt, synthetic construct
  source 1..21: mol_type = other RNA
ttaatgggtagcaagttgcac

SEQ ID NO: 642 - RNA, 21 nt, synthetic construct
  source 1..21: mol_type = other RNA
tctaatgggtagcaagttgcc

SEQ ID NO: 643 - RNA, 21 nt, synthetic construct
  source 1..21: mol_type = other RNA
tcctaatgggtagcaagttgg

SEQ ID NO: 644 - RNA, 21 nt, synthetic construct
  source 1..21: mol_type = other RNA
tttatcctaatgggtagcaac

SEQ ID NO: 645 - RNA, 21 nt, synthetic construct
  source 1..21: mol_type = other RNA
tattatcctaatgggtagcac

SEQ ID NO: 646 - RNA, 21 nt, synthetic construct
  source 1..21: mol_type = other RNA
ttaaaaccaactcagctcagc

SEQ ID NO: 647 - RNA, 21 nt, synthetic construct
  source 1..21: mol_type = other RNA
tcataaaaccaactcagctcc

SEQ ID NO: 648 - RNA, 21 nt, synthetic construct
  source 1..21: mol_type = other RNA
tctagcttttcataaaaccac

SEQ ID NO: 649 - RNA, 21 nt, synthetic construct
  source 1..21: mol_type = other RNA
acacctttttcacctaactaa

SEQ ID NO: 650 - RNA, 21 nt, synthetic construct
  source 1..21: mol_type = other RNA
gcttaccagagtgtctgatga

SEQ ID NO: 651 - RNA, 21 nt, synthetic construct
  source 1..21: mol_type = other RNA
ctgtctgatggggaaaacgta

SEQ ID NO: 652 - RNA, 21 nt, synthetic construct
  source 1..21: mol_type = other RNA
ggtacgacatctgccctaaaa

SEQ ID NO: 653 - RNA, 21 nt, synthetic construct
  source 1..21: mol_type = other RNA
cacatctgccctaaagtcaaa

SEQ ID NO: 654 - RNA, 21 nt, synthetic construct
  source 1..21: mol_type = other RNA
caaagacaaaggtggatacaa

SEQ ID NO: 655 - RNA, 21 nt, synthetic construct
  source 1..21: mol_type = other RNA
cgtgacatggcttccagataa

SEQ ID NO: 656 - RNA, 21 nt, synthetic construct
  source 1..21: mol_type = other RNA
ctgacatggcttccagatata

SEQ ID NO: 657 - RNA, 21 nt, synthetic construct
  source 1..21: mol_type = other RNA
gattcagctggttgggaaata

SEQ ID NO: 658 - RNA, 21 nt, synthetic construct
  source 1..21: mol_type = other RNA
gttcagctggttgggaaatga

SEQ ID NO: 659 - RNA, 21 nt, synthetic construct
  source 1..21: mol_type = other RNA
cccctattaatggtcagacta

SEQ ID NO: 660 - RNA, 21 nt, synthetic construct
  source 1..21: mol_type = other RNA
ggccttatccctccttcctta

SEQ ID NO: 661 - RNA, 21 nt, synthetic construct
  source 1..21: mol_type = other RNA
gcgaactttcttcatgtggaa

SEQ ID NO: 662 - RNA, 21 nt, synthetic construct
  source 1..21: mol_type = other RNA
gtgaaagacaaaggtggataa

SEQ ID NO: 663 - RNA, 21 nt, synthetic construct
  source 1..21: mol_type = other RNA
gggatacatgagcaagattta

SEQ ID NO: 664 - RNA, 21 nt, synthetic construct
  source 1..21: mol_type = other RNA
gacatgagcaagatttgcaaa

SEQ ID NO: 665 - RNA, 21 nt, synthetic construct
  source 1..21: mol_type = other RNA
gatgagcaagatttgcaacta

SEQ ID NO: 666 - RNA, 21 nt, synthetic construct
  source 1..21: mol_type = other RNA
gacttgctacccattaggata

SEQ ID NO: 667 - RNA, 21 nt, synthetic construct
  source 1..21: mol_type = other RNA
ggacatggcttccagatatga

SEQ ID NO: 668 - RNA, 21 nt, synthetic construct
  source 1..21: mol_type = other RNA
gccacctttcccagtttttca

SEQ ID NO: 669 - RNA, 21 nt, synthetic construct
  source 1..21: mol_type = other RNA
ccctctgagctgagttggtta

SEQ ID NO: 670 - RNA, 21 nt, synthetic construct
  source 1..21: mol_type = other RNA
cctgagttggttttatgaaaa

SEQ ID NO: 671 - RNA, 21 nt, synthetic construct
  source 1..21: mol_type = other RNA
gagagggtcccttactgacta

SEQ ID NO: 672 - RNA, 21 nt, synthetic construct
  source 1..21: mol_type = other RNA
gacacctttttcacctaacta

SEQ ID NO: 673 - RNA, 21 nt, synthetic construct
  source 1..21: mol_type = other RNA
gacctttttcacctaactaaa

SEQ ID NO: 674 - RNA, 21 nt, synthetic construct
  source 1..21: mol_type = other RNA
gaggtcctctcagatcttgta

SEQ ID NO: 675 - RNA, 21 nt, synthetic construct
  source 1..21: mol_type = other RNA
gtcttccatccatccttcaaa

SEQ ID NO: 676 - RNA, 21 nt, synthetic construct
  source 1..21: mol_type = other RNA
catggggaaaacgttctggta

SEQ ID NO: 677 - RNA, 21 nt, synthetic construct
  source 1..21: mol_type = other RNA
gggtccaaagacgaagtcgta

SEQ ID NO: 678 - RNA, 21 nt, synthetic construct
  source 1..21: mol_type = other RNA
ggtgacaacgtacccttcata

SEQ ID NO: 679 - RNA, 21 nt, synthetic construct
  source 1..21: mol_type = other RNA
cacaacgtacccttcattgaa

SEQ ID NO: 680 - RNA, 21 nt, synthetic construct
  source 1..21: mol_type = other RNA
gaagacaaaggtggatacata

SEQ ID NO: 681 - RNA, 21 nt, synthetic construct
  source 1..21: mol_type = other RNA
gtgcaacttgctacccattaa

SEQ ID NO: 682 - RNA, 21 nt, synthetic construct
  source 1..21: mol_type = other RNA
ggagctgagttggttttatga

SEQ ID NO: 683 - RNA, 21 nt, synthetic construct
  source 1..21: mol_type = other RNA
gttgctacccattaggataaa

SEQ ID NO: 684 - RNA, 21 nt, synthetic construct
  source 1..21: mol_type = other RNA
gtggttttatgaaaagctaga

SEQ ID NO: 685 - RNA, 21 nt, synthetic construct
  source 1..21: mol_type = other RNA
gctgagctgagttggttttaa

SEQ ID NO: 686 - RNA, 21 nt, synthetic construct
  source 1..21: mol_type = other RNA
ttagttaggtgaaaaaggtgt

SEQ ID NO: 687 - RNA, 21 nt, synthetic construct
  source 1..21: mol_type = other RNA
tcatcagacactctggtaagc

SEQ ID NO: 688 - RNA, 21 nt, synthetic construct
  source 1..21: mol_type = other RNA
tacgttttccccatcagacag

SEQ ID NO: 689 - RNA, 21 nt, synthetic construct
  source 1..21: mol_type = other RNA
ttttagggcagatgtcgtacc

SEQ ID NO: 690 - RNA, 21 nt, synthetic construct
  source 1..21: mol_type = other RNA
tttgactttagggcagatgtg

SEQ ID NO: 691 - RNA, 21 nt, synthetic construct
  source 1..21: mol_type = other RNA
ttgtatccacctttgtctttg

SEQ ID NO: 692 - RNA, 21 nt, synthetic construct
  source 1..21: mol_type = other RNA
ttatctggaagccatgtcacg

SEQ ID NO: 693 - RNA, 21 nt, synthetic construct
  source 1..21: mol_type = other RNA
tatatctggaagccatgtcag

SEQ ID NO: 694 - RNA, 21 nt, synthetic construct
  source 1..21: mol_type = other RNA
tatttcccaaccagctgaatc

SEQ ID NO: 695 - RNA, 21 nt, synthetic construct
  source 1..21: mol_type = other RNA
tcatttcccaaccagctgaac

SEQ ID NO: 696 - RNA, 21 nt, synthetic construct
  source 1..21: mol_type = other RNA
tagtctgaccattaatagggg

SEQ ID NO: 697 - RNA, 23 nt, synthetic construct
  source 1..23: mol_type = other RNA
taaggaaggagggataaggccac

SEQ ID NO: 698 - RNA, 21 nt, synthetic construct
  source 1..21: mol_type = other RNA
ttccacatgaagaaagttcgc

SEQ ID NO: 699 - RNA, 21 nt, synthetic construct
  source 1..21: mol_type = other RNA
ttatccacctttgtctttcac

SEQ ID NO: 700 - RNA, 21 nt, synthetic construct
  source 1..21: mol_type = other RNA
taaatcttgctcatgtatccc

SEQ ID NO: 701 - RNA, 21 nt, synthetic construct
  source 1..21: mol_type = other RNA
tttgcaaatcttgctcatgtc

SEQ ID NO: 702 - RNA, 21 nt, synthetic construct
  source 1..21: mol_type = other RNA
tagttgcaaatcttgctcatc

SEQ ID NO: 703 - RNA, 21 nt, synthetic construct
  source 1..21: mol_type = other RNA
tatcctaatgggtagcaagtc

SEQ ID NO: 704 - RNA, 21 nt, synthetic construct
  source 1..21: mol_type = other RNA
tcatatctggaagccatgtcc

SEQ ID NO: 705 - RNA, 21 nt, synthetic construct
  source 1..21: mol_type = other RNA
tgaaaaactgggaaaggtggc

SEQ ID NO: 706 - RNA, 21 nt, synthetic construct
  source 1..21: mol_type = other RNA
taaccaactcagctcagaggg

SEQ ID NO: 707 - RNA, 21 nt, synthetic construct
  source 1..21: mol_type = other RNA
ttttcataaaaccaactcagg

SEQ ID NO: 708 - RNA, 21 nt, synthetic construct
  source 1..21: mol_type = other RNA
tagtcagtaagggaccctctc

SEQ ID NO: 709 - RNA, 21 nt, synthetic construct
  source 1..21: mol_type = other RNA
tagttaggtgaaaaaggtgtc

SEQ ID NO: 710 - RNA, 21 nt, synthetic construct
  source 1..21: mol_type = other RNA
tttagttaggtgaaaaaggtc

SEQ ID NO: 711 - RNA, 21 nt, synthetic construct
  source 1..21: mol_type = other RNA
tacaagatctgagaggacctc

SEQ ID NO: 712 - RNA, 21 nt, synthetic construct
  source 1..21: mol_type = other RNA
tttgaaggatggatggaagac

SEQ ID NO: 713 - RNA, 21 nt, synthetic construct
  source 1..21: mol_type = other RNA
taccagaacgttttccccatg

SEQ ID NO: 714 - RNA, 21 nt, synthetic construct
  source 1..21: mol_type = other RNA
tacgacttcgtctttggaccc

SEQ ID NO: 715 - RNA, 21 nt, synthetic construct
  source 1..21: mol_type = other RNA
tatgaagggtacgttgtcacc

SEQ ID NO: 716 - RNA, 21 nt, synthetic construct
  source 1..21: mol_type = other RNA
ttcaatgaagggtacgttgtg

SEQ ID NO: 717 - RNA, 21 nt, synthetic construct
  source 1..21: mol_type = other RNA
tatgtatccacctttgtcttc

SEQ ID NO: 718 - RNA, 21 nt, synthetic construct
  source 1..21: mol_type = other RNA
ttaatgggtagcaagttgcac

SEQ ID NO: 719 - RNA, 21 nt, synthetic construct
  source 1..21: mol_type = other RNA
tcataaaaccaactcagctcc

SEQ ID NO: 720 - RNA, 21 nt, synthetic construct
  source 1..21: mol_type = other RNA
tttatcctaatgggtagcaac

SEQ ID NO: 721 - RNA, 21 nt, synthetic construct
  source 1..21: mol_type = other RNA
tctagcttttcataaaaccac

SEQ ID NO: 722 - RNA, 21 nt, synthetic construct
  source 1..21: mol_type = other RNA
ttaaaaccaactcagctcagc

SEQ ID NO: 723 - RNA, 21 nt, synthetic construct
  source 1..21: mol_type = other RNA
gacacctttttcacctaacta

SEQ ID NO: 724 - RNA, 21 nt, synthetic construct
  source 1..21: mol_type = other RNA
gacctttttcacctaactaaa

SEQ ID NO: 725 - RNA, 21 nt, synthetic construct
  source 1..21: mol_type = other RNA
gaagacaaaggtggatacata

SEQ ID NO: 726 - RNA, 21 nt, synthetic construct
  source 1..21: mol_type = other RNA
gtgcaacttgctacccattaa

SEQ ID NO: 727 - RNA, 21 nt, synthetic construct
  source 1..21: mol_type = other RNA
gtggttttatgaaaagctaga

SEQ ID NO: 728 - RNA, 21 nt, synthetic construct
  source 1..21: mol_type = other RNA
acacctttttcacctaactaa

SEQ ID NO: 729 - RNA, 21 nt, synthetic construct
  source 1..21: mol_type = other RNA
tagttaggtgaaaaaggtgtc

SEQ ID NO: 730 - RNA, 21 nt, synthetic construct
  source 1..21: mol_type = other RNA
tttagttaggtgaaaaaggtc

SEQ ID NO: 731 - RNA, 21 nt, synthetic construct
  source 1..21: mol_type = other RNA
tatgtatccacctttgtcttc

SEQ ID NO: 732 - RNA, 21 nt, synthetic construct
  source 1..21: mol_type = other RNA
ttaatgggtagcaagttgcac

SEQ ID NO: 733 - RNA, 21 nt, synthetic construct
  source 1..21: mol_type = other RNA
tctagcttttcataaaaccac

SEQ ID NO: 734 - RNA, 21 nt, synthetic construct
  source 1..21: mol_type = other RNA
ttagttaggtgaaaaaggtgt

SEQ ID NO: 735 - RNA, 21 nt, synthetic construct
  source 1..21: mol_type = other RNA
gacacctttttcacctaacta

SEQ ID NO: 736 - RNA, 21 nt, synthetic construct
  source 1..21: mol_type = other RNA
gacctttttcacctaactaaa

SEQ ID NO: 737 - RNA, 21 nt, synthetic construct
  source 1..21: mol_type = other RNA
gaagacaaaggtggatacata

SEQ ID NO: 738 - RNA, 21 nt, synthetic construct
  source 1..21: mol_type = other RNA
acacctttttcacctaactaa

SEQ ID NO: 739 - RNA, 21 nt, synthetic construct
  source 1..21: mol_type = other RNA
acacctttttcacctaactaa

SEQ ID NO: 740 - RNA, 21 nt, synthetic construct
  source 1..21: mol_type = other RNA
tagttaggtgaaaaaggtgtc

SEQ ID NO: 741 - RNA, 21 nt, synthetic construct
  source 1..21: mol_type = other RNA
tttagttaggtgaaaaaggtc

SEQ ID NO: 742 - RNA, 21 nt, synthetic construct
  source 1..21: mol_type = other RNA
tatgtatccacctttgtcttc

SEQ ID NO: 743 - RNA, 21 nt, synthetic construct
  source 1..21: mol_type = other RNA
ttagttaggtgaaaaaggtgt

SEQ ID NO: 744 - RNA, 21 nt, synthetic construct
  source 1..21: mol_type = other RNA
ttagttaggtgaaaaaggtgt

(19) United States
(12) Patent Application Publication
(10) Pub. No.: US 2026/0201387 A1
(43) Pub. Date: Jul. 16, 2026

(54) COMPOSITIONS AND METHODS FOR INHIBITING EXPRESSION OF PATATIN-LIKE PHOSPHOLIPASE DOMAIN-CONTAINING 3 (PNPLA3)

(71) Applicant: SHANGHAI ARGO BIOPHARMACEUTICAL CO., LTD., Shanghai (CN)

(72) Inventors: Dongxu Shu, Ningbo (CN); Shiwei Xia, Hangzhou (CN)

(21) Appl. No.: 19/135,601

(22) PCT Filed: Dec. 6, 2023
(86) PCT No.: PCT/CN2023/136623

§ 371 Date: Jun. 4, 2025

(30) Foreign Application Priority Data
Dec. 7, 2022 (WO) PCT/CN2022/137217

(51) Int. Cl. C12N 15/113; A61K 31/7115; A61K 31/712; A61K 31/7125

(52) U.S. Cl. CPC C12N 15/1137; A61K 31/7115; A61K 31/712; A61K 31/7125; C12N 2310/14; C12N 2310/312; C12N 2310/315; C12N 2310/321; C12N 2310/322; C12N 2310/332; C12N 2310/345; C12N 2310/346; C12N 2310/351

(57) ABSTRACT

Compositions and methods useful to reduce expression of Patatin-like Phospholipase Domain Containing 3 (PNPLA3) gene and for treatment of PNPLA3-associated diseases and conditions are provided. Provided are PNPLA3 dsRNA agents, PNPLA3 antisense polynucleotide agents, compositions comprising PNPLA3 dsRNA agents, and compositions comprising PNPLA3 antisense polynucleotide agents that can be used to reduce PNPLA3 expression in cells and subjects.

### FIELD OF THE INVENTION

The invention relates, in part, to compositions and methods that can be used to inhibit Patatin-Like Phospholipase Domain Containing 3 (PNPLA3) gene expression.

### BACKGROUND

Patatin-like phospholipase domain-containing 3 (PNPLA3), a type II transmembrane protein is expressed in various cells including in the liver. In hepatocytes, PNPLA3 is expressed on the endoplasmic reticulum and lipid membranes and predominantly exhibits triacylglycerol hydrolase activity.

The accumulation of excess triglyceride in the liver is known as hepatic steatosis (or fatty liver), and is associated with adverse metabolic consequences, including insulin resistance and dyslipidemia. NAFLD refers to a wide spectrum of liver diseases that can progress from simple fatty liver (steatosis), to nonalcoholic steatohepatitis (NASH), to cirrhosis (irreversible, advanced scarring of the liver). All of the stages of NAFLD have in common the accumulation of fat (fatty infiltration) in the liver cells (hepatocytes).

Many studies have identified a significant association between hepatic fat content and the Patatin-like Phospholipase Domain Containing 3 (PNPLA3) gene (see, for example, Romeo et al. (2008) Nat. Genet., 40(12): 1461-1465). Studies with knock-in mice have demonstrated that expression of a sequence polymorphism (rs738409, 1148M) in PNPLA3 causes NAFLD, and that the accumulation of catalytically inactive PNPLA3 on the surfaces of lipid droplets is associated with the accumulation of triglycerides in the liver (Smagris el al. (2015) Hepatology, 61:108-118). Specifically, the PNPLA3 I148M variant was associated with promoting the development of fibrogenesis by activating the hedgehog (Hh) signaling pathway, leading to the activation and proliferation of hepatic stellate cells and excessive generation and deposition of extracellular matrix (Chen et al. (2015) World J. Gastroenterol., 21(3):794-802).

Currently, there is a need for therapies for subjects suffering from NAFLD. The present invention represents a novel approach to reducing PNPLA3 levels and treating hepatologic diseases, such as NAFLD.

### SUMMARY OF THE INVENTION

According to an aspect of the invention, a double-stranded ribonucleic acid (dsRNA) agent for inhibiting expression of Patatin-like Phospholipase Domain Containing 3 (PNPLA3) is provided, wherein the dsRNA agent includes a sense strand and an antisense strand, wherein the sense strand comprises at least 15 contiguous nucleotides differing by no more than 3 nucleotides from the nucleotide sequence of SEQ ID NO: 1 and the antisense strand comprises at least 15 contiguous nucleotides differing by no more than 3 nucleotides from the nucleotide sequence of SEQ ID NO:2. In some embodiments, the dsRNA agent including a sense strand and an antisense strand, nucleotide positions 2 to 18 in the antisense strand including a region of complementarity to a PNPLA3 RNA transcript, wherein the region of complementarity includes at least 15 contiguous nucleotides that differ by 0, 1, 2, or 3 nucleotides from one of the antisense sequences listed in one of Tables 1-3, and optionally including a targeting ligand. In some embodiments, the region of complementarity to a PNPLA3 RNA transcript includes at least 15, 16, 17, 18, or 19 contiguous nucleotides that differ by no more than 3 nucleotides from one of the antisense sequences listed in one of Tables 1-3. In certain embodiments, the antisense strand of dsRNA is at least substantially complementary to any one of a target region of SEQ ID NO: 1 and is provided in any one of Tables 1-3. In some embodiments, the antisense strand of dsRNA is fully complementary to any one of a target region of SEQ ID NO: 1 and is provided in any one of Tables 1-3. In some embodiments, the dsRNA agent includes a sense strand sequence set forth in any one of Tables 1-3, wherein the sense strand sequence is at least substantially complementary to the antisense strand sequence in the dsRNA agent. In certain embodiments, the dsRNA agent includes a sense strand sequence set forth in any one of Tables 1-3, wherein the sense strand sequence is fully complementary to the antisense strand sequence in the dsRNA agent. In some embodiments, the dsRNA agent includes an antisense strand sequence set forth in any one of Tables 1-3. In some embodiments, the dsRNA agent includes the sequences set forth as a duplex sequence in any of Tables 1-3. In some embodiments, the dsRNA agent includes at least one modified nucleotide. In certain embodiments, all or substantially all of the nucleotides of the antisense strand are modified nucleotides. In certain embodiments, all or substantially all of the nucleotides of the sense strand and the antisense strand are modified nucleotides. In some embodiments, the at least one modified nucleotide comprises: a 2′-O-methyl nucleotide, 2′-fluoro nucleotide, 2′-deoxy nucleotide, 2′, 3′-seco nucleotide mimic, locked nucleotide, unlocked nucleic acid nucleotide (UNA), glycol nucleic acid nucleotide (GNA), 2′-F-Arabino nucleotide, 2′-methoyxyethyl nucleotide, abasic nucleotide, ribitol, inverted nucleotide, inverted abasic nucleotide, inverted 2′-OMe nucleotide, inverted 2′-deoxy nucleotide, isomannide nucleotide, 2′-amino-modified nucleotide, 2′-alkyl-modified nucleotide, mopholino nucleotide, and 3′-OMe nucleotide, a nucleotide including a 5′-phosphorothioate group, a 5′-phosphonate modified nucleotide, a nucleotide comprising vinyl phosphonate, or a terminal nucleotide linked to a cholesteryl derivative or dodecanoic acid bisdecylamide group, a 2′-amino-modified nucleotide, a phosphoramidite, or a non-natural base including nucleotide. In some embodiments, the dsRNA agent includes an E-vinylphosphonate nucleotide at the 5′ end of the guide strand. In certain embodiments, the dsRNA agent includes at least one phosphorothioate internucleoside linkage. In certain embodiments, the sense strand includes at least one phosphorothioate internucleoside linkage. In some embodiments, the antisense strand includes at least one phosphorothioate internucleoside linkage. In some embodiments, the sense strand includes 1, 2, 3, 4, 5, or 6, phosphorothioate internucleoside linkages. In some embodiments, the antisense strand includes 1, 2, 3, 4, 5, or 6, phosphorothioate internucleoside linkages. In some embodiments, the antisense strand comprises 15 or more modified nucleotides independently selected from a 2′-O-methyl nucleotide and a 2′-fluoro nucleotide, wherein less than 6 modified nucleotides are 2′-fluoro nucleotides. In certain embodiments, the antisense strand comprises 3 or 5 2′-fluoro nucleotides, preferably, the antisense strand comprises 5 2′-fluoro nucleotides. In certain embodiments, the antisense strand comprises 5 2′-fluoro nucleotides and a 5′-phosphonate modified nucleotide, preferably, wherein the 5′-phosphonate modified nucleotide is a nucleotide comprising vinyl phosphonate. In some embodiments, the sense strand comprises 15 or more modified nucleotides independently selected from a 2′-O-methyl nucleotide and a 2′-fluoro nucleotide, wherein less than 4 modified nucleotides are 2′-fluoro nucleotides. In certain embodiments, the sense strand comprises 3 2′-fluoro nucleotides. In some embodiments, the antisense strand comprises 15 or more modified nucleotides independently selected from a 2′-O-methyl nucleotide and a 2′-fluoro nucleotide, wherein at least 16 modified nucleotides are 2′-O-methyl nucleotide and the nucleotides at position 2, 5, 7, 12, 14, 16 and/or 18 from the 5′ end of the antisense strand are a 2′-fluoro nucleotide. In certain embodiments, the nucleotides at position 2, 7, 12, 14 and 16 from the 5′ end of the antisense strand are 2′-fluoro nucleotides. In certain embodiments, the nucleotides at position 2, 5, 12, 14 and 18 from the 5′ end of the antisense strand are 2′-fluoro nucleotides. In certain embodiments, the nucleotides at position 2, 7, 12, 14 and 16 from the 5′ end of the antisense strand are 2′-fluoro nucleotides and 5′ terminal nucleotide of the antisense strand is a nucleotide comprising vinyl phosphonate, preferably, wherein said nucleotide comprising vinyl phosphonate is VPu* as defined in this invention. In some embodiments, the sense strand comprises 15 or more modified nucleotides independently selected from a 2′-O-methyl nucleotide and a 2′-fluoro nucleotide, wherein at least 18 modified nucleotides are 2′-O-methyl nucleotide and the nucleotide at position 9, 11, 13 and/or 14 from the 5′ end of the sense strand are 2′-fluoro nucleotide. In certain embodiments, the nucleotides at position 9, 11 and 13 counting from the first matching position from the 3′ end of the sense strand are 2′-fluoro nucleotides. In certain embodiments, the nucleotides at position 8, 11 and 13 counting from the first matching position from the 3′ end of the sense strand are 2′-fluoro nucleotides. In some embodiments, the sense strand sequence may be represented by formula (I):

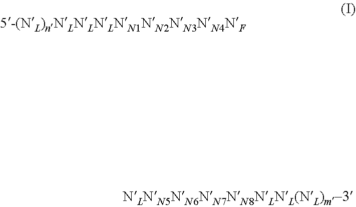

wherein:
each N′_{F} represents a 2′-fluoro-modified nucleotide; each of N′_{N1}, N′_{N2}, N′_{N3}, N′_{N4}, N′_{N5}, N′_{N6}, N′_{N7} and N′_{N8} independently represents a modified or unmodified nucleotide; each N′_{L} independently represents a modified or unmodified nucleotide but not a 2′-fluoro-modified nucleotide, and m′ and n′ are each independently an integer of 0 to 7.

In some embodiments, the antisense strand sequence may be represented by formula (II):

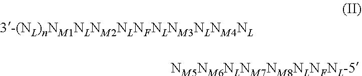

wherein:
each N_{F} represents a 2′-fluoro-modified nucleotide; each of N_{M1}, N_{M2}, N_{M3}, N_{M4}, N_{M5}, N_{M6}, N_{M7} and N_{M8} independently represents a modified or unmodified nucleotide, preferably, N_{M1}, N_{M2}, N_{M3}, N_{M6} and N_{M7} each independently represents a 2′-fluoro-modified nucleotide; each N_{L} independently represents a modified or unmodified nucleotide but not a 2′-fluoro-modified nucleotide, and n is an integer of 0 to 7.

In certain embodiment, n′ is 1 and m′ is 1, or n′ is 1 and m′ is 2, or n′ is 1 and m′ is 3, or n′ is 1 and m′ is 4, or n′ is 1 and m′ is 5, or n′ is 3 and m′ is 1, or n′ is 3 and m′ is 2, or n′ is 3 and m′ is 3, or n′ is 5 and m′ is 1.

In certain embodiment, n is 1, or n is 2, or n is 3.

In certain embodiment, the modified nucleotide is a modified nucleotide defined above.

In certain embodiment, the modified nucleotide is a 2′-OMe modified nucleotide or a 2′-F modified nucleotide.

In certain embodiment, N_{M6}, N_{M3} and N_{M2} each independently represents a 2′-fluoro-modified nucleotide, optionally, N_{M6}, N_{M3} and N_{M2} are all 2′-fluoro-modified nucleotides.

In certain embodiment, N_{M7}, N_{M3} and N_{M1} each independently represents a 2′-fluoro-modified nucleotide, optionally, N_{M7}, N_{M3} and N_{M1} are all 2′-fluoro-modified nucleotides.

In some embodiments, the antisense strand sequence may be represented by formula (II′):

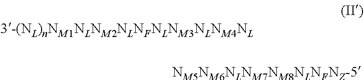

wherein:
each N_{F} represents a 2′-fluoro-modified nucleotide; each of N_{M1}, N_{M2}, N_{M3}, N_{M4}, N_{M5}, N_{M6}, N_{M7} and N_{M8} independently represents a modified or unmodified nucleotide, preferably, N_{M1}, N_{M2}, N_{M3}, N_{M6} and N_{M7} each independently represents a 2′-fluoro-modified nucleotide; each N_{L} independently represents a modified or unmodified nucleotide but not a 2′-fluoro-modified nucleotide; N_{Z} represents a nucleotide comprising phosphate mimic, preferably, N_{Z} represents a nucleotide comprising vinyl phosphonate; and n is an integer of 0 to 7.

In certain embodiment, n is 1, or n is 2, or n is 3.

In some embodiments, the modified nucleotide is a modified nucleotide defined above.

In some embodiments, the modified nucleotide is a 2′-OMe modified nucleotide or a 2′-F modified nucleotide.

In certain embodiment, N_{M6}, N_{M3} and N_{M2} each independently represents a 2′-fluoro-modified nucleotide, optionally, N_{M6}, N_{M3} and N_{M2} are all 2′-fluoro-modified nucleotides.

In certain embodiment, N_{M7}, N_{M3} and NMI each independently represents a 2′-fluoro-modified nucleotide, optionally, N_{M7}, N_{M3} and N_{M1} are all 2′-fluoro-modified nucleotides.

In certain embodiment, N_{Z} is a vinyl phosphonate modified nucleotide.

In certain embodiment, N_{Z} is VPu*, which has the structure

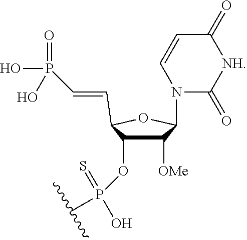

In some embodiments, the sense strand and the antisense strand form a dsRNA duplex,
wherein said sense strand complementary to the antisense strand, wherein said antisense strand comprises a region of complementarity to an mRNA encoding PNPLA3, wherein the region of complementarity comprises at least 15 contiguous nucleotides, the dsRNA duplex represented by formula (III):

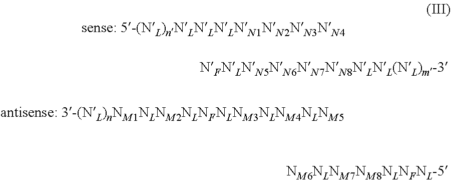

wherein:
each N_{F} and N′_{F} independently represents a 2′-fluoro-modified nucleotide; each of N_{M1}, N_{M2}, N_{M3}, N_{M4}, N_{M5}, N_{M6}, N_{M7}, N_{M8}, N′_{N1}, N′_{N2}, N′_{N3}, N′_{N4}, N′_{N5}, N′_{N6}, N′_{N7} and N′_{N8} each independently represents a modified or unmodified nucleotide; each N_{L} and N′_{L} independently represents a modified or unmodified nucleotide but not a 2′-fluoro-modified nucleotide, and m′, n′ and n are each independently an integer of 0 to 7.

In certain embodiment, n′ is 1 and m′ is 1, or n′ is 1 and m′ is 2, or n′ is 1 and m′ is 3, or n′ is 1 and m′ is 4, or n′ is 1 and m′ is 5, or n′ is 3 and m′ is 1, or n′ is 3 and m′ is 2, or n′ is 3 and m′ is 3, or n′ is 5 and m′ is 1.

In certain embodiment, n is 1, or n is 2, or n is 3.

In certain embodiment, the modified nucleotide is a modified nucleotide defined above.

In certain embodiment, the modified nucleotide is a 2′-OMe modified nucleotide or a 2′-F modified nucleotide.

In certain embodiment, N_{M6}, N_{M3} and N_{M2} each independently represents a 2′-fluoro-modified nucleotide, optionally, N_{M6}, N_{M3} and N_{M2} are all 2′-fluoro-modified nucleotides.

In certain embodiment, N_{M7}, N_{M3} and NMI each independently represents a 2′-fluoro-modified nucleotide, optionally, N_{M7}, N_{M3} and N_{M1} are all 2′-fluoro-modified nucleotides.

In some embodiments, the sense strand and the antisense strand form a dsRNA duplex,
wherein said sense strand complementary to the antisense strand, wherein said antisense strand comprises a region of complementarity to an mRNA encoding PNPLA3, wherein the region of complementarity comprises at least 15 contiguous nucleotides, wherein the dsRNA duplex may be represented by formula (III′):

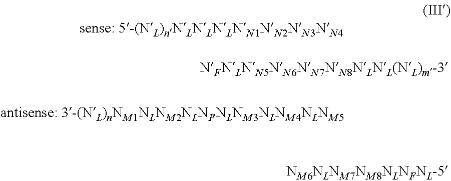

wherein:
each N_{F} and N′_{F} independently represents a 2′-fluoro-modified nucleotide; each of N_{M1}, N_{M2}, N_{M3}, N_{M4}, N_{M5}, N_{M6}, N_{M7}, N_{M8}, N′_{N1}, N′_{N2}, N′_{N3}, N′_{N4}, N′_{N5}, N′_{N6}, N′_{N7} and N′_{N8} each independently represents a modified or unmodified nucleotide; each N_{L} and N′_{L} independently represents a modified or unmodified nucleotide but not a 2′-fluoro-modified nucleotide; N_{Z} represents a nucleotide comprising phosphate mimic, preferably, N_{Z} represents a nucleotide comprising vinyl phosphonate; and m′, n′ and n are each independently an integer of 0 to 7.

In certain embodiment, n′ is 1 and m′ is 1, or n′ is 1 and m′ is 2, or n′ is 1 and m′ is 3, or n′ is 1 and m′ is 4, or n′ is 1 and m′ is 5, or n′ is 3 and m′ is 1, or n′ is 3 and m′ is 2, or n′ is 3 and m′ is 3, or n′ is 5 and m′ is 1.

In certain embodiment, n is 1, or n is 2, or n is 3.

In certain embodiment, the modified nucleotide is a modified nucleotide defined above.

In certain embodiment, the modified nucleotide is a 2′-OMe modified nucleotide or a 2′-F modified nucleotide.

In certain embodiment, N_{M6}, N_{M3} and N_{M2} each independently represents a 2′-fluoro-modified nucleotide, optionally, N_{M6}, N_{M3} and N_{M2} are all 2′-fluoro-modified nucleotides.

In certain embodiment, N_{M7}, N_{M3} and N_{M1} each independently represents a 2′-fluoro-modified nucleotide, optionally, N_{M7}, N_{M3} and N_{M1} are all 2′-fluoro-modified nucleotides.

In certain embodiment, N_{Z} is a vinyl phosphonate modified nucleotide.

In certain embodiment, N_{Z} is VPu*, which has the structure

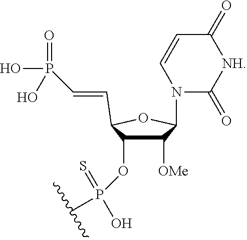

In certain embodiments, the sense strand is complementary or substantially complementary to the antisense strand, and the region of complementarity is between 16 and 23 nucleotides in length. In some embodiments, the region of complementarity is 19-21 nucleotides in length. In certain embodiments, the region of complementarity is 14, 15, 16, 17, 18, 19, 20, 21, 22, 23, 24, 25, 26, 27, 28, 29, or 30 nucleotides in length. In some embodiments, each strand is no more than 40 nucleotides in length. In some embodiments, each strand is no more than 30 nucleotides in length. In some embodiments, each strand is no more than 25 nucleotides in length. In some embodiments, each strand is no more than 23 nucleotides in length. In some embodiments, each strand is no more than 21 nucleotides in length. In certain embodiments, the dsRNA agent includes at least one modified nucleotide and further includes one or more targeting groups or linking groups. In some embodiments, the one or more targeting groups or linking groups are conjugated to the sense strand. In some embodiments, the targeting group or linking group includes N-acetyl-galactosamine (GalNAc).

In some embodiments, the targeting group has a structure as Formula (X):

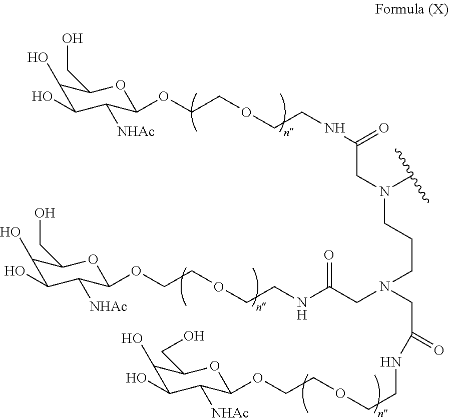

Each n″ is independently selected from 1 or 2.

In some embodiments, the targeting group has a structure:

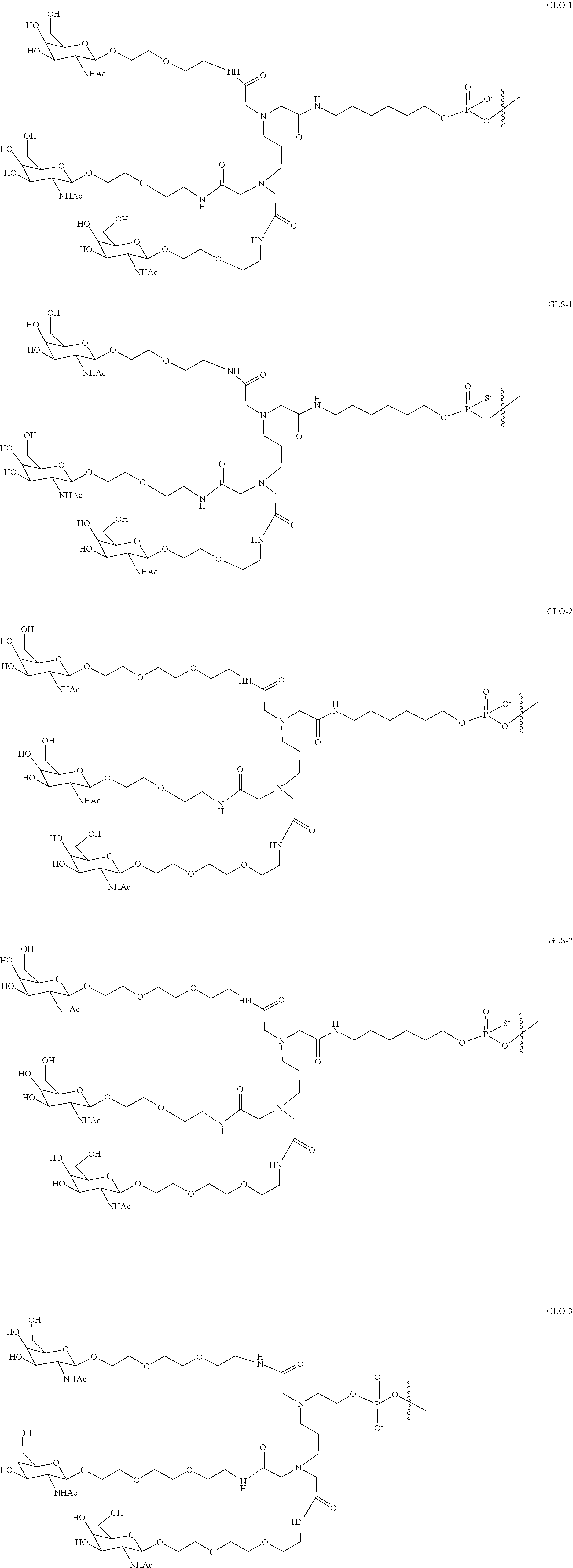

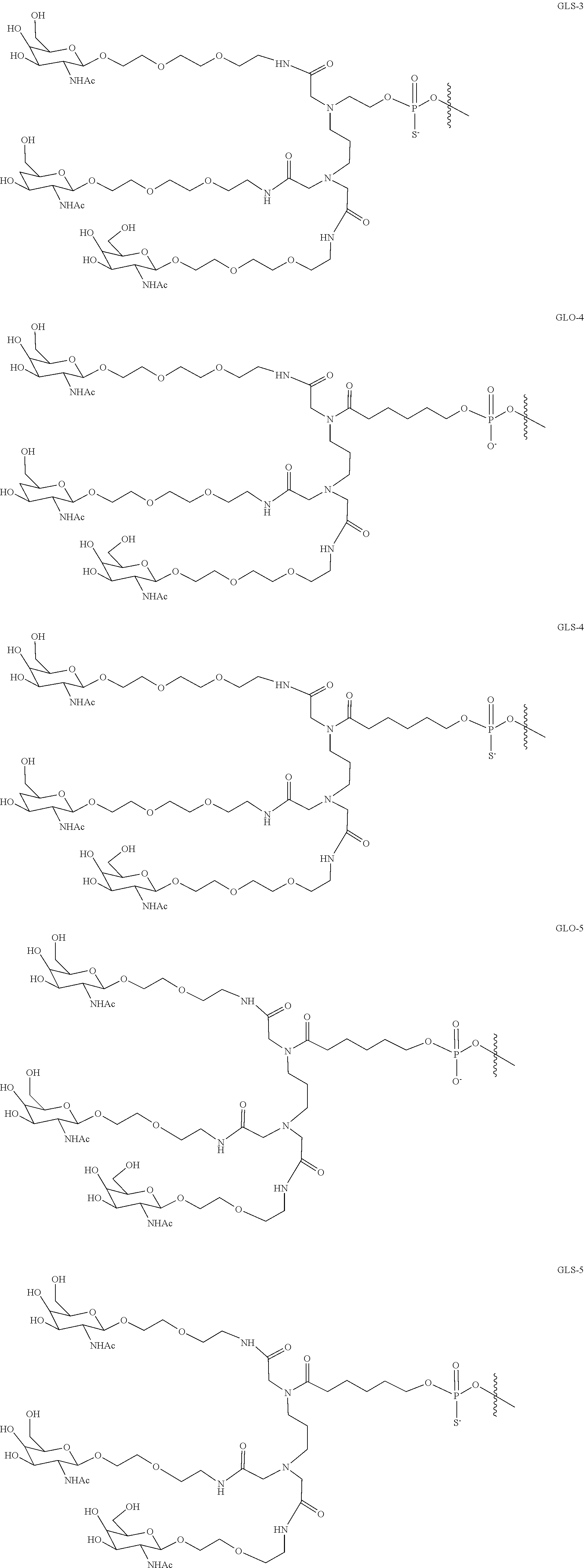

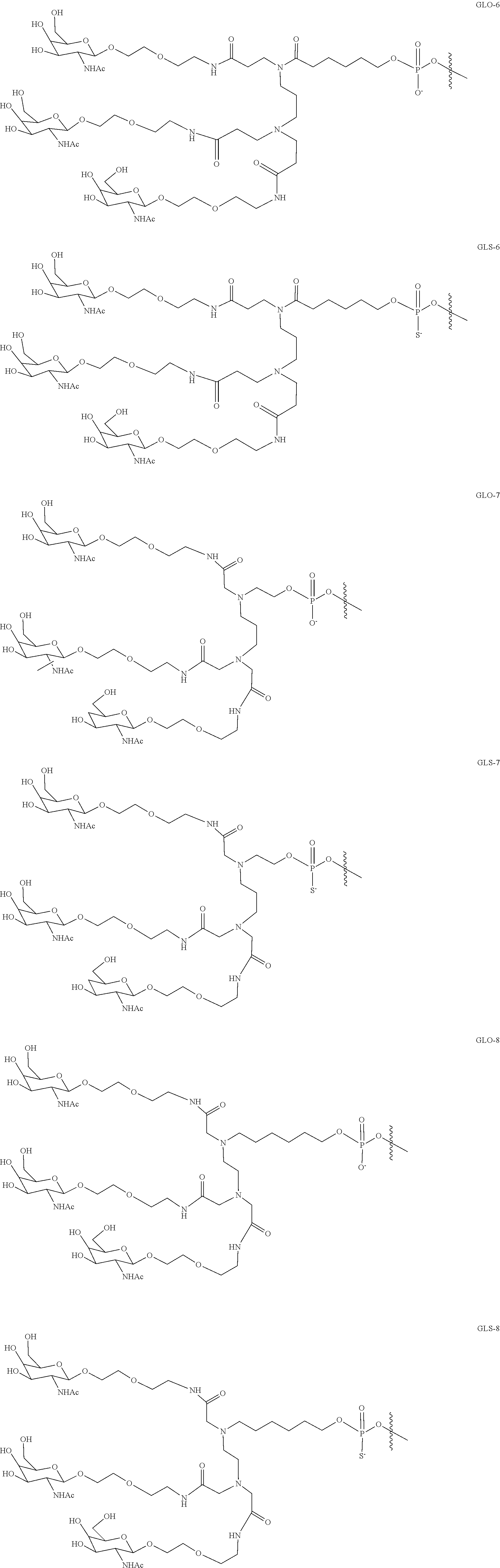

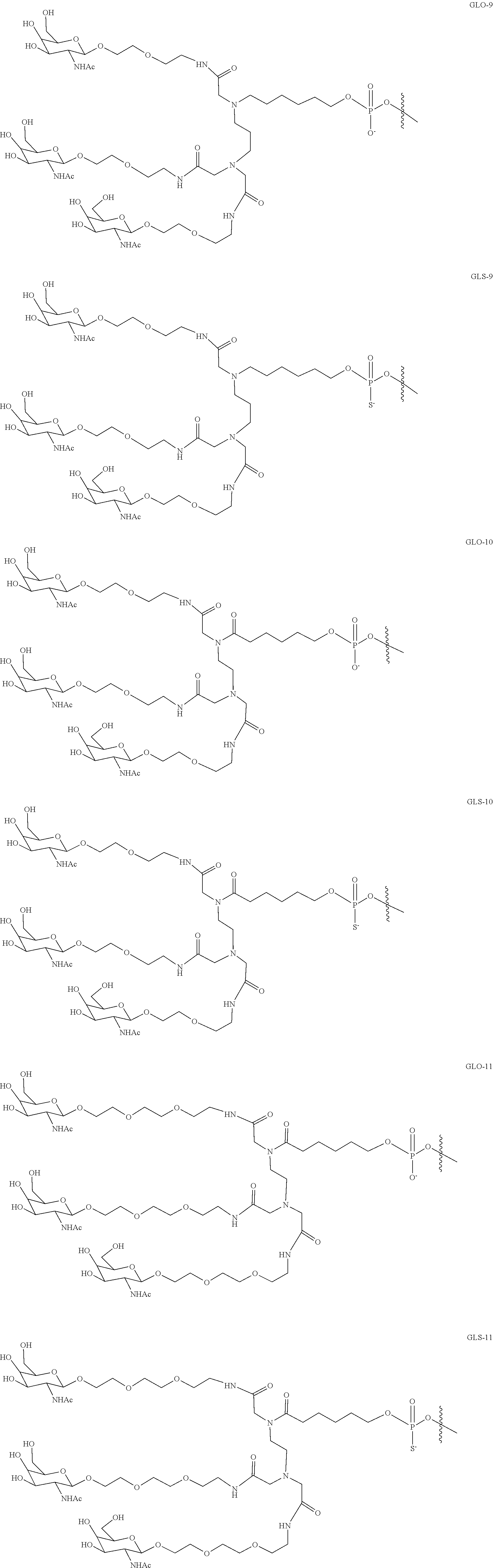

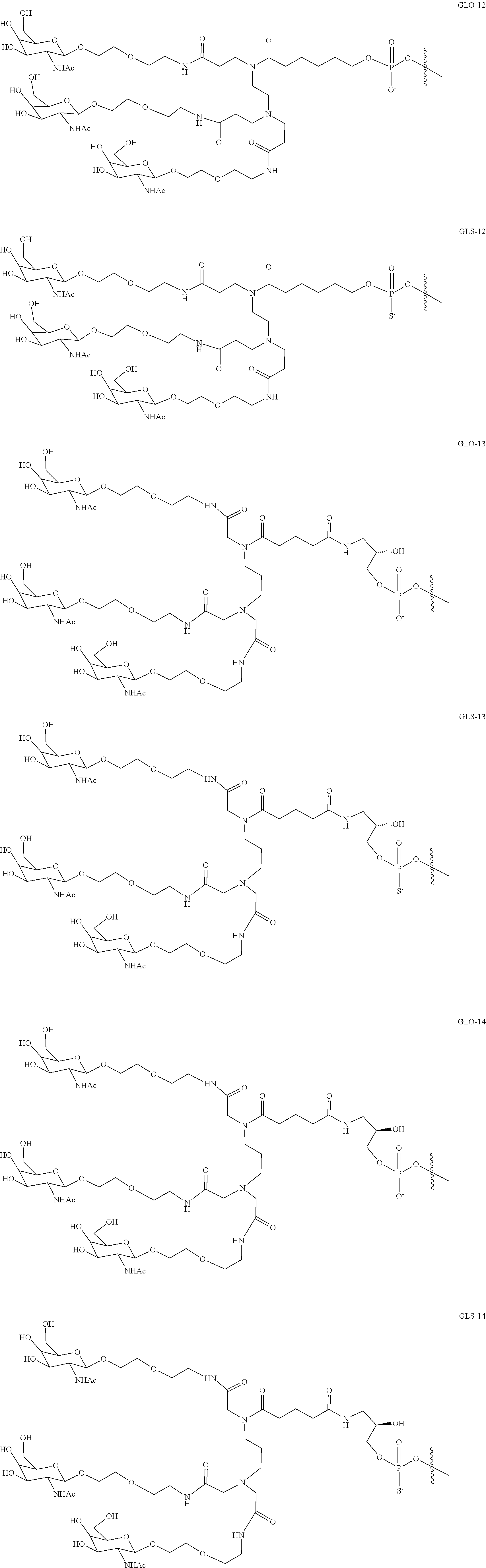

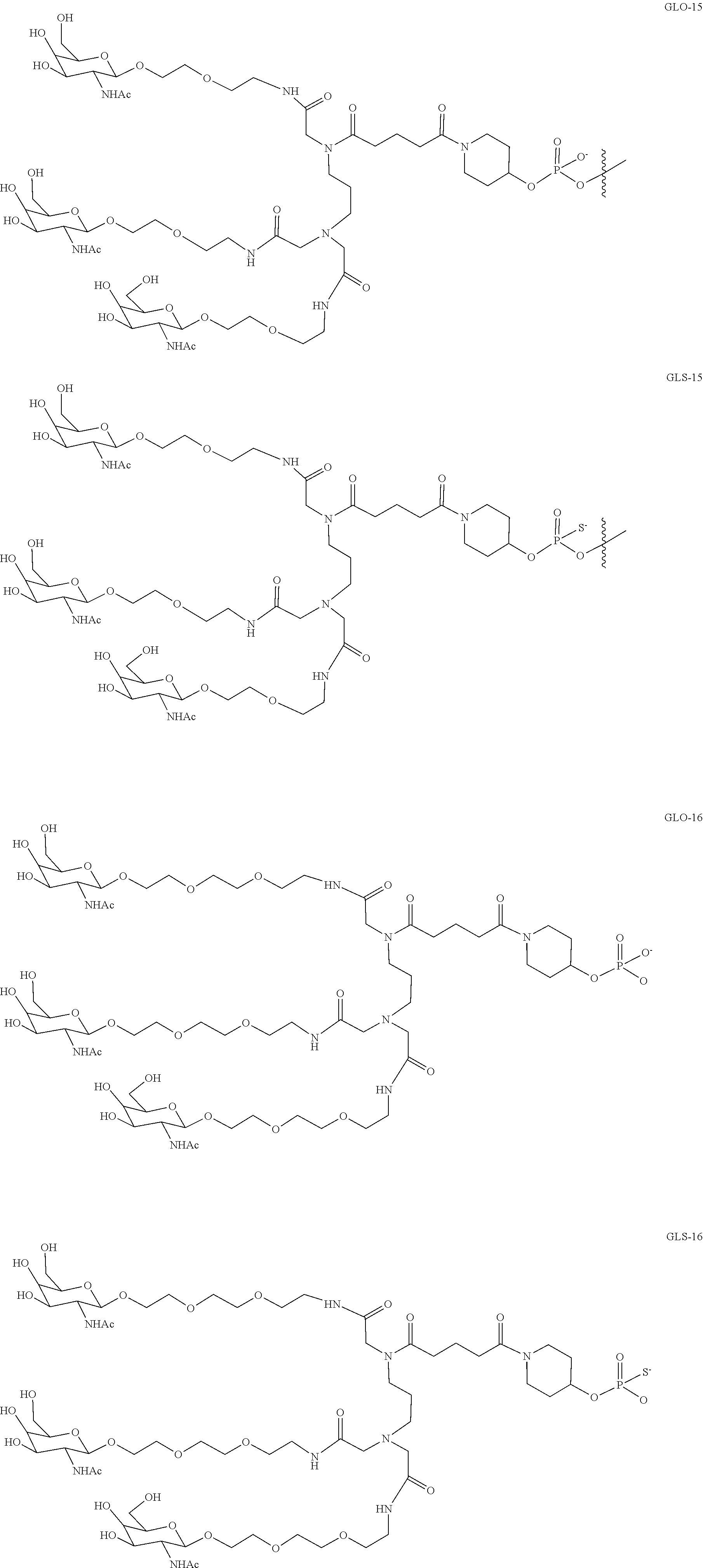

In certain embodiments, the dsRNA agent includes a targeting group that is conjugated to the 5′-terminal end of the sense strand. In some embodiments, the dsRNA agent includes a targeting group that is conjugated to the 3′-terminal end of the sense strand. In some embodiments, the antisense strand includes one inverted abasic residue at 3′-terminal end. In certain embodiments, the sense strand includes one or two inverted abasic residues at 3′ or/and 5′ terminal end. In certain embodiments, the sense strand includes one or two imann residues at 3′ or/and 5′ terminal end. In certain embodiments, each end of the sense strand includes one inverted abasic residue respectively. In certain embodiments, each end of the sense strand includes one imann residue respectively. In some embodiments, the dsRNA agent has two blunt ends. In some embodiments, at least one strand includes a 3′ overhang of at least 1 nucleotide. In some embodiments, at least one strand includes a 3′ overhang of at least 2 nucleotides. In some embodiments, at least one linkage of the sense strand and/or the antisense strand is a phosphodiester (PO) linkage. In some embodiments, at least one linkage of the sense strand and/or the antisense strand is a modified linkage. In some embodiments, at least one linkage of the sense strand and/or the antisense strand is a phosphorothioate (PS) linkage. In some embodiments, at least one phosphorothioate (PS) linkage is introduced at the 5′-end, 3′-end or both ends of the sense strand and/or the antisense strand. In some embodiments, 1, 2, 3, 4, 5, or 6 phosphorothioate (PS) linkages are introduced at the 5′-end, 3′-end or both ends of the sense strand and/or the antisense strand. In some embodiments, at least the terminal two modified or unmodified nucleotides at one end or both ends of the antisense strand are linked through phosphorothioate linkages. In some embodiments, the terminal three modified or unmodified nucleotides at one end or both ends of the antisense strand are linked through phosphorothioate linkages. In some embodiments, at least the terminal two modified or unmodified nucleotides at one end or both ends of the sense strand are linked through phosphorothioate linkages. In some embodiments, the terminal three modified or unmodified nucleotides at one end or both ends of the sense strand are linked through phosphorothioate linkages. In some embodiments, the terminal three modified or unmodified nucleotides at 5′ end of the sense strand are linked through phosphorothioate linkages and the terminal two modified or unmodified nucleotides at 3′ end of the sense strand are linked through phosphorothioate linkages. In some embodiments, the sense strand comprises phosphorothioate linkages between the targeting group and the inverted abasic residue or the imann residue, and between the inverted abasic residue or the imann residue and the terminal modified or unmodified nucleotide at 5′ end of the sense strand. In some embodiments, the modified sense strand has a modification pattern set forth in any one of Tables 2-3. In some embodiments, the modified antisense strand has a modification pattern set forth in any one of Tables 2-3. In some embodiments, the modified sense strand is a modified sense strand sequence set forth in one of Tables 2-3. In some embodiments, the modified antisense strand is a modified antisense strand sequence set forth in one of Tables 2-3. In certain embodiments, the dsRNA comprises a duplex selected from the group consisting of AD00652, AD00653, AD00654, AD00655, AD00656, AD00657, AD00658, AD00659, AD00660, AD00661, AD00662, AD00663, AD00664, AD00663-1, AD00664-1, AD00815-1, AD00816-1, AD00819-1, AD00444-1, AD00663-2, AD00664-2, AD00815-2, AD00745, AD00746, AD00747, AD00748, AD00749, AD00750, AD00815, AD00816, AD00817, AD00818, AD00819 and AD00820.

In some embodiments, any one of the sense strands in Table 1 may further be modified in a pattern shown in aforesaid Formula (I) or (III).

In some embodiments, any one of the antisense strands in Table 1 may further be modified in a pattern shown in aforesaid Formula (II) or (III).

In some embodiments, any one of the duplexes in Table 1 may further be modified in a pattern shown in aforesaid Formula (III).

According to an aspect of the invention, a composition is provided that includes any embodiment of the aforementioned dsRNA agent aspect of the invention. In certain embodiments, the composition also includes a pharmaceutically acceptable carrier. In some embodiments, the composition also includes one or more additional therapeutic agents. In certain embodiments, the composition is packaged in a kit, container, pack, dispenser, pre-filled syringe, or vial. In some embodiments, the composition is formulated for subcutaneous administration or is formulated for intravenous (IV) administration.

According to another aspect of the invention a cell is provided that includes any embodiment of an aforementioned dsRNA agent aspect of the invention. In some embodiments, the cell is a mammalian cell, optionally a human cell.

According to another aspect of the invention, a method of inhibiting the expression of a PNPLA3 gene in a cell, is provided, the method including: (i) preparing a cell including an effective amount of any embodiment of the aforementioned dsRNA agent aspect of the invention or any embodiment of an aforementioned composition of the invention. In certain embodiments, the method also includes: (ii) maintaining the prepared cell for a time sufficient to obtain degradation of the mRNA transcript of a PNPLA3 gene, thereby inhibiting expression of the PNPLA3 gene in the cell. In some embodiments, the cell is in a subject and the dsRNA agent is administered to the subject subcutaneously. In some embodiments, the cell is in a subject and the dsRNA agent is administered to the subject by IV administration. In certain embodiments, the method also includes assessing inhibition of the PNPLA3 gene, following the administration of the dsRNA agent to the subject, wherein a means for the assessing comprises: (i) determining one or more physiological characteristics of a PNPLA3-associated disease or condition in the subject and (ii) comparing the determined physiological characteristic(s) to a baseline pre-treatment physiological characteristic of the PNPLA3-associated disease or condition and/or to a control physiological characteristic of the PNPLA3-associated disease or condition, wherein the comparison indicates one or more of a presence or absence of inhibition of expression of the PNPLA3 gene in the subject. In some embodiments, expression of the PNPLA3 gene can be assessed based on the level or change in level of any variable associated with PNPLA3 gene expression, such as PNPLA3 mRNA level, PNPLA3 protein level, the fat level and/or the lipid droplets level in the liver, or the number or extent of amyloid deposits.

According to another aspect of the invention, a method of inhibiting expression of a PNPLA3 gene in a subject, is provided, the method including administering to the subject an effective amount of an embodiment of the aforementioned dsRNA agent aspect of the invention or an embodiment of an aforementioned composition of the invention. In some embodiments, the dsRNA agent is administered to the subject subcutaneously. In certain embodiments, the dsRNA agent is administered to the subject by IV administration. In some embodiments, the method also includes: assessing inhibition of the PNPLA3 gene, following the administration of the dsRNA agent, wherein a means for the assessing comprises: (i) determining one or more physiological characteristics of a PNPLA3-associated disease or condition in the subject and (ii) comparing the determined physiological characteristic(s) to a baseline pre-treatment physiological characteristic of the PNPLA3-associated disease or condition and/or to a control physiological characteristic of the PNPLA3-associated disease or condition, wherein the comparison indicates one or more of a presence or absence of inhibition of expression of the PNPLA3 gene in the subject. In some embodiments, expression of the PNPLA3 gene can be assessed based on the level or change in level of any variable associated with PNPLA3 gene expression, such as PNPLA3 mRNA level, PNPLA3 protein level, the fat level and/or the lipid droplets level in the liver, or the number or extent of amyloid deposits.

According to another aspect of the invention, a method of treating a disease or condition associated with the presence of PNPLA3 protein is provided, the method including: administering to a subject an effective amount of an embodiment of any aforementioned dsRNA agent aspect of the invention or an embodiment of any aforementioned composition of the invention, to inhibit PNPLA3 gene expression. In some embodiments, the disease or condition is one or more of: liver disease, fatty liver (steatosis), nonalcoholic steatohepatitis (NASH), alcoholic steatohepatitis (ASH), cirrhosis of the liver, accumulation of fat in the liver, inflammation of the liver, hepatocellular necrosis, hepatocellular carcinoma, liver fibrosis, obesity, alcoholic liver disease, HCV hepatitis, chronic hepatitis, hereditary hemochromatosis, primary sclerosing cholangitis or nonalcoholic fatty liver disease (NAFLD). In some embodiments, the method also includes: administering an additional therapeutic regimen to the subject. In some embodiments, the additional therapeutic regimen includes a treatment for the PNPLA3-associated disease or condition. In certain embodiments, the additional therapeutic regimen comprises: administering to the subject one or more PNPLA3 antisense polynucleotides of the invention, administering to the subject a non-PNPLA3 dsRNA therapeutic agent, and a behavioral modification in the subject. In some embodiments, the non-PNPLA3 dsRNA therapeutic agent is one or more of: an HMG-COA reductase inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IB AT inhibitor, a squalene synthase inhibitor, a monocyte chemoattractant protein (MCP)-I inhibitor, or fish oil. In some embodiments, the dsRNA agent is administered to the subject subcutaneously. In certain embodiments, the dsRNA agent is administered to the subject by IV administration. In some embodiments, the method also includes determining an efficacy of the administered double-stranded ribonucleic acid (dsRNA) agent in the subject. In some embodiments, a means of determining an efficacy of the treatment in the subject comprises: (i) determining one or more physiological characteristics of the PNPLA3-associated disease or condition in the subject and (ii) comparing the determined physiological characteristic(s) to a baseline pre-treatment physiological characteristic of the PNPLA3-associated disease or condition wherein the comparison indicates one or more of a presence, absence, and level of efficacy of the administration of the double-stranded ribonucleic acid (dsRNA) agent to the subject. In some embodiments, expression of the PNPLA3 gene can be assessed based on the level or change in level of any variable associated with PNPLA3 gene expression, such as PNPLA3 mRNA level, PNPLA3 protein level, the fat level and/or the lipid droplets level in the liver, or the number or extent of amyloid deposits.

According to another aspect of the invention, a method of decreasing a level of PNPLA3 protein in a subject compared to a baseline pre-treatment level of PNPLA3 protein in the subject, is provided, the method including administering to the subject an effective amount of an embodiment of any aforementioned dsRNA agent of the invention or an embodiment of any aforementioned composition of the invention, to decrease the level of PNPLA3 gene expression. In some embodiments, the dsRNA agent is administered to the subject subcutaneously or is administered to the subject by IV administration.

According to another aspect of the invention, a method of altering a physiological characteristic of a PNPLA3-associated disease or condition in a subject compared to a baseline pre-treatment physiological characteristic of the PNPLA3-associated disease or condition in the subject is provided, the method including administering to the subject an effective amount of an embodiment of any aforementioned dsRNA agent of the invention or an embodiment of any aforementioned composition of the invention, to alter the physiological characteristic of the PNPLA3-associated disease or condition in the subject. In some embodiments, the dsRNA agent is administered to the subject subcutaneously or is administered to the subject by IV administration. In certain embodiments, the physiological characteristic is one or more of: the level of PNPLA3 mRNA or PNPLA3 protein in a sample derived from fluid or tissue from the specific site within the subject (e.g., liver or blood).

According to another aspect of the invention, the aforementioned dsRNA agent for use in a method of treating a disease or condition associated with the presence of PNPLA3 protein is provided. In some embodiments, the disease or condition is one or more of: fatty liver (steatosis), nonalcoholic steatohepatitis (NASH), cirrhosis of the liver, accumulation of fat in the liver, inflammation of the liver, hepatocellular necrosis, liver fibrosis, obesity, or nonalcoholic fatty liver disease (NAFLD) liver disease, fatty liver (steatosis), nonalcoholic steatohepatitis (NASH), alcoholic steatohepatitis (ASH), cirrhosis of the liver, accumulation of fat in the liver, inflammation of the liver, hepatocellular necrosis, hepatocellular carcinoma, liver fibrosis, obesity, alcoholic liver disease, HCV hepatitis, chronic hepatitis, hereditary hemochromatosis, primary sclerosing cholangitis or nonalcoholic fatty liver disease (NAFLD).

According to another aspect of the invention, an antisense polynucleotide agent for inhibiting expression of PNPLA3 protein is provided, the agent including from 10 to 30 contiguous nucleotides, wherein at least one of the contiguous nucleotides is a modified nucleotide, and wherein the nucleotide sequence of the agent is about 80% complementary over its entire length to the equivalent region of the nucleotide sequence of SEQ ID NO: 1. In some embodiments, the equivalent region is any one of the target regions of SEQ ID NO: 1 and the complementary sequence is one provided in one of Tables 1-3. In certain embodiments, the antisense polynucleotide agent includes one of the antisense sequences provided in one of Tables 1-3.

According to another aspect of the invention, a composition including an embodiment of any aforementioned antisense polynucleotide agents is provided. In some embodiments, the composition also includes a pharmaceutically acceptable carrier. In some embodiments, the composition also includes one or more additional therapeutic agents for treatment of a PNPLA3-associated disease or condition. In certain embodiments, the composition is packaged in a kit, container, pack, dispenser, pre-filled syringe, or vial. In certain embodiments, the composition is formulated for subcutaneous or IV administration.

According to another aspect of the invention a cell that includes an embodiment of any of the aforementioned antisense polynucleotide agents is provided. In some embodiments, the cell is a mammalian cell, optionally a human cell.

According to another aspect of the invention, a method of inhibiting the expression of a PNPLA3 gene in a cell is provided, the method including: (i) preparing a cell including an effective amount of an embodiment of any aforementioned antisense polynucleotide agents. In some embodiments, the method also includes (ii) maintaining the cell prepared in (i) for a time sufficient to obtain degradation of the mRNA transcript of a PNPLA3 gene, thereby inhibiting expression of the PNPLA3 gene in the cell.

According to another aspect of the invention, a method of inhibiting expression of a PNPLA3 gene in a subject is provided, the method including administering to the subject an effective amount of an embodiment of any of the aforementioned antisense polynucleotide agent.

According to another aspect of the invention, a method of treating a disease or condition associated with the presence of PNPLA3 protein, the method including administering to a subject an effective amount of an embodiment of any of the aforementioned antisense polynucleotide agents or an embodiment of any aforementioned composition of the invention, to inhibit PNPLA3 gene expression. In certain embodiments, the disease or condition is one or more of: liver disease, fatty liver (steatosis), nonalcoholic steatohepatitis (NASH), alcoholic steatohepatitis (ASH), cirrhosis of the liver, accumulation of fat in the liver, inflammation of the liver, hepatocellular necrosis, hepatocellular carcinoma, liver fibrosis, obesity, alcoholic liver disease, HCV hepatitis, chronic hepatitis, hereditary hemochromatosis, primary sclerosing cholangitis or nonalcoholic fatty liver disease (NAFLD).

According to another aspect of the invention, a method of decreasing a level of PNPLA3 protein in a subject compared to a baseline pre-treatment level of PNPLA3 protein in the subject is provided, the method including administering to the subject an effective amount of an embodiment of any of the aforementioned antisense polynucleotide agents or an embodiment of any aforementioned composition of the invention, to decrease the level of PNPLA3 gene expression. In certain embodiments, the antisense polynucleotide agent is administered to the subject subcutaneously or by IV administration.

According to another aspect of the invention, an antisense polynucleotide agent for inhibiting expression of PNPLA3 gene, is provided, the agent including from 10 to 30 contiguous nucleotides, wherein at least one of the contiguous nucleotides is a modified nucleotide, and wherein the nucleotide sequence of the agent is about 80% or about 85% complementary over its entire length to the equivalent region of the nucleotide sequence of SEQ ID NO: 1.

According to another aspect of the invention, a method of altering a physiological characteristic of a PNPLA3-associated disease or condition in a subject compared to a baseline pre-treatment physiological characteristic of the PNPLA3-associated disease or condition in the subject is provided, the method including administering to the subject an effective amount of an embodiment of any of the aforementioned antisense polynucleotide agents or an embodiment of any aforementioned composition of the invention, to alter the physiological characteristic of the PNPLA3 disease or condition in the subject. In some embodiments, the antisense polynucleotide agent is administered to the subject subcutaneously or by IV administration. In some embodiments, the physiological characteristic is one or more of: the level of PNPLA3 mRNA or PNPLA3 protein in a sample derived from fluid or tissue from the specific site within the subject (e.g., liver or blood).

### BRIEF DESCRIPTION OF THE SEQUENCES

SEQ ID NO: 1 and SEQ ID NO: 2 (reverse complement) are Homo sapiens Patatin-like Phospholipase Domain Containing 3 (PNPLA3) mRNA [NCBI Reference Sequence: NM_025225.3].

SEQ ID NO: 3 and SEQ ID NO: 4 (reverse complement) are Homo sapiens Patatin-like Phospholipase Domain Containing 3 (PNPLA3) mRNA [Source: HGNC Symbol; Acc: HGNC: 18590; Transcript: ENST00000423180.2].

SEQ ID NO: 5 and SEQ ID NO: 6 (reverse complement) are Predicted Macaca fascicularis Patatin-like Phospholipase Domain Containing 3 (PNPLA3), mRNA [NCBI Reference Sequence: XM_005567051.2].

SEQ ID NO: 7 and SEQ ID NO: 8 (reverse complement) are Predicted Macaca fascicularis Patatin-like Phospholipase Domain Containing 3 (PNPLA3), mRNA [NCBI Reference Sequence: XM_015457081.1].

SEQ ID NO: 9 and SEQ ID NO: 10 (reverse complement) are Predicted Macaca fascicularis Patatin-like Phospholipase Domain Containing 3 (PNPLA3), mRNA [Source: HGNC Symbol; Acc: HGNC: 18590; Transcript: ENSMFAT00000025830.2].

SEQ ID NO: 11 and SEQ ID NO: 12 (reverse complement) are Predicted Macaca mulatta Patatin-like Phospholipase Domain Containing 3 (PNPLA3), mRNA [NCBI Reference Sequence: XM_001109144.4].

SEQ ID NO: 13 and SEQ ID NO: 14 (reverse complement) are Predicted Macaca mulatta Patatin-like Phospholipase Domain Containing 3 (PNPLA3), mRNA [NCBI Reference Sequence: XM_015150532.2].

SEQ ID NO: 15 and SEQ ID NO: 16 (reverse complement) are Predicted Macaca mulatta Patatin-like Phospholipase Domain Containing 3 (PNPLA3), mRNA [Source: VGNC Symbol; Acc: VGNC: 76061; Transcript: ENSMMUT00000023461.4].

SEQ ID NO: 17 and SEQ ID NO: 18 (reverse complement) are Mus musculus Patatin-like Phospholipase Domain Containing 3 (PNPLA3), mRNA [NCBI Reference Sequence: NM_054088.3].

SEQ ID NO: 19 and SEQ ID NO: 20 (reverse complement) are Mus musculus Patatin-like Phospholipase Domain Containing 3 (PNPLA3), mRNA [Source: MGI Symbol; Acc: MGI: 2151796; Transcript: ENSMUST00000045289.6].

SEQ ID NO: 21 and SEQ ID NO: 22 (reverse complement) are Rattus norvegicus Patatin-like Phospholipase Domain Containing 3 (PNPLA3), mRNA [NCBI Reference Sequence: NM_001282324.1].

SEQ ID NO: 23 and SEQ ID NO: 24 (reverse complement) are Rattus norvegicus Patatin-like Phospholipase Domain Containing 3 (PNPLA3), mRNA [Source: RGD Symbol; Acc: 1595843; Transcript: ENSRNOT00000015767.8].

SEQ ID NOs: 25-252 are shown in Table 1 and are sense strand sequences.

SEQ ID NOs: 253-480 are shown in Table 1 and are antisense strand sequences.

SEQ ID NOs: 481-648 are shown in Table 2 with chemical modifications indicated by upper case: 2′-Fluoro; lower case: 2′-OMe; and thiophosphate: *, and it can be understood by person skilled in the art that “*” is a symbol for indication of linkage relationship, wherein the presence of “*” means that monomers are linked to each other via a phosphorothioate diester linkage, wherein the absence of “*” between two monomers indicates that the monomers are linked to each other via a phosphodiester linkage; and invab=inverted abasic.

SEQ ID NOs: 649-742 are shown in Table 3. A delivery molecule is indicated as “GLX-_” the 3′ end or 5′ end of each sense strand. Chemical modifications are indicated as: upper case: 2′-Fluoro; lower case: 2′-OMe; and thiophosphate: *, and it can be understood by person skilled in the art that “*” is a symbol for indication of linkage relationship, wherein the presence of “*” means that monomers are linked to each other via a phosphorothioate diester linkage, wherein the absence of “*” between two monomers indicates that the monomers are linked to each other via a phosphodiester linkage; and invab=inverted abasic; imann:

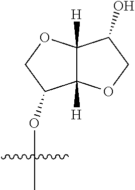 [SMILES: *O[C@@H]1CO[C@H]2[C@@H]1OC[C@H]2O]

when at the end of each strand or

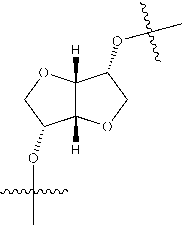 [SMILES: *O[C@@H]1CO[C@H]2[C@@H]1OC[C@H]2O*]

when further conjugated to a delivery molecule; VPu*:

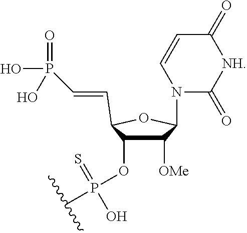

### DETAILED DESCRIPTION

The invention in part, includes RNAi agents, for example, though not limited to double stranded (ds) RNAi agents, which are capable of inhibiting Patatin-like Phospholipase Domain Containing 3 (PNPLA3) gene expression. The invention, in part also includes compositions comprising PNPLA3 RNAi agents and methods of use of the compositions. PNPLA3 RNAi agents disclosed herein may be attached to delivery compounds for delivery to cells, including to hepatocytes. Pharmaceutical compositions of the invention may include at least one ds PNPLA3 agent and a delivery compound. In some embodiments of compositions and methods of the invention, the delivery compound is a GalNAc-containing delivery compound. PNPLA3 RNAi agents delivered to cells are capable of inhibiting PNPLA3 gene expression, thereby reducing activity in the cell of the PNPLA3 protein product of the gene. DsRNAi agents of the invention can be used to treat PNPLA3-associated diseases and conditions.

In some embodiments of the invention reducing PNPLA3 expression in a cell or subject treats a disease or condition associated with PNPLA3 expression in the cell or subject, respectively. Non-limiting examples of diseases and conditions that may be treated by reducing PNPLA3 activity are: liver disease, fatty liver (steatosis), nonalcoholic steatohepatitis (NASH), alcoholic steatohepatitis (ASH), cirrhosis of the liver, accumulation of fat in the liver, inflammation of the liver, hepatocellular necrosis, hepatocellular carcinoma, liver fibrosis, obesity, alcoholic liver disease, HCV hepatitis, chronic hepatitis, hereditary hemochromatosis, primary sclerosing cholangitis or nonalcoholic fatty liver disease (NAFLD), or other diseases for which reducing a level and activity of PNPLA3 protein is medically beneficial.

As used herein, “G,” “C,” “A” and “U” each generally stands for a nucleotide that contains guanine, cytosine, adenine, and uracil as a base, respectively. However, it will be understood that the term “ribonucleotide” or “nucleotide” can also refer to a modified nucleotide, as further detailed below, or a surrogate replacement moiety. The skilled person understands that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the invention by a nucleotide containing, for example, inosine. Sequences comprising such replacement moieties are embodiments of the invention.

As used herein, “Patatin-Like Phospholipase Domain Containing 3,” used interchangeably with the term “PNPLA3” refers to the naturally occurring gene that encodes a triacylglycerol lipase that mediates triacyl glycerol hydrolysis in adipocytes. The amino acid and complete coding sequences of the reference sequence of the human PNPLA3 gene may be found in, for example, GenBank RefSeq Accession No. NM_025225.3 (SEQ ID NO: 1 and SEQ ID NO:2); HGNC Transcript: ENST00000423180.2 (SEQ ID NO: 3 and SEQ ID NO:4). Mammalian orthologs of the human PNPLA3 gene may be found in, for example, GenBank RefSeq Accession No. XM_005567051.2, cynomolgus monkey (SEQ ID NO:5 and SEQ ID NO: 6); RefSeq Accession No. XM_015457081.1, cynomolgus monkey (SEQ ID NO: 7 and SEQ ID NO: 8); HGNC Transcript: ENSMFAT00000025830.2, cynomolgus monkey (SEQ ID NO: 9 and SEQ ID NO: 10); GenBank RefSeq Accession No. XM_001109144.4, rhesus monkey (SEQ ID NO:11 and SEQ ID NO: 12); GenBank RefSeq Accession No. XM_015150532.2, rhesus monkey (SEQ ID NO:13 and SEQ ID NO: 14); HGNC Transcript: ENSMMUT00000023461.4, rhesus monkey (SEQ ID NO:15 and SEQ ID NO: 16); GenBank RefSeq Accession No. NM_054088.3, mouse, (SEQ ID NO:17 and SEQ ID NO:18); HGNC Transcript: ENSMUST00000045289.6, mouse, (SEQ ID NO:19 and SEQ ID NO:20); GenBank RefSeq Accession No. NM 001282324.1, rat (SEQ ID NO:21 and SEQ ID NO:22); HGNC Transcript: ENSRNOT00000015767.7, rat (SEQ ID NO:23 and SEQ ID NO:24). Additional examples of PNPLA3 mRNA sequences are readily available using publicly available databases, e.g., GenBank, UniProt, Ensembl and OMIM.

The following describes how to make and use compositions comprising PNPLA3 single-stranded (ssRNA) and dsRNA agents to inhibit PNPLA3 gene expression, as well as compositions and methods for treating diseases and conditions caused by or modulated by PNPLA3 gene expression. The term “RNAi” is also known in the art, and may be referred to as “siRNA”.

As used herein, the term “RNAi” refers to an agent that comprises RNA and mediates targeted cleavage of an RNA transcript via an RNA-induced silencing complex (RISC) pathway. As is known in the art, an RNAi target region, which is also defined as “target region” or “target portion”, refers to a contiguous portion of the nucleotide sequence of an mRNA molecule formed during the transcription of a gene, including messenger RNA (mRNA) that is a product of RNA processing of a primary transcription product. The target portion of the sequence will be at least long enough to serve as a substrate for RNAi-directed cleavage at or near that portion. A target sequence may be from 8-30 nucleotides long (inclusive), from 10-30 nucleotides long (inclusive), from 12-25 nucleotides long (inclusive), from 15-23 nucleotides long (inclusive), from 16-23 nucleotides long (inclusive), or from 18-23 nucleotides long (inclusive), including all shorter lengths within each stated range. In some embodiments of the invention, a target sequence is 9, 10, 11, 12, 13, 14, 15, 16, 17, 18, 19, 20, 21, 22, 23, 24, 25, or 26 nucleotides long. In certain embodiment a target sequence is between 9 and 26 nucleotides long (inclusive), including all sub-ranges and integers there between. For example, though not intended to be limiting, in certain embodiments of the invention a target sequence is 8, 9, 10, 11, 12, 13, 14, 15, 16, 17, 18, 19, 20, 21, 22, 23, 24, 25, 26, 27, 28, 29, or 30 nucleotides long, with the sequence fully or at least substantially complementary to at least part of an RNA transcript of a PNPLA3 gene. Some aspects of the invention include pharmaceutical compositions comprising one or more PNPLA3 dsRNA agents and a pharmaceutically acceptable carrier. In certain embodiments of the invention, a PNPLA3 RNAi as described herein inhibits expression of PNPLA3 protein.

As used herein, a “dsRNA agent” means a composition that contains an RNA or RNA-like (e.g., chemically modified RNA) oligonucleotide molecule that is capable of degrading or inhibiting translation of messenger RNA (mRNA) transcripts of a target mRNA in a sequence specific manner. Although not wishing to be limited to a particular theory, dsRNA agents of the invention may operate through the RNA interference mechanism (i.e., inducing RNA interference through interaction with the RNA interference pathway machinery (RNA-induced silencing complex or RISC) of mammalian cells), or by any alternative mechanism(s) or pathway(s). Methods for silencing genes in plant, invertebrate, and vertebrate cells are well known in the art [see, for example, (Sharp et al., Genes Dev. 2001, 15:485; Bernstein, et al., (2001) Nature 409:363; Nykanen, et al., (2001) Cell 107:309; and Elbashir, et al., (2001) Genes Dev. 15:188)], the disclosure of each of which is incorporated herein by reference in its entirety.]. Art-known gene silencing procedures can be used in conjunction with the disclosure provided herein to inhibit expression of PNPLA3.

DsRNA agents disclosed herein are comprised of a sense strand and an antisense strand, and include, but are not limited to: short interfering RNAs (siRNAs), RNAi agents, micro RNAs (miRNAs), short hairpin RNAs (shRNA), and dicer substrates. The antisense strand of the dsRNA agents described herein is at least partially complementary to the mRNA being targeted. It is understood in the art that different lengths of dsRNA duplex structure can be used to inhibit target gene expression. For example, dsRNAs having a duplex structure of 19, 20, 21, 22, and 23 base pairs are known to be effective to induce RNA interference (Elbashir et al., EMBO 2001, 20:6877-6888). It is also known in the art that shorter or longer RNA duplex structures are also effective to induce RNA interference. As used herein, the terms “double stranded region”, “duplex region” and “the region of complementarity” can be used interchangeably, and refer to the region that the sense strand is complementary or substantially complementary to the antisense strand as is known in the art. PNPLA3 dsRNAs in certain embodiments of the invention can include at least one strand of a length of minimally 21 nt or may have shorter duplexes based on one of the sequences set forth in any one of Tables 1-3, but minus 1, 2, 3, or 4 nucleotides on one or both ends may also be effective as compared to the dsRNAs set forth in Tables 1-3, respectively. In some embodiments of the invention, PNPLA3 dsRNA agents may have a partial sequence of at least 15, 16, 17, 18, 19, 20, or more contiguous nucleotides from one or more sequences of Tables 1-3, and differ in their ability to inhibit the expression of a PNPLA3 gene by not more than 5%, 10%, 15%, 20%, 25%, or 30% from the level of inhibition resulting from a dsRNA comprising the full sequence. A sense sequence, an antisense sequence and a duplex disclosed in Tables 1-3 may be referred to herein as a “parent” sequence, meaning that the sequences disclosed in Tables 1-3 may be modified, shorten, lengthened, include substitutions, etc. as set forth herein, with the resulting sequences retaining all or at least a portion of the efficacy of their parent sequences in methods and compositions of the invention. Sense and antisense strands included in a dsRNA of the invention are independently selected. As used herein the term “independently selected” means each of two or more like elements can be selected independent of the selection of the other elements. For example, though not intended to be limiting, in preparing a dsRNA of the invention, one may select the “elements” of the two strands to include in the duplex. One selected element, the sense sequence may be SEQ ID NO: 650 (shown in Table 3) and the other selected element, the antisense sequence, may be SEQ ID NO: 687, or may be SEQ ID NO: 687 that is modified, shortened, lengthened, and/or includes 1, 2, or 3 substitutions as compared to its parent sequence SEQ ID NO: 687. It will be understood that a duplex of the invention need not include both sense and antisense sequences shown as paired in duplexes in Tables 1-3. Each sense and antisense strand sequence in the tables is immediately followed by its SEQ ID NO.

Certain embodiments of compositions and methods of the invention comprise a single-strand RNA in a composition and/or administered to a subject. For example, an antisense strand such as one listed in any one of Tables 1-3 may be a composition or in a composition administered to a subject to reduce PNPLA3 polypeptide activity and/or expression of PNPLA3 gene in the subject. Tables 1-3 show certain PNPLA3 dsRNA agent antisense strand and sense strand core stretch base sequences. A single-strand antisense molecule that may be included in certain compositions and/or administered in certain methods of the invention are referred to herein as a “single-strand antisense agent” or an “antisense polynucleotide agent”. A single-strand sense molecule that may be included in certain compositions and/or administered in certain methods of the invention are referred to herein as a “single-strand sense agent” or a “sense polynucleotide agent”. The term “base sequence” is used herein in reference to a polynucleotide sequence without chemical modifications or delivery compounds. For example, the sense strand GAGGUCCUCUCAGAUCUUGUA (SEQ ID NO: 25) shown in Table 1 is the base sequence for SEQ ID NO: 481 in Table 2 and for SEQ ID NO: 674 in Table 3, with SEQ ID NO: 481 and SEQ ID NO: 674 shown with their chemical modifications and/or a delivery compound. Sequences disclosed herein may be assigned identifiers. For example, a single-stranded sense sequence may be identified with a “Sense strand SS #”; a single stranded antisense sequence may be identified with an “Antisense strand AS #” and a duplex that includes a sense strand and an antisense strand may be identified with a “Duplex AD #/AV #”.

Table 1 includes sense and antisense strands and provides the identification number of duplexes formed from the sense and antisense strand on the same line in Table 1. The sense strands SEQ ID Nos: 177-252 include a random nucleobase (n) at positions 1, 2, 3 and 21 from the 5′ end. The antisense strands SEQ ID Nos: 405-480 include a random nucleobase (n) at positions 1, 19, 20, and 21 from the 5′ end. In certain embodiments of the invention an antisense sequence includes nucleobase u or nucleobase a in position 1 of the antisense sequence. In certain embodiments of the invention an antisense sequence includes nucleobase u in position 1 of the antisense sequence. In the sequences shown in Table 1 “n” can represent a nucleotide comprising any one of nucleobases a, u, c, g, and t and can be independently selected for the sense and antisense strand, and each “n” in the sense strand or the antisense strand can be the same or different. As used in the context of “n” in sense and antisense strands, it will be understood that the nucleobase “n” selected and included in a position in a sense strand is not the same nucleobase as “n” in the antisense strand with which the sense strand pairs, but rather is generally complementary to the nucleobase “n” at the matching position in the opposite strand. As used herein, the term “matching position” in a sense and an antisense strand are the positions in each strand that “pair” when the two strands are duplexed strands. For example, in a 21 nucleobase sense strand and a 21 nucleobase antisense strand, nucleobase in position 1 of the sense strand and position 21 in the antisense strand are in “matching positions”. In yet another non-limiting example in a 23 nucleobase sense strand and a 23 nucleobase antisense strand, nucleobase 2 of the sense strand and position 22 of the antisense strand are in matching positions. In another non-limiting example, in an 18 nucleobase sense strand and an 18 nucleobase antisense strand, nucleobase in position 1 of the sense strand and nucleobase 18 in the antisense strand are in matching positions, and nucleobase 4 in the sense strand and nucleobase 15 in the antisense strand are in matching positions. A skilled artisan will understand how to identify matching positions in sense and antisense strands that are or will be duplexed strands and paired strands.

Although (n) can be any one of a, u, c, g or t, an “n” at position 1 of sense strand is generally complementary to (n) at position 21 of antisense strand. In two non-limiting examples, (1) if position 1 of sense strand is “g” then position 21 of antisense strand is “c”; and (2) if position 1 of sense strand is “a” then position 21 of antisense strand is “u” or “t”. This type of complimentary matching pairing applies to (n) at position 2 of sense strand and position 20 of antisense strand; (n) at position 21 of sense strand and position 1 of antisense strand. It will be understood that even though n can be any nucleotide at these positions, the nucleotides of sense and antisense strand are generally still complementary (match), however, in certain embodiments, they may have mismatch. For example, though not intended to be limiting, in some embodiments “n” can be “random”, meaning might but need not be complementary. In certain embodiments “n” is complementary. As a non-limiting example, “n” in position of 1 of antisense is “u” and “n” in position of 21 of sense strand is “a”. A skilled artisan will understand how to identify matching positions in sense and antisense strands that are or will be duplexed strands and paired strands.

The final column in Table 1 indicates a Duplex AD # for a duplex that includes the sense and antisense sequences in the same table row. For example, Table 1 discloses the duplex assigned Duplex AD #AD00448.um, which includes sense strand SEQ ID NO: 25 and antisense strand SEQ ID NO: 253. Thus, each row in Table 1 identifies a duplex of the invention, each comprising the sense and antisense sequences shown in the same row, with the assigned identifier for each duplex shown in the final column in the row.

In some embodiments of methods of the invention, an RNAi agent comprising a polynucleotide sequence shown in Table 1 is administered to a subject. In some embodiments of the invention an RNAi agent administered to a subject comprises is a duplex comprising at least one of the base sequences set forth in Table 1, including 0, 1, 2, 3, 4, 5, 6, 7, 8, 9, 10, 11, 12, 13, 14, 15, 16, 17, 18, 19, 20, 21, 22, 23, or 24 sequence modifications. In some embodiments of methods of the invention an RNAi agent comprising a polynucleotide sequence shown in Table 1 is attached to a delivery molecule, a non-limiting example of which is a delivery compound comprising a GalNAc compound, or a GLS-15 compound.

TABLE 1
Unmodified PNPLA3 RNAi agent antisense strand
and sense strand sequences.

| | SEQ | | SEQ | |
|---|---|---|---|---|
| Sense strand | ID NO | Antisense strand | ID NO | Duplex AD# |
| GAGGUCCUCUCAGAUCUUGUA | 25 | UACAAGAUCUGAGAGGACCUC | 253 | AD00448.um |
| GUCUUCCAUCCAUCCUUCAAA | 26 | UUUGAAGGAUGGAUGGAAGAC | 254 | AD00449.um |
| GAAUGUCCACCAGCUCAUCUA | 27 | UAGAUGAGCUGGUGGACAUUC | 255 | AD00450.um |
| GCUUACCAGAGUGUCUGAUGA | 28 | UCAUCAGACACUCUGGUAAGC | 256 | AD00451.um |
| CAGUGUCUGAUGGGGAAAACA | 29 | UGUUUUCCCCAUCAGACACUG | 257 | AD00452.um |
| CUGUCUGAUGGGGAAAACGUA | 30 | UACGUUUUCCCCAUCAGACAG | 258 | AD00453.um |
| GGUCUGAUGGGGAAAACGUUA | 31 | UAACGUUUUCCCCAUCAGACC | 259 | AD00454.um |
| GCUGAUGGGGAAAACGUUCUA | 32 | UAGAACGUUUUCCCCAUCAGC | 260 | AD00455.um |
| CAUGGGGAAAACGUUCUGGUA | 33 | UACCAGAACGUUUUCCCCAUG | 261 | AD00456.um |
| CAAAACGUUCUGGUGUCUGAA | 34 | UUCAGACACCAGAACGUUUUG | 262 | AD00457.um |
| GAACGUUCUGGUGUCUGACUA | 35 | UAGUCAGACACCAGAACGUUC | 263 | AD00458.um |
| GGGUCCAAAGACGAAGUCGUA | 36 | UACGACUUCGUCUUUGGACCC | 264 | AD00459.um |
| GUUCAGAGGCGUGCGAUAUGA | 37 | UCAUAUCGCACGCCUCUGAAC | 265 | AD00460.um |
| GGUGACAACGUACCCUUCAUA | 38 | UAUGAAGGGUACGUUGUCACC | 266 | AD00461.um |
| CUGACAACGUACCCUUCAUUA | 39 | UAAUGAAGGGUACGUUGUCAG | 267 | AD00462.um |
| CACAACGUACCCUUCAUUGAA | 40 | UUCAAUGAAGGGUACGUUGUG | 268 | AD00463.um |
| GUCUAUGGGGAGUACGACAUA | 41 | UAUGUCGUACUCCCCAUAGAC | 269 | AD00464.um |
| GGUACGACAUCUGCCCUAAAA | 42 | UUUUAGGGCAGAUGUCGUACC | 270 | AD00465.um |
| CUACGACAUCUGCCCUAAAGA | 43 | UCUUUAGGGCAGAUGUCGUAG | 271 | AD00466.um |
| GGACAUCUGCCCUAAAGUCAA | 44 | UUGACUUUAGGGCAGAUGUCC | 272 | AD00467.um |
| CACAUCUGCCCUAAAGUCAAA | 45 | UUUGACUUUAGGGCAGAUGUG | 273 | AD00468.um |
| GCAUCUGCCCUAAAGUCAAGA | 46 | UCUUGACUUUAGGGCAGAUGC | 274 | AD00469.um |
| GUCUGCCCUAAAGUCAAGUCA | 47 | UGACUUGACUUUAGGGCAGAC | 275 | AD00470.um |
| GCGAACUUUCUUCAUGUGGAA | 48 | UUCCACAUGAAGAAAGUUCGC | 276 | AD00471.um |
| GUUUCUUCAUGUGGACAUCAA | 49 | UUGAUGUCCACAUGAAGAAAC | 277 | AD00472.um |
| GUUCUUCAUGUGGACAUCACA | 50 | UGUGAUGUCCACAUGAAGAAC | 278 | AD00473.um |
| GUUCAUGUGGACAUCACCAAA | 51 | UUUGGUGAUGUCCACAUGAAC | 279 | AD00474.um |
| GGUGGACAUCACCAAGCUCAA | 52 | UUGAGCUUGGUGAUGUCCACC | 280 | AD00475.um |
| GGAUAUGCCUUCGAGGAUAUA | 53 | UAUAUCCUCGAAGGCAUAUCC | 281 | AD00476.um |
| CAUAUGCCUUCGAGGAUAUUA | 54 | UAAUAUCCUCGAAGGCAUAUG | 282 | AD00477.um |
| GUAUGCCUUCGAGGAUAUUUA | 55 | UAAAUAUCCUCGAAGGCAUAC | 283 | AD00478.um |
| GAUGCCUUCGAGGAUAUUUGA | 56 | UCAAAUAUCCUCGAAGGCAUC | 284 | AD00479.um |
| CCAUUCAGGUUCUUGGAAGAA | 57 | UUCUUCCAAGAACCUGAAUGG | 285 | AD00480.um |
| GUGAAAGACAAAGGUGGAUAA | 58 | UUAUCCACCUUUGUCUUUCAC | 286 | AD00481.um |
| CAAAGACAAAGGUGGAUACAA | 59 | UUGUAUCCACCUUUGUCUUUG | 287 | AD00482.um |
| CACAAAGGUGGAUACAUGAGA | 60 | UCUCAUGUAUCCACCUUUGUG | 288 | AD00483.um |
| GAAGGUGGAUACAUGAGCAAA | 61 | UUUGCUCAUGUAUCCACCUUC | 289 | AD00484.um |
| GGGAUACAUGAGCAAGAUUUA | 62 | UAAAUCUUGCUCAUGUAUCCC | 290 | AD00485.um |
| GACAUGAGCAAGAUUUGCAAA | 63 | UUUGCAAAUCUUGCUCAUGUC | 291 | AD00486.um |
| GAUGAGCAAGAUUUGCAACUA | 64 | UAGUUGCAAAUCUUGCUCAUC | 292 | AD00487.um |
| GUUUGCAACUUGCUACCCAUA | 65 | UAUGGGUAGCAAGUUGCAAAC | 293 | AD00488.um |
| GUGCAACUUGCUACCCAUUAA | 66 | UUAAUGGGUAGCAAGUUGCAC | 294 | AD00489.um |
| GGCAACUUGCUACCCAUUAGA | 67 | UCUAAUGGGUAGCAAGUUGCC | 295 | AD00490.um |
| CCAACUUGCUACCCAUUAGGA | 68 | UCCUAAUGGGUAGCAAGUUGG | 296 | AD00491.um |
| GAACUUGCUACCCAUUAGGAA | 69 | UUCCUAAUGGGUAGCAAGUUC | 297 | AD00492.um |
| GACUUGCUACCCAUUAGGAUA | 70 | UAUCCUAAUGGGUAGCAAGUC | 298 | AD00493.um |
| GUGCUACCCAUUAGGAUAAUA | 71 | UAUUAUCCUAAUGGGUAGCAC | 299 | AD00494.um |
| CAAUCUGCCAUUGCGAUUGUA | 72 | UACAAUCGCAAUGGCAGAUUG | 300 | AD00495.um |
| GUUGCGAUUGUCCAGAGACUA | 73 | UAGUCUCUGGACAAUCGCAAC | 301 | AD00496.um |
| CGUGACAUGGCUUCCAGAUAA | 74 | UUAUCUGGAAGCCAUGUCACG | 302 | AD00497.um |
| CUGACAUGGCUUCCAGAUAUA | 75 | UAUAUCUGGAAGCCAUGUCAG | 303 | AD00498.um |
| GGACAUGGCUUCCAGAUAUGA | 76 | UCAUAUCUGGAAGCCAUGUCC | 304 | AD00499.um |
| GCUUGGGCAAUAAAGUACCUA | 77 | UAGGUACUUUAUUGCCCAAGC | 305 | AD00500.um |
| GCCACCUUUCCCAGUUUUUCA | 78 | UGAAAAACUGGGAAAGGUGGC | 306 | AD00501.um |
| GACCUUUCCCAGUUUUUCACA | 79 | UGUGAAAAACUGGGAAAGGUC | 307 | AD00502.um |
| GUUCUUUCAGAGGUGCUAAAA | 80 | UUUUAGCACCUCUGAAAGAAC | 308 | AD00503.um |
| CCCUCUGAGCUGAGUUGGUUA | 81 | UAACCAACUCAGCUCAGAGGG | 309 | AD00504.um |
| GGAGCUGAGUUGGUUUUAUGA | 82 | UCAUAAAACCAACUCAGCUCC | 310 | AD00505.um |
| CCUGAGUUGGUUUUAUGAAAA | 83 | UUUUCAUAAAACCAACUCAGG | 311 | AD00506.um |
| GUUUAUGAAAAGCUAGGAAGA | 84 | UCUUCCUAGCUUUUCAUAAAC | 312 | AD00507.um |
| GAUUCAGCUGGUUGGGAAAUA | 85 | UAUUUCCCAACCAGCUGAAUC | 313 | AD00508.um |
| GUUCAGCUGGUUGGGAAAUGA | 86 | UCAUUUCCCAACCAGCUGAAC | 314 | AD00509.um |
| GAGAGGGUCCCUUACUGACUA | 87 | UAGUCAGUAAGGGACCCUCUC | 315 | AD00510.um |
| CCCCUAUUAAUGGUCAGACUA | 88 | UAGUCUGACCAUUAAUAGGGG | 316 | AD00511.um |
| GCUAUUAAUGGUCAGACUGUA | 89 | UACAGUCUGACCAUUAAUAGC | 317 | AD00512.um |
| GAUUAAUGGUCAGACUGUUCA | 90 | UGAACAGUCUGACCAUUAAUC | 318 | AD00513.um |
| GUAAUGGUCAGACUGUUCCAA | 91 | UUGGAACAGUCUGACCAUUAC | 319 | AD00514.um |
| GACACCUUUUUCACCUAACUA | 92 | UAGUUAGGUGAAAAAGGUGUC | 320 | AD00515.um |
| GACCUUUUUCACCUAACUAAA | 93 | UUUAGUUAGGUGAAAAAGGUC | 321 | AD00516.um |
| GCUUUUUCACCUAACUAAAAA | 94 | UUUUUAGUUAGGUGAAAAAGC | 322 | AD00517.um |
| GAAGACAAAGGUGGAUACAUA | 95 | UAUGUAUCCACCUUUGUCUUC | 323 | AD01072.um |
| CGUGGAUACAUGAGCAAGAUA | 96 | UAUCUUGCUCAUGUAUCCACG | 324 | AD01075.um |
| CUGGAUACAUGAGCAAGAUUA | 97 | UAAUCUUGCUCAUGUAUCCAG | 325 | AD01076.um |
| GUUGCUACCCAUUAGGAUAAA | 98 | UUUAUCCUAAUGGGUAGCAAC | 326 | AD01081.um |
| GCUGAGCUGAGUUGGUUUUAA | 99 | UUAAAACCAACUCAGCUCAGC | 327 | AD01083.um |
| GUGGUUUUAUGAAAAGCUAGA | 100 | UCUAGCUUUUCAUAAAACCAC | 328 | AD01085.um |
| GGUCCUCUCAGAUCUUGUA | 101 | UACAAGAUCUGAGAGGACC | 329 | AD00448-19- |
| | | | | 1.um |
| CUUCCAUCCAUCCUUCAAA | 102 | UUUGAAGGAUGGAUGGAAG | 330 | AD00449-19- |
| | | | | 1.um |
| AUGUCCACCAGCUCAUCUA | 103 | UAGAUGAGCUGGUGGACAU | 331 | AD00450-19- |
| | | | | 1.um |
| UUACCAGAGUGUCUGAUGA | 104 | UCAUCAGACACUCUGGUAA | 332 | AD00451-19- |
| | | | | 1.um |
| GUGUCUGAUGGGGAAAACA | 105 | UGUUUUCCCCAUCAGACAC | 333 | AD00452-19- |
| | | | | 1.um |
| GUCUGAUGGGGAAAACGUA | 106 | UACGUUUUCCCCAUCAGAC | 334 | AD00453-19- |
| | | | | 1.um |
| UCUGAUGGGGAAAACGUUA | 107 | UAACGUUUUCCCCAUCAGA | 335 | AD00454-19- |
| | | | | 1.um |
| UGAUGGGGAAAACGUUCUA | 108 | UAGAACGUUUUCCCCAUCA | 336 | AD00455-19- |
| | | | | 1.um |
| UGGGGAAAACGUUCUGGUA | 109 | UACCAGAACGUUUUCCCCA | 337 | AD00456-19- |
| | | | | 1.um |
| AAACGUUCUGGUGUCUGAA | 110 | UUCAGACACCAGAACGUUU | 338 | AD00457-19- |
| | | | | 1.um |
| ACGUUCUGGUGUCUGACUA | 111 | UAGUCAGACACCAGAACGU | 339 | AD00458-19- |
| | | | | 1.um |
| GUCCAAAGACGAAGUCGUA | 112 | UACGACUUCGUCUUUGGAC | 340 | AD00459-19- |
| | | | | 1.um |
| UCAGAGGCGUGCGAUAUGA | 113 | UCAUAUCGCACGCCUCUGA | 341 | AD00460-19- |
| | | | | 1.um |
| UGACAACGUACCCUUCAUA | 114 | UAUGAAGGGUACGUUGUCA | 342 | AD00461-19- |
| | | | | 1.um |
| GACAACGUACCCUUCAUUA | 115 | UAAUGAAGGGUACGUUGUC | 343 | AD00462-19- |
| | | | | 1.um |
| CAACGUACCCUUCAUUGAA | 116 | UUCAAUGAAGGGUACGUUG | 344 | AD00463-19- |
| | | | | 1.um |
| CUAUGGGGAGUACGACAUA | 117 | UAUGUCGUACUCCCCAUAG | 345 | AD00464-19- |
| | | | | 1.um |
| UACGACAUCUGCCCUAAAA | 118 | UUUUAGGGCAGAUGUCGUA | 346 | AD00465-19- |
| | | | | 1.um |
| ACGACAUCUGCCCUAAAGA | 119 | UCUUUAGGGCAGAUGUCGU | 347 | AD00466-19- |
| | | | | 1.um |
| ACAUCUGCCCUAAAGUCAA | 120 | UUGACUUUAGGGCAGAUGU | 348 | AD00467-19- |
| | | | | 1.um |
| CAUCUGCCCUAAAGUCAAA | 121 | UUUGACUUUAGGGCAGAUG | 349 | AD00468-19- |
| | | | | 1.um |
| AUCUGCCCUAAAGUCAAGA | 122 | UCUUGACUUUAGGGCAGAU | 350 | AD00469-19- |
| | | | | 1.um |
| CUGCCCUAAAGUCAAGUCA | 123 | UGACUUGACUUUAGGGCAG | 351 | AD00470-19- |
| | | | | 1.um |
| GAACUUUCUUCAUGUGGAA | 124 | UUCCACAUGAAGAAAGUUC | 352 | AD00471-19- |
| | | | | 1.um |
| UUCUUCAUGUGGACAUCAA | 125 | UUGAUGUCCACAUGAAGAA | 353 | AD00472-19- |
| | | | | 1.um |
| UCUUCAUGUGGACAUCACA | 126 | UGUGAUGUCCACAUGAAGA | 354 | AD00473-19- |
| | | | | 1.um |
| UCAUGUGGACAUCACCAAA | 127 | UUUGGUGAUGUCCACAUGA | 355 | AD00474-19- |
| | | | | 1.um |
| UGGACAUCACCAAGCUCAA | 128 | UUGAGCUUGGUGAUGUCCA | 356 | AD00475-19- |
| | | | | 1.um |
| AUAUGCCUUCGAGGAUAUA | 129 | UAUAUCCUCGAAGGCAUAU | 357 | AD00476-19- |
| | | | | 1.um |
| UAUGCCUUCGAGGAUAUUA | 130 | UAAUAUCCUCGAAGGCAUA | 358 | AD00477-19- |
| | | | | 1.um |
| AUGCCUUCGAGGAUAUUUA | 131 | UAAAUAUCCUCGAAGGCAU | 359 | AD00478-19- |
| | | | | 1.um |
| UGCCUUCGAGGAUAUUUGA | 132 | UCAAAUAUCCUCGAAGGCA | 360 | AD00479-19- |
| | | | | 1.um |
| AUUCAGGUUCUUGGAAGAA | 133 | UUCUUCCAAGAACCUGAAU | 361 | AD00480-19- |
| | | | | 1.um |
| GAAAGACAAAGGUGGAUAA | 134 | UUAUCCACCUUUGUCUUUC | 362 | AD00481-19- |
| | | | | 1.um |
| AAGACAAAGGUGGAUACAA | 135 | UUGUAUCCACCUUUGUCUU | 363 | AD00482-19- |
| | | | | 1.um |
| CAAAGGUGGAUACAUGAGA | 136 | UCUCAUGUAUCCACCUUUG | 364 | AD00483-19- |
| | | | | 1.um |
| AGGUGGAUACAUGAGCAAA | 137 | UUUGCUCAUGUAUCCACCU | 365 | AD00484-19- |
| | | | | 1.um |
| GAUACAUGAGCAAGAUUUA | 138 | UAAAUCUUGCUCAUGUAUC | 366 | AD00485-19- |
| | | | | 1.um |
| CAUGAGCAAGAUUUGCAAA | 139 | UUUGCAAAUCUUGCUCAUG | 367 | AD00486-19- |
| | | | | 1.um |
| UGAGCAAGAUUUGCAACUA | 140 | UAGUUGCAAAUCUUGCUCA | 368 | AD00487-19- |
| | | | | 1.um |
| UUGCAACUUGCUACCCAUA | 141 | UAUGGGUAGCAAGUUGCAA | 369 | AD00488-19- |
| | | | | 1.um |
| GCAACUUGCUACCCAUUAA | 142 | UUAAUGGGUAGCAAGUUGC | 370 | AD00489-19- |
| | | | | 1.um |
| CAACUUGCUACCCAUUAGA | 143 | UCUAAUGGGUAGCAAGUUG | 371 | AD00490-19- |
| | | | | 1.um |
| AACUUGCUACCCAUUAGGA | 144 | UCCUAAUGGGUAGCAAGUU | 372 | AD00491-19- |
| | | | | 1.um |
| ACUUGCUACCCAUUAGGAA | 145 | UUCCUAAUGGGUAGCAAGU | 373 | AD00492-19- |
| | | | | 1.um |
| CUUGCUACCCAUUAGGAUA | 146 | UAUCCUAAUGGGUAGCAAG | 374 | AD00493-19- |
| | | | | 1.um |
| GCUACCCAUUAGGAUAAUA | 147 | UAUUAUCCUAAUGGGUAGC | 375 | AD00494-19- |
| | | | | 1.um |
| AUCUGCCAUUGCGAUUGUA | 148 | UACAAUCGCAAUGGCAGAU | 376 | AD00495-19- |
| | | | | 1.um |
| UGCGAUUGUCCAGAGACUA | 149 | UAGUCUCUGGACAAUCGCA | 377 | AD00496-19- |
| | | | | 1.um |
| UGACAUGGCUUCCAGAUAA | 150 | UUAUCUGGAAGCCAUGUCA | 378 | AD00497-19- |
| | | | | 1.um |
| GACAUGGCUUCCAGAUAUA | 151 | UAUAUCUGGAAGCCAUGUC | 379 | AD00498-19- |
| | | | | 1.um |
| ACAUGGCUUCCAGAUAUGA | 152 | UCAUAUCUGGAAGCCAUGU | 380 | AD00499-19- |
| | | | | 1.um |
| UUGGGCAAUAAAGUACCUA | 153 | UAGGUACUUUAUUGCCCAA | 381 | AD00500-19- |
| | | | | 1.um |
| CACCUUUCCCAGUUUUUCA | 154 | UGAAAAACUGGGAAAGGUG | 382 | AD00501-19- |
| | | | | 1.um |
| CCUUUCCCAGUUUUUCACA | 155 | UGUGAAAAACUGGGAAAGG | 383 | AD00502-19- |
| | | | | 1.um |
| UCUUUCAGAGGUGCUAAAA | 156 | UUUUAGCACCUCUGAAAGA | 384 | AD00503-19- |
| | | | | 1.um |
| CUCUGAGCUGAGUUGGUUA | 157 | UAACCAACUCAGCUCAGAG | 385 | AD00504-19- |
| | | | | 1.um |
| AGCUGAGUUGGUUUUAUGA | 158 | UCAUAAAACCAACUCAGCU | 386 | AD00505-19- |
| | | | | 1.um |
| UGAGUUGGUUUUAUGAAAA | 159 | UUUUCAUAAAACCAACUCA | 387 | AD00506-19- |
| | | | | 1.um |
| UUAUGAAAAGCUAGGAAGA | 160 | UCUUCCUAGCUUUUCAUAA | 388 | AD00507-19- |
| | | | | 1.um |
| UUCAGCUGGUUGGGAAAUA | 161 | UAUUUCCCAACCAGCUGAA | 389 | AD00508-19- |
| | | | | 1.um |
| UCAGCUGGUUGGGAAAUGA | 162 | UCAUUUCCCAACCAGCUGA | 390 | AD00509-19- |
| | | | | 1.um |
| GAGGGUCCCUUACUGACUA | 163 | UAGUCAGUAAGGGACCCUC | 391 | AD00510-19- |
| | | | | 1.um |
| CCUAUUAAUGGUCAGACUA | 164 | UAGUCUGACCAUUAAUAGG | 392 | AD00511-19- |
| | | | | 1.um |
| UAUUAAUGGUCAGACUGUA | 165 | UACAGUCUGACCAUUAAUA | 393 | AD00512-19- |
| | | | | 1.um |
| UUAAUGGUCAGACUGUUCA | 166 | UGAACAGUCUGACCAUUAA | 394 | AD00513-19- |
| | | | | 1.um |
| AAUGGUCAGACUGUUCCAA | 167 | UUGGAACAGUCUGACCAUU | 395 | AD00514-19- |
| | | | | 1.um |
| CACCUUUUUCACCUAACUA | 168 | UAGUUAGGUGAAAAAGGUG | 396 | AD00515-19- |
| | | | | 1.um |
| CCUUUUUCACCUAACUAAA | 169 | UUUAGUUAGGUGAAAAAGG | 397 | AD00516-19- |
| | | | | 1.um |
| UUUUUCACCUAACUAAAAA | 170 | UUUUUAGUUAGGUGAAAAA | 398 | AD00517-19- |
| | | | | 1.um |
| AGACAAAGGUGGAUACAUA | 171 | UAUGUAUCCACCUUUGUCU | 399 | AD01072-19- |
| | | | | 1.um |
| UGGAUACAUGAGCAAGAUA | 172 | UAUCUUGCUCAUGUAUCCA | 400 | AD01075-19- |
| | | | | 1.um |
| GGAUACAUGAGCAAGAUUA | 173 | UAAUCUUGCUCAUGUAUCC | 401 | AD01076-19- |
| | | | | 1.um |
| UGCUACCCAUUAGGAUAAA | 174 | UUUAUCCUAAUGGGUAGCA | 402 | AD01081-19- |
| | | | | 1.um |
| UGAGCUGAGUUGGUUUUAA | 175 | UUAAAACCAACUCAGCUCA | 403 | AD01083-19- |
| | | | | 1.um |
| GGUUUUAUGAAAAGCUAGA | 176 | UCUAGCUUUUCAUAAAACC | 404 | AD01085-19- |
| | | | | 1.um |
| nnnGUCCUCUCAGAUCUUGUn | 177 | nACAAGAUCUGAGAGGACnnn | 405 | AD00448-n- |
| | | | | 1.um |
| nnnUUCCAUCCAUCCUUCAAn | 178 | nUUGAAGGAUGGAUGGAAnnn | 406 | AD00449-n- |
| | | | | 1.um |
| nnnUGUCCACCAGCUCAUCUn | 179 | nAGAUGAGCUGGUGGACAnnn | 407 | AD00450-n- |
| | | | | 1.um |
| nnnUACCAGAGUGUCUGAUGn | 180 | nCAUCAGACACUCUGGUAnnn | 408 | AD00451-n- |
| | | | | 1.um |
| nnnUGUCUGAUGGGGAAAACn | 181 | nGUUUUCCCCAUCAGACAnnn | 409 | AD00452-n- |
| | | | | 1.um |
| nnnUCUGAUGGGGAAAACGUn | 182 | nACGUUUUCCCCAUCAGAnnn | 410 | AD00453-n- |
| | | | | 1.um |
| nnnCUGAUGGGGAAAACGUUn | 183 | nAACGUUUUCCCCAUCAGnnn | 411 | AD00454-n- |
| | | | | 1.um |
| nnnGAUGGGGAAAACGUUCUn | 184 | nAGAACGUUUUCCCCAUCnnn | 412 | AD00455-n- |
| | | | | 1.um |
| nnnGGGGAAAACGUUCUGGUn | 185 | nACCAGAACGUUUUCCCCnnn | 413 | AD00456-n- |
| | | | | 1.um |
| nnnAACGUUCUGGUGUCUGAn | 186 | nUCAGACACCAGAACGUUnnn | 414 | AD00457-n- |
| | | | | 1.um |
| nnnCGUUCUGGUGUCUGACUn | 187 | nAGUCAGACACCAGAACGnnn | 415 | AD00458-n- |
| | | | | 1.um |
| nnnUCCAAAGACGAAGUCGUn | 188 | nACGACUUCGUCUUUGGAnnn | 416 | AD00459-n- |
| | | | | 1.um |
| nnnCAGAGGCGUGCGAUAUGn | 189 | nCAUAUCGCACGCCUCUGnnn | 417 | AD00460-n- |
| | | | | 1.um |
| nnnGACAACGUACCCUUCAUn | 190 | nAUGAAGGGUACGUUGUCnnn | 418 | AD00461-n- |
| | | | | 1.um |
| nnnACAACGUACCCUUCAUUn | 191 | nAAUGAAGGGUACGUUGUnnn | 419 | AD00462-n- |
| | | | | 1.um |
| nnnAACGUACCCUUCAUUGAn | 192 | nUCAAUGAAGGGUACGUUnnn | 420 | AD00463-n- |
| | | | | 1.um |
| nnnUAUGGGGAGUACGACAUn | 193 | nAUGUCGUACUCCCCAUAnnn | 421 | AD00464-n- |
| | | | | 1.um |
| nnnACGACAUCUGCCCUAAAn | 194 | nUUUAGGGCAGAUGUCGUnnn | 422 | AD00465-n- |
| | | | | 1.um |
| nnnCGACAUCUGCCCUAAAGn | 195 | nCUUUAGGGCAGAUGUCGnnn | 423 | AD00466-n- |
| | | | | 1.um |
| nnnCAUCUGCCCUAAAGUCAn | 196 | nUGACUUUAGGGCAGAUGnnn | 424 | AD00467-n- |
| | | | | 1.um |
| nnnAUCUGCCCUAAAGUCAAn | 197 | nUUGACUUUAGGGCAGAUnnn | 425 | AD00468-n- |
| | | | | 1.um |
| nnnUCUGCCCUAAAGUCAAGn | 198 | nCUUGACUUUAGGGCAGAnnn | 426 | AD00469-n- |
| | | | | 1.um |
| nnnUGCCCUAAAGUCAAGUCn | 199 | nGACUUGACUUUAGGGCAnnn | 427 | AD00470-n- |
| | | | | 1.um |
| nnnAACUUUCUUCAUGUGGAn | 200 | nUCCACAUGAAGAAAGUUnnn | 428 | AD00471-n- |
| | | | | 1.um |
| nnnUCUUCAUGUGGACAUCAn | 201 | nUGAUGUCCACAUGAAGAnnn | 429 | AD00472-n- |
| | | | | 1.um |
| nnnCUUCAUGUGGACAUCACn | 202 | nGUGAUGUCCACAUGAAGnnn | 430 | AD00473-n- |
| | | | | 1.um |
| nnnCAUGUGGACAUCACCAAn | 203 | nUUGGUGAUGUCCACAUGnnn | 431 | AD00474-n- |
| | | | | 1.um |
| nnnGGACAUCACCAAGCUCAn | 204 | nUGAGCUUGGUGAUGUCCnnn | 432 | AD00475-n- |
| | | | | 1.um |
| nnnUAUGCCUUCGAGGAUAUn | 205 | nAUAUCCUCGAAGGCAUAnnn | 433 | AD00476-n- |
| | | | | 1.um |
| nnnAUGCCUUCGAGGAUAUUn | 206 | nAAUAUCCUCGAAGGCAUnnn | 434 | AD00477-n- |
| | | | | 1.um |
| nnnUGCCUUCGAGGAUAUUUn | 207 | nAAAUAUCCUCGAAGGCAnnn | 435 | AD00478-n- |
| | | | | 1.um |
| nnnGCCUUCGAGGAUAUUUGn | 208 | nCAAAUAUCCUCGAAGGCnnn | 436 | AD00479-n- |
| | | | | 1.um |
| nnnUUCAGGUUCUUGGAAGAn | 209 | nUCUUCCAAGAACCUGAAnnn | 437 | AD00480-n- |
| | | | | 1.um |
| nnnAAAGACAAAGGUGGAUAn | 210 | nUAUCCACCUUUGUCUUUnnn | 438 | AD00481-n- |
| | | | | 1.um |
| nnnAGACAAAGGUGGAUACAn | 211 | nUGUAUCCACCUUUGUCUnnn | 439 | AD00482-n- |
| | | | | 1.um |
| nnnAAAGGUGGAUACAUGAGn | 212 | nCUCAUGUAUCCACCUUUnnn | 440 | AD00483-n- |
| | | | | 1.um |
| nnnGGUGGAUACAUGAGCAAn | 213 | nUUGCUCAUGUAUCCACCnnn | 441 | AD00484-n- |
| | | | | 1.um |
| nnnAUACAUGAGCAAGAUUUn | 214 | nAAAUCUUGCUCAUGUAUnnn | 442 | AD00485-n- |
| | | | | 1.um |
| nnnAUGAGCAAGAUUUGCAAn | 215 | nUUGCAAAUCUUGCUCAUnnn | 443 | AD00486-n- |
| | | | | 1.um |
| nnnGAGCAAGAUUUGCAACUn | 216 | nAGUUGCAAAUCUUGCUCnnn | 444 | AD00487-n- |
| | | | | 1.um |
| nnnUGCAACUUGCUACCCAUn | 217 | nAUGGGUAGCAAGUUGCAnnn | 445 | AD00488-n- |
| | | | | 1.um |
| nnnCAACUUGCUACCCAUUAn | 218 | nUAAUGGGUAGCAAGUUGnnn | 446 | AD00489-n- |
| | | | | 1.um |
| nnnAACUUGCUACCCAUUAGn | 219 | nCUAAUGGGUAGCAAGUUnnn | 447 | AD00490-n- |
| | | | | 1.um |
| nnnACUUGCUACCCAUUAGGn | 220 | nCCUAAUGGGUAGCAAGUnnn | 448 | AD00491-n- |
| | | | | 1.um |
| nnnCUUGCUACCCAUUAGGAn | 221 | nUCCUAAUGGGUAGCAAGnnn | 449 | AD00492-n- |
| | | | | 1.um |
| nnnUUGCUACCCAUUAGGAUn | 222 | nAUCCUAAUGGGUAGCAAnnn | 450 | AD00493-n- |
| | | | | 1.um |
| nnnCUACCCAUUAGGAUAAUn | 223 | nAUUAUCCUAAUGGGUAGnnn | 451 | AD00494-n- |
| | | | | 1.um |
| nnnUCUGCCAUUGCGAUUGUn | 224 | nACAAUCGCAAUGGCAGAnnn | 452 | AD00495-n- |
| | | | | 1.um |
| nnnGCGAUUGUCCAGAGACUn | 225 | nAGUCUCUGGACAAUCGCnnn | 453 | AD00496-n- |
| | | | | 1.um |
| nnnGACAUGGCUUCCAGAUAn | 226 | nUAUCUGGAAGCCAUGUCnnn | 454 | AD00497-n- |
| | | | | 1.um |
| nnnACAUGGCUUCCAGAUAUn | 227 | nAUAUCUGGAAGCCAUGUnnn | 455 | AD00498-n- |
| | | | | 1.um |
| nnnCAUGGCUUCCAGAUAUGn | 228 | nCAUAUCUGGAAGCCAUGnnn | 456 | AD00499-n- |
| | | | | 1.um |
| nnnUGGGCAAUAAAGUACCUn | 229 | nAGGUACUUUAUUGCCCAnnn | 457 | AD00500-n- |
| | | | | 1.um |
| nnnACCUUUCCCAGUUUUUCn | 230 | nGAAAAACUGGGAAAGGUnnn | 458 | AD00501-n- |
| | | | | 1.um |
| nnnCUUUCCCAGUUUUUCACn | 231 | nGUGAAAAACUGGGAAAGnnn | 459 | AD00502-n- |
| | | | | 1.um |
| nnnCUUUCAGAGGUGCUAAAn | 232 | nUUUAGCACCUCUGAAAGnnn | 460 | AD00503-n- |
| | | | | 1.um |
| nnnUCUGAGCUGAGUUGGUUn | 233 | nAACCAACUCAGCUCAGAnnn | 461 | AD00504-n- |
| | | | | 1.um |
| nnnGCUGAGUUGGUUUUAUGn | 234 | nCAUAAAACCAACUCAGCnnn | 462 | AD00505-n- |
| | | | | 1.um |
| nnnGAGUUGGUUUUAUGAAAn | 235 | nUUUCAUAAAACCAACUCnnn | 463 | AD00506-n- |
| | | | | 1.um |
| nnnUAUGAAAAGCUAGGAAGn | 236 | nCUUCCUAGCUUUUCAUAnnn | 464 | AD00507-n- |
| | | | | 1.um |
| nnnUCAGCUGGUUGGGAAAUn | 237 | nAUUUCCCAACCAGCUGAnnn | 465 | AD00508-n- |
| | | | | 1.um |
| nnnCAGCUGGUUGGGAAAUGn | 238 | nCAUUUCCCAACCAGCUGnnn | 466 | AD00509-n- |
| | | | | 1.um |
| nnnAGGGUCCCUUACUGACUn | 239 | nAGUCAGUAAGGGACCCUnnn | 467 | AD00510-n- |
| | | | | 1.um |
| nnnCUAUUAAUGGUCAGACUn | 240 | nAGUCUGACCAUUAAUAGnnn | 468 | AD00511-n- |
| | | | | 1.um |
| nnnAUUAAUGGUCAGACUGUn | 241 | nACAGUCUGACCAUUAAUnnn | 469 | AD00512-n- |
| | | | | 1.um |
| nnnUAAUGGUCAGACUGUUCn | 242 | nGAACAGUCUGACCAUUAnnn | 470 | AD00513-n- |
| | | | | 1.um |
| nnnAUGGUCAGACUGUUCCAn | 243 | nUGGAACAGUCUGACCAUnnn | 471 | AD00514-n- |
| | | | | 1.um |
| nnnACCUUUUUCACCUAACUn | 244 | nAGUUAGGUGAAAAAGGUnnn | 472 | AD00515-n- |
| | | | | 1.um |
| nnnCUUUUUCACCUAACUAAn | 245 | nUUAGUUAGGUGAAAAAGnnn | 473 | AD00516-n- |
| | | | | 1.um |
| nnnUUUUCACCUAACUAAAAn | 246 | nUUUUAGUUAGGUGAAAAnnn | 474 | AD00517-n- |
| | | | | 1.um |
| nnnGACAAAGGUGGAUACAUn | 247 | nAUGUAUCCACCUUUGUCnnn | 475 | AD01072-n- |
| | | | | 1.um |
| nnnGGAUACAUGAGCAAGAUn | 248 | nAUCUUGCUCAUGUAUCCnnn | 476 | AD01075-n- |
| | | | | 1.um |
| nnnGAUACAUGAGCAAGAUUn | 249 | nAAUCUUGCUCAUGUAUCnnn | 477 | AD01076-n- |
| | | | | 1.um |
| nnnGCUACCCAUUAGGAUAAn | 250 | nUUAUCCUAAUGGGUAGCnnn | 478 | AD01081-n- |
| | | | | 1.um |
| nnnGAGCUGAGUUGGUUUUAn | 251 | nUAAAACCAACUCAGCUCnnn | 479 | AD01083-n- |
| | | | | 1.um |
| nnnGUUUUAUGAAAAGCUAGn | 252 | nCUAGCUUUUCAUAAAACnnn | 480 | AD01085-n- |
| | | | | 1.um |

All sequences shown 5′ to 3′ direction.
Duplex AD#s are the number assigned to the duplex of the two strands in the same row in the table.

Table 2 shows certain chemically modified PNPLA3 RNAi agent antisense strand and sense strand sequences of the invention. In some embodiments of methods of the invention, an RNAi agent with a polynucleotide sequence shown in Table 2 is administered to a cell and/or subject. In some embodiments of methods of the invention, an RNAi agent with a polynucleotide sequence shown in Table 2 is administered to a subject. In some embodiments of the invention an RNAi agent administered to a subject comprises is a duplex identified in a row in Table 2, column one and includes the sequence modifications show in the sense and antisense strand sequences in the same row in Table 2, columns three and six, respectively. In some embodiments of methods of the invention, a sequence shown in Table 2 may be attached to (also referred to herein as “conjugated to”) a compound capable of delivering the RNAi agent to a cell and/or tissue in a subject. A non-limiting example of a delivery compound that may be used in certain embodiments of the invention is a GalNAc-containing compound or a GLS-15-containing compound. In Table 2, the first column indicates the Duplex AV # of the base sequences as shown in Table 1. Table 2 discloses Duplex AV # and also shows chemical modifications included in sense and antisense sequence of the duplexes. For example, Table 1 shows base single-strand sequences SEQ ID NO: 25 (sense) and SEQ ID NO: 253 (antisense), which together are the double-stranded duplex identified as: Duplex AD #AD00448.um and Table 2 lists Duplex AV #AV00448, which indicates that the duplex of SEQ ID NO: 481 and SEQ ID NO: 565 includes base sequences of SEQ ID NO: 25 and SEQ ID NO: 253, respectively, but with the chemical modifications shown in the sense and antisense sequences shown in columns three and six, respectively. The “Sense strand SS #” in Table 2 column two is the assigned identifier for the Sense Sequence (including modifications) shown column 3 in the same row. The “Antisense strand AS #” in Table 2 column five is the assigned identifier for the Antisense sequence (including modifications) shown in column six.

TABLE 2
provides chemically modified PNPLA3 RNAi agent antisense strand and sense strand sequences.

| Duplex | Sense strand | | SEQ | Antisense | | SEQ |
|---|---|---|---|---|---|---|
| AV# | SS# | Sense Sequence | ID NO | strand AS# | Antisense Sequence | ID NO |
| AV00448 | AV00448-SS | g*a*gguccuCuCaGaucuugu*a | 481 | AV00448-AS | u*A*caagAucugAgAgGacc*u*c | 565 |
| AV00449 | AV00449-SS | g*u*cuuccaUcCaUccuucaa*a | 482 | AV00449-AS | u*U*ugaaGgaugGaUgGaag*a*c | 566 |
| AV00450 | AV00450-SS | g*a*auguccAcCaGcucaucu*a | 483 | AV00450-AS | u*A*gaugAgcugGuGgAcau*u*c | 567 |
| AV00451 | AV00451-SS | g*c*uuaccaGaGuGucugaug*a | 484 | AV00451-AS | u*C*aucaGacacUcUgGuaa*g*c | 568 |
| AV00452 | AV00452-SS | c*a*gugucuGaUgGggaaaac*a | 485 | AV00452-AS | u*G*uuuuCcccaUcAgAcac*u*g | 569 |
| AV00453 | AV00453-SS | c*u*gucugaUgGgGaaaacgu*a | 486 | AV00453-AS | u*A*cguuUucccCaUcAgac*a*g | 570 |
| AV00454 | AV00454-SS | g*g*ucugauGgGgAaaacguu*a | 487 | AV00454-AS | u*A*acguUuuccCcAuCaga*c*c | 571 |
| AV00455 | AV00455-SS | g*c*ugauggGgAaAacguucu*a | 488 | AV00455-AS | u*A*gaacGuuuuCcCcAuca*g*C | 572 |
| AV00456 | AV00456-SS | c*a*uggggaAaAcGuucuggu*a | 489 | AV00456-AS | U*A*ccagAacguUuUcCcca*u*g | 573 |
| AV00457 | AV00457-SS | c*a*aaacguUcUgGugucuga*a | 490 | AV00457-AS | u*U*cagaCaccaGaAcGuuu*u*g | 574 |
| AV00458 | AV00458-SS | g*a*acguucUgGuGucugacu*a | 491 | AV00458-AS | u*A*gucaGacacCaGaAcgu*u*C | 575 |
| AV00459 | AV00459-SS | g*g*guccaaAgAcGaagucgu*a | 492 | AV00459-AS | u*A*cgacUucguCuUuGgac*c*c | 576 |
| AV00460 | AV00460-SS | g*u*ucagagGcGuGcgauaug*a | 493 | AV00460-AS | u*C*auauCgcacGcCuCuga*a*c | 577 |
| AV00461 | AV00461-SS | g*g*ugacaaCgUaCccuucau*a | 494 | AV00461-AS | u*A*ugaaGgguaCgUuGuca*c*c | 578 |
| AV00462 | AV00462-SS | c*u*gacaacGuAcCcuucauu*a | 495 | AV00462-AS | u*A*augaAggguAcGuUguc*a*g | 579 |
| AV00463 | AV00463-SS | c*a*caacguAcCcUucauuga*a | 496 | AV00463-AS | u*U*caauGaaggGuAcGuug*u*g | 580 |
| AV00464 | AV00464-SS | g*u*cuauggGgAgUacgacau*a | 497 | AV00464-AS | u*A*ugucGuacuCcCcAuag*a*c | 581 |
| AV00465 | AV00465-SS | g*g*uacgacAuCuGcccuaaa*a | 498 | AV00465-AS | u*U*uuagGgcagAuGuCgua*c*c | 582 |
| AV00466 | AV00466-SS | c*u*acgacaUcUgCccuaaag*a | 499 | AV00466-AS | u*C*uuuaGggcaGaUgUcgu*a*g | 583 |
| AV00467 | AV00467-SS | g*g*acaucuGcCcUaaaguca*a | 500 | AV00467-AS | u*U*gacuUuaggGcAgAugu*c*c | 584 |
| AV00468 | AV00468-SS | c*a*caucugCcCuAaagucaa*a | 501 | AV00468-AS | u*U*ugacUuuagGgCaGaug*u*g | 585 |
| AV00469 | AV00469-SS | g*c*aucugcCcUaAagucaag*a | 502 | AV00469-AS | u*C*uugaCuuuaGgGcAgau*g*C | 586 |
| AV00470 | AV00470-SS | g*u*cugcccUaAaGucaaguc*a | 503 | AV00470-AS | u*G*acuuGacuuUaGgGcag*a*c | 587 |
| AV00471 | AV00471-SS | g*c*gaacuuUcUuCaugugga*a | 504 | AV00471-AS | u*U*ccacAugaaGaAaGuuc*g*C | 588 |
| AV00472 | AV00472-SS | g*u*uucuucAuGuGgacauca*a | 505 | AV00472-AS | u*U*gaugUccacAuGaAgaa*a*c | 589 |
| AV00473 | AV00473-SS | g*u*ucuucaUgUgGacaucac*a | 506 | AV00473-AS | u*G*ugauGuccaCaUgAaga*a*c | 590 |
| AV00474 | AV00474-SS | g*u*ucauguGgAcAucaccaa*a | 507 | AV00474-AS | u*U*ugguGauguCcAcAuga*a*c | 591 |
| AV00475 | AV00475-SS | g*g*uggacaUcAcCaagcuca*a | 508 | AV00475-AS | u*U*gagcUugguGaUgUcca*c*c | 592 |
| AV00476 | AV00476-SS | g*g*auaugcCuUcGaggauau*a | 509 | AV00476-AS | u*A*uaucCucgaAgGcAuau*c*c | 593 |
| AV00477 | AV00477-SS | c*a*uaugccUuCgAggauauu*a | 510 | AV00477-AS | u*A*auauCcucgAaGgCaua*u*g | 594 |
| AV00478 | AV00478-SS | g*u*augccuUcGaGgauauuu*a | 511 | AV00478-AS | u*A*aauaUccucGaAgGcau*a*c | 595 |
| AV00479 | AV00479-SS | g*a*ugccuuCgAgGauauuug*a | 512 | AV00479-AS | u*C*aaauAuccuCgAaGgca*u*c | 596 |
| AV00480 | AV00480-SS | c*c*auucagGuUcUuggaaga*a | 513 | AV00480-AS | u*U*cuucCaagaAcCuGaau*g*g | 597 |
| AV00481 | AV00481-SS | g*u*gaaagaCaAaGguggaua*a | 514 | AV00481-AS | u*U*auccAccuuUgUcUuuc*a*c | 598 |
| AV00482 | AV00482-SS | c*a*aagacaAaGgUggauaca*a | 515 | AV00482-AS | u*U*guauCcaccUuUgUcuu*u*g | 599 |
| AV00483 | AV00483-SS | c*a*caaaggUgGaUacaugag*a | 516 | AV00483-AS | u*C*ucauGuaucCaCcUuug*u*g | 600 |
| AV00484 | AV00484-SS | g*a*agguggAuAcAugagcaa*a | 517 | AV00484-AS | u*U*ugcuCauguAuCcAccu*u*c | 601 |
| AV00485 | AV00485-SS | g*g*gauacaUgAgCaagauuu*a | 518 | AV00485-AS | u*A*aaucUugcuCaUgUauc*c*c | 602 |
| AV00486 | AV00486-SS | g*a*caugagCaAgAuuugcaa*a | 519 | AV00486-AS | u*U*ugcaAaucuUgCuCaug*u*c | 603 |
| AV00487 | AV00487-SS | g*a*ugagcaAgAuUugcaacu*a | 520 | AV00487-AS | u*A*guugCaaauCuUgCuca*u*c | 604 |
| AV00488 | AV00488-SS | g*u*uugcaaCuUgCuacccau*a | 521 | AV00488-AS | u*A*ugggUagcaAgUuGcaa*a*c | 605 |
| AV00489 | AV00489-SS | g*u*gcaacuUgCuAcccauua*a | 522 | AV00489-AS | u*U*aaugGguagCaAgUugc*a*c | 606 |
| AV00490 | AV00490-SS | g*g*caacuuGcUaCccauuag*a | 523 | AV00490-AS | u*C*uaauGgguaGcAaGuug*C*C | 607 |
| AV00491 | AV00491-SS | c*c*aacuugCuAcCcauuagg*a | 524 | AV00491-AS | u*C*cuaaUggguAgCaAguu*g*g | 608 |
| AV00492 | AV00492-SS | g*a*acuugcUaCcCauuagga*a | 525 | AV00492-AS | u*U*ccuaAugggUaGcAagu*u*c | 609 |
| AV00493 | AV00493-SS | g*a*cuugcuAcCcAuuaggau*a | 526 | AV00493-AS | u*A*uccuAauggGuAgCaag*u*c | 610 |
| AV00494 | AV00494-SS | g*u*gcuaccCaUuAggauaau*a | 527 | AV00494-AS | u*A*uuauCcuaaUgGgUagc*a*c | 611 |
| AV00495 | AV00495-SS | c*a*aucugcCaUuGcgauugu*a | 528 | AV00495-AS | u*A*caauCgcaaUgGcAgau*u*g | 612 |
| AV00496 | AV00496-SS | g*u*ugcgauUgUcCagagacu*a | 529 | AV00496-AS | u*A*gucuCuggaCaAuCgca*a*c | 613 |
| AV00497 | AV00497-SS | c*g*ugacauGgCuUccagaua*a | 530 | AV00497-AS | u*U*aucuGgaagCcAuGuca*c*g | 614 |
| AV00498 | AV00498-SS | c*u*gacaugGcUuCcagauau*a | 531 | AV00498-AS | u*A*uaucUggaaGcCaUguc*a*g | 615 |
| AV00499 | AV00499-SS | g*g*acauggCuUcCagauaug*a | 532 | AV00499-AS | u*C*auauCuggaAgCcAugu*c*c | 616 |
| AV00500 | AV00500-SS | g*c*uugggcAaUaAaguaccu*a | 533 | AV00500-AS | u*A*gguaCuuuaUuGcCcaa*g*c | 617 |
| AV00501 | AV00501-SS | g*c*caccuuUcCcAguuuuuc*a | 534 | AV00501-AS | u*G*aaaaAcuggGaAaGgug*g*c | 618 |
| AV00502 | AV00502-SS | g*a*ccuuucCcAgUuuuucac*a | 535 | AV00502-AS | u*G*ugaaAaacuGgGaAagg*u*c | 619 |
| AV00503 | AV00503-SS | g*u*ucuuucAgAgGugcuaaa*a | 536 | AV00503-AS | u*U*uuagCaccuCuGaAaga*a*c | 620 |
| AV00504 | AV00504-SS | c*c*cucugaGcUgAguugguu*a | 537 | AV00504-AS | u*A*accaAcucaGcUcAgag*g*g | 621 |
| AV00505 | AV00505-SS | g*g*agcugaGuUgGuuuuaug*a | 538 | AV00505-AS | u*C*auaaAaccaAcUcAgcu*c*c | 622 |
| AV00506 | AV00506-SS | c*c*ugaguuGgUuUuaugaaa*a | 539 | AV00506-AS | u*U*uucaUaaaaCcAaCuca*g*g | 623 |
| AV00507 | AV00507-SS | g*u*uuaugaAaAgCuaggaag*a | 540 | AV00507-AS | u*C*uuccUagcuUuUcAuaa*a*c | 624 |
| AV00508 | AV00508-SS | g*a*uucagcUgGuUgggaaau*a | 541 | AV00508-AS | u*A*uuucCcaacCaGcUgaa*u*c | 625 |
| AV00509 | AV00509-SS | g*u*ucagcuGgUuGggaaaug*a | 542 | AV00509-AS | u*C*auuuCccaaCcAgCuga*a*c | 626 |
| AV00510 | AV00510-SS | g*a*gaggguCcCuUacugacu*a | 543 | AV00510-AS | u*A*gucaGuaagGgAcCcuc*u*c | 627 |
| AV00511 | AV00511-SS | c*c*ccuauuAaUgGucagacu*a | 544 | AV00511-AS | u*A*gucuGaccaUuAaUagg*g*g | 628 |
| AV00512 | AV00512-SS | g*c*uauuaaUgGuCagacugu*a | 545 | AV00512-AS | u*A*caguCugacCaUuAaua*g*c | 629 |
| AV00513 | AV00513-SS | g*a*uuaaugGuCaGacuguuc*a | 546 | AV00513-AS | u*G*aacaGucugAcCaUuaa*u*c | 630 |
| AV00514 | AV00514-SS | g*u*aaugguCaGaCuguucca*a | 547 | AV00514-AS | u*U*ggaaCagucUgAcCauu*a*c | 631 |
| AV00515 | AV00515-SS | g*a*caccuuUuUcAccuaacu*a | 548 | AV00515-AS | u*A*guuaGgugaAaAaGgug*u*c | 632 |
| AV00516 | AV00516-SS | g*a*ccuuuuUcAcCuaacuaa*a | 549 | AV00516-AS | u*U*uaguUagguGaAaAagg*U*C | 633 |
| AV00517 | AV00517-SS | g*c*uuuuucAcCuAacuaaaa*a | 550 | AV00517-AS | u*U*uuuaGuuagGuGaAaaa*g*C | 634 |
| AV01072 | AV01072-SS | (invab)*g*aagacaaAgGugGa | 551 | AV01072-AS | u*A*ugUauccacCuUuguCu*u*c | 635 |
| | | uacau*a*(invab) | | | | |
| AV01073 | AV01073-SS | (invab)*c*acaaaggUgGauAc | 552 | AV01073-AS | u*C*ucAuguaucCaCcuuUg*u*g | 636 |
| | | augag*a*(invab) | | | | |
| AV01074 | AV01074-SS | (invab)*g*aagguggAuAcaUg | 553 | AV01074-AS | u*U*ugCucauguAuCcacCu*u*c | 637 |
| | | agcaa*a*(invab) | | | | |
| AV01075 | AV01075-SS | (invab)*c*guggauaCaUgaGc | 554 | AV01075-AS | u*A*ucUugcucaUgUaucCa*c*g | 638 |
| | | aagau*a*(invab) | | | | |
| AV01076 | AV01076-SS | (invab)*c*uggauacAuGagCa | 555 | AV01076-AS | u*A*auCuugcucAuGuauCc*a*g | 639 |
| | | agauu*a*(invab) | | | | |
| AV01077 | AV01077-SS | (invab)*g*uuugcaaCuUgcUa | 556 | AV01077-AS | u*A*ugGguagcaAgUugcAa*a*c | 640 |
| | | cccau*a*(invab) | | | | |
| AV01078 | AV01078-SS | (invab)*g*ugcaacuUgCuaCc | 557 | AV01078-AS | u*U*aaUggguagCaAguuGc*a*c | 641 |
| | | cauua*a*(invab) | | | | |
| AV01079 | AV01079-SS | (invab)*g*gcaacuuGcUacCc | 558 | AV01079-AS | u*C*uaAuggguaGcAaguUg*c*c | 642 |
| | | auuag*a*(invab) | | | | |
| AV01080 | AV01080-SS | (invab)*c*caacuugCuAccCa | 559 | AV01080-AS | u*C*cuAauggguAgCaagUu*g*g | 643 |
| | | uuagg*a*(invab) | | | | |
| AV01081 | AV01081-SS | (invab)*g*uugcuacCcAuuAg | 560 | AV01081-AS | u*U*uaUccuaauGgGuagCa*a*c | 644 |
| | | gauaa*a*(invab) | | | | |
| AV01082 | AV01082-SS | (invab)*g*ugcuaccCaUuaGg | 561 | AV01082-AS | U*A*uuAuccuaaUgGguaGc*a*c | 645 |
| | | auaau*a*(invab) | | | | |
| AV01083 | AV01083-SS | (invab)*g*cugagcuGaGuuGg | 562 | AV01083-AS | u*U*aaAaccaacUcAgcuCa*g*c | 646 |
| | | uuuua*a*(invab) | | | | |
| AV01084 | AV01084-SS | (invab)*g*gagcugaGuUggUu | 563 | AV01084-AS | u*C*auAaaaccaAcUcagCu*c*c | 647 |
| | | uuaug*a*(invab) | | | | |
| AV01085 | AV01085-SS | (invab)*g*ugguuuuAuGaaAa | 564 | AV01085-AS | u*C*uaGcuuuucAuAaaaCc*a*c | 648 |
| | | gcuag*a*(invab) | | | | |

All sequences shown 5′ to 3′.
These sequences were used in certain in vitro testing studies described herein.
The chemical modifications are indicated by: Upper case: 2′-Fluoro; lower case: 2′-OMe; thiophosphate: * , and it can be understood by person skilled in the art that “*” is a symbol for indication of linkage relationship, wherein the presence of “*” means that monomers are linked to each other via a phosphorothioate diester linkage, wherein the absence of “*” between two monomers indicates that the monomers are linked to each other via a phosphodiester linkage; and invab = inverted abasic.

Table 3 shows certain chemically modified PNPLA3 RNAi agent antisense strand and sense strand sequences of the invention. In some embodiments of methods of the invention, RNAi agents shown in Table 3 are administered to a cell and/or subject. In some embodiments of methods of the invention, an RNAi agent with a polynucleotide sequence shown in Table 3 is administered to a subject. In some embodiments of the invention an RNAi agent administered to a subject comprises is a duplex identified in a row in Table 3, column one and includes the sequence modifications and/or delivery compound show in the sense and antisense strand sequences in the same row in Table 3, columns three and six, respectively. The sequences were used in certain in vivo testing studies described elsewhere herein. In some embodiments of methods of the invention, a sequence shown in Table 3 may be attached to (also referred to herein as “conjugated to”) a compound for delivery, a non-limiting example of which is a GalNAc-containing compound, with a delivery compound identified in Table 3 as “GLX-n” on sense strands in column three. As used herein, “GLX-n” is used to represent either a “GLS-n” or a GLO-n″ delivery compound (“X” can be either “S” or “O”) and GLX-0 can be any of the “GLS-n” and “GLO-n” delivery compounds that can be attached to 3′-end or 5′-end of oligonucleotide during synthesis. As used herein and shown in Table 3, “GLX-n” is used to indicate the attached GalNAc-containing compound is any one of compounds GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, The structure of each of which is provided elsewhere herein. One skilled in the art will be able to prepare and use a dsRNA compound of the invention in which the attached delivery compound is one of GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16. Column one of Table 3 provides a Duplex AD # assigned to the duplex of the sense and antisense sequences in that row of the table. For example, Duplex AD #AD00451 is the duplex of sense strand SEQ ID NO: 650 and antisense strand SEQ ID NO: 687. Each line in Table 3 provides a sense strand and an antisense strand, and discloses the duplex of the sense and antisense strands shown. The “Sense strand SS #” in Table 3 column two is the assigned identifier for the Sense Sequence (including modifications) shown column 3 in the same row. The “Antisense strand AS #” in Table 3 column five is the assigned identifier for the Antisense sequence (including modifications) shown in column six. An identifier for certain attached GalNAc-containing “GLO-n” or “GLS-n” compounds is shown as GLS-5, GLS-15 or GLX-0, and it will be understood that another of the “GLO-n” or “GLS-n” compounds may be substituted for the compound shown as GLO-0, with the resulting compound included in an embodiment of a method and/or a composition of the invention. GLO-0 is refers to the compound GalNAc3 in Jayaprakash, et al., (2014) J. Am. Chem. Soc., 136, 16958-16961.

TABLE 3
provides chemically modified PNPLA3 RNAi agent antisense strand and sense strand sequences. All sequences shown 5′ to 3′.
The sequences were used in certain in vivo testing studies described elsewhere herein. A delivery molecule used in the in vivo studies is indicated
as “GLO-n” or “GLS-n” at the 3′-end or 5′-end of each sense strand. Chemical modifications are indicated as: upper case: 2′-Fluoro; lower case:
2′-OMe; thiophosphate: * , and it can be understood by person skilled in the art that “*” is a symbol for indication of linkage relationship, wherein
the presence of “*” means that monomers are linked to each other via a phosphorothioate diester linkage, wherein the absence of “*” between two
monomers indicates that the monomers are linked to each other via a phosphodiester linkage;

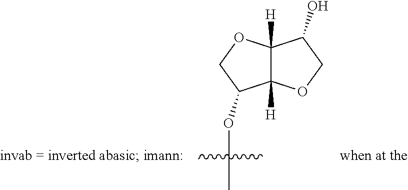 [SMILES: *O[C@@H]1CO[C@H]2[C@@H]1OC[C@H]2O]

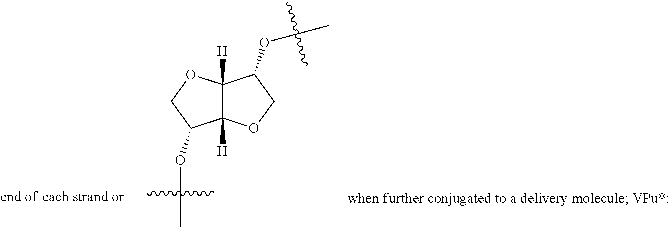 [SMILES: *O[C@@H]1CO[C@H]2[C@@H]1OC[C@H]2O*]

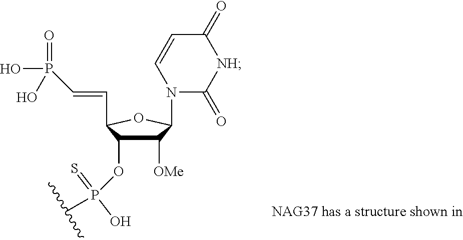

US20190256849 as “NAG37”, and person skilled in the art would know the linking mode when there is a phosphorothioate linkage (represented
by “*”).

| | Sense | | SEQ | Antisense | | SEQ |
|---|---|---|---|---|---|---|
| Duplex | strand | | ID | strand | | ID |
| AD# | SS# | Sense Sequence | NO | AS# | Antisense Sequence | NO |
| AD00444 | AD00444-SS | (GLS-5)*(invab)*acaccuuuUuCaCcuaacuaa*(invab) | 649 | AD00444-AS | u*U*aguuAggugAaAaAggu*g*u | 686 |
| AD00451 | AD00451-SS | (GLS-5)*(invab)*gcuuaccaGaGuGucugauga*(invab) | 650 | AD00451-AS | u*C*aucaGacacUcUgGuaa*g*c | 687 |
| AD00452 | AD00452-SS | (GLS-5)*(invab)*cugucugaUgGgGaaaacgua*(invab) | 651 | AD00452-AS | u*A*cguuUucccCaUcAgac*a*g | 688 |
| AD00453 | AD00453-SS | (GLS-5)*(invab)*gguacgacAuCuGcccuaaaa*(invab) | 652 | AD00453-AS | u*U*uuagGgcagAuGuCgua*c*c | 689 |
| AD00454 | AD00454-SS | (GLS-5)*(invab)*cacaucugCcCuAaagucaaa*(invab) | 653 | AD00454-AS | u*U*ugacUuuagGgCaGaug*u*g | 690 |
| AD00455 | AD00455-SS | (GLS-5)*(invab)*caaagacaAaGgUggauacaa*(invab) | 654 | AD00455-AS | u*U*guauCcaccUuUgUcuu*u*g | 691 |
| AD00456 | AD00456-SS | (GLS-5)*(invab)*cgugacauGgCuUccagauaa*(invab) | 655 | AD00456-AS | u*U*aucuGgaagCcAuGuca*c*g | 692 |
| AD00457 | AD00457-SS | (GLS-5)*(invab)*cugacaugGcUuCcagauaua*(invab) | 656 | AD00457-AS | u*A*uaucUggaaGcCaUguc*a*g | 693 |
| AD00458 | AD00458-SS | (GLS-5)*(invab)*gauucagcUgGuUgggaaaua*(invab) | 657 | AD00458-AS | u*A*uuucCcaacCaGcUgaa*u*C | 694 |
| AD00460 | AD00460-SS | (GLS-5)*(invab)*ccccuauuAaUgGucagacua*(invab) | 659 | AD00460-AS | U*A*gucuGaccaUuAaUagg*g*g | 696 |
| AD00585 | AD00585-SS | g*g*ccuuAuCCCuccuuccuua(GLO-0) | 660 | AD00585-AS | u*A*aggAaGGagggAuAaggcc*a*c | 697 |
| AD00652 | AD00652-SS | (GLS-15)*(invab)*gcgaacuuUcUuCaugugga*a*(invab) | 661 | AD00652-AS | u*U*ccacAugaaGaAaGuuc*g*C | 698 |
| AD00653 | AD00653-SS | (GLS- | 662 | AD00653-AS | u*U*auccAccuuUgUcUuuc*a*c | 699 |
| | | 15)*(invab)*gugaaagaCaAaGguggaua*a*(invab) | | | | |
| AD00654 | AD00654-SS | (GLS- | 663 | AD00654-AS | u*A*aaucUugcuCaUgUauc*c*c | 700 |
| | | 15)*(invab)*gggauacaUgAgCaagauuu*a*(invab) | | | | |
| AD00655 | AD00655-SS | (GLS-15)*(invab)*gacaugagCaAgAuuugcaa*a*(invab) | 664 | AD00655-AS | u*U*ugcaAaucuUgCuCaug*u*c | 701 |
| AD00656 | AD00656-SS | (GLS-15)*(invab)*gaugagcaAgAuUugcaacu*a*(invab) | 665 | AD00656-AS | u*A*guugCaaauCuUgCuca*u*c | 702 |
| AD00657 | AD00657-SS | (GLS-15)*(invab)*gacuugcuAcCcAuuaggau*a*(invab) | 666 | AD00657-AS | u*A*uccuAauggGuAgCaag*u*c | 703 |
| AD00658 | AD00658-SS | (GLS-15)*(invab)*ggacauggCuUcCagauaug*a*(invab) | 667 | AD00658-AS | u*C*auauCuggaAgCcAugu*c*c | 704 |
| AD00659 | AD00659-SS | (GLS-15)*(invab)*gccaccuuUcCcAguuuuuc*a*(invab) | 668 | AD00659-AS | u*G*aaaaAcuggGaAaGgug*g*c | 705 |
| AD00660 | AD00660-SS | (GLS-15)*(invab)*cccucugaGcUgAguugguu*a*(invab) | 669 | AD00660-AS | u*A*accaAcucaGcUcAgag*g*g | 706 |
| AD00661 | AD00661-SS | (GLS- | 670 | AD00661-AS | u*U*uucaUaaaaCcAaCuca*g*g | 707 |
| | | 15)*(invab)*ccugaguuGgUuUuaugaaa*a*(invab) | | | | |
| AD00662 | AD00662-SS | (GLS-15)*(invab)*gagaggguCcCuUacugacu*a*(invab) | 671 | AD00662-AS | u*A*gucaGuaagGgAcCcuc*u*c | 708 |
| AD00663 | AD00663-SS | (GLS-15)*(invab)*gacaccuuUuUcAccuaacu*a*(invab) | 672 | AD00663-AS | u*A*guuaGgugaAaAaGgug*u*C | 709 |
| AD00664 | AD00664-SS | (GLS-15)*(invab)*gaccuuuuUcAcCuaacuaa*a*(invab) | 673 | AD00664-AS | u*U*uaguUagguGaAaAagg*u*C | 710 |
| AD00745 | AD00745-SS | (GLS-15)*(invab)*gagguccuCuCagAucuugu*a*(invab) | 674 | AD00745-AS | u*A*caAgaucugAgAggaCc*u*c | 711 |
| AD00746 | AD00746-SS | (GLS-15)*(invab)*gucuuccaUcCauCcuucaa*a*(invab) | 675 | AD00746-AS | u*U*ugAaggaugGaUggaAg*a*c | 712 |
| AD00747 | AD00747-SS | (GLS- | 676 | AD00747-AS | u*A*ccAgaacguUuUcccCa*u*g | 713 |
| | | 15)*(invab)*cauggggaAaAcgUucuggu*a*(invab) | | | | |
| AD00748 | AD00748-SS | (GLS-15)*(invab)*ggguccaaAgAcgAagucgu*a*(invab) | 677 | AD00748-AS | u*A*cgAcuucguCuUuggAc*c*c | 714 |
| AD00749 | AD00749-SS | (GLS-15)*(invab)*ggugacaaCgUacCcuucau*a*(invab) | 678 | AD00749-AS | u*A*ugAaggguaCgUuguCa*c*c | 715 |
| AD00750 | AD00750-SS | (GLS-15)*(invab)*cacaacguAcCcuUcauuga*a*(invab) | 679 | AD00750-AS | u*U*caAugaaggGuAcguUg*u*g | 716 |
| AD00815 | AD00815-SS | (GLS-15)*(invab)*gaagacaaAgGuGgauacau*a*(invab) | 680 | AD00815-AS | u*A*uguaUccacCuUuGucu*u*c | 717 |
| AD00816 | AD00816-SS | (GLS-15)*(invab)*gugcaacuUgCuAcccauua*a*(invab) | 681 | AD00816-AS | u*U*aaugGguagCaAgUugc*a*C | 718 |
| AD00817 | AD00817-SS | (GLS- | 682 | AD00817-AS | u*C*auaaAaccaAcUcAgcu*c*c | 719 |
| | | 15)*(invab)*ggagcugaGuUgGuuuuaug*a*(invab) | | | | |
| AD00818 | AD00818-SS | (GLS-15)*(invab)*guugcuacCcAuUaggauaa*a*(invab) | 683 | AD00818-AS | u*U*uaucCuaauGgGuAgca*a*c | 720 |
| AD00819 | AD00819-SS | (GLS- | 684 | AD00819-AS | u*C*uagcUuuucAuAaAacc*a*c | 721 |
| | | 15)*(invab)*gugguuuuAuGaAaagcuag*a*(invab) | | | | |
| AD00820 | AD00820-SS | (GLS- | 685 | AD00820-AS | u*U*aaaaCcaacUcAgCuca*g*c | 722 |
| | | 15)*(invab)*gcugagcuGaGuUgguuuua*a*(invab) | | | | |
| AD00663-1 | AD00663-1- | (GLS- | 723 | AD00663-1-AS | u*A*guuaGgugaAaAaGgug*u*c | 729 |
| | SS | 15)*(imann)*gacaccuuUuUcAccuaacu*a*(imann) | | | | |
| AD00664-1 | AD00664-1- | (GLS- | 724 | AD00664-1-AS | u*U*uaguUagguGaAaAagg*u*C | 730 |
| | SS | 15)*(imann)*gaccuuuuUcAcCuaacuaa*a*(imann) | | | | |
| AD00815-1 | AD00815-1- | (GLS- | 725 | AD00815-1-AS | u*A*uguaUccacCuUuGucu*u*c | 731 |
| | SS | 15)*(imann)*gaagacaaAgGuGgauacau*a*(imann) | | | | |
| AD00816-1 | AD00816-1- | (GLS- | 726 | AD00816-1-AS | u*U*aaugGguagCaAgUugc*a*c | 732 |
| | SS | 15)*(imann)*gugcaacuUgCuAcccauua*a*(imann) | | | | |
| AD00819-1 | AD00819-1- | (GLS- | 727 | AD00819-1-AS | u*C*uagcUuuucAuAaAacc*a*c | 733 |
| | SS | 15)*(imann)*gugguuuuAuGaAaagcuag*a*(imann) | | | | |
| AD00444-1 | AD00444-1- | (GLS-15)*(invab)*acaccuuuUUCaccuaacuaa*(invab) | 728 | AD00444-1-AS | u*U*a*GuUaGgUgAaAaAgGuG*u | 734 |
| | SS | | | | | |
| AD00663-2 | AD00663-2- | (GLS-15)*(invab)*gacaccuuUuUcAccuaacu*a*(invab) | 735 | AD00663-2-AS | VPu*A*guuaGgugaAaAaGgug*u*c | 740 |
| | SS | | | | | |
| AD00664-2 | AD00664-2- | (GLS-15)*(invab)*gaccuuuuUcAcCuaacuaa*a*(invab) | 736 | AD00664-2-AS | VPu*U*uaguUagguGaAaAagg*u*c | 741 |
| | SS | | | | | |
| AD00815-2 | AD00815-2- | (GLS-15)*(invab)*gaagacaaAgGuGgauacau*a*(invab) | 737 | AD00815-2-AS | VPu*A*uguaUccacCuUuGucu*u*c | 742 |
| | SS | | | | | |
| AD02242 | AD02242-SS | (NAG37)*(invab)*acaccuuuUUCaccuaacuaa*(invab) | 738 | AD02242-AS | u*U*a*GuUaGgUgAaAaAgGuG*U | 743 |
| AD02250 | AD02250-SS | (GLS-15)*(invab)*acaccuuuUuCaCcuaacua*a*(invab) | 739 | AD02250-AS | u*U*aguuAggugAaAaAggu*g*U | 744 |

In certain embodiments of the invention a dsRNA (also referred to herein as a “duplex”) is one disclosed in one of Tables 1-3. Each row in Tables 1-3 discloses a duplex comprising the sequence of the sense strand and the sequence of the antisense strand in that table row. In addition to the duplexes disclosed in Tables 1-3, it will be understood that in some embodiments, a duplex of the invention may include sense and antisense sequences shown in Tables 1-3, that differ by zero, one, two, or three nucleotides shown in a sequence shown in Tables 1-3. Thus, as non-limiting examples, in some embodiments, an antisense strand in a duplex of the invention may be SEQ ID NO: 253, 565, 609, 635, 648, 702, 709, 710 or 717 with zero, one, two, or three different nucleotides than those in SEQ ID NO: 253, 565, 609, 635, 648, 702, 709, 710 or 717, respectively.

It will be understood that the sequence of the sense strand and the sequence of the antisense strand in a duplex of the invention may be independently selected. Thus, a dsRNA of the invention may comprise a sense strand and an antisense strand of a duplex disclosed in a row in Tables 1-3. Alternatively, in a dsRNA of the invention, one or both of the selected sense and antisense strand in the dsRNA may include sequences shown in Tables 1-3 but with one or both of the sense and antisense sequences including 1, 2, 3, or more nucleobase substitutions from the parent sequence. The selected sequences may in some embodiments be longer or shorter than their parent sequence. Thus, dsRNA agents included in the invention can but need not include exact sequences of the sense and antisense pairs disclosed as duplexes in Tables 1-3.

In some embodiments, a dsRNA agent comprises a sense strand and an antisense strand, nucleotide positions 2 to 18 in the antisense strand comprising a region of complementarity to a PNPLA3 RNA transcript, wherein the region of complementarity comprises at least 15 contiguous nucleotides that differ by 0, 1, 2, or 3 nucleotides from one of the antisense sequences listed in one of Tables 1-3, and optionally comprising a targeting ligand. In some instances, the region of complementarity to the PNPLA3 RNA transcript comprises at least 15, 16, 17, 18, or 19 contiguous nucleotides that differ by no more than 3 nucleotides from one of the antisense sequences listed in one of Tables 1-3. In some embodiments of a dsRNA agent of the invention, the antisense strand of the dsRNA is at least substantially complementary to any one of a target region of SEQ ID NO: 1 and is provided in any one of Tables 1-3. In some embodiments, an antisense strand of a dsRNA agent of the invention is fully complementary to any one of a target region of SEQ ID NO: 1 and is provided in any one of Tables 1-3. In some embodiments a dsRNA agent includes a sense strand sequence set forth in any one of Tables 1-3, and the sense strand sequence is at least substantially complementary to the antisense strand sequence in the dsRNA agent. In other embodiments, a dsRNA agent of the invention comprises a sense strand sequence set forth in any one of Tables 1-3, and the sense strand sequence is fully complementary to the antisense strand sequence in the dsRNA agent. In some instances, a dsRNA agent of the invention comprises an antisense strand sequence set forth in any one of Tables 1-3. Some embodiments of a dsRNA agent of the invention comprises the sense and antisense sequences disclosed as duplex in any of Tables 1-3. As described herein, it will be understood that the sense and antisense strands in a duplex of the invention may be independently selected.

### Mismatches

It is known to skilled in art, mismatches are tolerated for efficacy in dsRNA, especially the mismatches are within terminal region of dsRNA. Certain mismatches tolerate better, for example mismatches with wobble base pairs G: U and A: C are tolerated better for efficacy (Du et el., A systematic analysis of the silencing effects of an active siRNA at all single-nucleotide mismatched target sites. Nucleic Acids Res. 2005 Mar. 21; 33(5):1671-7. Doi: 10.1093/nar/gki312. Nucleic Acids Res. 2005; 33(11):3698). Some embodiments of methods and compounds of the invention a PNPLA3 dsRNA agent may contain one or more mismatches to the PNPLA3 target sequence. In some embodiments, PNPLA3 dsRNA agent of the invention includes no mismatches. In certain embodiments, PNPLA3 dsRNA agent of the invention includes no more than 1 mismatch. In some embodiments, PNPLA3 dsRNA agent of the invention includes no more than 2 mismatches. In certain embodiments, PNPLA3 dsRNA agent of the invention includes no more than 3 mismatches. In some embodiments of the invention, an antisense strand of a PNPLA3 dsRNA agent contains mismatches to a PNPLA3 target sequence that are not located in the center of the region of complementarity. In some embodiments, the antisense strand of the PNPLA3 dsRNA agent includes 1, 2, 3, 4, or more mismatches that are within the last 5, 4, 3, 2, or 1 nucleotides from one or both of the 5′ or 3′ end of the region of complementarity. Methods described herein and/or methods known in the art can be used to determine whether a PNPLA3 dsRNA agent containing a mismatch to a PNPLA3 target sequence is effective in inhibiting the expression of the PNPLA3 gene.

### Complementarity

As used herein, unless otherwise indicated, the term “complementary,” when used to describe a first nucleotide sequence (e.g., PNPLA3 dsRNA agent sense strand or targeted PNPLA3 mRNA) in relation to a second nucleotide sequence (e.g., PNPLA3 dsRNA agent antisense strand or a single-stranded antisense polynucleotide), means the ability of an oligonucleotide or polynucleotide including the first nucleotide sequence to hybridize [form base pair hydrogen bonds under mammalian physiological conditions (or similar conditions in vitro)] and form a duplex or double helical structure under certain conditions with an oligonucleotide or polynucleotide including the second nucleotide sequence. Other conditions, such as physiologically relevant conditions as can be encountered inside an organism, can apply. A skilled artisan will be able to determine the set of conditions most appropriate for a test of complementarity of two sequences in accordance with the ultimate application of the hybridized nucleotides. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification.

Complementary sequences, for example, within a PNPLA3 dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. It will be understood that in embodiments when two oligonucleotides are designed to form, upon hybridization, one or more single stranded overhangs, such overhangs are not regarded herein as mismatches with regard to the determination of complementarity. For example, a PNPLA3 dsRNA agent comprising one oligonucleotide 19 nucleotides in length and another oligonucleotide 20 nucleotides in length, wherein the longer oligonucleotide comprises a sequence of 19 nucleotides that is fully complementary to the shorter oligonucleotide, can yet be referred to as “fully complementary” for the purposes described herein. Thus, as used herein, “fully complementary” means that all (100%) of the bases in a contiguous sequence of a first polynucleotide will hybridize with the same number of bases in a contiguous sequence of a second polynucleotide. The contiguous sequence may comprise all or a part of a first or second nucleotide sequence.

The term “substantially complementary” as used herein means that in a hybridized pair of nucleobase sequences, at least about 85%, 86%, 87%, 88%, 89%, 90%, 91%, 92%, 93%, 94%, 95%, 96%, 97%, 98%, or 99%, but not all, of the bases in a contiguous sequence of a first polynucleotide will hybridize with the same number of bases in a contiguous sequence of a second polynucleotide. The term “substantially complementary” can be used in reference to a first sequence with respect to a second sequence if the two sequences include one or more, for example at least 1, 2, 3, 4, or 5 mismatched base pairs upon hybridization for a duplex up to 15, 16, 17, 18, 19, 20, 21, 22, 23, 24, 25, 26, 27, 28, 29 or 30 base pairs (bp), while retaining the ability to hybridize under the conditions most relevant to their ultimate application, e.g., inhibition of PNPLA3 gene expression via a RISC pathway.

The term, “partially complementary” may be used herein in reference to a hybridized pair of nucleobase sequences, in which at least 75%, but not all, of the bases in a contiguous sequence of a first polynucleotide will hybridize with the same number of bases in a contiguous sequence of a second polynucleotide. In some embodiments, “partially complementary” means at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the bases in a contiguous sequence of a first polynucleotide will hybridize with the same number of bases in a contiguous sequence of a second polynucleotide.

The terms “complementary,” “fully complementary,” “substantially complementary,” and “partially complimentary” are used herein in reference to the base matching between the sense strand and the antisense strand of a PNPLA3 dsRNA agent, between the antisense strand of a PNPLA3 dsRNA agent and a sequence of a target PNPLA3 mRNA, or between a single-stranded antisense oligonucleotide and a sequence of a target PNPLA3 mRNA. It will be understood that the term “antisense strand of a PNPLA3 dsRNA agent” may refer to the same sequence of an “PNPLA3 antisense polynucleotide agent”.

As used herein, the term “substantially identical” or “substantial identity” used in reference to a nucleic acid sequence means a nucleic acid sequence comprising a sequence with at least about 85% sequence identity or more, preferably at least 90%, at least 91%, at least 92%, at least 93%, at least 94%, at least 95%, at least 96%, at least 97%, at least 98%, or at least 99%, compared to a reference sequence. Percentage of sequence identity is determined by comparing two optimally aligned sequences over a comparison window. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity. The inventions disclosed herein encompasses nucleotide sequences substantially identical to those disclosed herein. e.g., in Tables 1-3. In some embodiments, the sequences disclosed herein are exactly identical, or at least about 85%, 86%, 87%, 88%, 89%, 90%, 91%, 92%, 93%, 94%, 95%, 96%, 97%, 98%, or 99% percent identical to those disclosed herein, e.g., in Tables 1-3.

As used herein, the term “strand comprising a sequence” means an oligonucleotide comprising a chain of nucleotides that is described by the sequence referred to using the standard nucleotide nomenclature. The term “double-stranded RNA” or “dsRNA,” as used herein, refers to an RNAi that includes an RNA molecule or complex of molecules having a hybridized duplex region comprising two anti-parallel and substantially or fully complementary nucleic acid strands, which are referred to as having “sense” and “antisense” orientations with respect to a target PNPLA3 RNA. The duplex region can be of any length that permits specific degradation of a desired target PNPLA3 RNA through a RISC pathway, but will typically range from 9 to 30 base pairs in length, e.g., 15-30 base pairs in length. Considering a duplex between 9 and 30 base pairs, the duplex can be any length in this range, for example, 9, 10, 11, 12, 13, 14, 15, 16, 17, 18, 19, 20, 21, 22, 23, 24, 25, 26, 27, 28, 29, or 30, and any sub-range therein between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. PNPLA3 dsRNA agents generated in the cell by processing with Dicer and similar enzymes are generally in the range of 19-22 base pairs in length. One strand of the duplex region of a PNPLA3 dsDNA agent comprises a sequence that is substantially complementary to a region of a target PNPLA3 RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure. In some embodiments of the invention, a hairpin look comprises at least 1, 2, 3, 4, 5, 6, 7, 8, 9, 10, 11, 12, 13, 14, 15, 16, 17, 18, 19, 20, or more unpaired nucleotides. Where the two substantially complementary strands of a PNPLA3 dsRNA agent are comprised by separate RNA molecules, those molecules need not, but can be covalently connected. Where the two strands are connected covalently by means other than a hairpin loop, the connecting structure is referred to as a “linker.” The term “siRNA” is also used herein to refer to a dsRNA agent as described herein.

In some embodiments of the invention a PNPLA3 dsRNA agent may include a sense and antisense sequence that have no-unpaired nucleotides or nucleotide analogs at one or both terminal ends of the dsRNA agent. An end with no unpaired nucleotides is referred to as a “blunt end” and as having no nucleotide overhang. If both ends of a dsRNA agent are blunt, the dsRNA is referred to as “blunt ended.” In some embodiments of the invention, a first end of a dsRNA agent is blunt, in some embodiments a second end of a dsRNA agent is blunt, and in certain embodiments of the invention, both ends of a PNPLA3 dsRNA agent are blunt.

In some embodiments of dsRNA agents of the invention, the dsRNA does not have one or two blunt ends. In such instances there is at least one unpaired nucleotide at the end of a strand of a dsRNA agent. For example, when a 3′-end of one strand of a dsRNA extends beyond the 5′-end of the other strand, or vice versa, there is a nucleotide overhang. A dsRNA can comprise an overhang of at least 1, 2, 3, 4, 5, 6, or more nucleotides. A nucleotide overhang can comprise or consist of a nucleotide/nucleoside analog, including a deoxynucleotide/nucleoside. It will be understood that in some embodiments a nucleotide overhang is on a sense strand of a dsRNA agent, on an antisense strand of a dsRNA agent, or on both ends of a dsRNA agent and nucleotide(s) of an overhang can be present on the 5′ end, 3′ end or both ends of either an antisense or sense strand of a dsRNA. In certain embodiments of the invention, one or more of the nucleotides in an overhang is replaced with a nucleoside thiophosphate.

As used herein, the term “antisense strand” or “guide strand” refers to the strand of a PNPLA3 dsRNA agent that includes a region that is substantially complementary to a PNPLA3 target sequence. As used herein the term “sense strand,” or “passenger strand” refers to the strand of a PNPLA3 dsRNA agent that includes a region that is substantially complementary to a region of the antisense strand of the PNPLA3 dsRNA agent.

### Modifications

In some embodiments of the invention the RNA of a PNPLA3 RNAi agent is chemically modified to enhance stability and/or one or more other beneficial characteristics. Nucleic acids in certain embodiments of the invention may be synthesized and/or modified by methods well established in the art, for example, those described in “Current protocols in Nucleic Acid Chemistry,” Beaucage, S. L. et al. (Eds.), John Wiley & Sons, Inc., New York, N.Y., USA, which is incorporated herein by reference. Modifications that can be present in certain embodiments of PNPLA3 dsRNA agents of the invention include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in certain embodiments of PNPLA3 dsRNA agents, PNPLA3 antisense polynucleotides, and PNPLA3 sense polynucleotides of the invention include, but are not limited to RNAs comprising modified backbones or non-natural internucleoside linkages. As a non-limiting example, an RNA having a modified backbone may not have a phosphorus atom in the backbone. RNAs that do not have a phosphorus atom in their internucleoside backbone may be referred to as oligonucleosides. In certain embodiments of the invention, a modified RNA has a phosphorus atom in its internucleoside backbone.

It will be understood that the term “RNA molecule” or “RNA” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. The terms “ribonucleoside” and “ribonucleotide” may be used interchangeably herein. An RNA molecule can be modified in the nucleobase structure or in the ribose-phosphate backbone structure, e.g., as described herein below, and molecules comprising ribonucleoside analogs or derivatives must retain the ability to form a duplex. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. In some embodiments of the invention, an RNA molecule comprises at least 1, 2, 3, 4, 5, 6, 7, 8, 9, 10, 11, 12, 13, 14, 15, 16, 17, 18, 19, 20, or up to the full length of the PNPLA3 dsRNA agent molecule's ribonucleosides that are modified ribonucleosides. The modifications need not be the same for each of such a plurality of modified ribonucleosides in an RNA molecule.

DsRNA agents, PNPLA3 antisense polynucleotides, and/or PNPLA3 sense polynucleotides of the invention may, in some embodiments comprise one or more independently selected modified nucleotide and/or one or more independently selected non-phosphodiester linkage. As used herein, the terms “internucleotide linkage”, “internucleoside linkage”, “linkage”, and “linker” may be used interchangeably, and refer to the linking groups between unmodified or modified nucleosides, and/or between an unmodified or modified nucleoside and one or more targeting groups. In some embodiments, the linkage may be independently selected from a phosphodiester (PO) linkage, a phosphorothioate (PS) linkage, and/or a phosphorodithioate (PS2) linkage of a dinucleotide at any position of single stranded or double stranded oligonucleotide. As used herein the term “independently selected” used in reference to a selected element, such as a modified nucleotide, non-phosphodiester linkage, etc., means that two or more selected elements can but need not be the same as each other.

As used herein, a “nucleotide base,” “nucleotide,” or “nucleobase” is a heterocyclic pyrimidine or purine compound, which is a standard constituent of all nucleic acids, and includes the bases that form the nucleotides adenine, guanine, cytosine, thymine, and uracil. A nucleobase may further be modified to include, though not intended to be limiting: universal bases, hydrophobic bases, promiscuous bases, size-expanded bases, and fluorinated bases. The term “ribonucleotide” or “nucleotide” may be used herein to refer to an unmodified nucleotide, a modified nucleotide, or a surrogate replacement moiety. Those in the art will recognize that guanine, cytosine, adenine, and uracil can be replaced by other moieties without substantially altering the base pairing properties of an oligonucleotide comprising a nucleotide bearing such replacement moiety.

As used herein, “optionally” or “optionally” means that the event or environment described later may, but need not, occur, including where the event or environment occurred or did not occur. For example, “C1-6 alkyl optionally substituted by halogen or cyano” means that halogen or cyano may, but not necessarily, be present, including the case where alkyl is substituted by halogen or cyano and the case where alkyl is not substituted by halogen and cyano.

As used herein, in the chemical structures of the compounds of the present disclosure, the bond “ [symbol] ” represents an unspecified configuration, i.e., if a chiral isomer is present in the chemical structure, the bond “ [symbol] ” can be “ [symbol] ” or “ [symbol] ” or both “ [symbol] ” and “ [symbol] ” two configurations. Although some of the above structural formulas are depicted as some isomeric forms for simplicity, the present disclosure may include all isomers, such as tautomers, rotamers, and mixtures thereof. Suitable chiral compounds include: geometric isomers, diastereomers, racemates and enantiomers.

As used herein, “ [symbol] ” or “ [symbol] ” used in the chemical formulas of the present disclosure may be attached to any one or more groups according to the scope of the invention described herein.

In one embodiment, modified RNAs contemplated for use in methods and compositions described herein are peptide nucleic acids (PNAs) that have the ability to form the required duplex structure and that permit or mediate the specific degradation of a target RNA via a RISC pathway. In certain embodiments of the invention, a PNPLA3 RNA interference agent includes a single stranded RNA that interacts with a target PNPLA3 RNA sequence to direct the cleavage of the target PNPLA3 RNA.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. Means of preparing phosphorus-containing linkages are routinely practiced in the art and such methods can be used to prepare certain modified PNPLA3 dsRNA agents, certain modified PNPLA3 antisense polynucleotides, and/or certain modified PNPLA3 sense polynucleotides of the invention.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Means of preparing modified RNA backbones that do not include a phosphorus atom are routinely practiced in the art and such methods can be used to prepare certain modified PNPLA3 dsRNA agents, certain modified PNPLA3 antisense polynucleotides, and/or certain modified PNPLA3 sense polynucleotides of the invention.

In certain embodiments of the invention, RNA mimetics are included in PNPLA3 dsRNAs, PNPLA3 antisense polynucleotides, and/or PNPLA3 sense polynucleotides, such as, but not limited to: replacement of the sugar and the internucleoside linkage, i.e., the backbone, of the nucleotide units with novel groups. In such embodiments, base units are maintained for hybridization with an appropriate PNPLA3 nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Means of preparing RNA mimetics are routinely practiced in the art and such methods can be used to prepare certain modified PNPLA3 dsRNA agents of the invention.

Some embodiments of the invention include RNAs with phosphorothioate backbones and oligonucleosides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂— and —N(CH₃)—CH₂-[wherein the native phosphodiester backbone is represented as —O—P—O—CH₂—]. Means of preparing RNAs with phosphorothioate backbones and oligonucleosides with heteroatom backbones are routinely practiced in the art and such methods can be used to prepare certain modified PNPLA3 dsRNA agents, certain PNPLA3 antisense polynucleotides, and/or certain PNPLA3 sense polynucleotides of the invention.

Modified RNAs can also contain one or more substituted sugar moieties. PNPLA3 dsRNAs, PNPLA3 antisense polynucleotides, and/or PNPLA3 sense polynucleotides of the invention may comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a PNPLA3 dsRNA agent, or a group for improving the pharmacodynamic properties of a PNPLA3 dsRNA agent, PNPLA3 antisense polynucleotide, and/or PNPLA3 sense polynucleotide, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂. Means of preparing modified RNAs such as those described are routinely practiced in the art and such methods can be used to prepare certain modified PNPLA3 dsRNA agents of the invention.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of a PNPLA3 dsRNA agent, PNPLA3 antisense polynucleotide, and/or PNPLA3 sense polynucleotide of the invention, particularly the 3′ position of the sugar on the 3′ terminal nucleotide or in 2′-5′ linked PNPLA3 dsRNAs, PNPLA3 antisense polynucleotides, or PNPLA3 sense polynucleotides, and the 5′ position of 5′ terminal nucleotide. PNPLA3 dsRNA agents, PNPLA3 antisense polynucleotides, and/or PNPLA3 sense polynucleotides may also have sugar mimetics such as cyclobutyl moieties in place of the pentofuranosyl sugar. Means of preparing modified RNAs such as those described are routinely practiced in the art and such methods can be used to prepare certain modified PNPLA3 dsRNA agents, PNPLA3 antisense polynucleotides, and/or PNPLA3 sense polynucleotides of the invention.

A PNPLA3 dsRNA agent, PNPLA3 antisense polynucleotide, and/or PNPLA3 sense polynucleotide may, in some embodiments, include nucleobase (often referred to in the art simply as “base”) modifications or substitutions. As used herein, “unmodified” or “natural” nucleobases include the purine bases adenine and guanine, and the pyrimidine bases thymine, cytosine and uracil. Modified nucleobases include other synthetic and natural nucleobases such as 5-methylcytosine (5-Me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Additional nucleobases that may be included in certain embodiments of PNPLA3 dsRNA agents of the invention are known in the art, see for example: Modified Nucleosides in Biochemistry, Biotechnology and Medicine, Herdewijn, P. Ed. Wiley-VCH, 2008; The Concise Encyclopedia Of Polymer Science And Engineering, pages 858-859, Kroschwitz, J. L, Ed. John Wiley & Sons, 1990, English et al., Angewandte Chemie, International Edition, 1991, 30, 613, Sanghvi, Y S., Chapter 15, dsRNA Research and Applications, pages 289-302, Crooke, S. T. and Lebleu, B., Ed., CRC Press, 1993. Means of preparing dsRNAs, PNPLA3 antisense strand polynucleotides and/or PNPLA3 sense strand polynucleotides that comprise nucleobase modifications and/or substitutions such as those described herein are routinely practiced in the art and such methods can be used to prepare certain modified PNPLA3 dsRNA agents, PNPLA3 sense polynucleotides, and/or PNPLA3 antisense polynucleotides of the invention. Teachings regarding the synthesis of particular modified oligonucleotides may be found in the following U.S. patents: U.S. Pat. No. 5,218,105, drawn to polyamine conjugated oligonucleotides; U.S. Pat. No. 5,541,307, drawn to oligonucleotides having modified backbones; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having (3-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups may be used as linkers to other moieties attached at any of a variety of positions of the nucleoside; U.S. Pat. No. 5,587,361 drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligo-nucleotides having 3-deazapurines; U.S. Pat. No. 5,608,046, both drawn to conjugated 4′-desmethyl nucleoside analogs; U.S. Pat. No. 5,610,289, drawn to backbone-modified oligonucleotide analogs; U.S. Pat. No. 6,262,241 drawn to, inter alia, methods of synthesizing 2′-fluoro-oligonucleotides.

Certain embodiments of PNPLA3 dsRNA agents, PNPLA3 antisense polynucleotides, and/or PNPLA3 sense polynucleotides of the invention include RNA modified to include one or more locked nucleic acids (LNA). A locked nucleic acid is a nucleotide with a modified ribose moiety comprising an extra bridge connecting the 2′ and 4′ carbons. This structure effectively “locks” the ribose in the 3′-endo structural conformation. The addition of locked nucleic acids in a PNPLA3 dsRNA agent, PNPLA3 antisense polynucleotides, and/or PNPLA3 sense polynucleotides of the invention may increase stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Means of preparing dsRNA agents, PNPLA3 antisense polynucleotides, and/or PNPLA3 sense polynucleotides that comprise locked nucleic acid(s) are routinely practiced in the art and such methods can be used to prepare certain modified PNPLA3 dsRNA agents of the invention.

Certain embodiments of PNPLA3 dsRNA compounds, sense polynucleotides, and/or antisense polynucleotides of the invention, include at least one modified nucleotide, wherein the at least one modified nucleotide comprises: 2′-O-methyl nucleotide, 2′-fluoro nucleotide, 2′-deoxy nucleotide, 2′3′-seco nucleotide mimic, locked nucleotide, 2′-F-Arabino nucleotide, 2′-methoyxyethyl nucleotide, 2′-amino-modified nucleotide, 2′-alkyl-modified nucleotide, mopholino nucleotide, and 3′-OMe nucleotide, a nucleotide comprising a 5′-phosphorothioate group, a nucleotide comprising vinyl phosphonate, a nucleotide comprising adenosine-glycol nucleic acid (GNA), a nucleotide comprising thymidine-glycol nucleic acid (GNA) S-Isomer, a nucleotide comprising 2′-deoxythymidine-3′phosphate, a nucleotide comprising 2′-deoxyguanosine-3′-phosphate, a nucleotide comprising 2′-deoxyadenosine-3′-phosphate, a nucleotide comprising 2′-deoxycytidine-3′-phosphate, a nucleotide comprising 2′-deoxyuridine-3′-phosphate, or a terminal nucleotide linked to a cholesteryl derivative or dodecanoic acid bisdecylamide group, a 2′-amino-modified nucleotide, a phosphoramidate, or a non-natural base comprising nucleotide. In some embodiments, a PNPLA3 dsRNA compound includes an E-vinylphosphonate nucleotide at the 5′ end of the antisense strand, also referred to herein as the guide strand.

Certain embodiments of PNPLA3 dsRNA compounds, 3′ and 5′ end of sense polynucleotides, and/or 3′ end of antisense polynucleotides of the invention, include at least one modified nucleotide, wherein the at least one modified nucleotide comprises: abasic nucleotide, ribitol, inverted nucleotide, inverted abasic nucleotide, inverted 2′-OMe nucleotide, inverted 2′-deoxy nucleotide. It is known to skilled in art, including an abasic or inverted abasic nucleotide at the end of oligonucleotide enhances stability (Czauderna et al. Structural variations and stabilizing modifications of synthetic siRNAs in mammalian cells. Nucleic Acids Res. 2003; 31(11):2705-2716. doi:10.1093/nar/gkg393). In some embodiments, a PNPLA3 dsRNA compound includes one or more inverted abasic residues (invab) at either 3′-end or 5′-end, or both 3′-end and 5′-end. Exemplified inverted abasic residues (invab) include, but are not limited to the following:
when at the 3′-end:

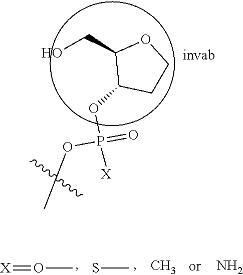

link to 3′-end of oligonucleotide

Certain embodiments of PNPLA3 dsRNA compounds, 3′ and 5′ end of sense polynucleotides, and/or 3′ end of antisense polynucleotides of the invention, include at least one modified nucleotide, wherein the at least one modified nucleotide comprises: isomannide nucleotide. Specific examples of isomannide nucleotides include, but are not limited to:

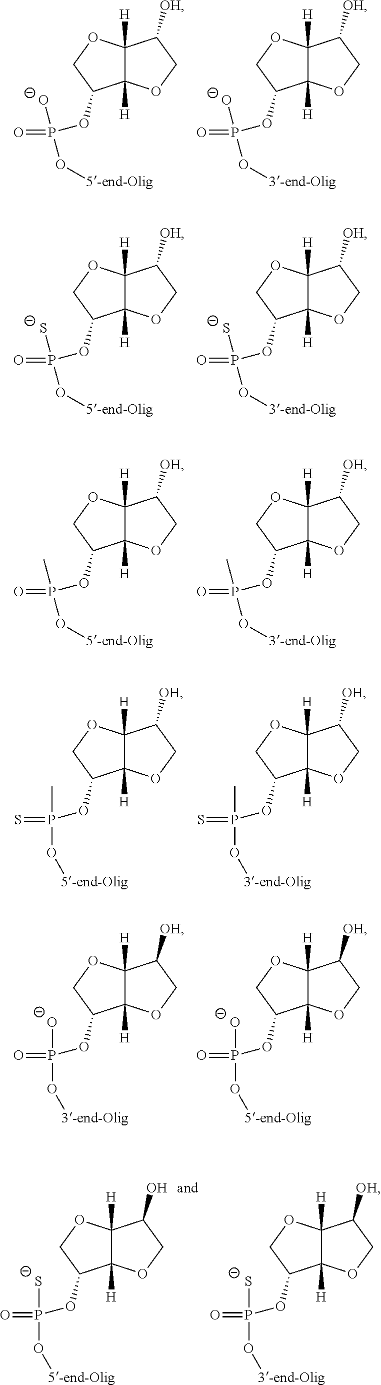

wherein each of the phrase “Olig” independently represents a polynucleotide moiety. Exemplified isomannide residues (imann) include, but are not limited to the following:

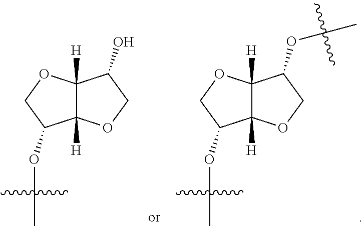 [SMILES: *O[C@@H]1CO[C@H]2[C@@H]1OC[C@H]2O ; *O[C@@H]1CO[C@H]2[C@@H]1OC[C@H]2O*]

Certain embodiments of PNPLA3 dsRNA compounds, antisense polynucleotides of the invention, include at least one modified nucleotide, wherein the at least one modified nucleotide comprises unlocked nucleic acid nucleotide (UNA) or/and glycol nucleic acid nucleotide (GNA). It is known to skilled in art, UNA and GNA are thermally destabilizing chemical modifications, can significantly improves the off-target profile of a siRNA compound (Janas, et al., Selection of GalNAc-conjugated siRNAs with limited off-target-driven rat hepatotoxicity. Nat Commun. 2018; 9(1):723. doi:10.1038/s41467-018-02989-4; Laursen et al., Utilization of unlocked nucleic acid (UNA) to enhance siRNA performance in vitro and in vivo. Mol BioSyst. 2010; 6:862-70).

Certain embodiments of PNPLA3 dsRNA compounds, antisense polynucleotides of the invention further comprise a phosphate moiety. As used herein, a phosphate moiety refers to a phosphate group including phosphates or phosphates mimics that attached to the sugar moiety (e.g., a ribose or deoxyribose or analog thereof) of a nucleotide. A nucleotide comprising a phosphate mimic may also be defined as a phosphonate modified nucleotide.

In some embodiments, the phosphate mimic is a 5′-vinyl phosphonate (VP). In exemplary embodiments, a vinyl phosphonate of the disclosure has the following structure:

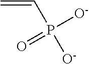

A vinyl phosphonate of the instant disclosure may be attached to either the antisense or the sense strand of a dsRNA of the disclosure. In certain preferred embodiments, a vinyl phosphonate of the instant disclosure is attached to the antisense strand of a dsRNA, optionally at the 5′ end of the antisense strand of the dsRNA.

In certain embodiments, a vinyl phosphonate modified nucleotide of the disclosure has the structure of formula (IV):

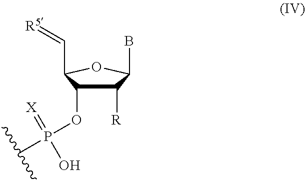

wherein X is O or S;
R is hydrogen, hydroxy, fluoro, or C₁₋₂₀ alkoxy (e.g., methoxy or n-hexadecyloxy);
R5′ is ═C(H)—P(O)(OH)₂ and the double bond between the C5′ carbon and R5′ is in the E or Z orientation (e.g., E orientation); and
B is a nucleobase or a modified nucleobase, optionally where B is adenine, guanine, cytosine, thymine, or uracil.

In certain embodiments, R5′ is ═C(H)—P(O)(OH)₂ and the double bond between the C5′ carbon and R5′ is in the E orientation. In certain embodiments, R is methoxy and R5′ is ═C(H)—P(O)(OH)₂ and the double bond between the C5′ carbon and R5′ is in the E orientation. In certain embodiments, X is S, R is methoxy, and R5′ is ═C(H)—P(O)(OH)₂ and the double bond between the C5′ carbon and R5′ is in the E orientation.

Vinyl phosphonate modifications are also contemplated for the dsRNAs, the compositions and methods of the instant disclosure. An exemplary vinyl phosphonate structure is:

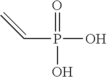

In certain embodiments, a vinyl phosphonate modified nucleotide is VPu* which has the structure of as follows:

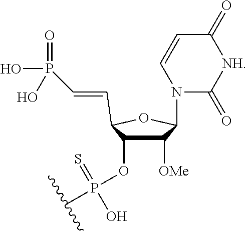

In many cases, protecting groups are used during the preparation of the compounds of the invention. As used herein, the term “protected” means that the indicated moiety has a protecting group appended thereon. In some embodiments of the invention, compounds contain one or more protecting groups. A wide variety of protecting groups can be employed in the methods of the invention. In general, protecting groups render chemical functionalities inert to specific reaction conditions, and can be appended to and removed from such functionalities in a molecule without substantially damaging the remainder of the molecule. Protecting groups in general and hydroxyl protecting groups in particular are well known in the art (Greene and Wuts, Protective Groups in Organic Synthesis, Chapter 2, 2d ed., John Wiley & Sons, New York, 1991).

As used herein, examples of protecting groups (e.g., hydroxyl protecting groups) include, but are not limited to, methyl, ethyl, benzyl (Bn), phenyl, isopropyl, tert-butyl, acetyl, chloroacetyl, trichloro acetyl, trifluoroacetyl, pivaloyl, tert-butoxymethyl, methoxymethyl, 1-ethoxyethyl, 1-(2-chloroethoxy)ethyl, allyl, cyclohexyl, 9-fluorenylmethoxycarbonyl (Fmoc), methanesulfonate, toluenesulfonate, triflate, benzoyl, benzoylformate, p-phenylbenzoyl, 4-methoxybenzyl, monomethoxytrityl, dimethoxytrityl, trimethoxytrityl, 4-chlorobenzyl, 4-nitrobenzyl, 2,4-dinitrophenyl, 4-acyloxybenzyl, 2-methylphenyl, 2,6-dimethylphenyl, 2-chlorophenyl, 2,6-dichlorobenzyl, diphenylmethyl, triphenylmethyl, 4-methylthio-1-butyl, S-acetylthioacetate (SATA), 2-cyanoethyl, 2-cyanol, 1-dimethylethyl (CDM), 4-cyano-2-butenyl, 2-(trimethylsilyl)ethyl (TSE), 2-(phenylthio)ethyl, 2-(triphenylsilyl)ethyl, 2-(benzylsulfonyl)ethyl, 2,2,2-trichloroethyl, 2,2,2-tribromoethyl, 2,3-dibromopropyl, 2,2,2-trifluoroethyl, phenylthio, 2-chloro-4-tritylphenyl, 2-bromophenyl, 2-[N-isopropyl-N-(4-methoxybenzoyl)amino]ethyl, 4-(N-trifluoroacetylamino)butyl, 4-oxopentyl, 4-tritylaminophenyl, 4-benzyl aminophenyl, tetrahydropyranyl, morpholino, trimethylsilyl, triethylsilyl, tert-butyldimethylsilyl, tert-butyldiphenylsilyl, triphenyl Silyl, triisopropylsilyl, pivaloyloxymethyl (POM) and 9-phenylxanthine-9-yl.

As used herein, examples of amino protecting groups include, but are not limited to, carbamate protecting groups, such as 2-trimethylsilylethoxycarbonyl (Teoc), 1-methyl-1-(4-biphenyl) ethoxycarbonyl (Bpoc), tert-butyloxycarbonyl (BOC), allyloxycarbonyl (Alloc), 9-fluorenyl-methoxycarbonyl (Fmoc), benzyloxycarbonyl (Cbz); amide protecting groups, such as formyl, acetyl, pivaloyl, trihaloacetyl, benzoyl, 2-nitrobenzenesulfonyl; and imine and cyclic imide protecting groups, such as phthalimido and dithiasuccinoyl. Equivalents of these amino-protecting groups are also encompassed by the compounds and methods of the invention.

Another modification that may be included in the RNA of certain embodiments of PNPLA3 dsRNA agents, PNPLA3 antisense polynucleotides, and/or PNPLA3 sense polynucleotides of the invention, comprises chemically linking to the RNA one or more ligands, moieties or conjugates that enhance one or more characteristics of the PNPLA3 dsRNA agent, PNPLA3 antisense polynucleotide, and/or PNPLA3 sense polynucleotide, respectively. Non-limiting examples of characteristics that may be enhanced are: PNPLA3 dsRNA agent, PNPLA3 antisense polynucleotide, and/or PNPLA3 sense polynucleotide activity, cellular distribution, delivery of a PNPLA3 dsRNA agent, pharmacokinetic properties of a PNPLA3 dsRNA agent, and cellular uptake of the PNPLA3 dsRNA agent. In some embodiments of the invention, a PNPLA3 dsRNA agent comprises one or more targeting groups or linking groups, which in certain embodiments of PNPLA3 dsRNA agents of the invention are conjugated to the sense strand. A non-limiting example of a targeting group is a compound comprising N-acetyl-galactosamine (GalNAc). The terms “targeting group”, “targeting agent”, “linking agent”, “targeting compound”, and “targeting ligand” may be used interchangeably herein. In certain embodiments of the invention a PNPLA3 dsRNA agent comprises a targeting compound that is conjugated to the 5′-terminal end of the sense strand. In certain embodiments of the invention a PNPLA3 dsRNA agent comprises a targeting compound that is conjugated to the 3′-terminal end of the sense strand. In some embodiments of the invention, a PNPLA3 dsRNA agent comprises a targeting group that comprises GalNAc. In certain embodiments of the invention a PNPLA3 dsRNA agent does not include a targeting compound conjugated to one or both of the 3′-terminal end and the 5′-terminal end of the sense strand. In certain embodiments of the invention a PNPLA3 dsRNA agent does not include a GalNAc containing targeting compound conjugated to one or both of the 5′-terminal end and the 3′-terminal end of the sense strand.

Additional targeting and linking agents are well known in the art, for example, targeting and linking agents that may be used in certain embodiments of the invention include but are not limited to lipid moieties such as a cholesterol moiety (Letsinger et al., Proc. Natl. Acid. Sci. USA, 1989, 86:6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

Certain embodiments of a composition comprising a PNPLA3 dsRNA agent, PNPLA3 antisense polynucleotide, and/or PNPLA3 sense polynucleotide may comprise a ligand that alters distribution, targeting, or etc. of the PNPLA3 dsRNA agent. In some embodiments of a composition comprising a PNPLA3 dsRNA agent of the invention, the ligand increases affinity for a selected target, e.g., molecule, cell or cell type, compartment, e.g., a cellular or organ compartment, tissue, organ or region of the body, as, e.g., compared to a species absent such a ligand. A ligand useful in a composition and/or method of the invention may be a naturally occurring substance, such as a protein (e.g., human serum albumin (HSA), low-density lipoprotein (LDL), or globulin); a carbohydrate (e.g., a dextran, pullulan, chitin, chitosan, inulin, cyclodextrin or hyaluronic acid); or a lipid. A ligand may also be a recombinant or synthetic molecule, such as a synthetic polymer, e.g., a synthetic polyamino acid or polyamine. Examples of polyamino acids are a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl) methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

A ligand included in a composition and/or method of the invention may comprise a targeting group, non-limiting examples of which are a cell or tissue targeting agent, e.g., a lectin, glycoprotein, lipid or protein, e.g., an antibody that binds to a specified cell type such as a kidney cell or a liver cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O (hexadecyl)glycerol, geranyloxyhexyl group, hexadecylglycerol, borneol, menthol, 1,3-propanediol, heptadecyl group, palmitic acid, myristic acid, O3-(oleoyl) lithocholic acid, 03-(oleoyl) cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

A ligand included in a composition and/or method of the invention may be a protein, e.g., glycoprotein, or peptide, for example a molecule with a specific affinity for a co-ligand, or an antibody, for example an antibody, that binds to a specified cell type such as a cancer cell, endothelial cell, cardiac cell, or bone cell. A ligand useful in an embodiment of a composition and/or method of the invention can be a hormone or hormone receptor. A ligand useful in an embodiment of a composition and/or method of the invention can be a lipid, lectin, carbohydrates, vitamin, cofactos, multivalent lactose, multivalent galactose, N-acetyl-galactosamine, N-acetyl-gulucosamine multivalent mannose, or multivalent fucose. A ligand useful in an embodiment of a composition and/or method of the invention can be a substance that can increase uptake of the PNPLA3 dsRNA agent into the cell, for example, by disrupting the cell's cytoskeleton, e.g., by disrupting the cell's microtubules, microfilaments, and/or intermediate filaments. Non-limiting examples of this type of agent are: taxon, vincristine, vinblastine, cytochalasin, nocodazole, japlakinolide, latrunculin A, phalloidin, swinholide A, indanocine, and myoservin.

In some embodiments, a ligand attached to a PNPLA3 dsRNA agent of the invention functions as a pharmacokinetic (PK) modulator. An example of a PK modulator that may be used in compositions and methods of the invention includes but is not limited to: a lipophiles, a bile acid, a steroid, a phospholipid analogue, a peptide, a protein binding agent, PEG, a vitamin, cholesterol, a fatty acid, cholic acid, lithocholic acid, dialkylglycerides, diacylglyceride, a phospholipid, a sphingolipid, naproxen, ibuprofen, vitamin E, biotin, an aptamer that binds a serum protein, etc. Oligonucleotides comprising a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbone may also be used in compositions and/or methods of the invention as ligands.

PNPLA3 dsRNA Agent Compositions

In some embodiments of the invention, a PNPLA3 dsRNA agent is in a composition. A composition of the invention may include one or more PNPLA3 dsRNA agent and optionally one or more of a pharmaceutically acceptable carrier, a delivery agent, a targeting agent, detectable label, etc. A non-limiting example of a targeting agent that may be useful according to some embodiments of methods of the invention is an agent that directs a PNPLA3 dsRNA agent of the invention to and/or into a cell to be treated. A targeting agent of choice will depend upon such elements as: the nature of the PNPLA3-associated disease or condition, and on the cell type being targeted. In a non-limiting example, in some embodiments of the invention it may be desirable to target a PNPLA3 dsRNA agent to and/or into a liver cell. It will be understood that in some embodiments of methods of the invention, a therapeutic agent comprises a PNPLA3 dsRNA agent with only a delivery agent, such as a delivery agent comprising N-Acetylgalactosamine (GalNAc), without any additional attached elements. For example, in some aspects of the invention a PNPLA3 dsRNA agent may be attached to a delivery compound comprising GalNAc and included in a composition comprising a pharmaceutically acceptable carrier and administered to a cell or subject without any detectable labels, or targeting agents, etc. attached to the PNPLA3 dsRNA agent.

In cases where a PNPLA3 dsRNA agent of the invention is administered with and/or attached to one or more delivery agents, targeting agents, labeling agents, etc. a skilled artisan will be aware of and able to select and use suitable agents for use in methods of the invention. Labeling agents may be used in certain methods of the invention to determine the location of a PNPLA3 dsRNA agent in cells and tissues and may be used to determine a cell, tissue, or organ location of a treatment composition comprising a PNPLA3 dsRNA agent that has been administered in methods of the invention. Procedures for attaching and utilizing labeling agents such as enzymatic labels, dyes, radiolabels, etc. are well known in the art. It will be understood that in some embodiments of compositions and methods of the invention, a labeling agent is attached to one or both of a sense polynucleotide and an antisense polynucleotide included in a PNPLA3 dsRNA agent.

Delivery of PNPLA3 dsRNA Agents and PNPLA3 Antisense Polynucleotide Agents

Certain embodiments of methods of the invention, includes delivery of a PNPLA3 dsRNA agent into a cell. As used herein the term, “delivery” means facilitating or effecting uptake or absorption into the cell. Absorption or uptake of a PNPLA3 dsRNA agent can occur through unaided diffusive or active cellular processes, or by use of delivery agents, targeting agents, etc. that may be associated with a PNPLA3 dsRNA agent of the invention. Delivery means that are suitable for use in methods of the invention include, but are not limited to: in vivo delivery, in which a PNPLA3 dsRNA agent is in injected into a tissue site or administered systemically. In some embodiments of the invention, a PNPLA3 dsRNA agent is attached to a delivery agent.

Non-limiting examples of methods that can be used to deliver PNPLA3 dsRNA agents to cells, tissues and/or subjects include: PNPLA3 dsRNA-GalNAc conjugates, SAMiRNA technology, LNP-based delivery methods, and naked RNA delivery. These and other delivery methods have been used successfully in the art to deliver therapeutic RNAi agents for treatment of various diseases and conditions, such as but not limited to: liver diseases, acute intermittent porphyria (AIP), hemophilia, pulmonary fibrosis, etc. Details of various delivery means are found in publications such as: Nikam, R. R. & K. R. Gore (2018) Nucleic Acid Ther, 28 (4), 209-224 August 2018; Springer A. D. & S. F. Dowdy (2018) Nucleic Acid Ther. June 1; 28(3): 109-118; Lee, K. et al., (2018) Arch Pharm Res, 41(9), 867-874; and Nair, J. K. et al., (2014) J. Am. Chem. Soc. 136:16958-16961, the content each of which is incorporated by reference herein.

Some embodiments of the invention comprise use of lipid nanoparticles (LNPs) to deliver a PNPLA3 dsRNA agent of the invention to a cell, tissue, and/or subject. LNPs are routinely used for in vivo delivery of PNPLA3 dsRNA agents, including therapeutic PNPLA3 dsRNA agents. One benefit of using an LNP or other delivery agent is an increased stability of the PNPLA3 RNA agent when it is delivered to a subject using the LNP or other delivery agent. In some embodiments of the invention an LNP comprises a cationic LNP that is loaded with one or more PNPLA3 RNAi molecules of the invention. The LNP comprising the PNPLA3 RNAi molecule(s) is administered to a subject, the LNPs and their attached PNPLA3 RNAi molecules are taken up by cells via endocytosis, their presence results in release of RNAi trigger molecules, which mediate RNAi.

Another non-limiting example of a delivery agent that may be used in embodiments of the invention to delivery a PNPLA3 dsRNA agent of the invention to a cell, tissue and/or subject is an agent comprising at least one GalNAc targeting ligand that is attached to a PNPLA3 dsRNA agent of the invention and delivers the PNPLA3 dsRNA agent to a cell, tissue, and/or subject. Examples of certain additional delivery agents comprising GalNAc that can be used in certain embodiments of methods and composition of the invention are disclosed in PCT Application: WO2020191183A1 (incorporated herein in its entirety). A non-limiting example of a GalNAc targeting ligand that can be used in compositions and methods of the invention to deliver a PNPLA3 dsRNA agent to a cell is a targeting ligand cluster. Examples of targeting ligand clusters that are presented herein are referred to as: GalNAc Ligand with phosphodiester link (GLO) and GalNAc Ligand with phosphorothioate link (GLS). The term “GLX-n” may be used herein to indicate the attached GalNAC-containing compound is any one of compounds GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16, the structure of each of which is shown below, with the below with location of attachment of the GalNAc-targeting ligand to an RNAi agent of the invention at far right of each (shown with “ [symbol] ”). It will be understood that any RNAi and dsRNA molecule of the invention can be attached to the GLS-1, GLS-2, GLS-3, GLS-4, GLS-5, GLS-6, GLS-7, GLS-8, GLS-9, GLS-10, GLS-11, GLS-12, GLS-13, GLS-14, GLS-15, GLS-16, GLO-1, GLO-2, GLO-3, GLO-4, GLO-5, GLO-6, GLO-7, GLO-8, GLO-9, GLO-10, GLO-11, GLO-12, GLO-13, GLO-14, GLO-15, and GLO-16. GLO-1 through GLO-16 and GLS-1 through GLS-16 structures are shown as below.

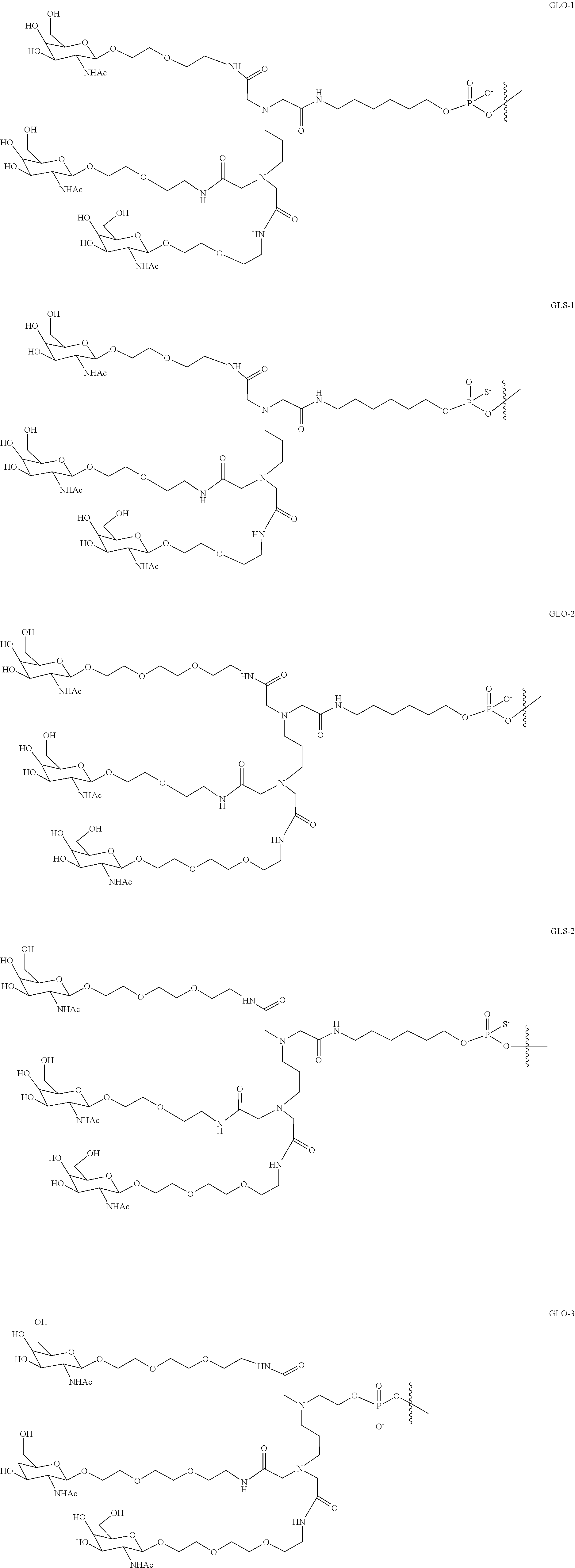

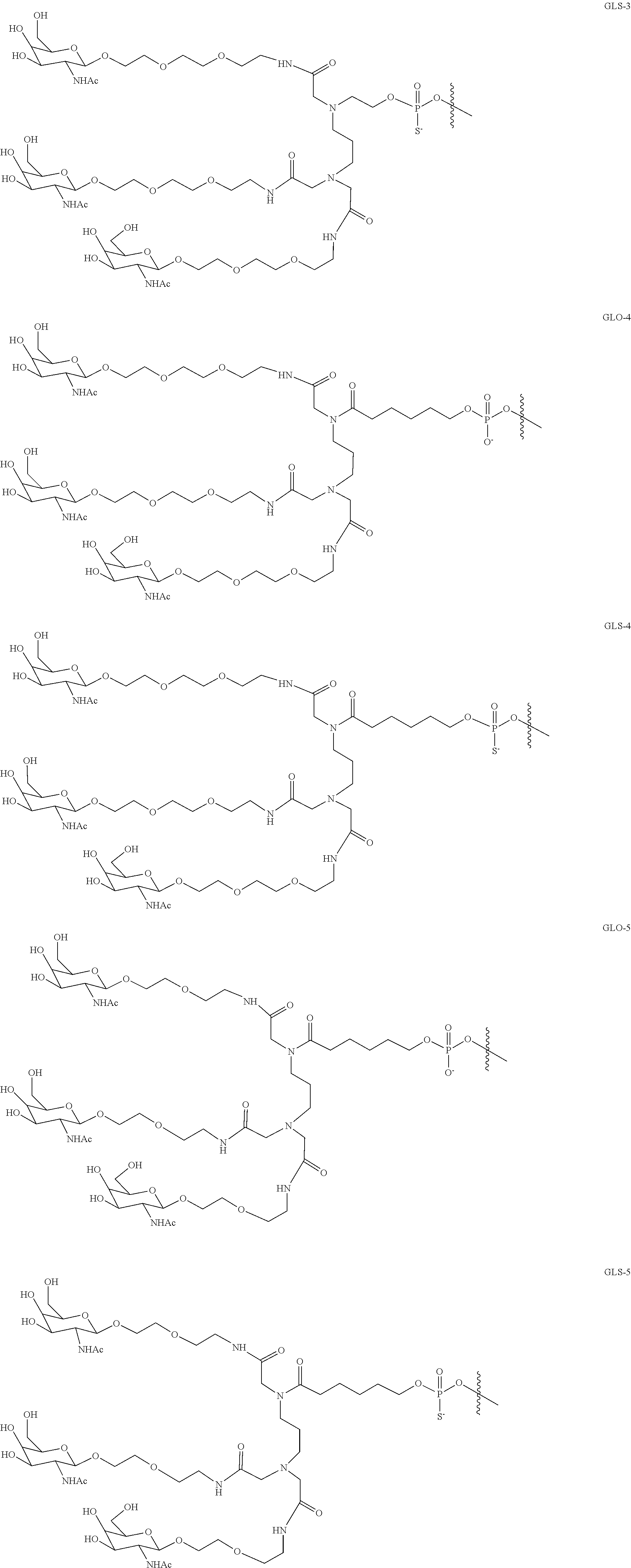

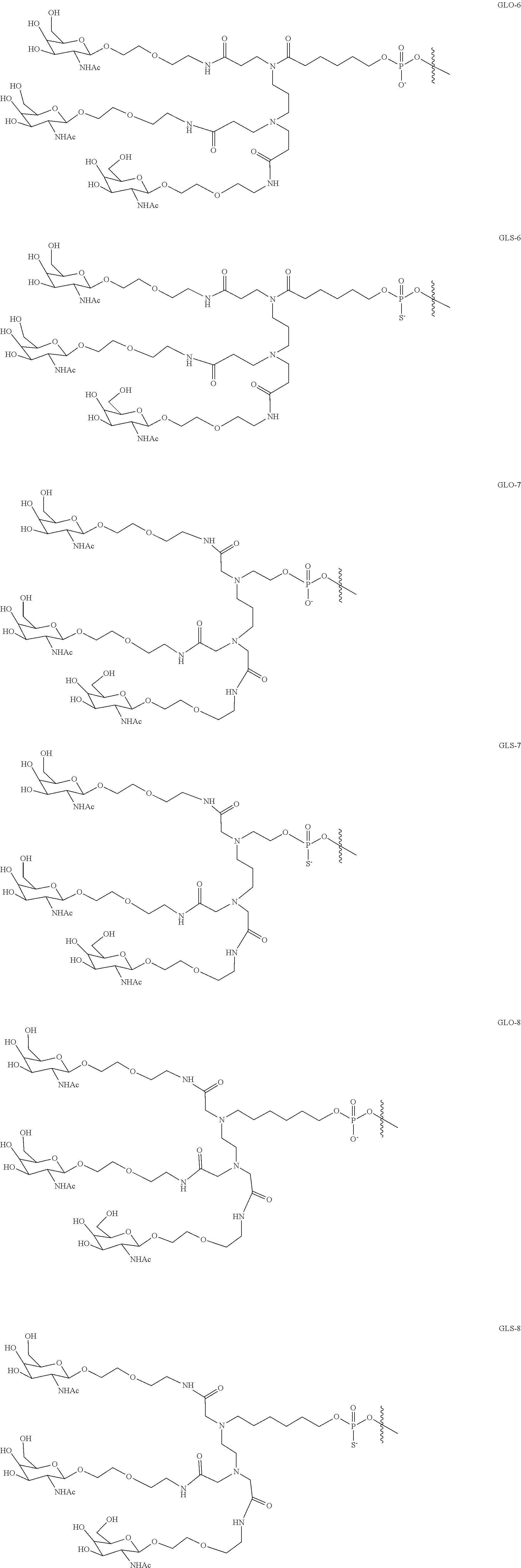

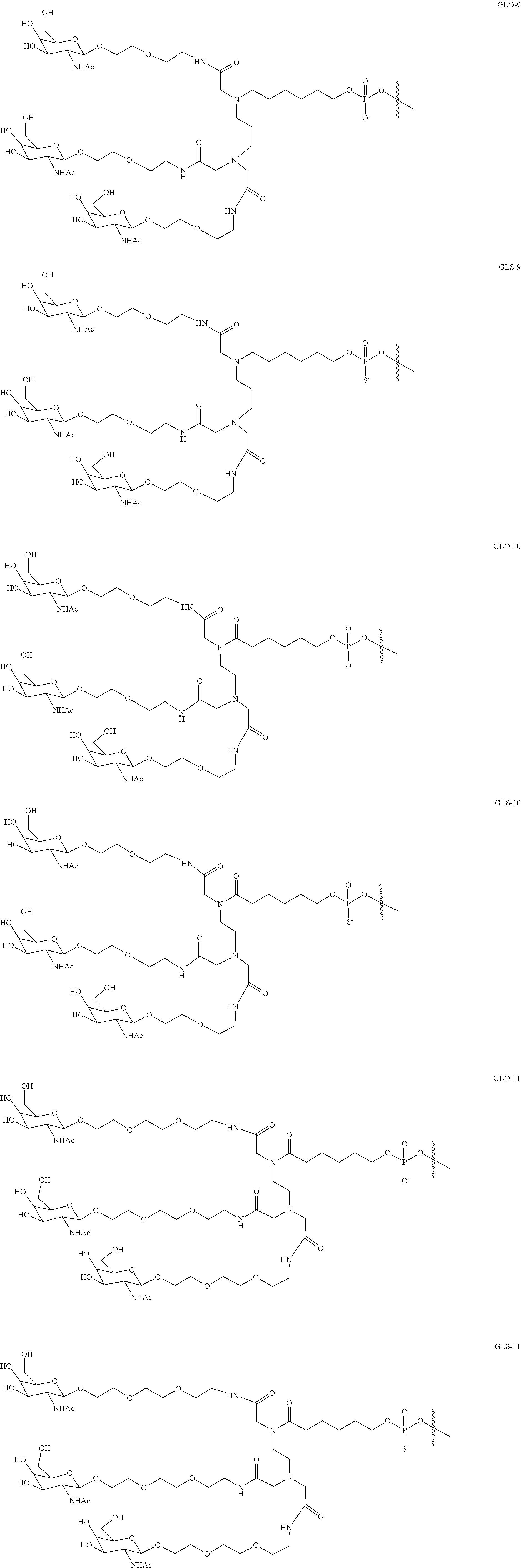

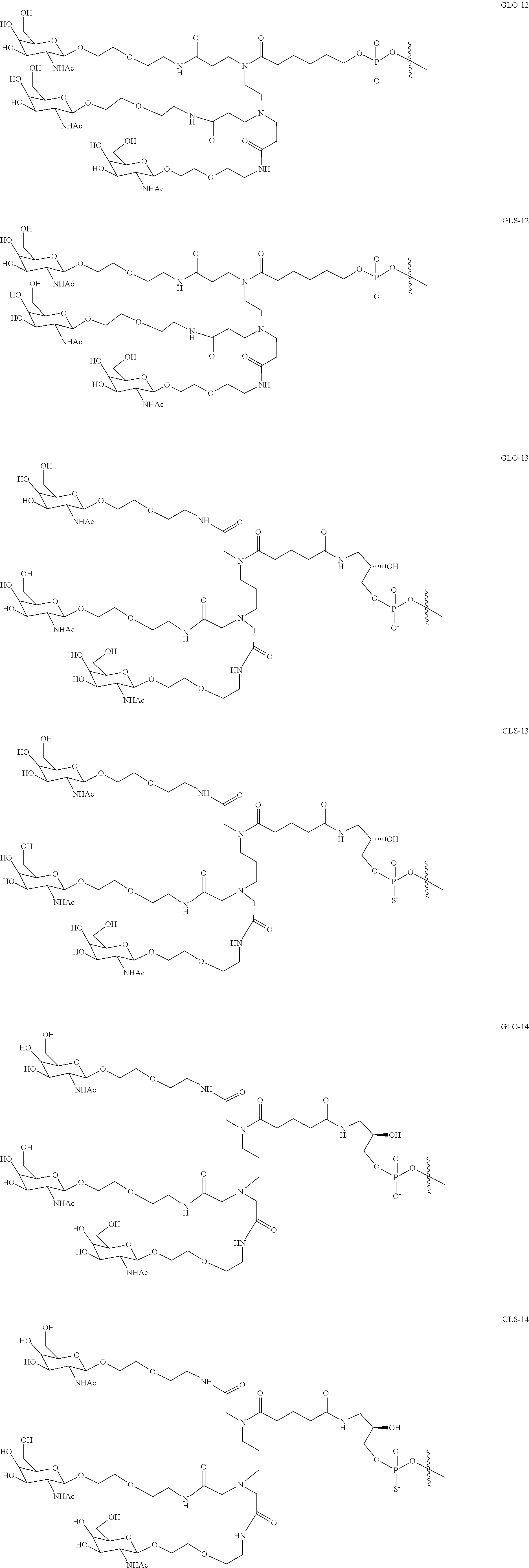

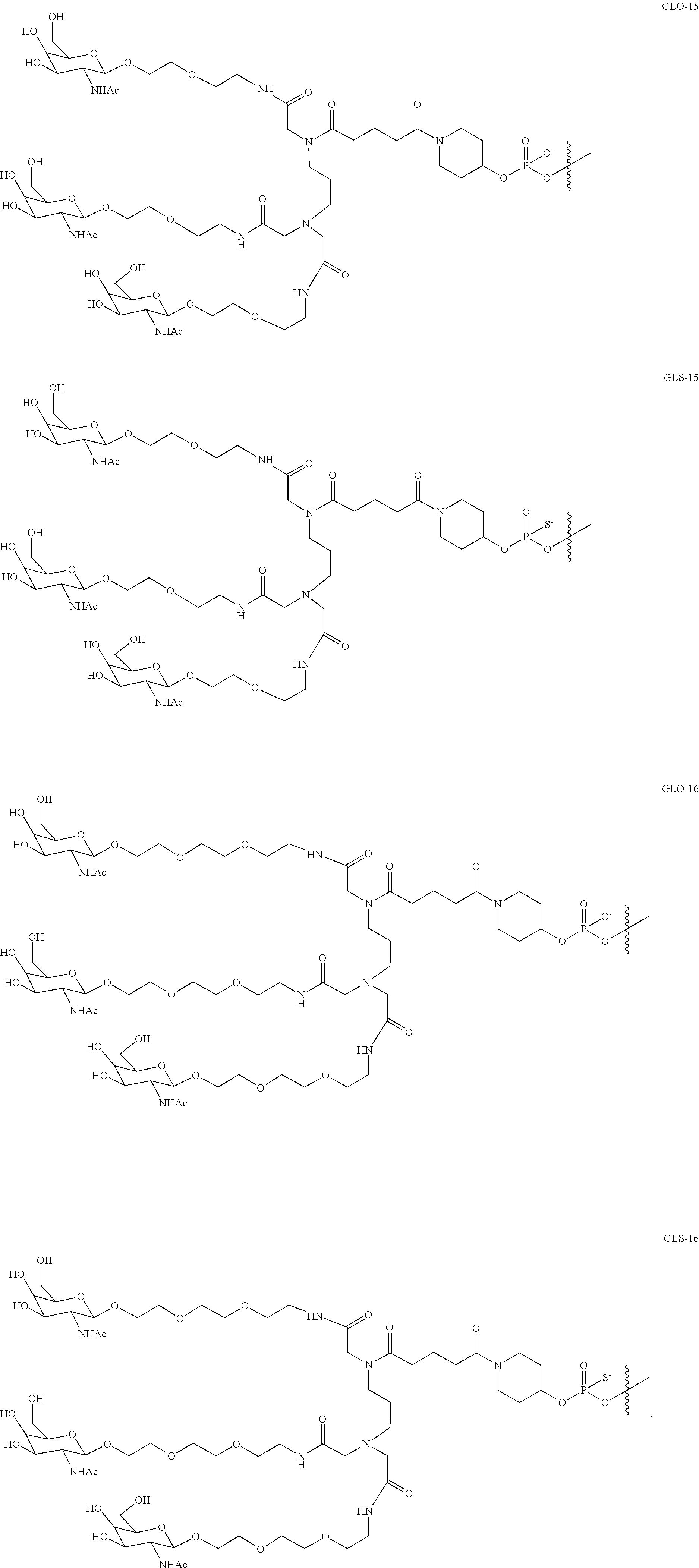

In certain embodiments, the aforesaid isomannide nucleotides may further conjugate to one or more GalNAc targeting ligands. Specific examples of isomannide nucleotides conjugated to a GalNAc targeting ligand include, but are not limited to:

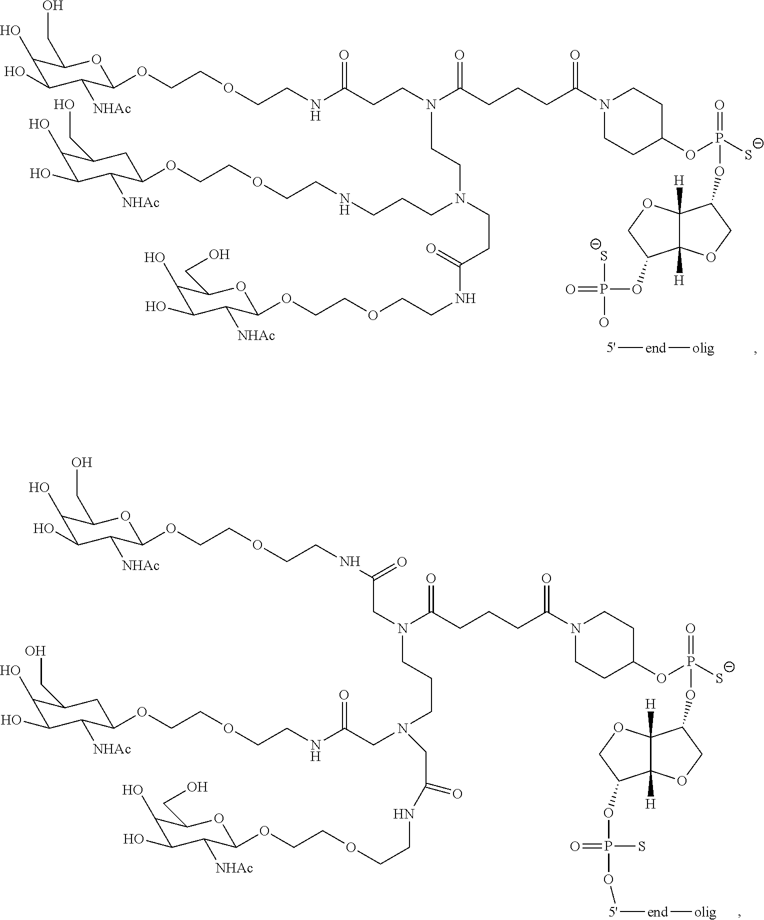

wherein the phrase “olig” each independently represents a polynucleotide moiety.

In some embodiments of the invention, in vivo delivery can also be by a beta-glucan delivery system, such as those described in U.S. Pat. Nos. 5,032,401 and 5,607,677, and U.S. Publication No. 2005/0281781, which are hereby incorporated by reference in their entirety. In vitro introduction of a PNPLA3 RNAi agent into a cell may also be done using art-known methods such as electroporation and lipofection. In certain embodiments of methods of the invention, a PNPLA3 dsRNA is delivered without a targeting agent. These RNAs may be delivered as “naked” RNA molecules. As a non-limiting example, a PNPLA3 dsRNA of the invention may be administered to a subject to treat a PNPLA3-associated disease or condition in the subject, such as a liver disease, in a pharmaceutical composition comprising the RNAi agent, but not including a targeting agent such as a GalNAc targeting compound.

In addition to certain delivery means described herein, it will be understood that RNAi delivery means, such as but not limited to those described herein and those used in the art, can be used in conjunction with embodiments of PNPLA3 RNAi agents and treatment methods described herein.

PNPLA3 dsRNA agents of the invention may be administered to a subject in an amount and manner effective to reduce a level and activity of PNPLA3 polypeptide in a cell and/or subject. In some embodiments of methods of the invention one or more PNPLA3 dsRNA agents are administered to a cell and/or subject to treat a disease or condition associated with PNPLA3 expression and activity. Methods of the invention, in some embodiments, include administering one or more PNPLA3 dsRNA agents to a subject in need of such treatment to reduce a disease or condition associated with PNPLA3 expression in the subject. PNPLA3 dsRNA agents or PNPLA3 antisense polynucleotide agents of the invention can be administered to reduce PNPLA3 expression and/or activity in one more of in vitro, ex vivo, and in vivo cells.

In some embodiments of the invention, a level, and thus an activity, of PNPLA3 polypeptide in a cell is reduced by delivering (e.g. introducing) a PNPLA3 dsRNA agent or PNPLA3 antisense polynucleotide agent into a cell. Targeting agents and methods may be used to aid in delivery of a PNPLA3 dsRNA agent or PNPLA3 antisense polynucleotide agent to a specific cell type, cell subtype, organ, spatial region within a subject, and/or to a sub-cellular region within a cell. A PNPLA3 dsRNA agent can be administered in certain methods of the invention singly or in combination with one or more additional PNPLA3 dsRNA agents. In some embodiments, 2, 3, 4, or more independently selected PNPLA3 dsRNA agents are administered to a subject.

In certain embodiments of the invention, a PNPLA3 dsRNA agent is administered to a subject to treat a PNPLA3-associated disease or condition in conjunction with one or more additional therapeutic regimens for treating the PNPLA3-associate disease or condition. Non-limiting examples of additional therapeutic regimens are: administering one or more PNPLA3 antisense polynucleotides of the invention, administering a non-PNPLA3 dsRNA therapeutic agent, and a behavioral modification. An additional therapeutic regimen may be administered at a time that is one or more of: prior to, simultaneous with, and following administration of a PNPLA3 dsRNA agent of the invention. It will be understood that simultaneous with as used herein, within five minutes of time zero, within 10 minutes of time zero, within 30 minutes of time zero, within 45 minutes of time zero, and within 60 minutes of time zero, with “time zero” the time of administration of the PNPLA3 dsRNA agent of the invention to the subject. Non-limiting examples of non-PNPLA3 dsRNA therapeutic agents are: an HMG-COA reductase inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IB AT inhibitor, a squalene synthase inhibitor, a monocyte chemoattractant protein (MCP)-I inhibitor, or fish oil; a therapeutic agent capable of reducing PNPLA3 levels and/or accumulation in a subject. Non-limiting examples of behavioral modifications are: a dietary regimen, counseling, and an exercise regimen. These and other therapeutic agents and behavior modifications are known in the art and used to treat a PNPLA3 disease or condition in a subject and may be administered to a subject in combination with the administration of one or more PNPLA3 dsRNA agents of the invention to treat the PNPLA3 disease or condition. A PNPLA3 dsRNA agent of the invention administered to a cell or subject to treat a PNPLA3-associated disease or condition may act in a synergistic manner with one or more other therapeutic agents or activities and increase the effectiveness of the one or more therapeutic agents or activities and/or to increase the effectiveness of the PNPLA3 dsRNA agent at treating the PNPLA3-associated disease or condition.

Treatment methods of the invention that include administration of a PNPLA3 dsRNA agent can be used prior to the onset of a PNPLA3-associated disease or condition and/or when a PNPLA3-associated disease or condition is present, including at an early stage, mid-stage, and late stage of the disease or condition and all times before and after any of these stages. Methods of the invention may also be to treat subjects who have previously been treated for a PNPLA3-associated disease or condition with one or more other therapeutic agents and/or therapeutic activities that were not successful, were minimally successful, and/or are no longer successful at treating the PNPLA3-associated disease or condition in the subject.

Vector Encoded dsRNAs

In certain embodiments of the invention, a PNPLA3 dsRNA agent can be delivered into a cell using a vector. PNPLA3 dsRNA agent transcription units can be included in a DNA or RNA vector. Prepare and use of such vectors encoding transgenes for delivering sequences into a cell and or subject are well known in the art. Vectors can be used in methods of the invention that result in transient expression of PNPLA3 dsRNA, for example for at least 1, 2, 3, 4, 5, 6, 7, 8, 9, 10 or more hours, 1, 2, 3, 4, 5, 6, 7, 8, 9, 10 or more weeks. The length of the transient expression can be determined using routine methods based on elements such as, but not limited to the specific vector construct selected and the target cell and/or tissue. Such transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

An individual strand or strands of a PNPLA3 dsRNA agent can be transcribed from a promoter on an expression vector. Where two separate strands are to be expressed to generate, for example, a dsRNA, two separate expression vectors can be co-introduced to a cell using means such as transfection or infection. In certain embodiments each individual strand of a PNPLA3 dsRNA agent of the invention can be transcribed by promoters that are both included on the same expression vector. In certain embodiments of the invention a PNPLA3 dsRNA agent is expressed as inverted repeat polynucleotides joined by a linker polynucleotide sequence such that the PNPLA3 dsRNA agent has a stem and loop structure.

Non-limiting examples of RNA expression vectors are DNA plasmids or viral vectors. Expression vectors useful in embodiments of the invention can be compatible with eukaryotic cells. Eukaryotic cell expression vectors are routinely used in the art and are available from a number of commercial sources. Delivery of PNPLA3 dsRNA expressing vectors can be systemic, such as by intravenous or intramuscular administration, by administration to target cells ex-planted from a subject followed by reintroduction into the subject, or by any other means that allows for introduction into a desired target cell.

Viral vector systems that may be included in an embodiment of a method of the include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Constructs for the recombinant expression of a PNPLA3 dsRNA agent may include regulatory elements, such as promoters, enhancers, etc., which may be selected to provide constitutive or regulated/inducible expression. Viral vector systems, and the use of promoters and enhancers, etc. are routine in the art and can be used in conjunction with methods and compositions described herein.

Certain embodiments of the invention include use of viral vectors for delivery of PNPLA3 dsRNA agents into cells. Numerous adenovirus-based delivery systems are routinely used in the art for deliver to, for example, lung, liver, the central nervous system, endothelial cells, and muscle. Non-limiting examples of viral vectors that may be used in methods of the invention are: AAV vectors, a pox virus such as a vaccinia virus, a Modified Virus Ankara (MVA), NYVAC, an avipox such as fowl pox or canary pox.

Certain embodiments of the invention include methods of delivering PNPLA3 dsRNA agents into cells using a vector and such vectors may be in a pharmaceutically acceptable carrier that may, but need not, include a slow release matrix in which the gene delivery vehicle is imbedded. In some embodiments, a vector for delivering a PNPLA3 dsRNA can be produced from a recombinant cell, and a pharmaceutical composition of the invention may include one or more cells that produced the PNPLA3 dsRNA delivery system.

Pharmaceutical Compositions Containing PNPLA3 dsRNA or ssRNA Agents

Certain embodiments of the invention include use of pharmaceutical compositions containing a PNPLA3 dsRNA agent or PNPLA3 antisense polynucleotide agent and a pharmaceutically acceptable carrier. The pharmaceutical composition containing the PNPLA3 dsRNA agent or PNPLA3 antisense polynucleotide agent can be used in methods of the invention to reduce PNPLA3 gene expression and PNPLA3 activity in a cell and is useful to treat a PNPLA3-associated disease or condition. Such pharmaceutical compositions can be formulated based on the mode of delivery. Non-limiting examples of formulations for modes of delivery are: a composition formulated for subcutaneous delivery, a composition formulated for systemic administration via parenteral delivery, a composition formulated for intravenous (IV) delivery, a composition formulated for intrathecal delivery, a composition formulated for direct delivery into brain, etc. Administration of a pharmaceutic composition of the invention to deliver a PNPLA3 dsRNA agent or PNPLA3 antisense polynucleotide agent into a cell may be done using one or more means such as: topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration. A PNPLA3 dsRNA agent or PNPLA3 antisense polynucleotide agent can also be delivered directly to a target tissue, for example directly into the liver, directly into a kidney, etc. It will be understood that “delivering a PNPLA3 dsRNA agent” or “delivering a PNPLA3 antisense polynucleotide agent” into a cell encompasses delivering a PNPLA3 dsRNA agent or PNPLA3 antisense polynucleotide agent, respectively, directly as well as expressing a PNPLA3 dsRNA agent in a cell from an encoding vector that is delivered into a cell, or by any suitable means with which the PNPLA3 dsRNA or PNPLA3 antisense polynucleotide agent becomes present in a cell. Preparation and use of formulations and means for delivering inhibitory RNAs are well known and routinely used in the art.

As used herein, a “pharmaceutical composition” comprises a pharmacologically effective amount of a PNPLA3 dsRNA agent or PNPLA3 antisense polynucleotide agent of the invention and a pharmaceutically acceptable carrier. The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Agents included in drug formulations are described further herein below.

As used herein terms such as: “pharmacologically effective amount,” “therapeutically effective amount” and “effective amount” refers to that amount of a PNPLA3 dsRNA agent or PNPLA3 antisense polynucleotide agent of the invention to produce the intended pharmacological, therapeutic or preventive result. For example, if a given clinical treatment is considered effective when there is at least a 10% reduction in a measurable parameter associated with a disease or disorder, a therapeutically effective amount of a drug for the treatment of that disease or disorder is the amount necessary to effect at least a 10% reduction in that parameter. For example, a therapeutically effective amount of a PNPLA3 dsRNA agent or PNPLA3 antisense polynucleotide agent can reduce PNPLA3 polypeptide levels by at least 10%.

### Effective Amounts

Methods of the invention, in some aspects comprise contacting a cell with a PNPLA3 dsRNA agent or PNPLA3 antisense polynucleotide agent in an effective amount to reduce PNPLA3 gene expression in the contacted cell. Certain embodiments of methods of the invention comprise administering a PNPLA3 dsRNA agent or a PNPLA3 antisense polynucleotide agent to a subject in an amount effective to reduce PNPLA3 gene expression and treat a PNPLA3-associated disease or condition in the subject. An “effective amount” used in terms of reducing expression of PNPLA3 and/or for treating a PNPLA3-associated disease or condition, is an amount necessary or sufficient to realize a desired biologic effect. For example, an effective amount of a PNPLA3 dsRNA agent or PNPLA3 antisense polynucleotide agent to treat a PNPLA3-associated disease or condition could be that amount necessary to (i) slow or halt progression of the disease or condition; or (ii) reverse, reduce, or eliminate one or more symptoms of the disease or condition. In some aspects of the invention, an effective amount is that amount of a PNPLA3 dsRNA agent or PNPLA3 antisense polynucleotide agent that when administered to a subject in need of a treatment of a PNPLA3-associated disease or condition, results in a therapeutic response that prevents and/or treats the disease or condition. According to some aspects of the invention, an effective amount is that amount of a PNPLA3 dsRNA agent or PNPLA3 antisense polynucleotide agent of the invention that when combined or co-administered with another therapeutic treatment for a PNPLA3-associated disease or condition, results in a therapeutic response that prevents and/or treats the disease or condition. In some embodiments of the invention, a biologic effect of treating a subject with a PNPLA3 dsRNA agent or PNPLA3 antisense polynucleotide agent of the invention may be the amelioration and or absolute elimination of symptoms resulting from the PNPLA3-associated disease or condition. In some embodiments of the invention, a biologic effect is the complete abrogation of the PNPLA3-associated disease or condition, as evidenced for example, by a diagnostic test that indicates the subject is free of the PNPLA3-associated disease or condition. A non-limiting example of a physiological symptom that may be detected includes a reduction in PNPLA3 level in liver of a subject following administration of an agent of the invention. Additional art-known means of assessing the status of a PNPLA3-associated disease or condition can be used to determine an effect of an agent and/or methods of the invention on a PNPLA3-associated disease or condition.

Typically an effective amount of a PNPLA3 dsRNA agent or PNPLA3 antisense polynucleotide agent to decrease PNPLA3 polypeptide activity to a level to treat a PNPLA3-associated disease or condition will be determined in clinical trials, establishing an effective dose for a test population versus a control population in a blind study. In some embodiments, an effective amount will be that results in a desired response, e.g., an amount that diminishes a PNPLA3-associated disease or condition in cells, tissues, and/or subjects with the disease or condition. Thus, an effective amount of a PNPLA3 dsRNA agent or PNPLA3 antisense polynucleotide agent to treat a PNPLA3-associated disease or condition that can be treated by reducing PNPLA3 polypeptide activity may be the amount that when administered decreases the amount of PNPLA3 polypeptide activity in the subject to an amount that is less than the amount that would be present in the cell, tissue, and/or subject without the administration of the PNPLA3 dsRNA agent or PNPLA3 antisense polynucleotide agent. In certain aspects of the invention the level of PNPLA3 polypeptide activity, and/or PNPLA3 gene expression present in a cell, tissue, and/or subject that has not been contacted with or administered a PNPLA3 dsRNA agent or PNPLA3 antisense polynucleotide agent of the invention is referred to as a “control” amount. In some embodiments of methods of the invention a control amount for a subject is a pre-treatment amount for the subject, in other words, a level in a subject before administration of a PNPLA3 agent can be a control level for that subject and compared to a level of PNPLA3 polypeptide activity and/or PNPLA3 gene expression in the subject following siRNA administered to the subject. In the case of treating a PNPLA3-associated disease or condition the desired response may be reducing or eliminating one or more symptoms of the disease or condition in the cell, tissue, and/or subject. The reduction or elimination may be temporary or may be permanent. It will be understood that the status of a PNPLA3-associated disease or condition can be monitored using methods of determining PNPLA3 polypeptide activity, PNPLA3 gene expression, symptom evaluation, clinical testing, etc. In some aspects of the invention, a desired response to treatment of a PNPLA3-associated disease or condition is delaying the onset or even preventing the onset of the disease or condition.

An effective amount of a compound that decreases PNPLA3 polypeptide activity may also be determined by assessing physiological effects of administration of a PNPLA3 dsRNA agent or PNPLA3 antisense polynucleotide agent on a cell or subject, such as a decrease of a PNPLA3-associated disease or condition following administration. Assays and/or symptomatic monitoring of a subject can be used to determine efficacy of a PNPLA3 dsRNA agent or PNPLA3 antisense polynucleotide agent of the invention, which may be administered in a pharmaceutical compound of the invention, and to determine the presence or absence of a response to the treatment. A non-limiting example, is that one or more art-known tests of alanine aminotransferase (ALT) or aspartate aminotransferase (AST) profile. Another non-limiting example, is that one or more art-known tests of liver function can be used to determine the status of the PNPLA3-associated liver disease or condition in a subject before and after treatment of the subject with a PNPLA3 dsRNA agent of the invention.

Some embodiments of the invention include methods of determining an efficacy of an dsRNA agent or PNPLA3 antisense polynucleotide agent of the invention administered to a subject, to treat a PNPLA3-associated disease or condition by assessing and/or monitoring one or more “physiological characteristics” of the PNPLA3-associated disease or condition in the subject. Non-limiting examples of physiological characteristics of a PNPLA3-associated disease or condition are PNPLA3 mRNA level, PNPLA3 protein level, or the number or extent of amyloid deposits, etc. Standard means of determining such physiological characteristic are known in the art and include, but are not limited to, blood tests, imaging studies, physical examination, etc.

It will be understood that the amount of a PNPLA3 dsRNA agent or PNPLA3 antisense polynucleotide agent administered to a subject can be modified based, at least in part, on such determinations of disease and/or condition status and/or physiological characteristics determined for a subject. The amount of a treatment may be varied for example by increasing or decreasing the amount of a PNPLA3-dsRNA agent or PNPLA3 antisense polynucleotide agent, by changing the composition in which the PNPLA3 dsRNA agent or PNPLA3 antisense polynucleotide agent, respectively, is administered, by changing the route of administration, by changing the dosage timing and so on. The effective amount of a PNPLA3 dsRNA agent or PNPLA3 antisense polynucleotide agent will vary with the particular condition being treated, the age and physical condition of the subject being treated; the severity of the condition, the duration of the treatment, the nature of the concurrent therapy (if any), the specific route of administration, and additional factors within the knowledge and expertise of the health practitioner. For example, an effective amount may depend upon the desired level of PNPLA3 polypeptide activity and or PNPLA3 gene expression that is effective to treat the PNPLA3-associated disease or condition. A skilled artisan can empirically determine an effective amount of a particular PNPLA3 dsRNA agent or PNPLA3 antisense polynucleotide agent of the invention for use in methods of the invention without necessitating undue experimentation. Combined with the teachings provided herein, by selecting from among various PNPLA3 dsRNA agents or PNPLA3 antisense polynucleotide agents of the invention, and weighing factors such as potency, relative bioavailability, patient body weight, severity of adverse side-effects and preferred mode of administration, an effective prophylactic or therapeutic treatment regimen can be planned that is effective to treat the particular subject. As used in embodiments of the invention, an effective amount of a PNPLA3 dsRNA agent or PNPLA3 antisense polynucleotide agent of the invention can be that amount that when contacted with a cell results in a desired biological effect in the cell.

It will be recognized that PNPLA3 gene silencing may be determined in any cell expressing PNPLA3, either constitutively or by genomic engineering, and by any appropriate assay. In some embodiments of the invention, PNPLA3 gene expression is reduced by at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% by administration of a PNPLA3 dsRNA agent of the invention. In some embodiments of the invention, PNPLA3 gene expression is reduced by at between 5% and 10%, 5% and 25%, 10% and 50%, 10% and 75%, 25% and 75%, 25% and 100%, or 50% and 100% by administration of a PNPLA3 dsRNA agent of the invention.

### Dosing

PNPLA3 dsRNA agents and PNPLA3 antisense polynucleotide agents are delivered in pharmaceutical compositions in dosages sufficient to inhibit expression of PNPLA3 genes. In certain embodiments of the invention, a dose of PNPLA3 dsRNA agent or PNPLA3 antisense polynucleotide agent is in a range of 0.01 to 200.0 milligrams per kilogram body weight of the recipient per day, generally in the range of 1 to 50 mg per kilogram body weight, 5 to 40 mg/kg body weight, 10 to 30 mg/kg body weight, 1 to 20 mg/kg body weight, 1 to 10 mg/kg body weight, 4 to 15 mg/kg body weight per day, inclusive. For example, the PNPLA3 dsRNA agent or PNPLA3 antisense polynucleotide agent can be administered in an amount that is from about 0.01 mg/kg, 0.05 mg/kg, 0.1 mg/kg, 0.2 mg/kg, 0.3 mg/kg, 0.4 mg/kg, 0.5 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, through 50 mg/kg body per single dose.

Various factors may be considered in the determination of dosage and timing of delivery of a PNPLA3 dsRNA agent of the invention. The absolute amount of a PNPLA3 dsRNA agent or PNPLA3 antisense polynucleotide agent delivered will depend upon a variety of factors including a concurrent treatment, the number of doses and the individual subject parameters including age, physical condition, size and weight. These are factors well known to those of ordinary skill in the art and can be addressed with no more than routine experimentation. In some embodiments, a maximum dose can be used, that is, the highest safe dose according to sound medical judgment.

Methods of the invention may in some embodiments include administering to a subject 1, 2, 3, 4, 5, 6, 7, 8, 9, 10, or more doses of a PNPLA3 dsRNA agent or PNPLA3 antisense polynucleotide agent. In some instances, a pharmaceutical compound, (e.g., comprising a PNPLA3 dsRNA agent or comprising a PNPLA3 antisense polynucleotide agent) can be administered to a subject at least daily, every other day, weekly, every other week, monthly, etc. Doses may be administered once per day or more than once per day, for example, 2, 3, 4, 5, or more times in one 24 hour period. A pharmaceutical composition of the invention may be administered once daily, or the PNPLA3 dsRNA agent or PNPLA3 antisense polynucleotide agent may be administered as two, three, or more sub-doses at appropriate intervals throughout the day or even using continuous infusion or delivery through a controlled release formulation. In some embodiments of methods of the invention, a pharmaceutical composition of the invention is administered to a subject one or more times per day, one or more times per week, one or more times per month, or one or more times per year.

Methods of the invention, in some aspects, include administration of a pharmaceutical compound alone, in combination with one or more other PNPLA3 dsRNA agents or PNPLA3 antisense polynucleotide agents, and/or in combination with other drug therapies or treatment activities or regimens that are administered to subjects with a PNPLA3-associated disease or condition. Pharmaceutical compounds may be administered in pharmaceutical compositions. Pharmaceutical compositions used in methods of the invention may be sterile and contain an amount of a PNPLA3 dsRNA agent or PNPLA3 antisense polynucleotide agent that will reduce activity of a PNPLA3 polypeptide to a level sufficient to produce the desired response in a unit of weight or volume suitable for administration to a subject. A dose administered to a subject of a pharmaceutical composition that includes a PNPLA3 dsRNA agent or PNPLA3 antisense polynucleotide agent to reduce PNPLA3 protein activity can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

### Treatment

PNPLA3-associated diseases and conditions in which a decrease in a level and/or activity of PNPLA3 polypeptide is effective to treat the disease or condition, can be treated using methods and PNPLA3 dsRNA agents of the invention to inhibit PNPLA3 expression. Examples of diseases and conditions that may be treated with a PNPLA3 dsRNA agent or PNPLA3 antisense polynucleotide agent of the invention and a treatment method of the invention, include, but are not limited to: liver disease, fatty liver (steatosis), nonalcoholic steatohepatitis (NASH), alcoholic steatohepatitis (ASH), cirrhosis of the liver, accumulation of fat in the liver, inflammation of the liver, hepatocellular necrosis, hepatocellular carcinoma, liver fibrosis, obesity, alcoholic liver disease, HCV hepatitis, chronic hepatitis, hereditary hemochromatosis, primary sclerosing cholangitis or nonalcoholic fatty liver disease (NAFLD). Such diseases and conditions may be referred to herein as “PNPLA3-associated diseases and conditions” and “diseases and conditions caused and/or modulated by PNPLA3.”

In certain aspects of the invention, a subject may be administered a PNPLA3 dsRNA agent or PNPLA3 antisense polynucleotide agent of the invention at a time that is one or more of before or after diagnosis of a PNPLA3-associated disease or condition. In some aspects of the invention, a subject is at risk of having or developing a PNPLA3-associated disease or condition. A subject at risk of developing a PNPLA3-associated disease or condition is one who has an increased probability of developing the PNPLA3-associated disease or condition, compared to a control risk of developing the PNPLA3-associated disease or condition. In some embodiments of the invention, a level of risk may be statistically significant compared to a control level of risk. A subject at risk may include, for instance, a subject who is, or will be, a subject who has a preexisting disease and/or a genetic abnormality that makes the subject more susceptible to a PNPLA3-associated disease or condition than a control subject without the preexisting disease or genetic abnormality; a subject having a family and/or personal medical history of the PNPLA3-associated disease or condition; and a subject who has previously been treated for a PNPLA3-associated disease or condition. It will be understood that a preexisting disease and/or a genetic abnormality that makes the subject more susceptible to a PNPLA3-associated disease or condition, may be a disease or genetic abnormality that when present has been previously identified as having a correlative relation to a higher likelihood of developing a PNPLA3-associated disease or condition.

It will be understood that a PNPLA3 dsRNA agent or PNPLA3 antisense polynucleotide agent may be administered to a subject based on a medical status of the individual subject. For example, a health-care provided for a subject may assess a PNPLA3 level measured in a sample obtained from a subject and determine it is desirable to reduce the subject's PNPLA3 level or the level of hepatic lipid droplets, by administration of a PNPLA3 dsRNA agent or PNPLA3 antisense polynucleotide agent of the invention. In this example, the PNPLA3 level may be considered to be a physiological characteristic of a PNPLA3-associated condition, even if the subject is not diagnosed as having a PNPLA3-associated disease such as one disclosed herein. A healthcare provider may monitor changes in the subject's PNPLA3 level, as a measure of efficacy of the administered PNPLA3 dsRNA agent or PNPLA3 antisense polynucleotide agent of the invention. In a non-limiting example, a biological sample, such as a liver or serum sample may be obtained from a subject and a PNPLA3 level for the subject determined in the sample. A PNPLA3 dsRNA agent or PNPLA3 antisense polynucleotide agent is administered to the subject and a liver or serum sample is obtained from the subject following the administration and the PNPLA3 level determined using the sample and the results compared to the results determined in the subject's pre-administration (prior) sample. A reduction in the subject's PNPLA3 level in the later sample compared to the pre-administration level indicates the administered PNPLA3 dsRNA agent or PNPLA3 antisense polynucleotide agent efficacy in reducing the lipid level, liver fat or hepatic lipid droplets in the subject.

Certain embodiments of methods of the invention include adjusting a treatment that includes administering a dsRNA agent or a PNPLA3 antisense polynucleotide agent of the invention to a subject based at least in part on assessment of a change in one or more of the subject's physiological characteristics of a PNPLA3-associated disease or condition resulting from the treatment. For example, in some embodiments of the invention, an effect of an administered dsRNA agent or PNPLA3 antisense polynucleotide agent of the invention may be determined for a subject and used to assist in adjusting an amount of a dsRNA agent or PNPLA3 antisense polynucleotide agent of the invention subsequently administered to the subject. In a non-limiting example, a subject is administered a dsRNA agent or PNPLA3 antisense polynucleotide agent of the invention, the subject's PNPLA3 level is determined after the administration, and based at least in part on the determined level, a greater amount of the dsRNA agent or PNPLA3 antisense polynucleotide agent is determined to be desirable in order to increase the physiological effect of the administered agent, for example to reduce or further reduce the subject's PNPLA3 level. In another non-limiting example, a subject is administered a dsRNA agent or PNPLA3 antisense polynucleotide agent of the invention, the subject's PNPLA3 level is determined after the administration and based at least in part on the determined level, a lower amount of the dsRNA agent or PNPLA3 antisense polynucleotide agent is desirable to administer to the subject.

Thus, some embodiments of the invention include assessing a change in one or more physiological characteristics of resulting from a subject's previous treatment to adjust an amount of a dsRNA agent or PNPLA3 antisense polynucleotide agent of the invention subsequently administered to the subject. Some embodiments of methods of the invention include 1, 2, 3, 4, 5, 6, or more determinations of a physiological characteristic of a PNPLA3-associated disease or condition to assess and/or monitor the efficacy of an administered PNPLA3 dsRNA agent or PNPLA3 antisense polynucleotide agent of the invention, and optionally using the determinations to adjust one or more of: a dose, administration regimen, and or administration frequency of a dsRNA agent or PNPLA3 antisense polynucleotide agent of the invention to treat a PNPLA3-associated disease or condition in a subject. In some embodiments of methods of the invention, a desired result of administering an effective amount of a dsRNA agent or PNPLA3 antisense polynucleotide agent of the invention to a subject is a reduction of the subject's PNPLA3 mRNA level, PNPLA3 protein level, the fat level and/or the lipid droplets level in the liver, or the number or extent of amyloid deposits, etc., as compared to a prior level determined for the subject, or to a control level.

As used herein, the terms “treat”, “treated”, or “treating” when used with respect to a PNPLA3-associated disease or condition may refer to a prophylactic treatment that decreases the likelihood of a subject developing the PNPLA3-associated disease or condition, and also may refer to a treatment after the subject has developed a PNPLA3-associated disease or condition in order to eliminate or reduce the level of the PNPLA3-associated disease or condition, prevent the PNPLA3-associated disease or condition from becoming more advanced (e.g., more severe), and/or slow the progression of the PNPLA3-associated disease or condition in a subject compared to the subject in the absence of the therapy to reduce activity in the subject of PNPLA3 polypeptide.

Certain embodiments of agents, compositions, and methods of the invention can be used to inhibit PNPLA3 gene expression. As used herein in reference to expression of a PNPLA3 gene, the terms “inhibit,” “silence,” “reduce,” “down-regulate,” and “knockdown” mean the expression of the PNPLA3 gene, as measured by one or more of: a level of RNA transcribed from the gene, a level of activity of PNPLA3 expressed, and a level of PNPLA3 polypeptide, protein or protein subunit translated from the mRNA in a cell, group of cells, tissue, organ, or subject in which the PNPLA3 gene is transcribed, is reduced when the cell, group of cells, tissue, organ, or subject is contacted with (e.g., treated with) a PNPLA3 dsRNA agent or PNPLA3 antisense polynucleotide agent of the invention, compared to a control level of RNA transcribed from the PNPLA3 gene, a level of activity of expressed PNPLA3, or a level of PNPLA3 translated from the MRNA, respectively. In some embodiments, a control level is a level in a cell, tissue, organ or subject that has not been contacted with (e.g. treated with) the PNPLA3 dsRNA agent or PNPLA3 antisense polynucleotide agent.

### Administration Methods

A variety of administration routes for a PNPLA3 dsRNA agent or PNPLA3 antisense polynucleotide agent are available for use in methods of the invention. The particular delivery mode selected will depend at least in part, upon the particular condition being treated and the dosage required for therapeutic efficacy. Methods of this invention, generally speaking, may be practiced using any mode of administration that is medically acceptable, meaning any mode that produces effective levels of treatment of a PNPLA3-associated disease or condition without causing clinically unacceptable adverse effects. In some embodiments of the invention, a PNPLA3 dsRNA agent or PNPLA3 antisense polynucleotide agent may be administered via an oral, enteral, mucosal, subcutaneous, and/or parenteral route. The term “parenteral” includes subcutaneous, intravenous, intrathecal, intramuscular, intraperitoneal, and intrasternal injection, or infusion techniques. Other routes include but are not limited to nasal (e.g., via a gastro-nasal tube), dermal, vaginal, rectal, sublingual, and inhalation. Delivery routes of the invention may include intrathecal, intraventricular, or intracranial. In some embodiments of the invention, a PNPLA3 dsRNA agent or PNPLA3 antisense polynucleotide agent may be placed within a slow release matrix and administered by placement of the matrix in the subject. In some aspects of the invention, a PNPLA3 dsRNA agent or PNPLA3 antisense polynucleotide agent may be delivered to a subject cell using nanoparticles coated with a delivery agent that targets a specific cell or organelle. Various delivery means, methods, agents are known in the art. Non-limiting examples of delivery methods and delivery agents are additionally provided elsewhere herein. In some aspects of the invention, the term “delivering” in reference to a PNPLA3 dsRNA agent or PNPLA3 antisense polynucleotide agent may mean administration to a cell or subject of one or more “naked” PNPLA3 dsRNA agent or PNPLA3 antisense polynucleotide agent sequences and in certain aspects of the invention “delivering” means administration to a cell or subject via transfection means, delivering a cell comprising a PNPLA3 dsRNA agent or PNPLA3 antisense polynucleotide agent to a subject, delivering a vector encoding a PNPLA3 dsRNA agent or PNPLA3 antisense polynucleotide agent into a cell and/or subject, etc. Delivery of a PNPLA3 dsRNA agent or PNPLA3 antisense polynucleotide agent using a transfection means may include administration of a vector to a cell and/or subject.

In some methods of the invention one or more PNPLA3 dsRNA agents or PNPLA3 antisense polynucleotide agents may be administered in formulations, which may be administered in pharmaceutically acceptable solutions, which may routinely contain pharmaceutically acceptable concentrations of salt, buffering agents, preservatives, compatible carriers, adjuvants, and optionally other therapeutic ingredients. In some embodiments of the invention a PNPLA3 dsRNA agent or PNPLA3 antisense polynucleotide agent may be formulated with another therapeutic agent for simultaneous administration. According to methods of the invention, a PNPLA3 dsRNA agent or PNPLA3 antisense polynucleotide agent may be administered in a pharmaceutical composition. In general, a pharmaceutical composition comprises a PNPLA3 dsRNA agent or PNPLA3 antisense polynucleotide agent and optionally, a pharmaceutically-acceptable carrier. Pharmaceutically-acceptable carriers are well-known to those of ordinary skill in the art. As used herein, a pharmaceutically-acceptable carrier means a non-toxic material that does not interfere with the effectiveness of the biological activity of the active ingredients, e.g., the ability of the PNPLA3 dsRNA agent or PNPLA3 antisense polynucleotide agent to inhibit PNPLA3 gene expression in a cell or subject. Numerous methods to administer and deliver dsRNA agents or PNPLA3 antisense polynucleotide agents for therapeutic use are known in the art and may be utilized in methods of the invention.

Pharmaceutically acceptable carriers include diluents, fillers, salts, buffers, stabilizers, solubilizers and other materials that are well-known in the art. Exemplary pharmaceutically acceptable carriers are described in U.S. Pat. No. 5,211,657 and others are known by those skilled in the art. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Some embodiments of methods of the invention include administering one or more PNPLA3 dsRNA agents or PNPLA3 antisense polynucleotide agents directly to a tissue. In some embodiments, the tissue to which the compound is administered is a tissue in which the PNPLA3-associated disease or condition is present or is likely to arise, non-limiting examples of which are the liver or kidney. Direct tissue administration may be achieved by direct injection or other means. Many orally delivered compounds naturally travel to and through the liver and kidneys and some embodiments of treatment methods of the invention include oral administration of one or more PNPLA3 dsRNA agents to a subject. PNPLA3 dsRNA agents or PNPLA3 antisense polynucleotide agents, either alone or in conjunction with other therapeutic agents, may be administered once, or alternatively they may be administered in a plurality of administrations. If administered multiple times, the PNPLA3 dsRNA agent or PNPLA3 antisense polynucleotide agent may be administered via different routes. For example, though not intended to be limiting, a first (or first several) administrations may be made via subcutaneous means and one or more additional administrations may be oral and/or systemic administrations.

For embodiments of the invention in which it is desirable to administer a PNPLA3 dsRNA agent or PNPLA3 antisense polynucleotide agent systemically, the PNPLA3 dsRNA agent or PNPLA3 antisense polynucleotide agent may be formulated for parenteral administration by injection, e.g., by bolus injection or continuous infusion. Formulations for injection may be presented in unit dosage form, e.g., in ampoules or in multi-dose containers, with or without an added preservative. PNPLA3 dsRNA agent formulations (also referred to as pharmaceutical compositions) may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day may be used as needed to achieve appropriate systemic or local levels of one or more PNPLA3 dsRNA agents or PNPLA3 antisense polynucleotide agents and to achieve appropriate reduction in PNPLA3 protein activity.

In yet other embodiments, methods of the invention include use of a delivery vehicle such as biocompatible microparticle, nanoparticle, or implant suitable for implantation into a recipient, e.g., a subject. Exemplary bioerodible implants that may be useful in accordance with this method are described in PCT Publication No. WO 95/24929 (incorporated by reference herein), which describes a biocompatible, biodegradable polymeric matrix for containing a biological macromolecule.

Both non-biodegradable and biodegradable polymeric matrices can be used in methods of the invention to deliver one or more PNPLA3 dsRNA agents or PNPLA3 antisense polynucleotide agents to a subject. In some embodiments, a matrix may be biodegradable. Matrix polymers may be natural or synthetic polymers. A polymer can be selected based on the period of time over which release is desired, generally in the order of a few hours to a year or longer. Typically, release over a period ranging from between a few hours and three to twelve months can be used. The polymer optionally is in the form of a hydrogel that can absorb up to about 90% of its weight in water and further, optionally is cross-linked with multivalent ions or other polymers.

In general, PNPLA3 dsRNA agents or PNPLA3 antisense polynucleotide agents may be delivered in some embodiments of the invention using the bioerodible implant by way of diffusion, or by degradation of the polymeric matrix. Exemplary synthetic polymers for such use are well known in the art. Biodegradable polymers and non-biodegradable polymers can be used for delivery of PNPLA3 dsRNA agents or PNPLA3 antisense polynucleotide agents using art-known methods. Bioadhesive polymers such as bioerodible hydrogels (see H. S. Sawhney, C. P. Pathak and J. A. Hubell in Macromolecules, 1993, 26, 581-587, the teachings of which are incorporated by reference herein) may also be used to deliver PNPLA3 dsRNA agents or PNPLA3 antisense polynucleotide agents for treatment of a PNPLA3-associated disease or condition. Additional suitable delivery systems can include time-release, delayed release or sustained release delivery systems. Such systems can avoid repeated administrations of a PNPLA3 dsRNA agent or PNPLA3 antisense polynucleotide agent, increasing convenience to the subject and the medical care professional. Many types of release delivery systems are available and known to those of ordinary skill in the art. (See for example: U.S. Pat. Nos. 5,075,109; 4,452,775; 4,675,189; 5,736,152; 3,854,480; 5,133,974; and 5,407,686 (the teaching of each of which is incorporated herein by reference). In addition, pump-based hardware delivery systems can be used, some of which are adapted for implantation. Use of a long-term sustained release implant may be suitable for prophylactic treatment of subjects and for subjects at risk of developing a recurrent PNPLA3-associated disease or condition. Long-term release, as used herein, means that the implant is constructed and arranged to deliver a therapeutic level of a PNPLA3 dsRNA agent or PNPLA3 antisense polynucleotide agent for at least up to 10 days, 20 days, 30 days, 60 days, 90 days, six months, a year, or longer. Long-term sustained release implants are well-known to those of ordinary skill in the art and include some of the release systems described above.

Therapeutic formulations of PNPLA3 dsRNA agents or PNPLA3 antisense polynucleotide agents may be prepared for storage by mixing the molecule or compound having the desired degree of purity with optional pharmaceutically acceptable carriers, excipients or stabilizers [Remington's Pharmaceutical Sciences 21st edition, (2006)], in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

### Cells, Subjects, and Controls

Methods of the invention may be used in conjunction with cells, tissues, organs and/or subjects. In some aspects of the invention a subject is a human or vertebrate mammal including but not limited to a dog, cat, horse, cow, goat, mouse, rat, and primate, e.g., monkey. Thus, the invention can be used to treat PNPLA3-associated diseases or conditions in human and non-human subjects. In some aspects of the invention a subject may be a farm animal, a zoo animal, a domesticated animal or non-domesticated animal and methods of the invention can be used in veterinary prevention and treatment regimens. In some embodiments of the invention, the subject is a human and methods of the invention can be used in human prevention and treatment regimens.

Non-limiting examples of subjects to which the present invention can be applied are subjects who are diagnosed with, suspected of having, or at risk of having a disease or condition associated with a higher than desirable PNPLA3 expression and/or activity, also referred to as “elevated levels of PNPLA3 expression”. Non-limiting examples of diseases and conditions associated with a higher than desirable levels of PNPLA3 expression and/or activity are described elsewhere herein. Methods of the invention may be applied to a subject who, at the time of treatment, has been diagnosed as having the disease or condition associated with a higher than desirable PNPLA3 expression and/or activity, or a subject who is considered to be at risk for having or developing a disease or condition associated with a higher than desirable PNPLA3 expression and/or activity. In some aspects of the invention a disease or condition associated with a higher than desirable PNPLA3 level of expression and/or activity is an acute disease or condition, and in certain aspects of the invention a disease or condition associated with a higher than desirable PNPLA3 level of expression and/or activity is a chronic disease or condition.

In a non-limiting example, a PNPLA3 dsRNA agent of the invention is administered to a subject diagnosed with, suspected of having, or at risk of having, nonalcoholic steatohepatitis (NASH), which is a disease in which it is desirable to reduce PNPLA3 expression. Methods of the invention may be applied to the subject who, at the time of treatment, has been diagnosed as having the disease or condition, or a subject who is considered to be at risk for having or developing the disease or condition.

In another non-limiting example, a PNPLA3 dsRNA agent of the invention is administered to a subject diagnosed with, suspected of having, or at risk of having, nonalcoholic fatty liver disease (NAFLD), which is a disease in which it is desirable to reduce PNPLA3 expression. Methods of the invention may be applied to the subject who, at the time of treatment, has been diagnosed as having the disease or condition, or a subject who is considered to be at risk for having or developing the disease or condition.

A cell to which methods of the invention may be applied include cells that are in vitro, in vivo, ex vivo cells. Cells may be in a subject, in culture, and/or in suspension, or in any other suitable state or condition. A cell to which a method of the invention may be applied can be a liver cell, a hepatocyte, a cardiac cell, a pancreatic cell, a cardiovascular cell, kidney cell or other type of vertebrate cell, including human and non-human mammalian cells. In certain aspects of the invention, a cell to which methods of the invention may be applied is a healthy, normal cell that is not known to be a disease cell. In certain embodiments of the invention a cell to which methods and compositions of the invention are applied to a liver cell, a hepatocyte, a cardiac cell, a pancreatic cell, a cardiovascular cell, and/or a kidney cell. In certain aspects of the invention, a control cell is a normal cell, but it will be understood that a cell having a disease or condition may also serve as a control cell in particular circumstances for example to compare results in a treated cell having a disease or condition versus an untreated cell having the disease or condition, etc.

A level of PNPLA3 polypeptide activity can be determined and compared to control level of PNPLA3 polypeptide activity, according to methods of the invention. A control may be a predetermined value, which can take a variety of forms. It can be a single cut-off value, such as a median or mean. It can be established based upon comparative groups, such as in groups having normal levels of PNPLA3 polypeptide and/or PNPLA3 polypeptide activity and groups having increased levels of PNPLA3 polypeptide and/or PNPLA3 polypeptide activity. Another non-limiting example of comparative groups may be groups having one or more symptoms of or a diagnosis of a PNPLA3-associated disease or condition; groups without having one or more symptoms of or a diagnosis of the disease or condition; groups of subjects to whom an siRNA treatment of the invention has been administered; groups of subjects to whom an siRNA treatment of the invention has not been administered. Typically, a control may be based on apparently healthy normal individuals in an appropriate age bracket or apparently healthy cells. It will be understood that controls according to the invention may be, in addition to predetermined values, samples of materials tested in parallel with the experimental materials. Examples include samples from control populations or control samples generated through manufacture to be tested in parallel with the experimental samples. In some embodiments of the invention, a control may include a cell or subject not contacted or treated with a PNPLA3 dsRNA agent of the invention and in such instances, a control level of PNPLA3 polypeptide and/or PNPLA3 polypeptide activity can be compared to a level of PNPLA3 polypeptide and/or PNPLA3 polypeptide activity in a cell or subject contacted with a PNPLA3 dsRNA agent or PNPLA3 antisense polynucleotide agent of the invention.

In some embodiments of the invention a level of PNPLA3 polypeptide determined for a subject can be a control level against which a level of PNPLA3 polypeptide determined for the same subject at a different time is compared. In a non-limiting example, a level of PNPLA3 is determined in a biological sample obtained from a subject who has not been administered a PNPLA3 treatment of the invention. In some embodiments, the biological sample is a serum sample. The level of PNPLA3 polypeptide determined in the sample obtained from the subject can serve as a baseline or control value for the subject. After one or more administrations of a PNPLA3 dsRNA agent to the subject in a treatment method of the invention, one or more additional serum samples can be obtained from the subject and the level of PNPLA3 polypeptide in the subsequent sample or samples can be compared to the control/baseline level for the subject. Such comparisons can be used to assess onset, progression, or recession of a PNPLA3 associated disease or condition in the subject. For example, a level of PNPLA3 polypeptide in the baseline sample obtained from the subject that is higher than a level obtained from the same subject after the subject has been administered a PNPLA3 dsRNA agent or PNPLA3 antisense polynucleotide agent of the invention indicates regression of the PNPLA3-associated disease or condition and indicates efficacy of the administered PNPLA3 dsRNA agent of the invention for treatment of the PNPLA3-associated disease or condition.

In some aspects of the invention, values of one or more of a level of PNPLA3 polypeptide and/or PNPLA3 polypeptide activity determined for a subject may serve as control values for later comparison of levels of PNPLA3 polypeptide and/or PNPLA3 activity, in that same subject, thus permitting assessment of changes from a “baseline” PNPLA3 polypeptide activity in a subject. Thus, an initial PNPLA3 polypeptide level and/or initial PNPLA3 polypeptide activity level may be present and/or determined in a subject and methods and compounds of the invention may be used to decrease the level of PNPLA3 polypeptide and/or PNPLA3 polypeptide activity in the subject, with the initial level serving as a control level for that subject.

Using methods of the invention, PNPLA3 dsRNA agents and/or PNPLA3 antisense polynucleotide agents of the invention may be administered to a subject. Efficacy of the administration and treatment of the invention can be assessed when a level of PNPLA3 polypeptide in a serum sample obtained from a subject is decreased by at least 0.5%, 1%, 5%, 10%, 20%, 30%, 40%, 50%, 60%, 70%, 80%, 90%, 95%, or more compared to a pre-administration level of PNPLA3 polypeptide in a serum sample obtained from the subject at a prior time point, or compared to a non-contacted control level, for example a level of PNPLA3 polypeptide in a control serum sample. It will be understood that a level of PNPLA3 polypeptide and a level of PNPLA3 polypeptide activity both correlate with a level of PNPLA3 gene expression. Certain embodiments of methods of the invention comprise administering a PNPLA3 dsRNA and/or PNPLA3 antisense agent of the invention to a subject in an amount effective to inhibit PNPLA3 gene expression and thereby reduce a level of PNPLA3 polypeptide and reduce a level of PNPLA3 polypeptide activity in the subject.

Some embodiments of the invention, include determining presence, absence, and/or an amount (also referred to herein as a level) of PNPLA3 polypeptide in one or more biological samples obtained from one or more subjects. The determination can be used to assess efficacy of a treatment method of the invention. For example, methods and compositions of the invention can be used to determine a level of PNPLA3 polypeptide in a biological sample obtained from a subject previously treated with administration of a PNPLA3 dsRNA agent and/or a PNPLA3 antisense agent of the invention. A level of PNPLA3 polypeptide determined in a serum sample obtained from the treated subject that is lower by at least 0.5%, 1%, 5%, 10%, 20%, 30%, 40%, 50%, 60%, 70%, 80%, 90%, 95%, or more compared to a pretreatment level of PNPLA3 polypeptide determined for the subject, or compared to a non-contacted control biological sample level, indicates a level of efficacy of the treatment administered to the subject.

In some embodiments of the invention a physiological characteristic of a PNPLA3-associated disease or condition determined for a subject can be a control determination against which a determination of the physiological characteristic in the same subject at a different time is compared. In a non-limiting example, a physiological characteristic such as PNPLA3 mRNA level, PNPLA3 protein level, or the number or extent of amyloid deposits is determined in a biological sample, such as a liver or serum sample, obtained from a subject who has not been administered a PNPLA3 treatment of the invention. The PNPLA3 mRNA level (and/or other physiological characteristic of a PNPLA3 disease or condition) determined in the sample obtained from the subject can serve as a baseline or control value for the subject. After one or more administrations of a PNPLA3 dsRNA agent to the subject in a treatment method of the invention, one or more additional liver or serum samples can be obtained from the subject and PNPLA3 mRNA level and/or PNPLA3 protein level in the subsequent sample or samples are compared to the control/baseline level and/or ratio, respectively, for the subject. Such comparisons can be used to assess onset, progression, or recession of a PNPLA3 associated disease or condition in the subject. For example, PNPLA3 mRNA level in the baseline sample obtained from the subject that is higher than PNPLA3 mRNA level determined in a sample obtained from the same subject after the subject has been administered a PNPLA3 dsRNA agent or PNPLA3 antisense polynucleotide agent of the invention indicates regression of the PNPLA3-associated disease or condition and indicates efficacy of the administered PNPLA3 dsRNA agent of the invention for treatment of the PNPLA3-associated disease or condition.

In some aspects of the invention, values of one or more of a physiological characteristic of a PNPLA3-associated disease or condition determined for a subject may serve as control values for later comparison of the physiological characteristics in that same subject, thus permitting assessment of changes from a “baseline” physiological characteristic in a subject. Thus, an initial physiological characteristic may be present and/or determined in a subject and methods and compounds of the invention may be used to decrease the level of PNPLA3 polypeptide and/or PNPLA3 polypeptide activity in the subject, with the initial physiological characteristic determination serving as a control for that subject.

Using methods of the invention, PNPLA3 dsRNA agents and/or PNPLA3 antisense polynucleotide agents of the invention may be administered to a subject in an effective amount to treat a PNPLA3 disease or condition. Efficacy of the administration and treatment of the invention can be assessed by determining a change in one or more physiological characteristics of the PNPLA3 disease or condition. In a non-limiting example, a PNPLA3 mRNA level in a serum sample obtained from a subject is decreased by at least 0.5%, 1%, 5%, 10%, 20%, 30%, 40%, 50%, 60%, 70%, 80%, 90%, 95%, or more compared to a pre-administration lipid in a serum sample obtained from the subject at a prior time point, or compared to a non-contacted control level, for example PNPLA3 mRNA level in a control serum sample. It will be understood that PNPLA3 mRNA level, PNPLA3 protein level, or the number or extent of amyloid deposits in a subject each correlates with a level of PNPLA3 gene expression. Certain embodiments of methods of the invention comprise administering a PNPLA3 dsRNA and/or PNPLA3 antisense agent of the invention to a subject in an amount effective to inhibit PNPLA3 gene expression and thereby reduce PNPLA3 mRNA level, PNPLA3 protein level, or the number or extent of amyloid deposits in a subject, or otherwise positively impact a physiological characteristic of a PNPLA3-associated disease or condition in the subject.

Some embodiments of the invention, include determining presence, absence, and/or a change in a physiological characteristic of a PNPLA3-associated disease or condition using methods such as but not limited to: (1) assessing one or more biological samples obtained from one or more subjects for the physiological characteristic; (2) imaging a subject (for example but not limited to obtaining a liver image); and (3) or physical examination of the subject. The determination can be used to assess efficacy of a treatment method of the invention.

### Kits

Also within the scope of the invention are kits that comprise one or more PNPLA3 dsRNA agents and/or PNPLA3 antisense polynucleotide agents and instructions for its use in methods of the invention. Kits of the invention may include one or more of a PNPLA3 dsRNA agent, PNPLA3 sense polynucleotide, and PNPLA3 antisense polynucleotide agent that may be used to treat a PNPLA3-associated disease or condition. Kits containing one or more PNPLA3 dsRNA agents, PNPLA3 sense polynucleotides, and PNPLA3 antisense polynucleotide agents can be prepared for use in treatment methods of the invention. Components of kits of the invention may be packaged either in aqueous medium or in lyophilized form. A kit of the invention may comprise a carrier being compartmentalized to receive in close confinement therein one or more container means or series of container means such as test tubes, vials, flasks, bottles, syringes, or the like. A first container means or series of container means may contain one or more compounds such as a PNPLA3 dsRNA agent and/or PNPLA3 sense or antisense polynucleotide agent. A second container means or series of container means may contain a targeting agent, a labelling agent, a delivery agent, etc. that may be included as a portion of a PNPLA3 dsRNA agent and/or PNPLA3 antisense polynucleotide to be administered in an embodiment of a treatment method of the invention.

A kit of the invention may also include instructions. Instructions typically will be in written form and will provide guidance for carrying-out a treatment embodied by the kit and for making a determination based upon that treatment.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1. Preparation of Intermediate-A and Intermediate-B

As shown in Scheme 1 below, Intermediate-A was synthesized by treating commercially available galactosamine pentaacetate with trimethylsilyl trifluoromethanesulfonate (TMSOTf) in dichloromethane (DCM). This was followed by glycosylation with Cbz protected 2-(2-aminoethoxy) ethan-1-ol to give Compound II. The Cbz protecting group was removed by hydrogenation to afford Intermediate-A as a trifluoroacetate (TFA) salt. Intermediate B was synthesized based on the same scheme except Cbz protected 2-(2-(2-aminoethoxy) ethoxy) ethan-1-ol was used as the starting material.

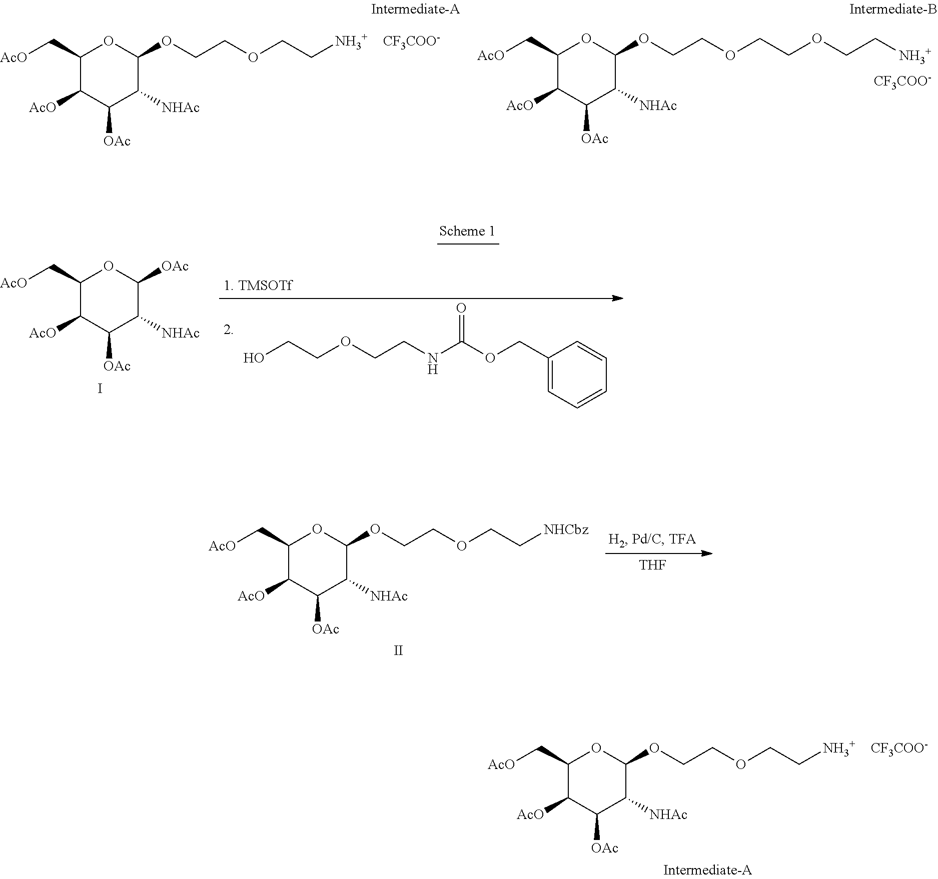

To a solution of Compound I (20.0 g, 51.4 mmol) in 100 mL 1,2-dichloroethane (DCE) was added TMSOTf (17.1 g, 77.2 mmol). The resulting reaction solution was stirred at 60° C. for 2 hrs, and then at 25° C. for 1 hr. Cbz protected 2-(2-aminoethoxy) ethan-1-ol (13.5 g, 56.5 mmol) in DCE (100 mL) dried over 4 Å powder molecular sieves (10 g) was added dropwise to the above mentioned reaction solution at 0° C. under N₂ atmosphere. The resulting reaction mixture was stirred at 25° C. for 16 hrs under N₂ atmosphere. The reaction mixture was filtered and washed with sat. NaHCO₃ (200 mL), water (200 mL) and sat. brine (200 mL). The organic layer was dried over anhydrous Na₂SO₄, filtered and concentrated under reduced pressure to give a crude product, which was triturated with 2-Methyltetrahydrofuran/heptane (5/3, v/v, 1.80 L) for 2 hrs. Resulting mixture was filtered and dried to give Compound II (15.0 g, 50.3% yield) as a white solid.

To a dried and argon purged hydrogenation bottle was carefully added 10% Pd/C (1.50 g), followed by 10 mL tetrahydrofuran (THF) and then a solution of Compound II (15.0 g, 26.4 mmol) in THF (300 mL) and TFA (trifluoroacetic acid, 3.00 g, 26.4 mmol). The resulting mixture was degassed and purged with H₂ three times and stirred at 25° C. for 3 hrs under H₂ (45 psi) atmosphere. Thin-layer chromatography (TLC, solvent: DCM:MeOH=10:1) indicated Compound II was consumed completely. The reaction mixture was filtered and concentrated under reduced pressure. Residue was dissolved in anhydrous DCM (500 mL) and concentrated. This process was repeated 3 times to give Intermediate-A (14.0 g, 96.5% yield) as a foamy white solid. ¹H NMR (400 MHz DMSO-d₆): δ ppm 7.90 (d, J=9.29 Hz, 1H), 7.78 (br s, 3H), 5.23 (d, J=3.26 Hz, 1H), 4.98 (dd, J=11.29, 3.26 Hz, 1H), 4.56 (d, J=8.53 Hz, 1H), 3.98-4.07 (m, 3H), 3.79-3.93 (m, 2H), 3.55-3.66 (m, 5H), 2.98 (br d, J=4.77 Hz, 2H), 2.11 (s, 3H), 2.00 (s, 3H), 1.90 (s, 3H), 1.76 (s, 3H).

Intermediate-B was synthesized using similar procedures for synthesis of Intermediate-A. ¹H NMR (400 MHz DMSO-d₆): δ ppm 7.90 (br d, J=9.03 Hz, 4H), 5.21 (d, J=3.51 Hz, 1H), 4.97 (dd, J=11.1 Hz, 1H), 4.54 (d, J=8.53 Hz, 1H), 3.98-4.06 (m, 3H), 3.88 (dt, J=10.9 Hz, 1H), 3.76-3.83 (m, 1H), 3.49-3.61 (m, 9H), 2.97 (br s, 2H), 2.10 (s, 3H), 1.99 (s, 3H), 1.88 (s, 3H), 1.78 (s, 3H). Mass calc. for C₂₀H₃₄N₂O₁₁: 478.22; found: 479.3 (M+H⁺).

### Example 2. Synthesis of GalNAc Ligand Cluster Phosphoramidite GLPA1, GLPA2 and GLPA15

Scheme 2 below was followed to prepare GLPA1 and GLPA2. Starting from benzyl protected propane-1,3-diamine, it was alkylated with tert-butyl 2-bromoacetate to afford triester Compound I. The benzyl protecting group was removed by hydrogenation to afford secondary amine Compound II. Amide coupling with 6-hydroxyhexanoic acid afforded Compound III. tert-Butyl protecting groups were then removed upon treatment of HCl in dioxane to generate triacid Compound IV. Amide coupling between triacid compound IV and Intermediate-A or Intermediate-B was performed to afford Compound Va or Vb. Phosphoramidite GLPA1 or GLPA2 was synthesized by phosphitylation of Compound Va or Vb with 2-Cyanoethyl N,N-diisopropylchlorophosphoramidite and a catalytic amount of 1H-tetrazole.

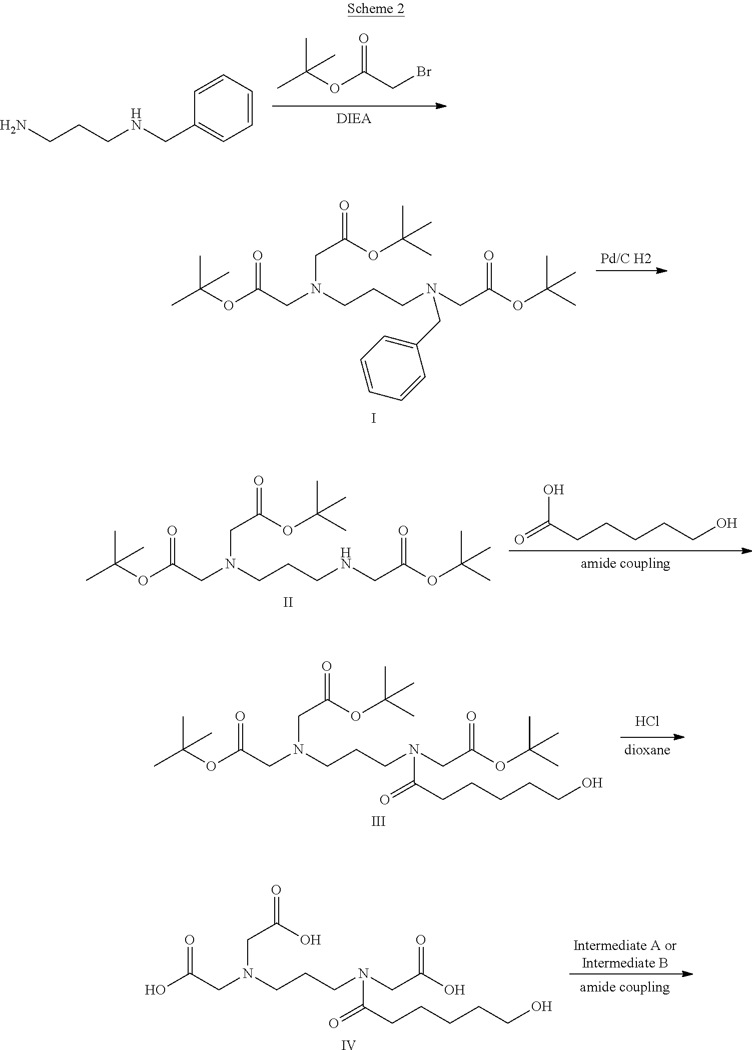

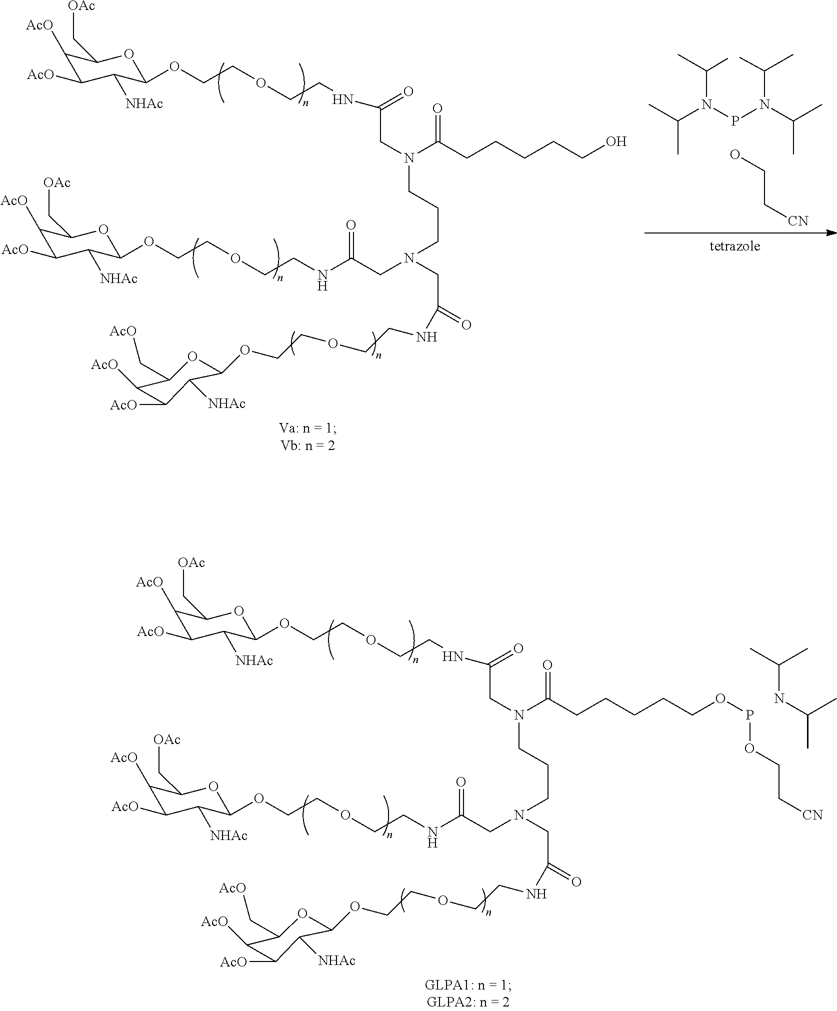

To a solution of N-Benzyl-1,3-propanediamine (5.00 g, 30.4 mmol) in dimethylformamide (DMF, 100 mL) was added tert-butyl 2-bromoacetate (23.7 g, 121 mmol), followed by addition of diisopropylethylamine (DIEA, 23.61 g, 182 mmol) dropwise. The resulting reaction mixture was stirred at 25-30° C. for 16 hrs. LCMS showed N-Benzyl-1,3-propanediamine was consumed completely. Reaction mixture was diluted with H₂O (500 mL) and extracted with EtOAc (500 mL×2). The combined organics were washed with sat. brine (1 L), dried over anhydrous Na₂SO₄, filtered, and concentrated under reduced pressure to give crude product, which was purified by silica gel column chromatography (gradient: petroleum ether:ethyl acetate from 20:1 to 5:1). Compound I (12.1 g, 78.4% yield) was obtained as a colorless oil. ¹H NMR (400 MHz, CDCl₃): δ ppm 7.26-7.40 (m, 5H), 3.79 (s, 2H), 3.43 (s, 4H), 3.21 (s, 2H), 2.72 (dt, J=16.9, 7.34 Hz, 4H), 1.70 (quin, J=7.2 Hz, 2H), 1.44-1.50 (m, 27H).

A dried hydrogenation bottle was purged with Argon three times. Pd/C (200 mg, 10%) was added, followed by MeOH (5 mL) and then a solution of Compound I (1.00 g, 1.97 mmol) in MeOH (5 mL). The reaction mixture was degassed under vacuum and refilled with H₂. This process was repeated three times. The mixture was stirred at 25° C. for 12 hrs under H₂ (15 psi) atmosphere. LCMS showed Compound I was consumed completely. The reaction mixture was filtered under reduced pressure under N₂ atmosphere. Filtrate was concentrated under reduced pressure to give Compound II (655 mg, 79.7% yield) as yellow oil, which was used for the next step without further purification. ¹H NMR (400 MHz, CDCl₃): δ ppm 3.44 (s, 4H), 3.31 (s, 2H), 2.78 (t, J=7.1 Hz, 2H), 2.68 (t, J=6.9 Hz, 2H), 1.88 (br s, 1H), 1.69 (quin, J=7.03 Hz, 2H), 1.44-1.50 (s, 27H).

A mixture of Compound II (655 mg, 1.57 mmol), 6-hydroxyhexanoic acid (249 mg, 1.89 mmol), DIEA (1.02 g, 7.86 mmol), 1-ethyl-3-(3-dimethylaminopropyl) carbodiimide hydrochloride (EDCI, 904 mg, 4.72 mmol), and 1-Hydroxybenzotriazole (HOBt, 637 mg, 4.72 mmol) in DMF (6 mL) was degassed and purged with N₂ three times, and then was stirred at 25° C. for 3 hrs under N₂ atmosphere. LCMS indicated desired product. The reaction mixture was diluted with H₂O (10 mL) and extracted with EtOAc 20 mL (10 mL×2). Organics were combined and washed with sat. brine (20 mL), dried over anhydrous Na₂SO₄, filtered, and concentrated to give crude product, which was purified by silica gel column chromatography (gradient: petroleum ether:ethyl acetate from 5:1 to 1:1) to afford Compound III (650 mg, 77.8% yield) as a yellow oil. ¹H NMR (400 MHz, CDCl₃): δ ppm 3.90-3.95 (s, 2H), 3.63 (t, J=6.40 Hz, 2H), 3.38-3.45 (m, 6H), 2.72 (t, J=6.65 Hz, 2H), 2.40 (t, J=7.28 Hz, 2H), 1.55-1.75 (m, 8H), 1.44 (s, 27H). Mass calc, for C₂₇H₅₀N₂O₈: 530.36; found: 531.3 (M+H⁺).

A mixture of Compound III (5.5 g, 10.3 mmol) in HCl/dioxane (2M, 55 mL) was stirred at 25° C. for 3 hrs. LCMS showed complete consumption of Compound III. Reaction mixture was filtered, washed with EtOAc (50 mL), and dried under reduced pressure to give crude product. It was dissolved in CH₃CN (50 mL), volatiles were removed under vacuum. This process was repeated three times to give Compound IV (2.05 g, 54.5% yield) as a white solid. ¹H NMR (400 MHz, D₂O): δ ppm 4.21 (s, 1H), 4.07 (d, J=4.5 Hz, 4H), 3.99 (s, 1H), 3.45-3.52 (m, 3H), 3.42 (t, J=6.5 Hz, 1H), 3.32-3.38 (m, 1H), 3.24-3.31 (m, 1H), 2.37 (t, J=7.4 Hz, 1H), 2.24 (t, J=7.4 Hz, 1H), 1.99 (dt, J=15.5, 7.53 Hz, 1H), 1.85-1.94 (m, 1H), 1.85-1.94 (m, 1H), 1.39-1.56 (m, 4H), 1.19-1.31 (m, 2H).

A mixture of Compound IV (500 mg, 1.05 mmol), Intermediate-A (2.02 g, 3.67 mmol), DIEA (813 mg, 6.30 mmol), EDCI (704 mg, 3.67 mmol) and HOBt (496 mg, 3.67 mmol) in DMF (10 mL) was degassed and purged with N₂ for 3 times, and then the mixture was stirred at 25° C. for 3 hrs under N₂ atmosphere. LCMS indicated desired product. The reaction mixture was quenched by addition of H₂O (10 mL), extracted with DCM (10 mL×2). The combined organics were extracted with 10% citric acid (20 mL). The aqueous phase was neutralized with saturated NaHCO₃ solution and re-extracted with DCM (10 mL×2). Organics were dried over sodium sulfate, filtered and concentrated under reduced pressure to give Compound Va (570 mg, 0.281 mmol, 26.8% yield) as a white solid. ¹H NMR: (400 MHz, CDCl₃) ppm δ 7.84-8.12 (m, 3H), 6.85-7.15 (m, 2H), 6.66-6.81 (m, 1H), 5.36 (br d, J=2.7 Hz, 3H), 5.11-5.27 (m, 3H), 4.63-4.85 (m, 3H), 3.90-4.25 (m, 18H), 3.37-3.75 (m, 28H), 3.15-3.28 (m, 4H), 2.64 (br d, J=6.53 Hz, 2H), 2.30-2.46 (m, 2H), 2.13-2.18 (m, 9H), 2.05 (s, 9H), 1.94-2.03 (m, 18H), 1.68 (br s, 2H), 1.45 (br s, 2H), 1.12 (br t, J=7.0 Hz, 2H).

To a solution of Compound Va (260 mg, 0.161 mmol) in anhydrous DCM (5 mL) was added diisopropylammonium tetrazolide (30.3 mg, 0.177 mmol), followed by dropwise addition of 3-bis(diisopropylamino)phosphanyloxypropanenitrile (194 mg, 0.645 mmol) at ambient temperature under N₂. The reaction mixture was stirred at 20~ 25° C. for 2 hrs. LCMS indicated Compound Va was consumed completely. After cooling to −20° C., the reaction mixture was added to a stirred solution of brine/saturated aq. NaHCO₃ (1:1, 5 mL) at 0° C. After stirring for 1 min, DCM (5 mL) was added. Layers were separated. Organics were washed with brine/saturated aq. NaHCO₃ solution (1:1, 5 mL), dried over Na₂SO₄, filtered, and concentrated to ~1 mL of volume. The residue solution was added dropwise to 20 mL methyl tert-butyl ether (MTBE) with stirring. This resulted in precipitation of white solid. The mixture was centrifuged, and solid was collected. The solid was redissolved in 1 mL of DCM and precipitated by addition of MTBE (20 mL). Solid was again isolated by centrifuge. The solid collected was dissolved in anhydrous CH₃CN. Volatiles were removed. This process was repeated two more times to afford GalNAc ligand phosphoramidite compound GLPA1 (153 mg, 84.4 μmol) as a white solid. ¹H NMR (400 MHz, CDCl₃): ppm δ 7.71-8.06 (m, 2H), 6.60-7.06 (m, 3H), 5.37 (br d, J=3.0 Hz, 3H), 5.18-5.32 (m, 3H), 4.70-4.86 (m, 3H), 3.92-4.25 (m, 18H), 3.42-3.85 (m, 30H), 3.25 (m, 4H), 2.59-2.75 (m, 4H), 2.27-2.44 (m, 2H), 2.15-2.20 (s, 9H) 2.07 (s, 9H), 1.96-2.03 (m, 18H), 1.65 (br s, 4H), 1.44 (br d, J=7.28 Hz, 2H), 1.14-1.24 (m, 12H). ³¹P NMR (CDCl₃): ppm δ 147.15.

GalNAc ligand phosphoramidite compound GLPA2 was synthesized using the same procedure except Intermediate-B was used. ¹H NMR (400 MHz, CDCl₃): ppm δ 7.94-8.18 (m, 1H), 7.69 (br s, 1H), 6.66-7.10 (m, 3H), 5.35 (d, J=3.5 Hz, 3H), 5.07-5.25 (m, 3H), 4.76-4.86 (m, 3H), 4.01-4.31 (m, 10H), 3.91-4.01 (m, 8H), 3.74-3.86 (m, 4H), 3.52-3.71 (m, 30H), 3.42-3.50 (m, 6H), 3.15-3.25 (m, 4H), 2.52-2.70 (m, 4H), 2.22-2.45 (m, 2H), 2.15-2.22 (s, 9H), 2.06 (s, 9H), 1.95-2.03 (m, 18H), 1.77 (br s, 2H), 1.58-1.66 (m, 4H), 1.40 (m, 2H), 1.08-1.24 (m, 12H). ³¹P NMR (CDCl₃): ppm δ 147.12.

Scheme 3 below was followed to prepare GLPA15.

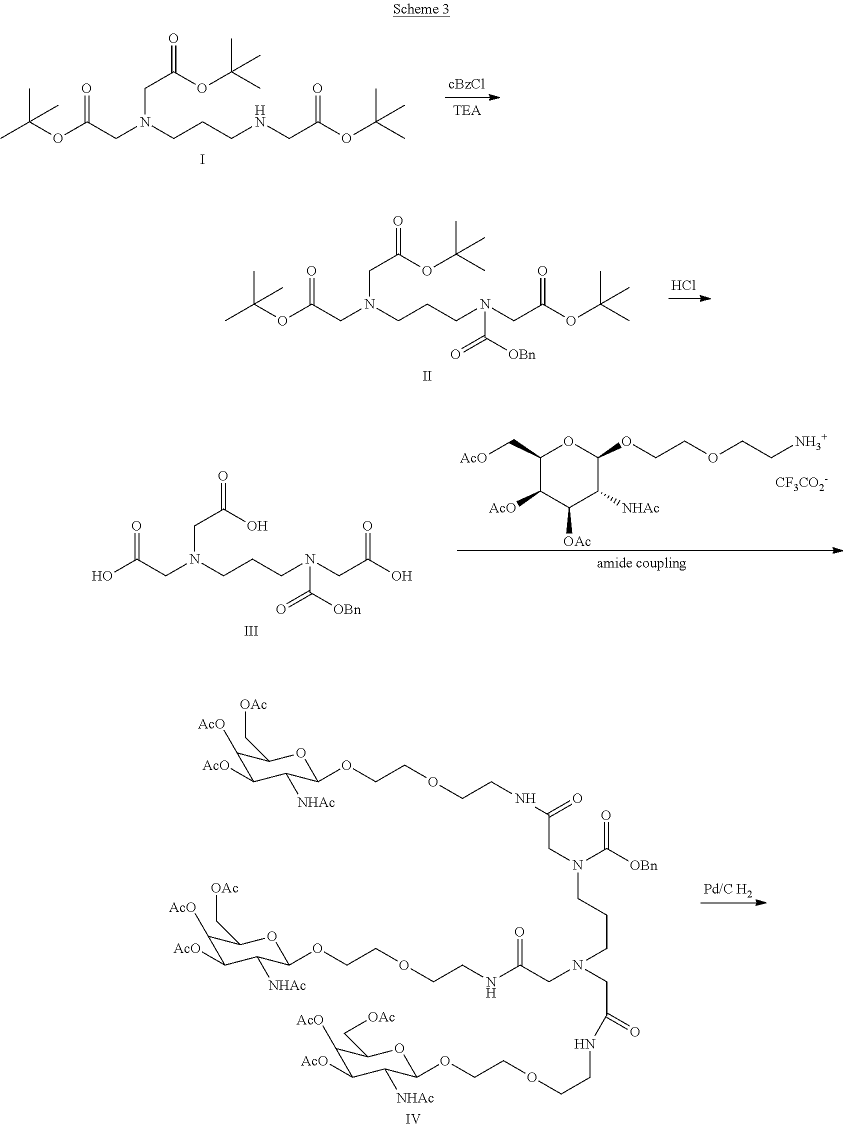

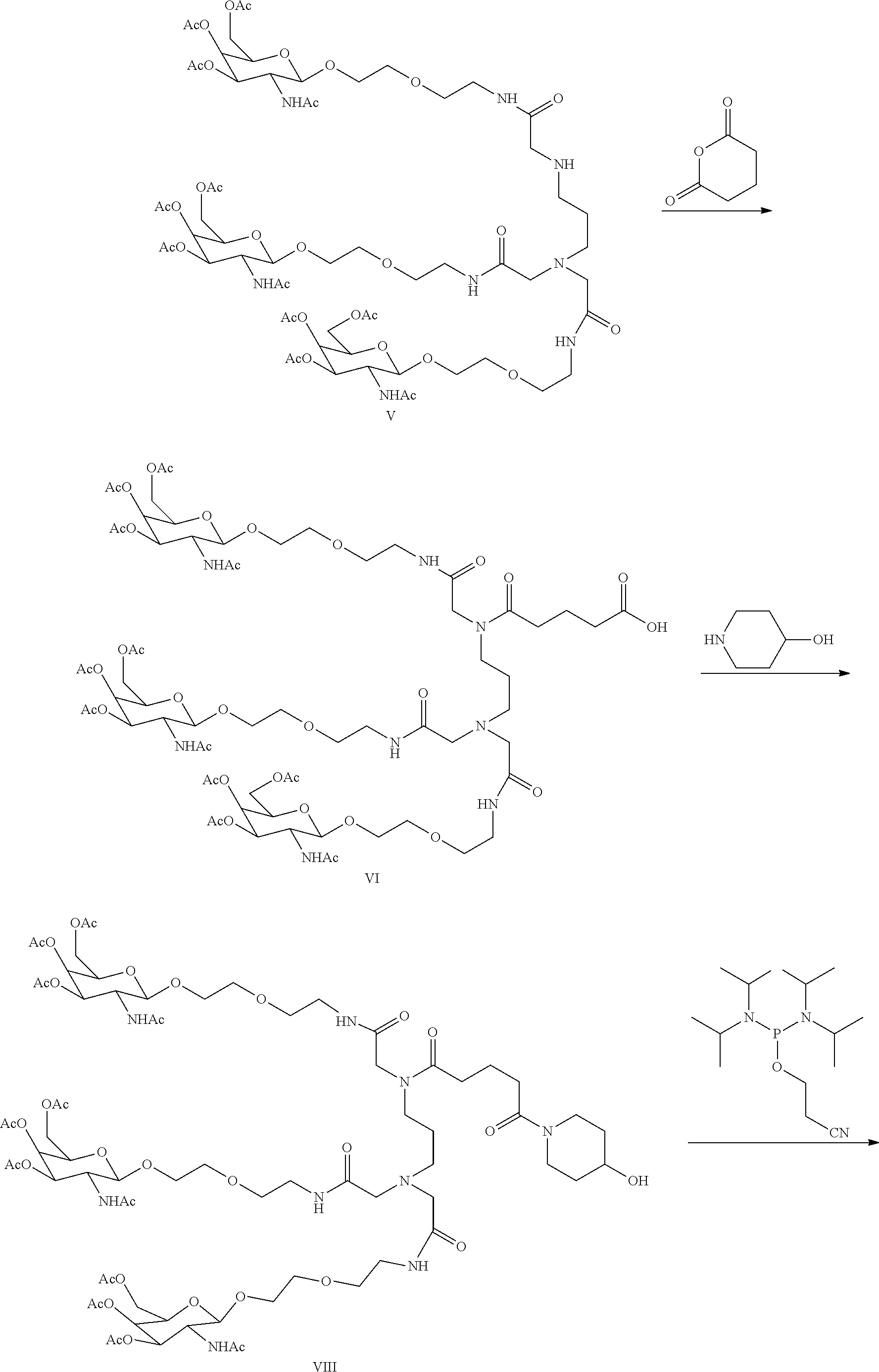

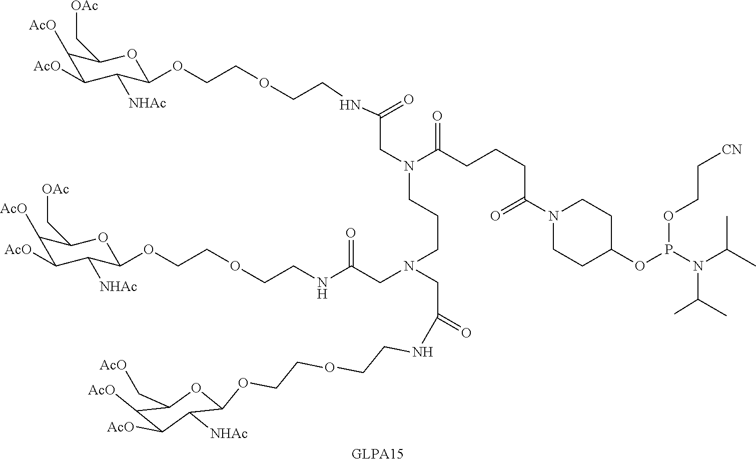 [SMILES: CC(=O)NC1C(OCCOCCNC(=O)CN(CCCN(CC(=O)NCCOCCOC2OC(COC(C)=O)C(OC(C)=O)C(OC(C)=O)C2NC(C)=O)C(=O)CCCC(=O)N2CCC(OP(OCCC#N)N(C(C)C)C(C)C)CC2)CC(=O)NCCOCCOC2OC(COC(C)=O)C(OC(C)=O)C(OC(C)=O)C2NC(C)=O)OC(COC(C)=O)C(OC(C)=O)C1OC(C)=O ; "GLPA15"]

Starting from secondary amine Compound I (Compound II in Scheme 2), Cbz protection was introduced to afford Compound II. The tert-Butyl groups of Compound II were removed by treatment with acid to give triacid Compound III. Amide coupling of Compound III with Intermediate-A afforded Compound IV. The Cbz protecting group of Compound IV was removed by hydrogenation to afford secondary amine Compound V, which was reacted with glutaric anhydride to afford carboxylic Compound VI. Compound VI reacted with piperidin-4-ol under amide coupling reaction condition to affords Compound VII. Phosphoramidite Compound GLPA15 was synthesized by treating Compound VII with 2-Cyanoethyl N,N diisopropylchlorophosphoramidite and a catalytic amount of 1H-tetrazole.

¹H NMR (400 MHz in DMSO-d₆): δ ppm 8.05 (br d, J=6.50 Hz, 2H), 7.81 (br d, J=9.01 Hz, 3H), 5.22 (d, J=3.25 Hz, 3H), 4.98 (dd, J=11.26, 3.25 Hz, 3H), 4.55 (br d, J=8.50 Hz, 3H), 4.03 (s, 9H), 3.64-3.97 (m, 12H), 3.55-3.63 (m, 6H), 3.50 (br s, 5H), 3.40 (br d, J=6.13 Hz, 6H), 3.17-3.30 (m, 9H), 3.07 (br d, J=14.26 Hz, 4H), 2.76 (t, J=5.82 Hz, 2H), 2.18-2.47 (m, 6H), 2.10 (s, 9H), 1.99 (s, 9H), 1.89 (s, 9H), 1.78 (s, 9H), 1.52-1.74 (m, 6H), 1.12-1.19 (m, 12H). 31P NMR (DMSO-d₆): ppm δ 145.25.

In certain studies, a method used to attach a targeting group comprising GalNAc (also referred to herein as a GalNAc delivery compound) to the 5′-end of a sense strand included use of a GalNAc phosphoramidite (GLPA1) in the last coupling step in the solid phase synthesis, using a synthetic process such as the process used if oligonucleotide chain propagation of adding a nucleotide to the 5′-end of the sense strand is performed.

In some studies a method of attaching a targeting group comprising GalNAc to the 3′-end of a sense strand comprised use of a solid support (CPG) that included a GLO-n. In some studies, a method of attaching a targeting group comprising GalNAc to the 3′-end of a sense strand comprises attaching a GalNAc targeting group to CPG solid support through an ester bond and using the resulting CPG with the attached GalNAc targeting group when synthesizing the sense strand, which results in the GalNAc targeting group attached at the 3′-end of the sense strand.

### Example 3

### Phosphoramidite Compound 2

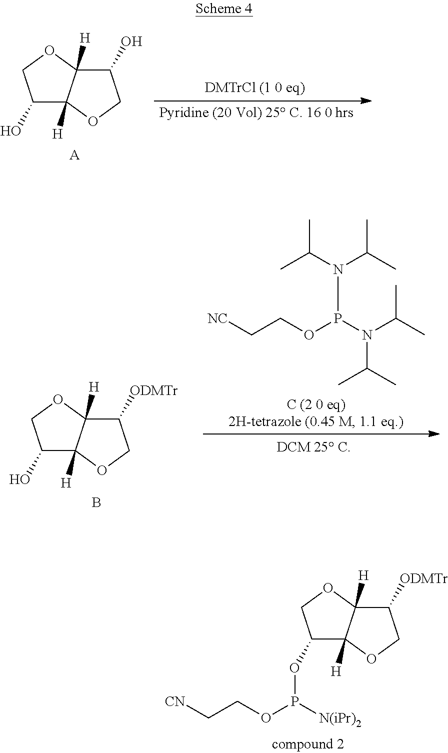

DMTrCl (232 g, 684 mmol, 1.0 eq) in pyridine (400 mL) was added to the solution of isomannide compound A (100 g, 684 mmol, 1.0 eq) in pyridine (600 mL), and the mixture was stirred at 25° C. for 12 hrs. LC-MS showed compound A was consumed completely and one main peak with desired mass was detected. The resulting reaction mixture was diluted with water (500 mL), extracted with DCM (500 mL*2), and the combined organic phases were washed with brine (500 mL), dried over Na₂SO₄ and concentrated in vacuum to get the residue. The residue was purified by column chromatography (DCM/MeOH=100/1 to 50/1, 0.1% Et₃N) to give compound B (150 g, 48.9% yield) a yellow solid.

¹H NMR: EC4783-404-P1B1_C (400 MHz, DMSO-d₆) δ ppm 7.46 (br d, J=7.63 Hz, 2H) 7.28-7.37 (m, 6H) 7.19-7.25 (m, 1H) 6.90 (br d, J=7.88 Hz, 4H) 4.70 (d, J-6.50 Hz, 1H) 3.99-4.09 (m, 6H) 3.88-3.96 (m, 2H) 3.83 (br dd, J=7.82, 6.94 Hz, 1H) 3.74 (s, 6H) 3.41 (br t, J=8.13 Hz, 1H) 3.05 (t, J=8.44 Hz, 1H) 2.85 (br t, J=7.50 Hz, 1H).

To a solution of compound B (80.0 g, 178 mmol, 1.0 eq) in DCM (800 mL) at 25° C. under N₂ atmosphere was added dropwise 2H-tetrazole (0.45 M, 436 mL, 1.1 eq), then compound C (80.6 g, 267 mmol, 85.0 mL, 1.5 eq) in DCM (200 mL) was added dropwise to the mixture. The reaction mixture was stirred at 25° C. under for 1.0 hr. LC-MS showed compound B was consumed completely and one main peak with desired mass was detected. The resulting reaction mixture was cooled to −20° C. and poured into ice cold sat.NaHCO₃ (500 mL), extracted with DCM (500 mL*3), the combined organic layers were washed with sat.NaHCO₃/brine=1:1 (3 00 mL/300 mL), dried over Na₂SO₄ and concentrated in vacuum (35° C.) to get the residue (100 mL). The residue was purified by column chromatography (Al₂O₃, DCM/MeOH=100/1 to 50/1, 0.1% Et₃N) to give compound2 (77 g, 119 mmol, 66.5% yield) as a white solid.

¹H NMR: EC4783-423-P1B1_C (400 MHz, DMSO-d₆) ô ppm 7.22 (br d, J=7.50 Hz, 2H) 7.05-7.14 (m, 6H) 6.96-7.02 (m, 1H) 6.67 (br dd, J=8.82, 1.81 Hz, 4H) 3.95-4.07 (m, 2H) 3.73-3.83 (m, 1H) 3.62-3.72 (m, 2H) 3.48-3.53 (m, 6H) 3.27-3.37 (m, 3H) 3.11 (s, 6H) 2.82 (td, J=8.54, 2.31 Hz, 1H) 2.47-2.63 (m, 3H) 2.28 (br d, J=1.63 Hz, 3H) 0.82-1.00 (m, 13H).

### Phosphoramidite Compound 1

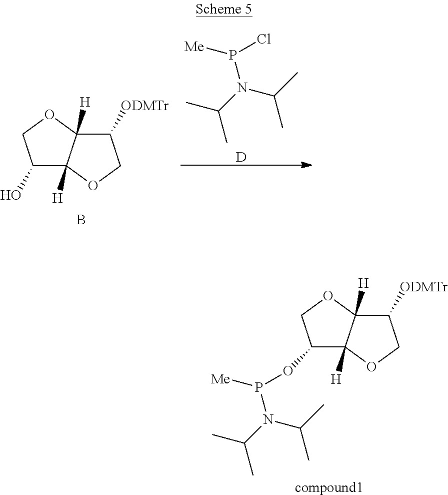

To a solution of compound B (500 mg, 1.11 mmol, 1.0 eq) in DCM (5.0 mL) was added compound D (607 mg, 3.34 mmol, 3.0 eq) and DIEA (432 mg, 3.34 mmol, 582 μL, 3.0 eq) under N₂ atmosphere at 0-5° C., the mixture was stirred at 25° C. for 1.0 hrs. LC-MS showed Compound B was consumed completely, several new peaks were shown on LC-MS and ~70.9% of desired compound was detected. The resulting reaction mixture was cooled to −20° C. and poured into cold (0-5° C.) sat.NaHCO₃ (5.0 mL) solution, extracted with DCM (5.0 mL*2), the combined organic layers were washed with cold (0-5° C.) sat.NaHCO₃/brine=1:1 (5.0 mL/5.0 mL), dried over Na₂SO₄ and concentrated in vacuum to get the residue (~5 mL). The residue was purified by column chromatography (alkaline Al₂O₃, Petroleum ether/Ethyl acetate=10/1 to 5/1, 0.1% Et₃N) to give compound 1 (280 mg, 471 μmol, 42.3% yield) was obtained as a white solid.

¹H NMR: EC10615-49-PIN (400 MHz, DMSO-d₆) δ ppm 7.44 (br d, J=7.63 Hz, 2H), 7.31 (br t, J=7.94 Hz, 6H), 7.18-7.26 (m, 1H), 6.89 (brd, J=8.00 Hz, 4H), 4.08-4.13 (m, 1H), 3.95-4.03 (m, 1H), 3.84-3.93 (m, 1H), 3.77-3.83 (m, 1H), 3.74 (s, 6H), 3.43-3.53 (m, 3H), 3.38 (br d, J=6.75 Hz, 1H), 2.94-3.04 (m, 1H), 2.70-2.85 (m, 1H), 1.09-1.15 (m, 12H), 1.07 (br s, 3H).

Other phosphoramidites may be prepared according to procedures described herein and/or prior arts such as, but are not limited to, U.S. Pat. No. 426,220 and WO02/36743.

### Example 4. Preparation of a Solid Support Comprising Phosphoramidites Monomers of the Present Invention

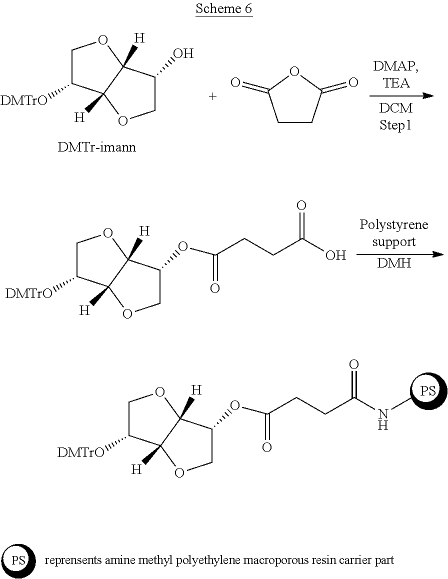

Dichloromethane (19.50 kg) was added to the 50 L glass kettle under the protection of nitrogen and started stirring. The temperature was controlled at 20~30° C., and DMTr imann (1.47 kg), triethylamine (1.50 kg), 4-dimethylaminopyridine (0.164 kg) and succinic anhydride (1.34 kg) was added to the glass kettle. The system was kept at 20~30° C. for 18 h, samples were taken and the reaction was ended. Saturated sodium bicarbonate solution (22.50 kg) was added into the reaction system, stirred for 10-20 min, and allowed to separate into layers. The organic phase was separated, and the aqueous phase was extracted twice with dichloromethane, and the organic phase was combined and dried over anhydrous sodium sulfate, filtered, and concentrated in vacuum to get the residue forming a gray to off-white solid of 1.83 kg.

N, N-dimethylformamide (23.50 kg) was added into a 100 L glass kettle and stirred. The temperature was controlled at 20~30° C. Under the protection of nitrogen, the products of the previous step, O-benzotriazole tetramethylurea hexafluorophosphate (0.33 kg) and N, N-diisopropylethylamine (0.13 kg) were added into the aforesaid 100 L glass kettle through the solid feeding funnel and stirred for 10~30 minutes and were discharged into a 50 L zinc barrel for use. Macroporous amine methyl resin (3.25 kg) (purchased from Tianjin Nankai Hecheng Science and Technology Co., Ltd., batch number HA2X1209, load capacity 0.48 mmol/g) were added into the aforesaid 100 L solid phase synthesis reactor through the solid feeding funnel, the temperature was controlled at 20~30° C., N, N-dimethylformamide (21.00 kg+21.00 kg) and the reaction solution in the zinc barrel of the previous step were add into the solid phase synthesis reactor. The system was subject to thermal insulation reaction, and the solid load was tracked to ≥250 μmol/g, and the load detection method was UV. The system was filtered under the pressure of nitrogen, the filter cake was washed with N, N-dimethylformamide for three times (26.00 kg+26.10 kg+26.00 kg), and the filter cake was left in the kettle. CAP.A (50% acetonitrile and 50% acetic anhydride, 4.40 kg+4.42 kg+4.30 kg) and CAP.B (20% pyridine and 30% N-methylimidazole and 50% acetonitrile, 4.40 kg+4.40 kg+4.47 kg) were added into the 80 L glass kettle, and stirred for 3~8 min before use. This operation was repeated for three times to cap, and acetonitrile (18.00 kg+18.00 kg+18.00 kg+17.50 kg+17.50 kg) was added into the solid phase synthesis kettle. Filter-pressed after nitrogen bubbling for 10~30 min. This operation was repeated for four times, the filter cake was purged in the solid phase synthesis kettle with nitrogen for 2-4 h, and then was transferred to a 50 L filter press tank. The temperature was controlled at 15~30° C., continued drying, and obtained yellow to white solid product after drying, weight: 3.516 kg.

### Example 5. Synthesis of PNPLA3 RNAi Agents

PNPLA3 RNAi agent duplexes shown in Table 2-3, above, were synthesized in accordance with the following general procedures:

Sense and antisense strand sequences of siRNA were synthesized on oligonucleotide synthesizers using a well-established solid phase synthesis method based on phosphoramidite chemistry. Oligonucleotide chain propagation is achieved through 4-step cycles: a deprotection, a condensation, a capping and an oxidation or a sulfurization step for addition of each nucleotide. Syntheses were performed on a solid support made of controlled pore glass (CPG, 1000 Å). Monomer phosphoramidites may be purchased from commercial sources or may be the phosporamidite compounds in example 3 and in WO2016/028649. The phosporamidite compounds herein may be attached to the 3′-end as a monomeric phosphoramidite, and further be attached to the CPG solid support. In the case of attachment at the 5′-end, the phosphoramidite compounds may be used for the final coupling reaction, and can be further conjugated to target ligands if necessary.

Phosphoramidites with GalNAc ligand cluster (GLPA1, GLPA2 and GLPA15 as non-limiting examples) were synthesized according to the procedures of Examples 1-2 herein. For siRNAs used for in vitro screening (Table 2), syntheses were carried out at 2 μmol scale, and for siRNAs used for in vivo testing (Table 3), syntheses were carried out at scale of 5 μmol or larger. In the case where the GalNAc ligand (GLO-0 as a non-limiting example) is attached at 3′-end of sense strand, GalNAc ligand attached CPG solid support was used. In the case where the GalNAc ligand (GLS-5 or GLS-15 as non-limiting example) is attached at 5′-end of sense strand, a GalNAc phosphoramidite (GLPA1, GLPA2 or GLPA15 as a non-limiting example) was used for the last coupling reaction.

The sense strands and the antisense strands were synthesis by solid phase synthesis with 4-step cycles, which detailed as below: Trichloroacetic acid (TCA) 3% in dichloromethane or Dichloroacetic acid (DCA) 10% in toluene was used for deprotection of 4,4′-dimethoxytrityl protecting group (DMT). 5-Ethylthio-1H-tetrazole was used as an activator in coupling step. Capping with CapA (Acetic Anhydride in Acetonitrile)/CapB (Pyridine/NMI/Acetonitrile) (v/v, 1:1). I2 in Py/H₂O and phenylacetyl disulfide (PADS) in pyridine/MeCN or Xanthane Hydride (DDTT) in pyridine was used for oxidation and sulfurization reactions, respectively.

After the final solid phase synthesis step, solid support bound oligomer was cleaved and protecting groups were removed by treating with a 1:1 volume solution of 40 wt. % methylamine in water and 28% ammonium hydroxide solution. Solid support bound oligomer containing monomer phosphate mimic was treated with MeCN:TMSI:Pyridine=50:2:2 (v/v/v) before C&D (cleave and protecting) if necessary. For the synthesis of siRNAs used for in vitro screening, crude mixture was concentrated. The remaining solid was dissolved in 1.0 M NaOAc, and ice cold EtOH was added to precipitate out the single strand product as the sodium salt, which was used for annealing without further purification. For the synthesis of multi-targeted molecules used for in vivo testing, crude single strand product was further purified by ion pairing reversed phase HPLC (IP-RP-HPLC). Purified single strand oligonucleotide product from IP-RP-HPLC was converted to sodium salt by dissolving in 1.0 M NaOAc and precipitation by addition of ice cold EtOH. Annealing of equimolar complementary sense stand and antisense strand oligonucleotide in water was performed to form the double strand siRNA product, which was lyophilized to afford a fluffy white solid.

### Example 6. In Vitro Screening of PNPLA3 siRNA Duplexes

Hep3B cells were trypsinized and adjusted to appropriate density, and seeded into 96-well plates. Cells were transfected with test siRNAs or a control siRNA using Lipofectamine RNAiMax (Invitrogen—13778-150) at the same time of seeding following the protocol according to manufacturer's recommendation. The siRNAs were tested at two concentrations (0.2 nM and 1.0 nM) in triplicate.

Day 0, psiCHECK™-2 Vector Transfection (One Plate)
(1) Transfer 2.5 μg psiCHECK™-2 Vector plasmid into an RNASE free Eppendorf tube (solution mix #1)
(2) Add trypsin to disassociate Hep3B cells in one flask, and count cells using Vi-Cell counting machine, adjust the cell density to 1*10\hat{ }5/ml
(3) Transfer 7.5 μL Fugene-HD into solution mix #1 tube, mix.
(4) Add solution in Step 3 into cell suspension, mix, and dispense suspension into the 96 well plate (100 μl/well)
   Day 1, siRNAs Transfection
(1) Dilute Lipofectamine®RNAiMAX Reagent with Opti-MEM® Medium.
(2) Dilute the siRNA with RNA-free water to make 12× stock.
(3) Mix equal volume of diluted RNAiMax and siRNA. Incubate the mixture at RT for 15 min to allow complex formation.
(4) Add 45 μl/well compound Lipofectamine® RNAiMAX(Opti-MEM) mix into 225 μl/well DMEM fresh medium, and discard the supernatants in assay plate, add 120 μl/well compound mix into 96 well plates.
(5) No compound control well was defined as cells transfected with psiCHECK™-2 Vector and without siRNA treatment; blank control was cell only wells.

### Day 2, Dual-Glo® Luciferase Assay

(1) Add Reagent to assay plate, wait 10 minutes to allow for cell lysis to occur.
(2) Transfer 100 μl cell lysates into a plate, then measure the firefly luminescence.
(3) Add 50 μl of Dual-Glo® Stop & Glo® Reagent to the assay plates and mix, wait 10 minutes, then measure Renilla luminescence.
(4) Calculate the relative expression

### Data Analysis

$$Ratio of sample well = ( sample Renilla luminescence - background blank ) / \\ ( sample Fireflyluminescence - background blank )$$

$$Ratio of no compound control well = ( control Renilla luminescence - background blank ) / \\ ( control sample Fireflyluminescence - background blank )$$

$$\% inhibition = 100 - ( Ratio of sample well / the average Ratio of no compound control ) \times 100 \%$$

Table 4 provides experimental results of in vitro studies using various PNPLA3 RNAi agents to inhibit PNPLA3 expression. The duplex sequences used correspond to those shown in Table 2.

| | Average Inhibition % | | | |
|---|---|---|---|---|
| Duplex | 1 nM | | 0.2 nM | |
| AV# | Average | SD | Average | SD |
| AV00468 | 65.72 | 2.77 | 58.69 | 1.03 |
| AV00469 | 37.00 | 4.94 | 29.03 | 3.20 |
| AV00470 | 37.15 | 7.02 | 32.58 | 4.43 |
| AV00472 | 10.34 | 4.59 | 25.03 | 1.74 |
| AV00473 | 28.96 | 3.02 | 26.41 | 0.64 |
| AV00474 | 31.90 | 4.45 | 27.54 | 3.46 |
| AV00475 | 16.72 | 5.96 | 39.88 | 4.00 |
| AV00476 | 34.27 | 3.24 | 11.15 | 2.89 |
| AV00477 | 20.33 | 5.89 | 7.51 | 11.45 |
| AV00478 | 59.61 | 3.73 | 40.16 | 5.11 |
| AV00479 | 57.24 | 2.41 | 31.26 | 16.60 |
| AV00480 | 40.42 | 2.69 | 9.73 | 10.23 |
| AV00481 | 36.34 | 1.67 | 39.85 | 3.99 |
| AV00482 | 36.58 | 2.65 | 34.76 | 1.48 |
| AV00483 | 35.35 | 5.30 | 35.93 | 1.95 |
| AV00484 | 21.80 | 12.21 | 27.87 | 4.88 |
| AV00485 | 48.20 | 11.79 | 52.01 | 5.09 |
| AV00486 | 68.18 | 1.27 | 59.15 | 2.99 |
| AV00487 | 76.10 | 2.17 | 57.25 | 3.05 |
| AV00488 | 37.11 | 4.09 | 30.34 | 2.37 |
| AV00489 | 28.32 | 4.34 | 40.24 | 5.18 |
| AV00490 | 41.43 | 1.29 | 8.99 | 7.10 |
| AV00493 | 71.35 | 4.66 | 53.39 | 6.46 |
| AV00494 | 53.08 | 2.00 | 34.88 | 5.44 |
| AV00495 | 33.25 | 2.40 | 8.85 | 9.72 |
| AV00496 | 14.27 | 9.93 | 32.43 | 1.39 |
| AV00497 | 38.29 | 10.36 | 52.79 | 1.43 |
| AV00498 | 51.85 | 5.22 | 59.54 | 1.27 |
| AV00499 | 34.61 | 12.10 | 38.33 | 6.16 |
| AV00500 | -2.98 | 7.95 | 15.90 | 4.51 |
| AV00501 | 58.76 | 2.92 | 48.66 | 2.58 |
| AV00502 | 27.66 | 2.19 | 18.22 | 5.96 |
| AV00503 | 27.41 | 0.45 | 24.08 | 1.26 |
| AV00504 | 75.39 | 1.30 | 47.93 | 2.68 |
| AV00505 | 43.81 | 6.22 | 20.66 | 10.50 |
| AV00506 | 86.14 | 1.71 | 50.04 | 13.30 |
| AV00507 | 60.55 | 2.67 | 35.53 | 13.99 |
| AV00508 | 52.01 | 7.83 | 24.31 | 11.92 |
| AV00509 | 58.92 | 2.20 | 29.99 | 13.98 |
| AV00510 | 63.79 | 2.33 | 45.60 | 1.47 |
| AV00511 | 47.75 | 4.80 | 44.94 | 1.04 |
| AV00512 | 36.03 | 8.65 | 46.47 | 1.61 |
| AV00513 | 32.79 | 8.43 | 41.26 | 3.13 |
| AV00514 | 28.15 | 4.02 | 34.29 | 3.59 |
| AV00515 | 86.40 | 0.97 | 58.50 | 1.64 |
| AV00517 | 44.65 | 0.55 | 33.01 | 4.54 |
| AV01072 | 82.19 | 1.92 | 77.96 | 1.62 |
| AV01073 | 32.49 | 7.43 | 39.43 | 4.13 |
| AV01074 | 25.10 | 11.86 | 28.52 | 3.95 |
| AV01075 | 26.87 | 7.81 | 26.00 | 0.57 |
| AV01076 | 37.93 | 8.67 | 37.08 | 2.17 |
| AV01077 | 64.51 | 4.26 | 52.05 | 4.31 |
| AV01078 | 81.16 | 3.93 | 71.14 | 3.07 |
| AV01079 | 37.18 | 12.11 | 9.86 | 5.81 |
| AV01080 | 26.72 | 9.05 | 6.92 | 14.21 |
| AV01081 | 72.69 | 1.41 | 42.58 | 5.70 |
| AV01082 | 56.77 | 2.01 | 25.82 | 12.50 |
| AV01083 | 72.19 | 1.38 | 59.63 | 3.52 |
| AV01084 | 75.22 | 1.64 | 61.47 | 2.02 |
| AV01085 | 72.51 | 0.90 | 60.94 | 2.12 |

Table 5 provides experimental results of in vitro studies using various PNPLA3 RNAi agents to inhibit PNPLA3 expression. The duplex sequences used correspond to those shown in Table 2.

| | Average Inhibition % | | | |
|---|---|---|---|---|
| Duplex | 1 nM | | 0.2 nM | |
| AV# | Average | SD | Average | SD |
| AV00448 | 76.19 | 2.70 | 66.04 | 1.58 |
| AV00449 | 62.31 | 9.12 | 62.43 | 3.77 |
| AV00450 | 60.22 | 1.13 | 54.40 | 2.18 |
| AV00451 | 21.59 | 18.63 | 31.75 | 8.05 |
| AV00452 | 26.85 | 15.01 | 34.61 | 5.51 |
| AV00453 | 36.33 | 7.35 | 32.32 | 8.61 |
| AV00454 | 24.87 | 5.15 | 27.85 | 12.62 |
| AV00455 | 19.20 | 10.64 | 26.11 | 11.34 |
| AV00456 | 71.92 | 5.62 | 53.72 | 6.55 |
| AV00457 | 71.96 | 3.99 | 47.58 | 11.15 |
| AV00458 | 72.76 | 1.96 | 44.18 | 8.91 |
| AV00459 | 69.71 | 1.53 | 52.36 | 6.10 |
| AV00460 | 33.72 | 1.96 | 13.11 | 9.28 |
| AV00461 | 69.89 | 1.79 | 65.72 | 4.08 |
| AV00462 | 26.52 | 1.33 | 26.20 | 3.96 |
| AV00463 | 58.98 | 7.50 | 61.74 | 5.73 |
| AV00464 | 22.41 | 13.05 | 29.15 | 11.74 |
| AV00465 | 50.42 | 2.19 | 51.89 | 5.34 |
| AV00466 | 34.44 | 15.09 | 40.45 | 7.18 |
| AV00467 | 60.51 | 3.35 | 54.05 | 4.49 |
| AV00471 | 57.13 | 5.37 | 31.17 | 4.65 |
| AV00491 | 15.24 | 4.93 | −1.61 | 4.79 |
| AV00492 | 43.03 | 4.09 | 7.62 | 12.37 |
| AV00516 | 85.39 | 1.42 | 60.27 | 2.44 |

### Example 7. In Vivo Testing of PNPLA3 siRNA Duplexes

At 7 days before dosing of siRNAs, female C57BL/6J mice (4 in each group) were infected by intravenous administration of a solution of adeno-associated virus 8 (AAV8) vector encoding human PNPLA3 and luciferase gene. At day 1, mice were subcutaneously administered a single dose of 6 mg/kg PNPLA3 siRNA agents or saline. Blood samples were collected at day 1, before dosing of siRNA and at day 15, at day 22 and/or at the terminal day 29. Plasma samples were isolated and luciferase activity was measured per manufacturer's recommended protocol. Since expression of human PNPLA3 level correlates with expression level of luciferase, percent of remaining PNPLA3 was calculated as the ratio of luciferase signaling between post-dosing plasma sample and pre-dosing plasma sample in siRNA-treated group, and normalized by the ratio of luciferase signaling between post-dosing sample and pre-dosing sample in saline-treated group. SiRNA duplexes tested herein, which has a modification pattern of the invention, achieved significant knockdown of PNPLA3 mRNA and showed longer duration of activity.

Table 6 provides experimental results of in vivo studies using various PNPLA3 RNAi agents to inhibit PNPLA3 expression. The duplex sequences used correspond to those shown in Table 3.

| | D15 | D22 | D29 | |
|---|---|---|---|---|
| | remaining | remaining | remaining | |
| | percent | percent | percent | |
| | relative to | relative to | relative to | |
| Duplex | D8 | D8 | D8 | Dosage |
| AD# | (Mean ± SD) | (Mean ± SD) | (Mean ± SD) | (mg/kg) |
| AD00663 | 0.24 ± 0.13 | 0.31 ± 0.13 | 0.46 ± 0.24 | 6 |
| AD00664 | 0.40 ± 0.20 | 0.32 ± 0.1 | 0.39 ± 0.13 | 6 |
| AD00746 | 0.45 ± 0.09 | 0.72 ± 0.29 | 0.68 ± 0.32 | 6 |
| AD00815 | 0.39 ± 0.25 | 0.31 ± 0.12 | 0.35 ± 0.14 | 6 |
| AD00816 | 0.30 ± 0.18 | 0.26 ± 0.16 | 0.33 ± 0.19 | 6 |
| AD00817 | 0.61 ± 0.14 | 0.6 ± 0.16 | 0.59 ± 0.07 | 6 |
| AD00818 | 0.64 ± 0.21 | 0.54 ± 0.32 | 0.64 ± 0.21 | 6 |
| AD00819 | 0.42 ± 0.15 | 0.28 ± 0.1 | 0.41 ± 0.1 | 6 |
| AD00820 | 0.65 ± 0.42 | 0.38 ± 0.13 | 0.44 ± 0.18 | 6 |

Table 7 provides experimental results of in vivo studies using various PNPLA3 RNAi agents to inhibit PNPLA3 expression. The duplex sequences used correspond to those shown in Table 3.

| | D15 remaining | D22 remaining | |
|---|---|---|---|
| Duplex | percent relative to | percent relative to | Dosage |
| AD# | D8 (Mean ± SD) | D8 (Mean ± SD) | (mg/kg) |
| AD00663 | 0.58 ± 0.25 | 0.52 ± 0.21 | 6 |
| AD00653 | 0.40 ± 0.25 | 0.45 ± 0.20 | 6 |
| AD00654 | 0.58 ± 0.13 | 0.47 ± 0.18 | 6 |
| AD00655 | 0.45 ± 0.17 | 0.35 ± 0.06 | 6 |
| AD00656 | 0.35 ± 0.13 | 0.31 ± 0.15 | 6 |
| AD00657 | 0.55 ± 0.26 | 0.35 ± 0.12 | 6 |
| AD00658 | 0.51 ± 0.28 | 0.41 ± 0.14 | 6 |
| AD00659 | 0.66 ± 0.24 | 0.45 ± 0.30 | 6 |
| AD00660 | 0.63 ± 0.30 | 0.36 ± 0.17 | 6 |
| AD00661 | 0.44 ± 0.21 | 0.33 ± 0.14 | 6 |
| AD00662 | 0.70 ± 0.34 | 0.55 ± 0.15 | 6 |

Table 8 provides experimental results of in vivo studies using various PNPLA3 RNAi agents to inhibit PNPLA3 expression. The duplex sequences used correspond to those shown in Table 3.

| | D15 remaining | D22 remaining | |
|---|---|---|---|
| Duplex | percent relative to | percent relative to | Dosage |
| AD# | D8 (Mean ± SD) | D8 (Mean ± SD) | (mg/kg) |
| AD00663-2 | 0.18 ± 0.02 | 0.15 ± 0.03 | 6 |
| AD00664-2 | 0.28 ± 0.06 | 0.29 ± 0.17 | 6 |
| AD00815-2 | 0.31 ± 0.06 | 0.43 ± 0.09 | 6 |

Table 9 provides experimental results of in vivo studies using various PNPLA3 RNAi agents to inhibit PNPLA3 expression. The duplex sequences used correspond to those shown in Table 3.

| | D15 remaining | D22 remaining | |
|---|---|---|---|
| Duplex | percent relative to | percent relative to | Dosage |
| AD# | D8 (Mean ± SD) | D8 (Mean ± SD) | (mg/kg) |
| AD02242 | 0.23 ± 0.05 | 0.49 ± 0.12 | 6 |
| AD02250 | 0.40 ± 0.11 | 0.28 ± 0.04 | 6 |

### Example 8 In Vivo Testing of PNPLA3 siRNA Agents in Cynomolgus Monkeys

18 healthy non-naive male cynomolgus monkeys (2-6 years old) were selected and randomized into 6 groups (3 monkeys per group) to receive a single subcutaneous injection of saline or test articles at 4 mg/kg on day 0, and liver biopsy samples were collected on day 0 (pre-dosing), 21 and 42. PNPLA3 mRNA level in liver tissues were measured by QPCR method. Gene remaining of PNPLA3 mRNA (normalized to pre-dosing level on day 0) for each group is shown in Table 10.

### Data Analysis

$$Δ CT = average Ct of target gene - average Ct of GAPDH$$

$$ΔΔ CT = Δ CT ( sample ) - Δ CT ( pre - dosing ) ;$$

$$mRNA relative expression = {2}^{- Δ Δ CT}$$

Table 10 provides experimental results of in vivo studies using various PNPLA3 RNAi agents to inhibit PNPLA3 expression. The duplex sequences used correspond to those shown in Table

| | PNPLA3 remaining relative to | | |
|---|---|---|---|
| | pre-dosing | | |
| Duplex | (Mean ± SD) | | Dosage |
| AD# | D21 | D42 | (mg/kg) |
| AD00663-1 | 0.27 ± 0.06 | 0.23 ± 0.13 | 4 |
| AD00664-1 | 0.42 ± 0.15 | 0.32 ± 0.09 | 4 |
| AD00815-1 | 0.36 ± 0.14 | 0.22 ± 0.05 | 4 |
| AD00816-1 | 0.68 ± 0.31 | 0.32 ± 0.1 | 4 |
| AD00819-1 | 0.62 ± 0.04 | 0.45 ± 0.25 | 4 |
| AD00444-1 | 1.28 ± 0.95 | 0.75 ± 0.87 | 4 |

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The term “or” as used herein means “and/or,” and is used interchangeably with the latter, unless clearly excluded from context. The phrase “and/or,” as used herein in the specification and in the claims, should be understood to mean “either or both” of the elements so conjoined, i.e., elements that are conjunctively present in some cases and disjunctively present in other cases. If there are more than two elements and are separated by commas, the commas before “and/or” have the same meaning as “and/or”, correspondingly representing “and” or “or”. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

1. A double-stranded ribonucleic acid (dsRNA) agent for inhibiting expression of Patatin-like Phospholipase Domain Containing 3 (PNPLA3), wherein the dsRNA agent includes a sense strand and an antisense strand, wherein the sense strand comprises at least 15 contiguous nucleotides differing by no more than 3 nucleotides from the nucleotide sequence of SEQ ID NO: 1 and the antisense strand comprises at least 15 contiguous nucleotides differing by no more than 3 nucleotides from the nucleotide sequence of SEQ ID NO:2, wherein the sense strand and the antisense strand can be partially, substantially, or fully complementary to each other, and optionally comprising a targeting ligand.

2. (canceled)

3. The dsRNA agent of claim 1 wherein the antisense strand comprises a region of complementarity to a PNPLA3 RNA transcript of at least 15, 16, 17, 18, or 19 contiguous nucleotides that differ by no more than 3 nucleotides from one of the antisense sequences listed in one of Tables 1-3;
preferably, the antisense strand of dsRNA is at least substantially complementary to any one of a target region of SEQ ID NO: 1 and is provided in any one of Tables 1-3; or the antisense strand of dsRNA is fully complementary to any one of a target region of SEQ ID NO: 1 and is provided in any one of Tables 1-3;
preferably, the dsRNA agent comprises a sense strand sequence set forth in any one of Tables 1-3, wherein the sense strand sequence is at least substantially complementary to the antisense strand sequence in the dsRNA agent; or
the dsRNA agent comprises a sense strand sequence set forth in any one of Tables 1-3, wherein the sense strand sequence is fully complementary to the antisense strand sequence in the dsRNA agent;
optionally, the dsRNA agent comprises an antisense strand sequence set forth in any one of Tables 1-3; and
optionally, the dsRNA agent comprises the sequences set forth as a duplex sequence in any of Tables 1-3.

4-9. (canceled)

10. The dsRNA of claim 1, wherein the dsRNA agent comprises at least one modified nucleotide;
optionally, all or substantially all of the nucleotides of the antisense strand are modified nucleotides;
preferably, the at least one modified nucleotide comprises: a 2′-O-methyl nucleotide, 2′-fluoro nucleotide, 2′-deoxy nucleotide, 2′3′-seco nucleotide mimic, locked nucleotide, unlocked nucleic acid nucleotide (UNA), glycol nucleic acid nucleotide (GNA), 2′-F-Arabino nucleotide, 2′-methoyxyethyl nucleotide, abasic nucleotide, ribitol, inverted nucleotide, inverted abasic nucleotide, inverted 2′-Ome nucleotide, inverted 2′-deoxy nucleotide, isomannide nucleotide, 2′-amino-modified nucleotide, 2′-alkyl-modified nucleotide, mopholino nucleotide, and 3′-OMe nucleotide, a nucleotide comprising a 5′-phosphorothioate group, a nucleotide comprising vinyl phosphonate, or a terminal nucleotide linked to a cholesteryl derivative or dodecanoic acid bisdecylamide group, a 2′-amino-modified nucleotide, a phosphoramidate, or a non-natural base comprising nucleotide.

11-12. (canceled)

13. A double-stranded ribonucleic acid (dsRNA) agent for inhibiting expression of Patatin-like Phospholipase Domain Containing 3 (PNPLA3), wherein the dsRNA agent including a sense strand and an antisense strand, wherein the sense strand is complementary to the antisense strand, wherein the antisense strand comprises a region complementary to part of an mRNA encoding PNPLA3, wherein each strand is about 15 to about 30 nucleotides in length, wherein the sense strand sequence is represented by formula (I): 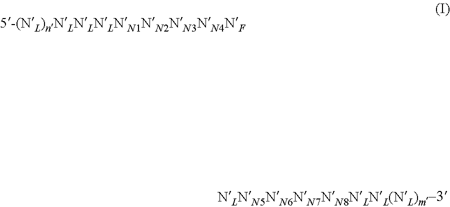
wherein:
each N′_{F} represents a 2′-fluoro-modified nucleotide; each of N′_{N1}, N′_{N2}, N′_{N3}, N′_{N4}, N′_{N5}, N′_{N6}, N′_{N7} and N′_{N8} independently represents a modified or unmodified nucleotide; each N′_{L} independently represents a modified or unmodified nucleotide but not a 2′-fluoro-modified nucleotide, and m′ and n′ are each independently an integer of 0 to 7.

14. A double-stranded ribonucleic acid (dsRNA) agent for inhibiting expression of Patatin-like Phospholipase Domain Containing 3 (PNPLA3), wherein the dsRNA agent including a sense strand and an antisense strand, wherein the sense strand is complementary to the antisense strand, wherein the antisense strand comprises a region complementary to part of an mRNA encoding PNPLA3, wherein each strand is about 18 to about 30 nucleotides in length, wherein the antisense strand sequence is represented by formula (II): 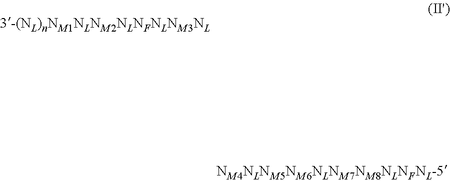
wherein:
each N_{F} represents a 2′-fluoro-modified nucleotide; each of N_{M1}, N_{M2}, N_{M3}, N_{M4}, N_{M5}, N_{M6}, N_{M7} and N_{M8} independently represents a modified or unmodified nucleotide, preferably, N_{M1}, N_{M2}, N_{M3}, N_{M6} and N_{M7} each independently represents a 2′-fluoro-modified nucleotide; each N_{L} independently represents a modified or unmodified nucleotide but not a 2′-fluoro-modified nucleotide, and n is an integer of 0 to 7.

15. (canceled)

16. The dsRNA agent of claim 1, wherein the dsRNA agent comprises an E-vinylphosphonate nucleotide at the 5′ end of the guide strand; optionally, the antisense strand sequence may be represented by formula (II′); 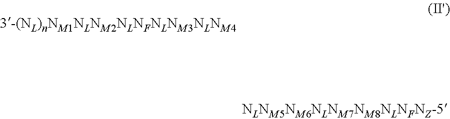
wherein:
each N_{F} represents a 2′-fluoro-modified nucleotide; each of N_{M1}, N_{M2}, N_{M3}, N_{M4}, N_{M5}, N_{M6}, N_{M7} and N_{M8} independently represents a modified or unmodified nucleotide, preferably, N_{M1}, N_{M2}, N_{M3}, N_{M6} and N_{M7} each independently represents a 2′-fluoro-modified nucleotide; each N_{L} independently represents a modified or unmodified nucleotide but not a 2′-fluoro-modified nucleotide; N_{Z} represents a nucleotide comprising vinyl phosphonate; and n is an integer of 0 to 7; or
the sense strand and the antisense strand form a dsRNA duplex, wherein said sense strand complementary to the antisense strand, wherein said antisense strand comprises a region of complementarity to an mRNA encoding PNPLA3, wherein the region of complementarity comprises at least 15 contiguous nucleotides, the dsRNA duplex represented by formula (III′); sense: 5′-(N′_{L})_{n′} N′_{L} N′_{L} N′_{L} N′_{N1} N′_{N2} N′_{N3} N′_{N4} N′_{F} N′_{L} N′_{N5}N′_{N6} N′_{N7} N′_{N8} N′_{L} N′_{L} (N′_{L})_{m′}-3′ antisense: 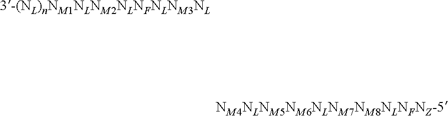
wherein:
each N_{F} and N′_{F} independently represents a 2′-fluoro-modified nucleotide; each of N_{M1}, N_{M2}, N_{M3}, N_{M4}, N_{M5}, N_{M6}, N_{M7}, N_{M8}, N′_{N1}, N′_{N2}, N′_{N3}, N′_{N4}, N′_{N5}, N′_{N6}, N′_{N7} and N′_{N8} each independently represents a modified or unmodified nucleotide; each N_{L} and N′_{L} independently represents a modified or unmodified nucleotide but not a 2′-fluoro-modified nucleotide; N_{Z} represents a nucleotide comprising vinyl phosphonate; and m′, n′ and n are each independently an integer of 0 to 7;
preferably, N_{Z} is a vinyl phosphonate modified nucleotide, preferably, N_{Z} is VPu*, which has the structure

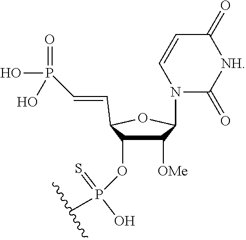

17-19. (canceled)

20. The dsRNA agent of claim 1, wherein the dsRNA agent comprises at least one phosphorothioate internucleoside linkage; or
the sense strand comprises at least one phosphorothioate internucleoside linkage; or
the antisense strand comprises at least one phosphorothioate internucleoside linkage;
optionally, the sense strand comprises 1, 2, 3, 4, 5, or 6 phosphorothioate internucleoside linkages, preferably, the 1, 2, 3, 4, 5, or 6 phosphorothioate internucleoside linkages are introduced at the 5′-end, 3′-end or both ends of the sense strand; or
the antisense strand comprises 1, 2, 3, 4, 5, or 6, phosphorothioate internucleoside linkages, preferably, the 1, 2, 3, 4, 5, or 6 phosphorothioate internucleoside linkages are introduced at the 5′-end, 3′-end or both ends of the antisense strand.

21-24. (canceled)

25. The dsRNA agent of claim 1, wherein all or substantially all of the nucleotides of the sense strand and the antisense strand are modified nucleotides;
optionally, the modified sense strand is a modified sense strand sequence set forth in one of Tables 2-3; or
the modified antisense strand is a modified antisense strand sequence set forth in one of Tables 2-3;
optionally, the sense strand is complementary or substantially complementary to the antisense strand, and the region of complementarity is between 16 and 23 nucleotides in length;
optionally, the region of complementarity is 19-21 nucleotides in length;
optionally, each strand is no more than 30 nucleotides in length;
optionally, each strand is no more than 25 nucleotides in length;
optionally, each strand is no more than 23 nucleotides in length.

26-32. (canceled)

33. The dsRNA agent of claim 1, wherein the dsRNA agent comprises at least one modified nucleotide and further comprises one or more targeting groups or linking groups;
optionally, the one or more targeting groups or linking groups are conjugated to the sense strand;
preferably, the targeting group or linking group comprises N-acetyl-galactosamine (GalNAc);
preferably, the targeting group has a structure:

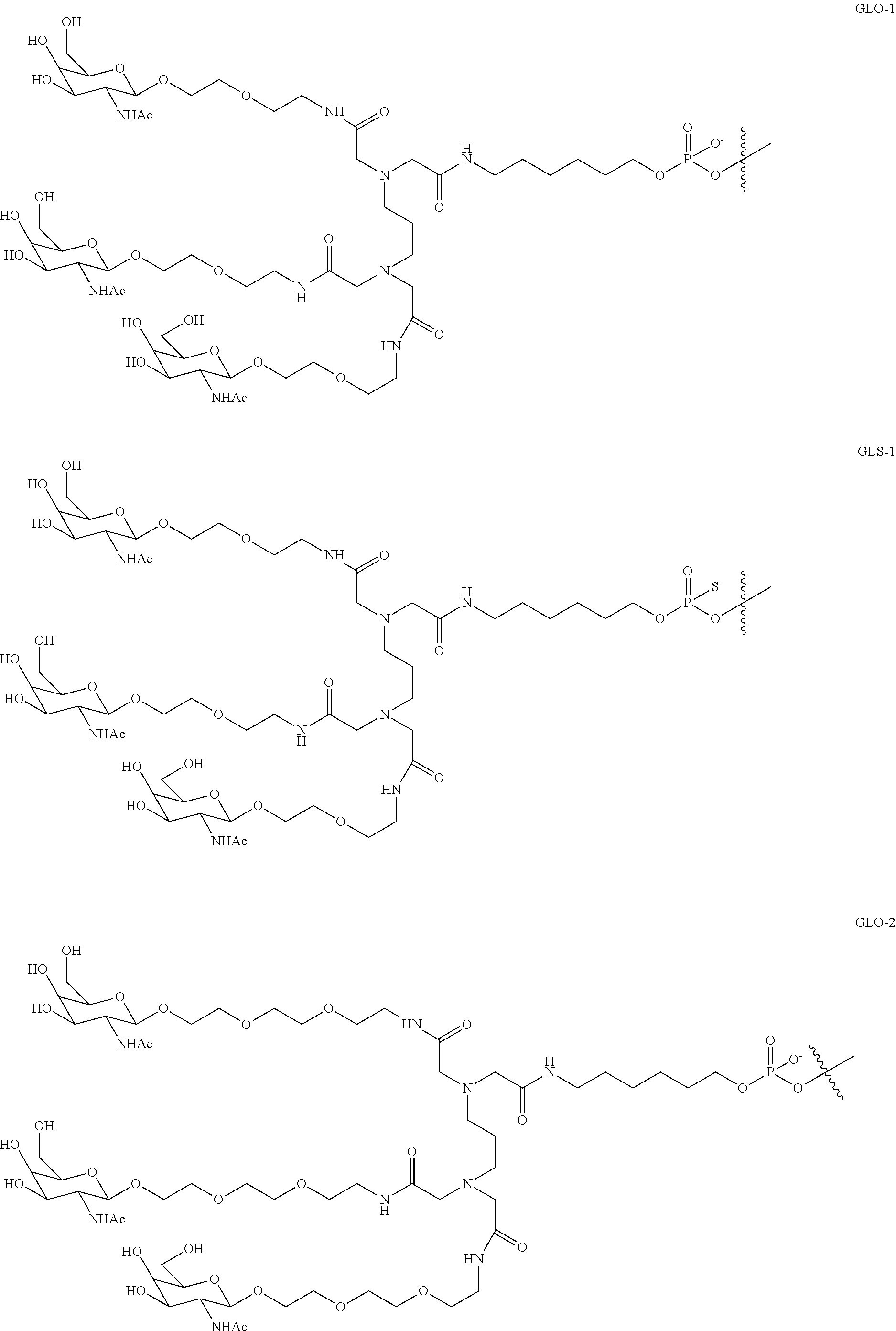 [SMILES: *OP(=O)([O-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCNC(=O)CN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

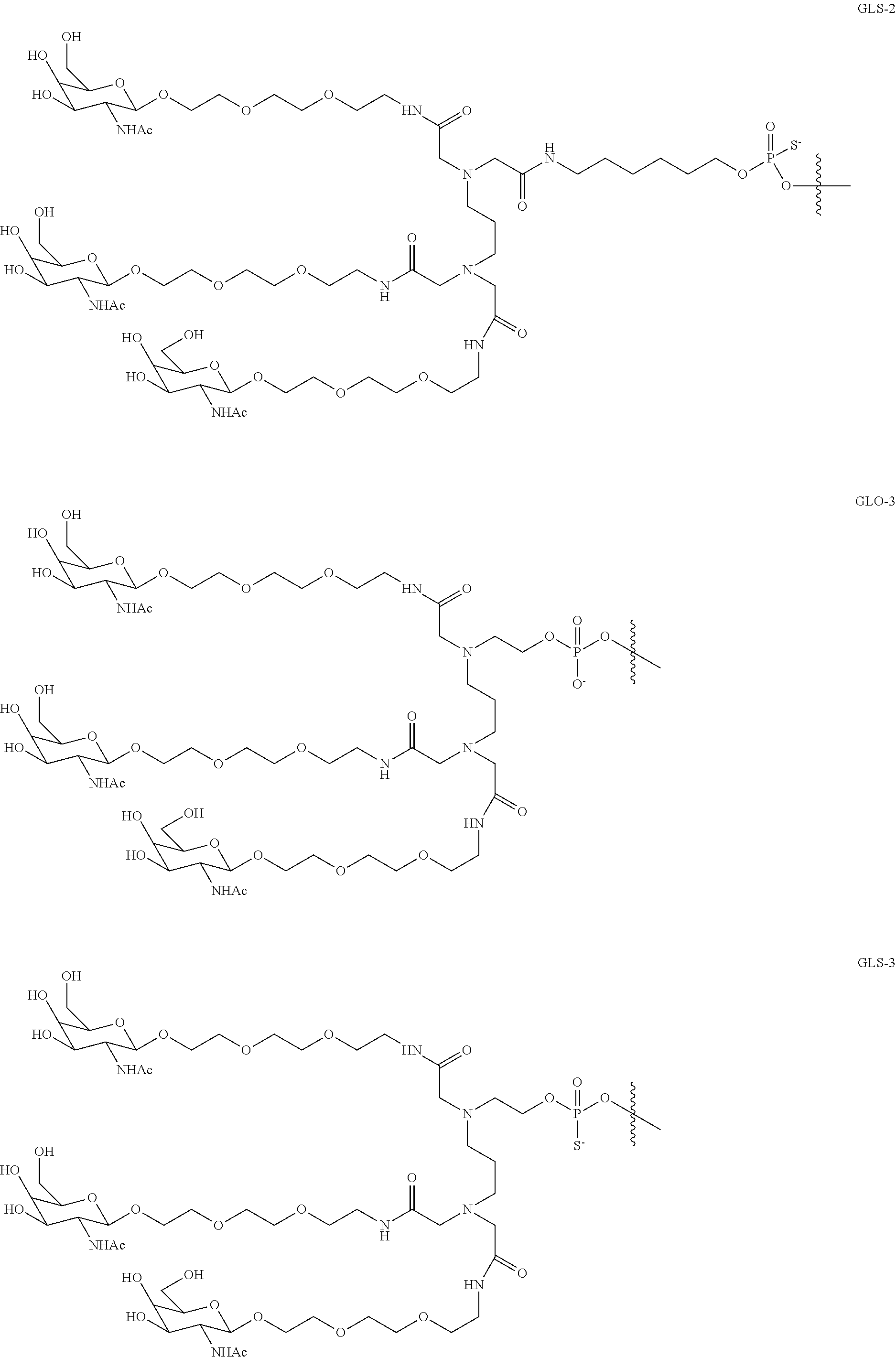 [SMILES: *OP(=O)([S-])OCCCCCCNC(=O)CN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([O-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCN(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

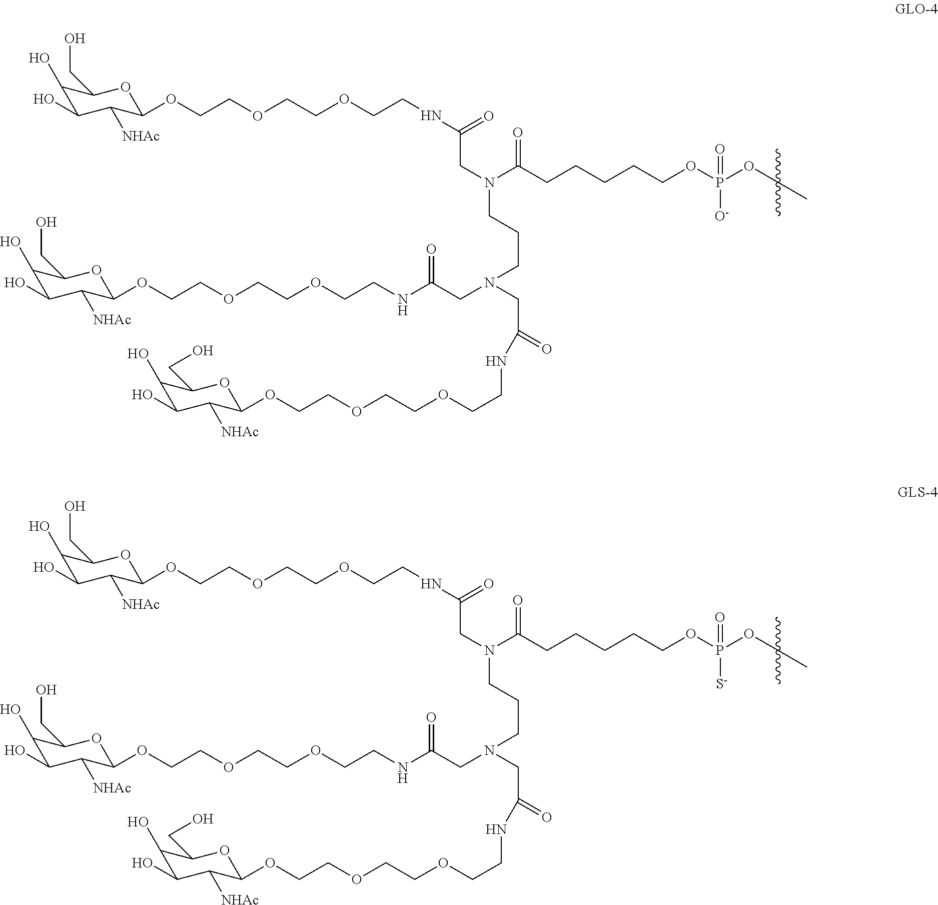 [SMILES: *OP(=O)([O-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

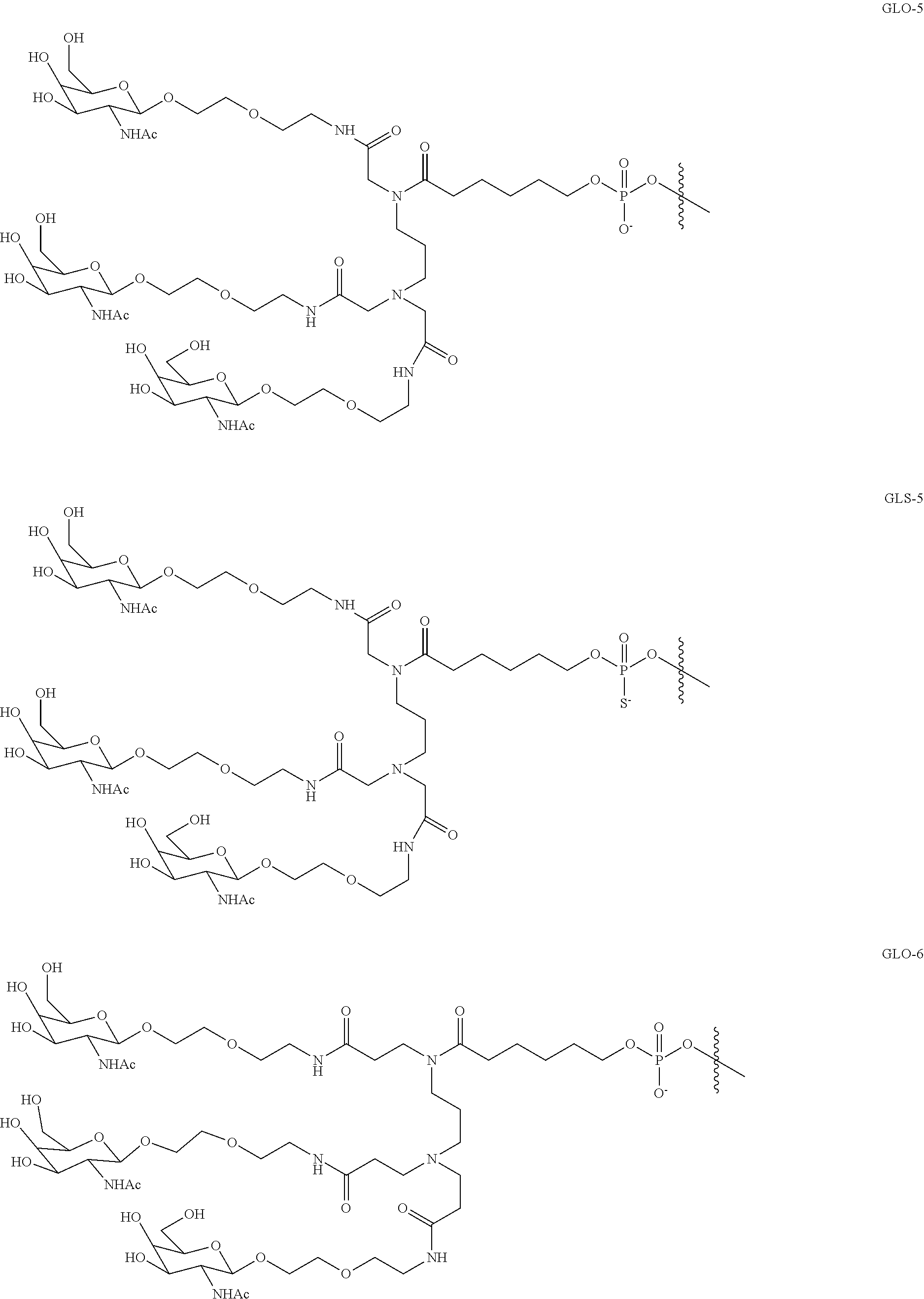 [SMILES: *OP(=O)([O-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O]

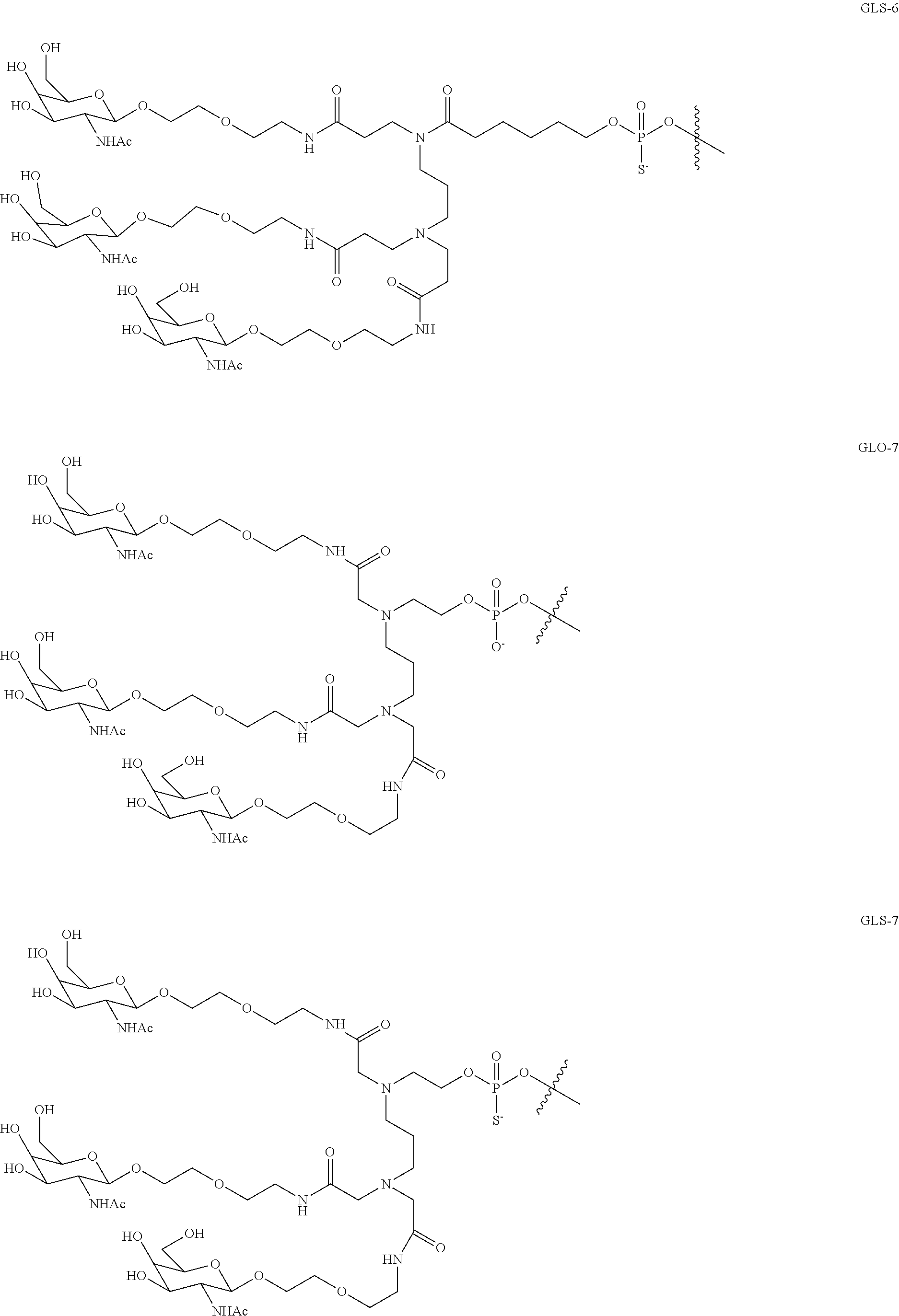 [SMILES: *OP(=O)([S-])OCCCCCC(=O)N(CCCN(CCC(=O)NCCOCCOC1OC(CO)C(O)C(O)C1NC(C)=O)CCC(=O)NCCOCCOC1OC(CO)C(O)C(O)C1NC(C)=O)CCC(=O)NCCOCCOC1OC(CO)C(O)C(O)C1NC(C)=O ; *OP(=O)([O-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

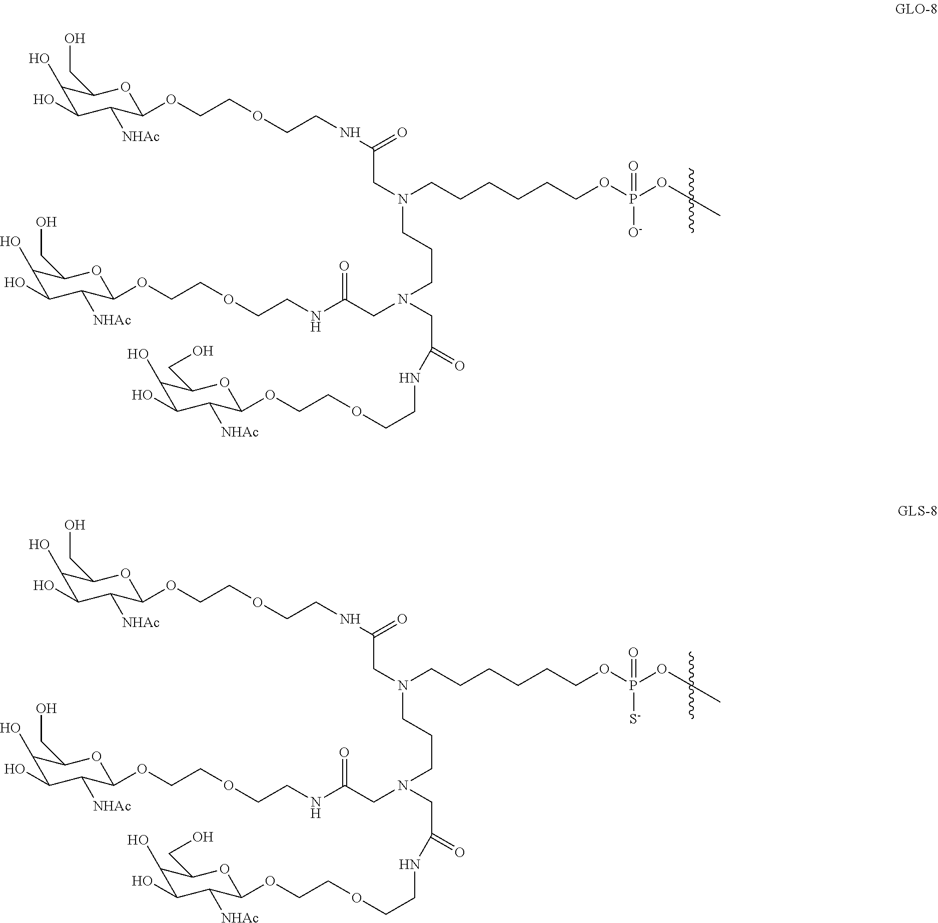 [SMILES: *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

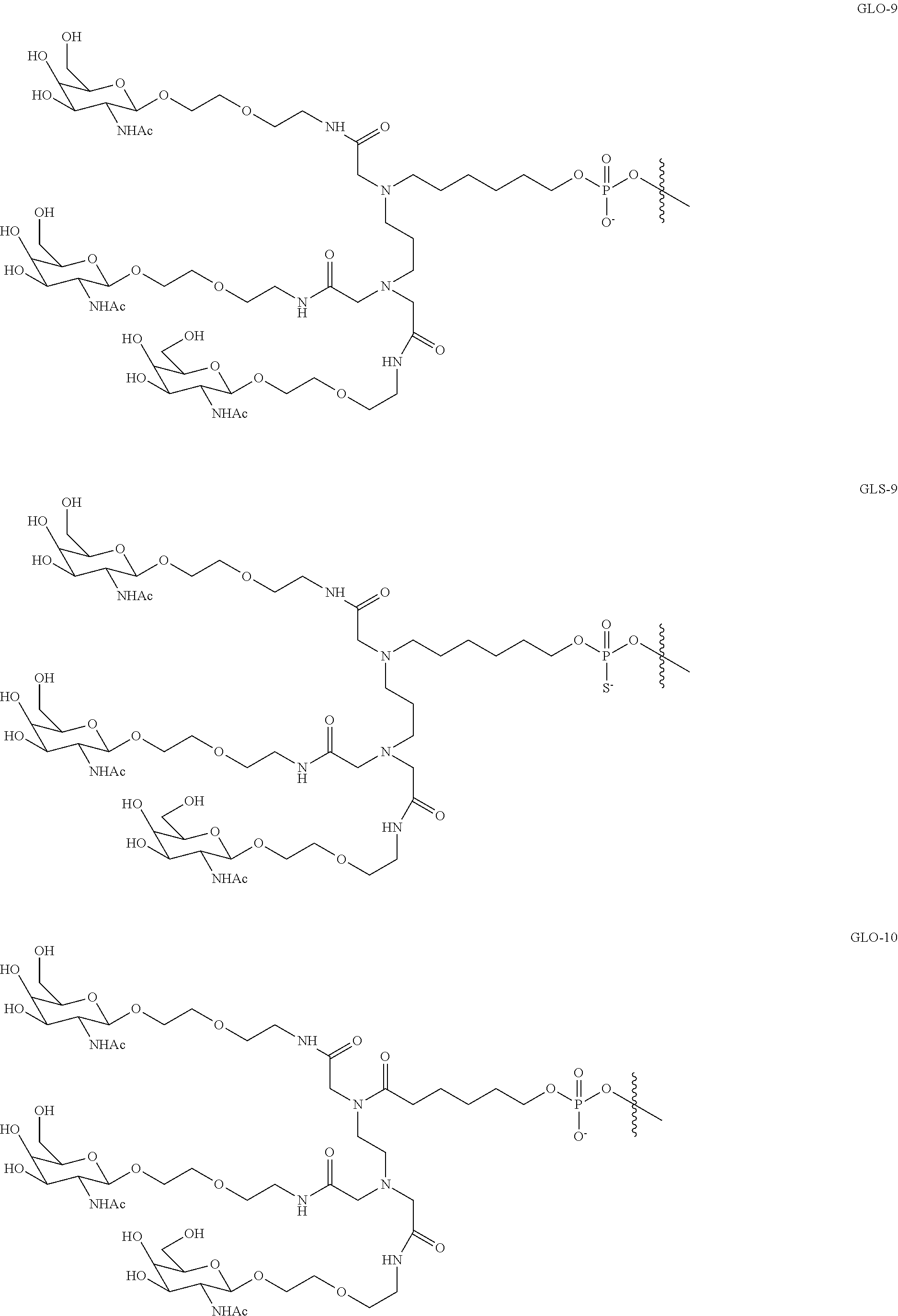 [SMILES: *OP(=O)([O-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OCCCCCCN(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

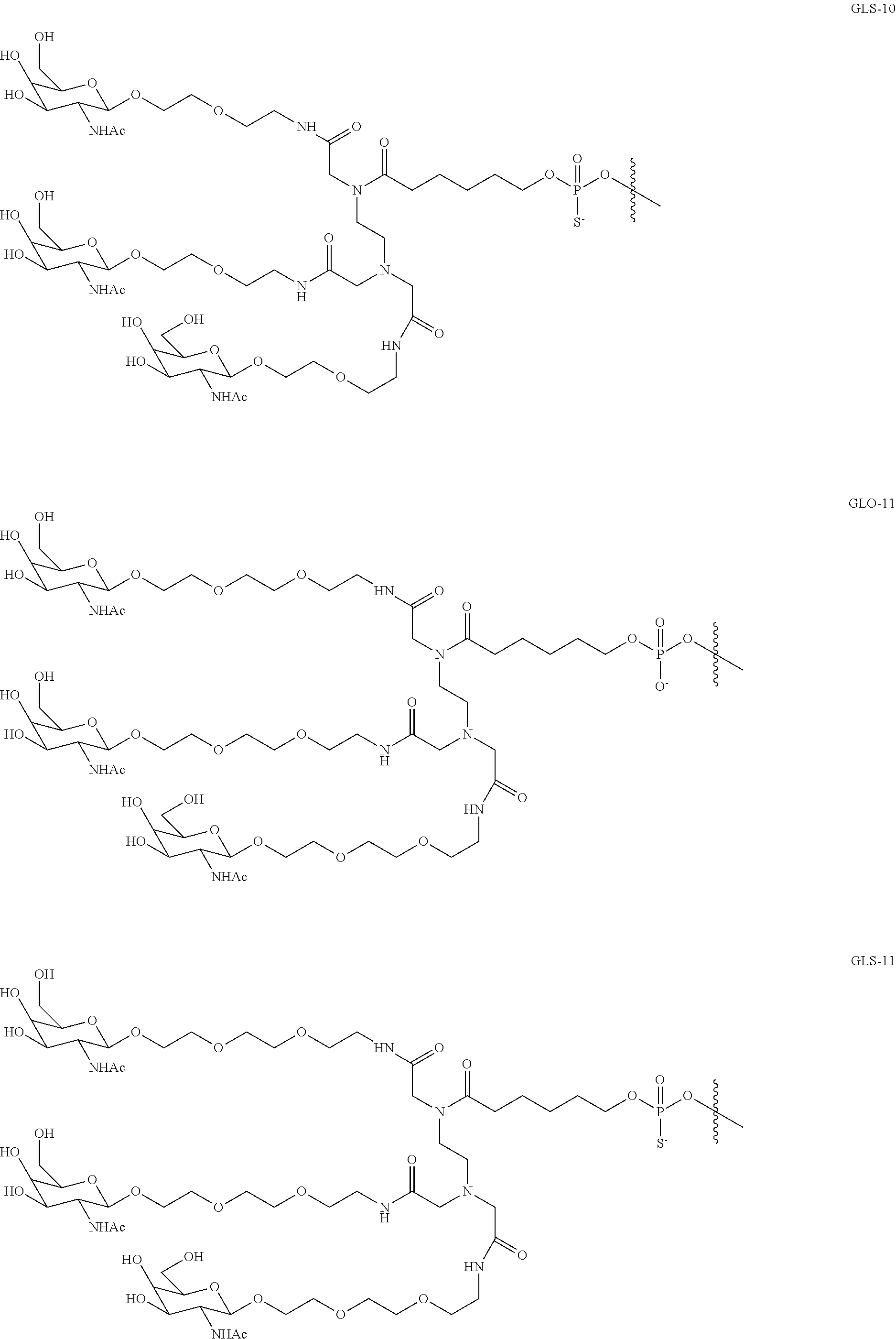 [SMILES: *OP(=O)([S-])OCCCCCC(=O)N(CCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O ; *OP(=O)([S-])OCCCCCC(=O)N(CCN(CC(=O)NCCOCCOCCOC1OC(CO)C(O)C(O)C1NC(C)=O)CC(=O)NCCOCCOCCOC1OC(CO)C(O)C(O)C1NC(C)=O)CC(=O)NCCOCCOCCOC1OC(CO)C(O)C(O)C1NC(C)=O]

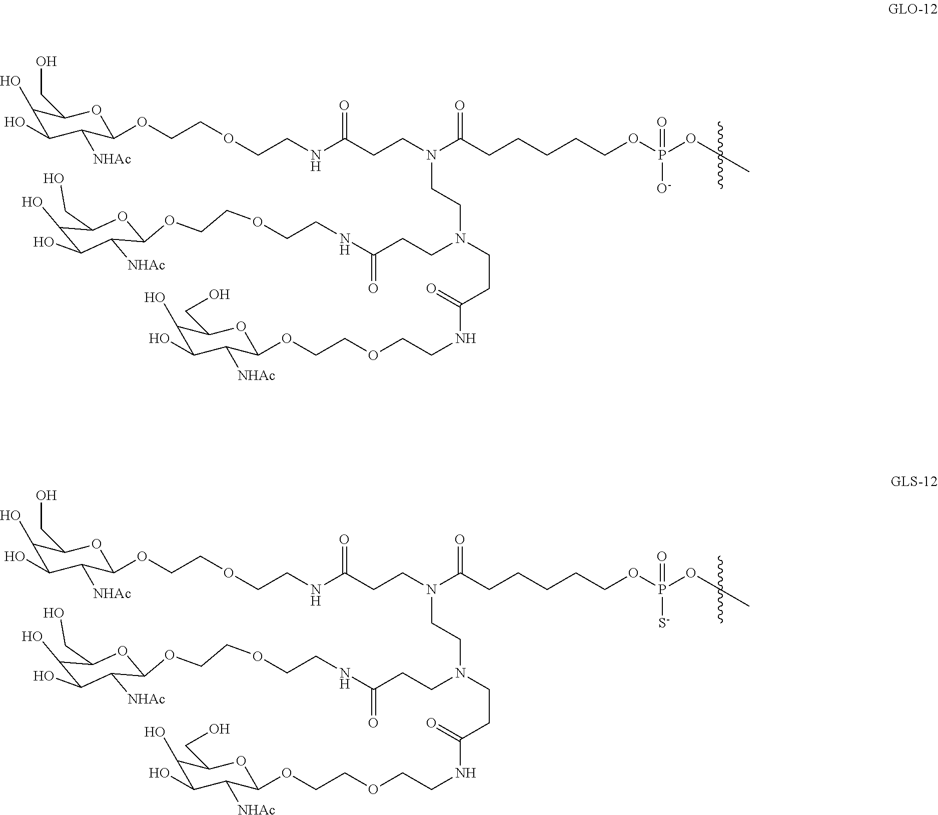 [SMILES: *OP(=O)([O-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O ; *OP(=O)([S-])OCCCCCC(=O)N(CCC(=O)NCCOCCOC1OC(CO)C(O)C(O)C1NC(C)=O)CCN(CCC(=O)NCCOCCOC1OC(CO)C(O)C(O)C1NC(C)=O)CCC(=O)NCCOCCOC1OC(CO)C(O)C(O)C1NC(C)=O]

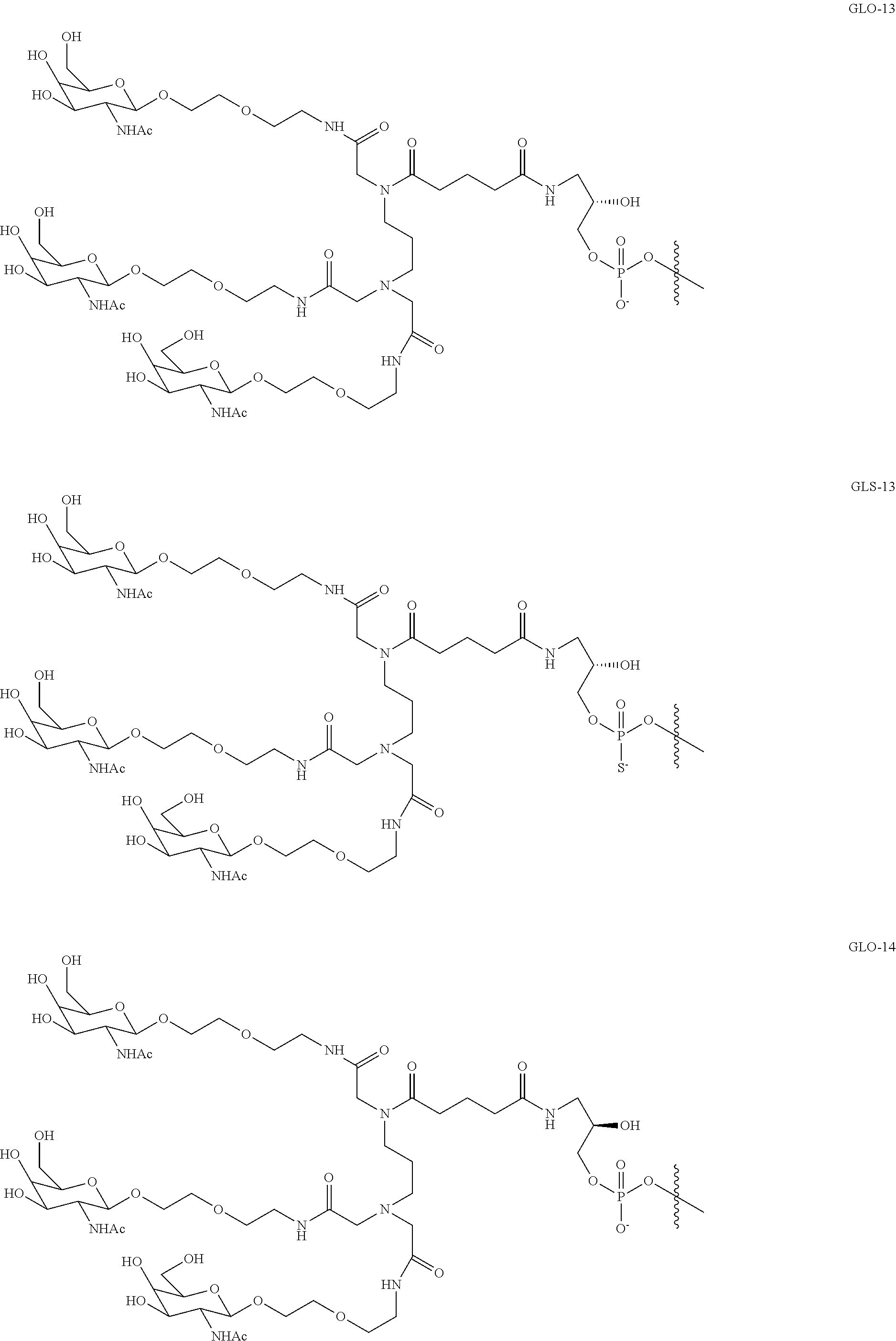 [SMILES: *OP(=O)([O-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([S-])OC[C@@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O]

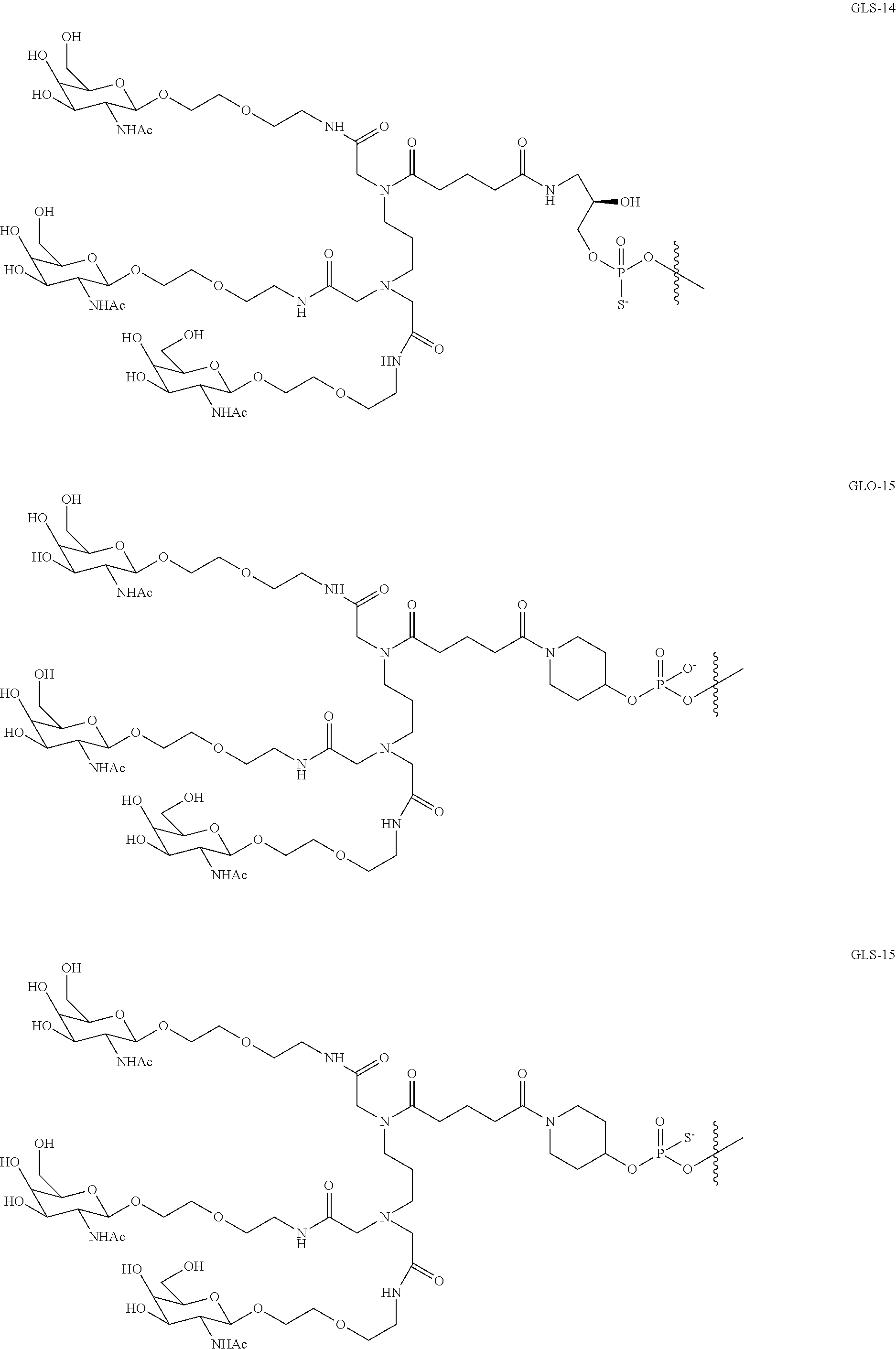 [SMILES: *OP(=O)([S-])OC[C@H](O)CNC(=O)CCCC(=O)N(CCCN(CC(=O)NCCOCCOC1OC(CO)C(O)C(O)C1NC(C)=O)CC(=O)NCCOCCOC1OC(CO)C(O)C(O)C1NC(C)=O)CC(=O)NCCOCCOC1OC(CO)C(O)C(O)C1NC(C)=O ; *OP(=O)([O-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1 ; *OP(=O)([S-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1]

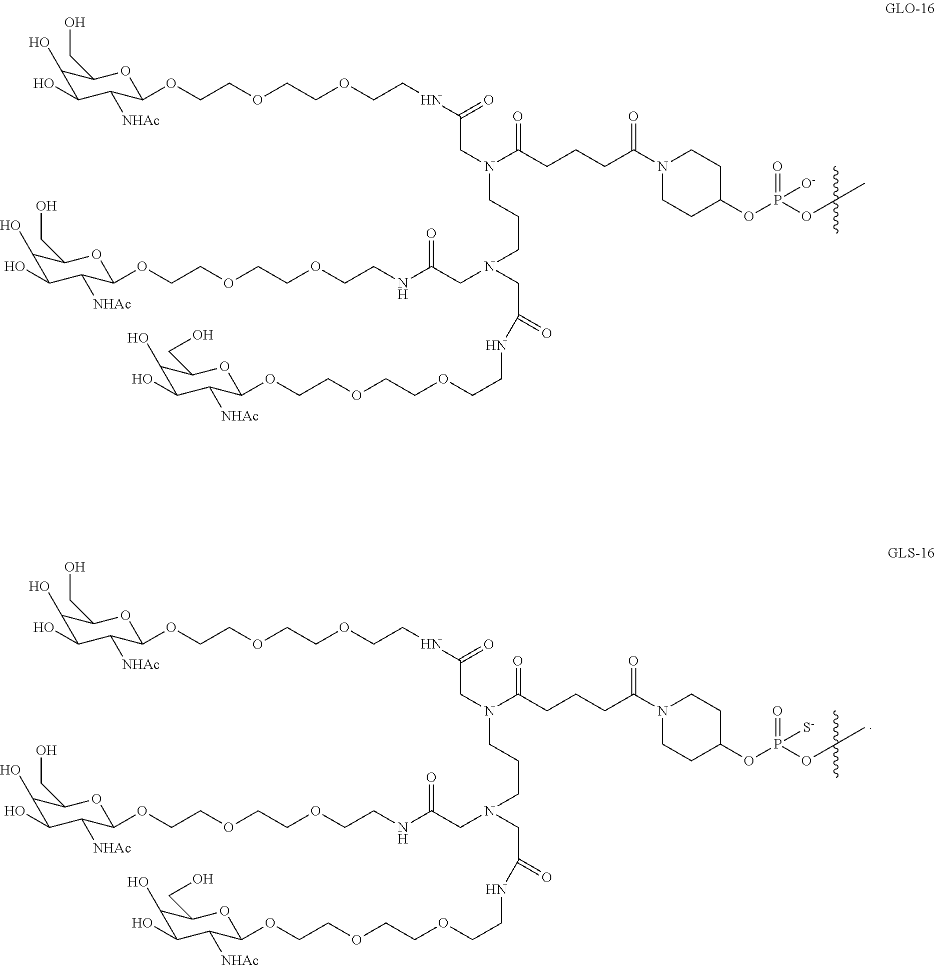 [SMILES: *OP(=O)([O-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1 ; *OP(=O)([S-])OC1CCN(C(=O)CCCC(=O)N(CCCN(CC(=O)NCCOCCOCCOC2OC(CO)C(O)C(O)C2NC(C)=O)CC(=O)NCCOCCOCCOC2OC(CO)C(O)C(O)C2NC(C)=O)CC(=O)NCCOCCOCCOC2OC(CO)C(O)C(O)C2NC(C)=O)CC1]

34-36. (canceled)

37. The dsRNA agent of claim 33, wherein the dsRNA agent comprises a targeting group that is conjugated to the 5′-terminal end of the sense strand; or
the dsRNA agent comprises a targeting group that is conjugated to the 3′-terminal end of the sense strand.

38. (canceled)

39. The dsRNA agent of claim 1, wherein the antisense strand comprises one inverted abasic residue at 3′-terminal end; or
the sense strand comprises one or two inverted abasic residues at 3′ or/and 5′ terminal end or one or two imann residues at 3′ or/and 5′ terminal end;
optionally, the dsRNA agent has two blunt ends; or
at least one strand comprises a 3′ overhang of at least 1 nucleotide; or
at least one strand comprises a 3′ overhang of at least 2 nucleotides.

40-43. (canceled)

44. A composition comprising a dsRNA agent of claim 1;
preferably, the composition further comprises a pharmaceutically acceptable carrier;
preferably, the composition further comprises one or more additional therapeutic agents;
preferably, the composition is packaged in a kit, container, pack, dispenser, pre-filled syringe, or vial;
preferably, the composition is formulated for subcutaneous administration or is formulated for intravenous (IV) administration.

45-48. (canceled)

49. A cell comprising a dsRNA agent of claim 1;
preferably, the cell is a mammalian cell, optionally a human cell.

50. (canceled)

51. A method of inhibiting the expression of a PNPLA3 gene in a cell, the method comprising:
(i) preparing a cell comprising an effective amount of a double-stranded ribonucleic acid (dsRNA) agent of claim 1;
preferably, the method further comprises (ii) maintaining the cell prepared in step (i) for a time sufficient to obtain degradation of the mRNA transcript of a PNPLA3 gene, thereby inhibiting expression of the PNPLA3 gene in the cell;
preferably, the cell is in a subject and the dsRNA agent is administered to the subject subcutaneously; or
the cell is in a subject and the dsRNA agent is administered to the subject by IV administration;
preferably, the method further comprises assessing inhibition of the PNPLA3 gene, following the administration of the dsRNA agent to the subject, wherein a means for the assessing comprises:
(i) determining one or more physiological characteristics of a PNPLA3-associated disease or condition in the subject and
(ii) comparing the determined physiological characteristic(s) to a baseline pre-treatment physiological characteristic of the PNPLA3-associated disease or condition and/or to a control physiological characteristic of the PNPLA3-associated disease or condition,
wherein the comparison indicates one or more of a presence or absence of inhibition of expression of the PNPLA3 gene in the subject;
preferably, the determined physiological characteristic is one or more of: PNPLA3 mRNA level, PNPLA3 protein level, the fat level and/or the lipid droplets level in the liver, or the number or extent of amyloid deposits in the subject;
preferably, a reduction in one or more of the subject's PNPLA3 mRNA level, the subject's PNPLA3 protein level, and the number or extent of amyloid deposits in the subject indicates reduction of PNPLA3 gene expression in the subject.

52-57. (canceled)

58. A method of inhibiting expression of a PNPLA3 gene in a subject, the method comprising administering to the subject an effective amount of a double-stranded ribonucleic acid (dsRNA) agent of claim 1;
preferably, the dsRNA agent is administered to the subject subcutaneously; or
the dsRNA agent is administered to the subject by IV administration;
preferably, the method further comprises assessing inhibition of the PNPLA3 gene, following the administration of the dsRNA agent, wherein a means for the assessing comprises:
(i) determining one or more physiological characteristics of a PNPLA3-associated disease or condition in the subject and
(ii) comparing the determined physiological characteristic(s) to a baseline pre-treatment physiological characteristic of the PNPLA3-associated disease or condition and/or to a control physiological characteristic of the PNPLA3-associated disease or condition,
wherein the comparison indicates one or more of a presence or absence of inhibition of expression of the PNPLA3 gene in the subject;
preferably, the determined physiological characteristic is one or more of: PNPLA3 mRNA level, PNPLA3 protein level, the fat level and/or the lipid droplets level in the liver, or the number or extent of amyloid deposits;
preferably, a reduction in one or more of the subject's PNPLA3 mRNA level, the subject's PNPLA3 protein level, and/or the number or extent of amyloid deposits indicates reduction of PNPLA3 gene expression in the subject.

59-63. (canceled)

64. A method of treating a disease or condition associated with the presence of PNPLA3 protein, the method comprising administering to a subject an effective amount of a double-stranded ribonucleic acid (dsRNA) agent of claim 1, to inhibit PNPLA3 gene expression;
preferably, the disease or condition is one or more of: liver disease, fatty liver (steatosis), nonalcoholic steatohepatitis (NASH), alcoholic steatohepatitis (ASH), cirrhosis of the liver, accumulation of fat in the liver, inflammation of the liver, hepatocellular necrosis, hepatocellular carcinoma, liver fibrosis, obesity, alcoholic liver disease, HCV hepatitis, chronic hepatitis, hereditary hemochromatosis, primary sclerosing cholangitis or nonalcoholic fatty liver disease NAFLD);
preferably, the method further comprises administering an additional therapeutic regimen to the subject:
optionally, the additional therapeutic regimen comprises: administering to the subject one or more PNPLA3 antisense polynucleotides of the invention, administering to the subject a non-PNPLA3 dsRNA therapeutic agent, and a behavioral modification in the subject;
preferably, the non-PNPLA3 dsRNA therapeutic agent is one of more of: an HMG-COA reductase inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein Ilb/IIIa inhibitor, aspirin or an aspirin-like compound, an IB AT inhibitor, a squalene synthase inhibitor, a monocyte chemoattractant protein (MCP)-I inhibitor, or fish oil;
preferably, the dsRNA agent is administered to the subject subcutaneously; or
the dsRNA agent is administered to the subject by IV administration;
preferably, the method further comprises determining an efficacy of the administered double-stranded ribonucleic acid (dsRNA) agent in the subject;
preferably, a means of determining an efficacy of the treatment in the subject comprises:
(i) determining one or more physiological characteristics of the PNPLA3-associated disease or condition in the subject and
(ii) comparing the determined physiological characteristic(s) to a baseline pre-treatment physiological characteristic of the PNPLA3-associated disease or condition
wherein the comparison indicates one or more of a presence, absence, and level of efficacy of the administration of the double-stranded ribonucleic acid (dsRNA) agent to the subject;
preferably, the determined physiological characteristic is: PNPLA3 mRNA level, PNPLA3 protein level, the fat level and/or the lipid droplets level in the liver, or the number or extent of amyloid deposits in the subject;
preferably, a reduction in one or more of the PNPLA3 mRNA level, the PNPLA3 protein level, or the number or extent of amyloid deposits in the subject indicates the presence of efficacy of the administration of the double-stranded ribonucleic acid (dsRNA) agent to the subject.

65-74. (canceled)

75. A method of decreasing a level of PNPLA3 protein in a subject compared to a baseline pre-treatment level of PNPLA3 protein in the subject, the method comprising administering to the subject an effective amount of a double-stranded ribonucleic acid (dsRNA) agent of claim 1, to decrease the level of PNPLA3 gene expression;
preferably, the dsRNA agent is administered to the subject subcutaneously or is administered to the subject by IV administration.

76. (canceled)

77. A method of altering a physiological characteristic of a PNPLA3-associated disease or condition in a subject compared to a baseline pre-treatment physiological characteristic of the PNPLA3-associated disease or condition in the subject, the method comprising administering to the subject an effective amount of a double-stranded ribonucleic acid (dsRNA) agent of claim 1, to alter the physiological characteristic of the PNPLA3-associated disease or condition in the subject;
preferably, the dsRNA agent is administered to the subject subcutaneously or is administered to the subject by IV administration;
preferably, the physiological characteristic is one or more of: the PNPLA3 mRNA level, the PNPLA3 protein level, the fat level and/or the lipid droplets level in the liver, or the number or extent of amyloid deposits in the subject.

78-79. (canceled)
